SEQUENCE LISTING (40 sequences)

SEQ ID NO: 1 - DNA, 3399 bp, Artificial
  Synthetic
atgcccagagcccccagatgcagagccgtgagaagcctgctgagaagccactacagagaggtgctgcccctggccaccttcgtgagaagactgggcccccagggctggagactggtgcagagaggcgaccccgccgccttcagagccctggtggcccagtgcctggtgtgcgtgccctgggacgccagaccccctcccgccgcccccagcttcagacaggtgagctgcctgaaggagctggtggccagagtgctgcagagactgtgcgagagaggcgccaagaacgtgctggccttcggcttcgccctgctggacggcgccagaggcggccctcccgaggccttcaccaccagcgtgagaagctacctgcccaacaccgtgaccgacgccctgagaggcagcggcgcctggggcctgctgctgagaagagtgggcgacgacgtgctggtgcacctgctggccagatgcgccctgttcgtgctggtggcccccagctgcgcctaccaggtgtgcggccctcccctgtaccagctgggcgccgccacccaggccagaccccctccccacgccagcggccccagaagaagactgggctgcgagagagcctggaaccacagcgtgagagaggccggcgtgcccctgggcctgcccgcccccggcgccagaagaagaggcggcagcgccagcagaagcctgcccctgcccaagagacccagaagaggcgccgcccccgagcccgagagaacccccgtgggccagggcagctgggcccaccccggcagaaccagaggccccagcgacagaggcttctgcgtggtgagccccgccagacccgccgaggaggccaccagcctggagggcgccctgagcggcaccagacacagccaccccagcgtgggcagacagcaccacgccggccctcccagcaccagcagacctcccagaccctgggacaccccctgccctcccgtgtacgccgagaccaagcacttcctgtacagcagcggcgacaaggagcagctgagacccagcttcctgctgagcagcctgagacccagcctgaccggcgccagaagactggtggagaccatcttcctgggcagcagaccctggatgcccggcacccccagaagactgcccagactgccccagagatactggcagatgagacccctgttcctggagctgctgggcaaccacgcccagtgcccctacggcgtgctgctgaagacccactgccccctgagagccgccgtgacccccgccgccggcgtgtgcgccagagagaagccccagggcagcgtggccgcccccgaggaggaggacaccgaccccagaagactggtgcagctgctgagacagcacagcagcccctggcaggtgtacggcttcgtgagagcctgcctgagaagactggtgcctcccggcctgtggggcagcagacacaacgagagaagattcctgagaaacaccaagaagttcatcagcctgggcaagcacgccaagctgagcctgcaggagctgacctggaagatgagcgtgagagactgcgcctggctgagaagaagccccggcgtgggctgcgtgcccgccgccgagcacagactgagagaggagatcctggccaagttcctgcactggctgatgagcgtgtacgtggtggagctgctgagaagcttcttctacgtgaccgagaccaccttccagaagaacagactgttcttctacagaaagagcgtgtggagcaagctgcagagcatcggcatcagacagcacctgaagagagtgcagctgagagagctgagcgaggccgaggtgagacagcacagagaggccagacccgccctgctgaccagcagactgagattcatccccaagcccgacggcctgagacccatcgtgaacatggactacgtggtgggcgccagaaccttcagaagagagaagagagccgagagactgaccagcagagtgaaggccctgttcagcgtgctgaactacgagagagccagaagacccggcctgctgggcgccagcgtgctgggcctggacgacatccacagagcctggagaaccttcgtgctgagagtgagagcccaggatccccctcccgagctgtacttcgtgaaggtggacgtgaccggcgcctacgacaccatcccccaggacagactgaccgaggtgatcgccagcatcatcaagccccagaacacctactgcgtgagaagatacgccgtggtgcagaaggccgcccacggccacgtgagaaaggccttcaagagccacgtgagcaccctgaccgacctgcagccctacatgagacagttcgtggcccacctgcaggagaccagccccctgagagacgccgtggtgatcgagcagagcagcagcctgaacgaggccagcagcggcctgttcgacgtgttcctgagattcatgtgccaccacgccgtgagaatcagaggcaagagctacgtgcagtgccagggcatcccccagggcagcatcctgagcaccctgctgtgcagcctgtgctacggcgacatggagaacaagctgttcgccggcatcagaagagacggcctgctgctgagactggtggacgacttcctgctggtgacaccccacctgacccacgccaagaccttcctgagaaccctggtgagaggcgtgcccgagtacggctgcgtggtgaacctgagaaagaccgtggtgaacttccccgtggaggacgaggccctgggcggcaccgccttcgtgcagatgcccgcccacggcctgttcccctggtgcggcctgctgctggacaccagaaccctggaggtgcagagcgactacagcagctacgccagaaccagcatcagagccagcctgaccttcaacagaggcttcaaggccggcagaaacatgagaagaaagctgttcggcgtgctgagactgaagtgccacagcctgttcctggacctgcaggtgaacagcctgcagaccgtgtgcaccaacatctacaagatcctgctgctgcaggcctacagattccacgcctgcgtgctgcagctgcccttccaccagcaggtgtggaagaaccccaccttcttcctgagagtgatcagcgacaccgccagcctgtgctacagcatcctgaaggccaagaacgccggcatgagcctgggcgccaagggcgccgccggccccctgcccagcgaggccgtgcagtggctgtgccaccaggccttcctgctgaagctgaccagacacagagtgacctacgtgcccctgctgggcagcctgagaaccgcccagacccagctgagcagaaagctgcccggcaccaccctgaccgccctggaggccgccgccaaccccgccctgcccagcgacttcaagaccatcctggactga

SEQ ID NO: 2 - DNA, 3399 bp, Artificial
  Synthetic
atgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgttctttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactga

SEQ ID NO: 3 - DNA, 3369 bp, Artificial
  Synthetic
atgaccagggcccctaggtgccctgccgtgaggagcctgctgaggagcaggtacagggaggtgtggcctctggccaccttcgtgaggaggctgggccctgagggcaggaggctggtgcagcctggcgaccctaagatctacaggaccctggtggcccagtgcctggtgtgcatgcactggggcagccagcctcctcctgccgacctgagcttccaccaggtgagcagcctgaaggagctggtggccagggtggtgcagaggctgtgcgagaggaacgagaggaacgtgctggccttcggcttcgagctgctgaacgaggccaggggcggccctcctatggccttcaccagcagcgtgaggagctacctgcctaacaccgtgatcgagaccctgagggtgagcggcgcctggatgctgctgctgagcagggtgggcgacgacctgctggtgtacctgctggcccactgcgccctgtacctgctggtgcctcctagctgcgcctaccaggtgtgcggcagccctctgtaccagatctgcgccaccaccgacatctggcctagcgtgagcgccagctacaggcctaccaggcctgtgggcaggaacttcaccaacctgaggttcctgcagcagatcaagagcagcagcaggcaggaggcccctaagcctctggccctgcctagcaggggcaccaagaggcacctgagcctgaccagcaccagcgtgcctagcgccaagaaggccaggtgctaccctgtgcctagggtggaggagggccctcacaggcaggtgctgcctacccctagcggcaagagctgggtgcctagccctgccaggagccctgaggtgcctaccgccgagaaggacctgagcagcaagggcaaggtgagcgacctgagcctgagcggcagcgtgtgctgcaagcacaagcctagcagcaccagcctgctgagccctcctaggcagaacgccttccagctgaggcctttcatcgagaccaggcacttcctgtacagcaggggcgacggccaggagaggctgaaccctagcttcctgctgagcaacctgcagcctaacctgaccggcgccaggaggctggtggagatcatcttcctgggcagcaggcctaggaccagcggccctctgtgcaggacccacaggctgagcaggaggtactggcagatgaggcctctgttccagcagctgctggtgaaccacgccgagtgccagtacgtgaggctgctgaggagccactgcaggttcaggaccgccaaccagcaggtgaccgacgccctgaacaccagccctcctcacctgatggacctgctgaggctgcacagcagcccttggcaggtgtacggcttcctgagggcctgcctgtgcaaggtggtgagcgccagcctgtggggcaccaggcacaacgagaggaggttcttcaagaacctgaagaagttcatcagcctgggcaagtacggcaagctgagcctgcaggagctgatgtggaagatgaaggtggaggactgccactggctgaggagcagccctggcaaggacagggtgcctgccgccgagcacaggctgagggagaggatcctggccaccttcctgttctggctgatggacacctacgtggtgcagctgctgaggagcttcttctacatcaccgagagcaccttccagaagaacaggctgttcttctacaggaagagcgtgtggagcaagctgcagagcatcggcgtgaggcagcacctggagagggtgaggctgagggagctgagccaggaggaggtgaggcaccaccaggacacctggctggccatgcctatctgcaggctgaggttcatccctaagcctaacggcctgaggcctatcgtgaacatgagctacagcatgggcaccagggccctgggcaggaggaagcaggcccagcacttcacccagaggctgaagaccctgttcagcatgctgaactacgagaggaccaagcaccctcacctgatgggcagcagcgtgctgggcatgaacgacatctacaggacctggagggccttcgtgctgagggtgagggccctggaccagacccctaggatgtacttcgtgaaggccgacgtgaccggcgcctacgacgccatccctcagggcaagctggtggaggtggtggccaacatgatcaggcacagcgagagcacctactgcatcaggcagtacgccgtggtgaggagggacagccagggccaggtgcacaagagcttcaggaggcaggtgaccaccctgagcgacctgcagccttacatgggccagttcctgaagcacctgcaggacagcgacgccagcgccctgaggaacagcgtggtgatcgagcagagcatcagcatgaacgagagcagcagcagcctgttcgacttcttcctgcacttcctgaggcacagcgtggtgaagatcggcgacaggtgctacacccagtgccagggcatccctcagggcagcagcctgagcaccctgctgtgcagcctgtgcttcggcgacatggagaacaagctgttcgccgaggtgcagagggacggcctgctgctgaggttcgtggacgacttcctgctggtgacccctcacctggaccaggccaagaccttcctgagcaccctggtgcacggcgtgcctgagtacggctgcatgatcaacctgcagaagaccgtggtgaacttccctgtggagcctggcaccctgggcggcgccgccccttaccagctgcctgcccactgcctgttcccttggtgcggcctgctgctggacacccagaccctggaggtgttctgcgactacagcggctacgcccagaccagcatcaagaccagcctgaccttccagagcgtgttcaaggccggcaagaccatgaggaacaagctgctgagcgtgctgaggctgaagtgccacggcctgttcctggacctgcaggtgaacagcctgcagaccgtgtgcatcaacatctacaagatcttcctgctgcaggcctacaggttccacgcctgcgtgatccagctgcctttcgaccagagggtgaggaagaacctgaccttcttcctgggcatcatcagcagccaggccagctgctgctacgccatcctgaaggtgaagaaccctggcatgaccctgaaggccagcggcagcttccctcctgaggccgcccactggctgtgctaccaggccttcctgctgaagctggccgcccacagcgtgatctacaagtgcctgctgggccctctgaggaccgcccagaagctgctgtgcaggaagctgcctgaggccaccatgaccatcctgaaggccgccgccgaccctgccctgagcaccgacttccagaccatcctggactga

SEQ ID NO: 4 - DNA, 3369 bp, Artificial
  Synthetic
atgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggtgtgtgggtctcccctgtaccaaatttgtgccaccacggatatctggccctctgtgtccgctagttacaggcccacccgacccgtgggcaggaatttcactaaccttaggttcttacaacagatcaagagcagtagtcgccaggaagcaccgaaacccctggccttgccatctcgaggtacaaagaggcatctgagtctcaccagtacaagtgtgccttcagctaagaaggccagatgctatcctgtcccgagagtggaggagggaccccacaggcaggtgctaccaaccccatcaggcaaatcatgggtgccaagtcctgctcggtcccccgaggtgcctactgcagagaaagatttgtcttctaaaggaaaggtgtctgacctgagtctctctgggtcggtgtgctgtaaacacaagcccagctccacatctctgctgtcaccaccccgccaaaatgcctttcagctcaggccatttattgagaccagacatttcctttactccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccggggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctttgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaa

SEQ ID NO: 5 - DNA, 3369 bp, Artificial
  Synthetic
atgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggtgtgtgggtctcccctgtaccaaatttgtgccaccacggatatctggccctctgtgtccgctagttacaggcccacccgacccgtgggcaggaatttcactaaccttaggttcttacaacagatcaagagcagtagtcgccaggaagcaccgaaacccctggccttgccatctcgaggtacaaagaggcatctgagtctcaccagtacaagtgtgccttcagctaagaaggccagatgctatcctgtcccgagagtggaggagggaccccacaggcaggtgctaccaaccccatcaggcaaatcatgggtgccaagtcctgctcggtcccccgaggtgcctactgcagagaaagatttgtcttctaaaggaaaggtgtctgacctgagtctctctgggtcggtgtgctgtaaacacaagcccagctccacatctctgctgtcaccaccccgccaaaatgcctttcagctcaggccatttattgagaccagacatttcctttactccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccggggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgttctttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaa

SEQ ID NO: 6 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 7 - DNA, 4039 bp, Homo sapiens
ctctcctcgcggcgcgagtttcaggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 8 - protein, 1069 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 9 - DNA, 3850 bp, Homo sapiens
ctctcctcgcggcgcgagtttcaggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 10 - protein, 1122 aa, Mus musculus
MTRAPRCPAVRSLLRSRYREVWPLATFVRRLGPEGRRLVQPGDPKIYRTLVAQCLVCMHWGSQPPPADLSFHQVSSLKELVARVVQRLCERNERNVLAFGFELLNEARGGPPMAFTSSVRSYLPNTVIETLRVSGAWMLLLSRVGDDLLVYLLAHCALYLLVPPSCAYQVCGSPLYQICATTDIWPSVSASYRPTRPVGRNFTNLRFLQQIKSSSRQEAPKPLALPSRGTKRHLSLTSTSVPSAKKARCYPVPRVEEGPHRQVLPTPSGKSWVPSPARSPEVPTAEKDLSSKGKVSDLSLSGSVCCKHKPSSTSLLSPPRQNAFQLRPFIETRHFLYSRGDGQERLNPSFLLSNLQPNLTGARRLVEIIFLGSRPRTSGPLCRTHRLSRRYWQMRPLFQQLLVNHAECQYVRLLRSHCRFRTANQQVTDALNTSPPHLMDLLRLHSSPWQVYGFLRACLCKVVSASLWGTRHNERRFFKNLKKFISLGKYGKLSLQELMWKMKVEDCHWLRSSPGKDRVPAAEHRLRERILATFLFWLMDTYVVQLLRSFFYITESTFQKNRLFFYRKSVWSKLQSIGVRQHLERVRLRELSQEEVRHHQDTWLAMPICRLRFIPKPNGLRPIVNMSYSMGTRALGRRKQAQHFTQRLKTLFSMLNYERTKHPHLMGSSVLGMNDIYRTWRAFVLRVRALDQTPRMYFVKADVTGAYDAIPQGKLVEVVANMIRHSESTYCIRQYAVVRRDSQGQVHKSFRRQVTTLSDLQPYMGQFLKHLQDSDASALRNSVVIEQSISMNESSSSLFDFFLHFLRHSVVKIGDRCYTQCQGIPQGSSLSTLLCSLCFGDMENKLFAEVQRDGLLLRFVDDFLLVTPHLDQAKTFLSTLVHGVPEYGCMINLQKTVVNFPVEPGTLGGAAPYQLPAHCLFPWCGLLLDTQTLEVFCDYSGYAQTSIKTSLTFQSVFKAGKTMRNKLLSVLRLKCHGLFLDLQVNSLQTVCINIYKIFLLQAYRFHACVIQLPFDQRVRKNLTFFLGIISSQASCCYAILKVKNPGMTLKASGSFPPEAAHWLCYQAFLLKLAAHSVIYKCLLGPLRTAQKLLCRKLPEATMTILKAAADPALSTDFQTILD

SEQ ID NO: 11 - protein, 729 aa, Mus musculus
MRPLFQQLLVNHAECQYVRLLRSHCRFRTANQQVTDALNTSPPHLMDLLRLHSSPWQVYGFLRACLCKVVSASLWGTRHNERRFFKNLKKFISLGKYGKLSLQELMWKMKVEDCHWLRSSPGKDRVPAAEHRLRERILATFLFWLMDTYVVQLLRSFFYITESTFQKNRLFFYRKSVWSKLQSIGVRQHLERVRLRELSQEEVRHHQDTWLAMPICRLRFIPKPNGLRPIVNMSYSMGTRALGRRKQAQHFTQRLKTLFSMLNYERTKHPHLMGSSVLGMNDIYRTWRAFVLRVRALDQTPRMYFVKADVTGAYDAIPQGKLVEVVANMIRHSESTYCIRQYAVVRRDSQGQVHKSFRRQVTTLSDLQPYMGQFLKHLQDSDASALRNSVVIEQSISMNESSSSLFDFFLHFLRHSVVKIGDRCYTQCQGIPQGSSLSTLLCSLCFGDMENKLFAEVQRDGLLLRFVDDFLLVTPHLDQAKTFLSTLVHGVPEYGCMINLQKTVVNFPVEPGTLGGAAPYQLPAHCLFPWCGLLLDTQTLEVFCDYSGYAQTSIKTSLTFQSVFKAGKTMRNKLLSVLRLKCHGLFLDLQVNSLQTVCINIYKIFLLQAYRFHACVIQLPFDQRVRKNLTFFLGIISSQASCCYAILKVKNPGMTLKASGSFPPEAAHWLCYQAFLLKLAAHSVIYKCLLGPLRTAQKLLCRKLPEATMTILKAAADPALSTDFQTILD

SEQ ID NO: 12 - protein, 584 aa, Mus musculus
MDTYVVQLLRSFFYITESTFQKNRLFFYRKSVWSKLQSIGVRQHLERVRLRELSQEEVRHHQDTWLAMPICRLRFIPKPNGLRPIVNMSYSMGTRALGRRKQAQHFTQRLKTLFSMLNYERTKHPHLMGSSVLGMNDIYRTWRAFVLRVRALDQTPRMYFVKADVTGAYDAIPQGKLVEVVANMIRHSESTYCIRQYAVVRRDSQGQVHKSFRRQVTTLSDLQPYMGQFLKHLQDSDASALRNSVVIEQSISMNESSSSLFDFFLHFLRHSVVKIGDRCYTQCQGIPQGSSLSTLLCSLCFGDMENKLFAEVQRDGLLLRFVDDFLLVTPHLDQAKTFLSTLVHGVPEYGCMINLQKTVVNFPVEPGTLGGAAPYQLPAHCLFPWCGLLLDTQTLEVFCDYSGYAQTSIKTSLTFQSVFKAGKTMRNKLLSVLRLKCHGLFLDLQVNSLQTVCINIYKIFLLQAYRFHACVIQLPFDQRVRKNLTFFLGIISSQASCCYAILKVKNPGMTLKASGSFPPEAAHWLCYQAFLLKLAAHSVIYKCLLGPLRTAQKLLCRKLPEATMTILKAAADPALSTDFQTILD

SEQ ID NO: 13 - protein, 1125 aa, Rattus norvegicus
MPRAPRCPAVRSLLRSRYREVWPLATFVRRLGLEGSRLVQPGDPKVFRTLVAQCLVCVPWGSQPPPADLSFHQVSSLKELVSRVVQKLCERGERNVLAFGFALLNGARGGPPMAFTTSVHSYLPNSVTESLCVSGAWMLLLSRVGDDLLVYLLSHCALYLLVPPSCAYQVCGSPLYQICATTDTWSSVPAGYRPTRPVGGNFTNLGSAHQIKNSGHQEAPKPQALPSRGTKRLLSLTSTNVPSAKKARFEPALRVDKGPHRQVVPTPSGKTWAPSPAASPKVPPAAKNLSLKGKASDPSLSGSVCCKHKPSSSSLLSSPPQDAEKLRPFTETRHFLYSRGGGQEELNPSFLLNSLPPSLTGARRLVEIIFLGSRPRTSGPFCRTRRLPRRYWQMRPLFQQLLMNHAKCQYVRFLRSHCRFRTANQRVPDAMDTSPSHLTSLLRLHSSPWQVYGFLRACLRELVPAGLWGTRHNERRFLKNVKKFISLGKYAKLSLQELMWRVKVEDCHWLRSSPEKDTVPAAEHRLRERILAMFLFWLMDTYVVQLLRSFFYITETTFQKNRLFFYRKSVWSKLQSIGIRQQLERVQLRELSQEEVKHHQDTWLAMPICRLRFIPKLNGLRPIVNMSYGMDTRAFGKKKQTQCFTQSLKTLFSVLNYERTKHPNLMGASVLGTSDSYRIWRTFVLRVRALDQTPRMYFVKADVTGAYDAIPQDKLVEIVANIIRRSESMYCIRQYAVVQKDSQGQVHKSFRRQVSTLSDLQPYMGQFTKHLQDSDASALRNSVVIEQSISMNETGSSLLHFFLRFVRHSVVKIDGRFYVQCQGIPQGSSLSTLLCSLCFGDMENKLFAEVQQDGLLLRFVDDFLLVTPHLAHAKAFLSTLVHGVPEYGCMINLQKTVVNFPVETGALGGAAPHQLPAHCLFPWCGLLLDTRTLEVFCDYSGYGRTSIKMSLTFQGVSRAGKTMRYKLLSVLRLKCHGLFLDLQVNSLQTVCINIYKIFLLQAYRFHACVIRLPFGQHVRKNHAFFLGIISNLASCCYAILKVKNPGVSLRAKGAPGSFPPEATRWLCYQAFLLKLAAHSVTYKCLLGPLRTAQKQLCRKLPEATMTLLKTAADPALSTDFQTILD

SEQ ID NO: 14 - DNA, 4335 bp, Mus musculus
gttcccagcctcatctttttcgtcgtggactctcagtggcctgggtcctggctgttttctaagcacacccttgcatcttggttcccgcacgtgggaggcccatcccggccttgagcacaatgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggtgtgtgggtctcccctgtaccaaatttgtgccaccacggatatctggccctctgtgtccgctagttacaggcccacccgacccgtgggcaggaatttcactaaccttaggttcttacaacagatcaagagcagtagtcgccaggaagcaccgaaacccctggccttgccatctcgaggtacaaagaggcatctgagtctcaccagtacaagtgtgccttcagctaagaaggccagatgctatcctgtcccgagagtggaggagggaccccacaggcaggtgctaccaaccccatcaggcaaatcatgggtgccaagtcctgctcggtcccccgaggtgcctactgcagagaaagatttgtcttctaaaggaaaggtgtctgacctgagtctctctgggtcggtgtgctgtaaacacaagcccagctccacatctctgctgtcaccaccccgccaaaatgcctttcagctcaggccatttattgagaccagacatttcctttactccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccggggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaaccctgtctccttccgctagatgaacatgggcattgtagcctcagcactcctggatccacgtcacaagagggactggtcagttgtgaggctaggtcatcctccaaacctctgtgtcatgggtggtatgggagattgtcccagtgccttgtttcctgtaacaggcttgatttctttcctgatgccctcagggaggcagatcctatcccttttagtggcagggatccactagcaccagcacatgaggagtgcacccagtgcacatgggcactgggacagtggacaggtgtgagattcctgggccctggagtcttttcacacctaaccatggagcctgtcccagtacatcagagtgcctcggagatgaaaaaggacatcgagccagtgacctaaattacagcctgaatatactctgaattcatgtgactgccttagctacttctctactgctgtgtagtaaaacaccaagccaacttataaaagcaggattttcctactggagcagcagctgagagtttacatcttgatccataagcacaaaagcacaagacagagagagagagagagagagagagagagagagagagagagagagagagagagagagagagtcagtcagtcagtctaacaaataactaagaaaggtgaagggtgatgaagtccacagggatcacgctagggatgttccatgccttctctgaagctaagattccttggcagcgtttgacagtaaccatagtgggtacctactgagatcactataaagataaaatagggggaagcgtatttgtactgaactggaaaaacatacaaataaagagtaaatcatggaaaaaaaaaaaaaaaaaaa

SEQ ID NO: 15 - DNA, 3826 bp, Mus musculus
gttcccagcctcatctttttcgtcgtggactctcagtggcctgggtcctggctgttttctaagcacacccttgcatcttggttcccgcacgtgggaggcccatcccggccttgagcacaatgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccggggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaaccctgtctccttccgctagatgaacatgggcattgtagcctcagcactcctggatccacgtcacaagagggactggtcagttgtgaggctaggtcatcctccaaacctctgtgtcatgggtggtatgggagattgtcccagtgccttgtttcctgtaacaggcttgatttctttcctgatgccctcagggaggcagatcctatcccttttagtggcagggatccactagcaccagcacatgaggagtgcacccagtgcacatgggcactgggacagtggacaggtgtgagattcctgggccctggagtcttttcacacctaaccatggagcctgtcccagtacatcagagtgcctcggagatgaaaaaggacatcgagccagtgacctaaattacagcctgaatatactctgaattcatgtgactgccttagctacttctctactgctgtgtagtaaaacaccaagccaacttataaaagcaggattttcctactggagcagcagctgagagtttacatcttgatccataagcacaaaagcacaagacagagagagagagagagagagagagagagagagagagagagagagagagagagagagagagtcagtcagtcagtctaacaaataactaagaaaggtgaagggtgatgaagtccacagggatcacgctagggatgttccatgccttctctgaagctaagattccttggcagcgtttgacagtaaccatagtgggtacctactgagatcactataaagataaaatagggggaagcgtatttgtactgaactggaaaaacatacaaataaagagtaaatcatggaaaaaaaaaaaaaaaaaaa

SEQ ID NO: 16 - DNA, 3633 bp, Mus musculus
gttcccagcctcatctttttcgtcgtggactctcagtggcctgggtcctggctgttttctaagcacacccttgcatcttggttcccgcacgtgggaggcccatcccggccttgagcacaatgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcagggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaaccctgtctccttccgctagatgaacatgggcattgtagcctcagcactcctggatccacgtcacaagagggactggtcagttgtgaggctaggtcatcctccaaacctctgtgtcatgggtggtatgggagattgtcccagtgccttgtttcctgtaacaggcttgatttctttcctgatgccctcagggaggcagatcctatcccttttagtggcagggatccactagcaccagcacatgaggagtgcacccagtgcacatgggcactgggacagtggacaggtgtgagattcctgggccctggagtcttttcacacctaaccatggagcctgtcccagtacatcagagtgcctcggagatgaaaaaggacatcgagccagtgacctaaattacagcctgaatatactctgaattcatgtgactgccttagctacttctctactgctgtgtagtaaaacaccaagccaacttataaaagcaggattttcctactggagcagcagctgagagtttacatcttgatccataagcacaaaagcacaagacagagagagagagagagagagagagagagagagagagagagagagagagagagagagagagtcagtcagtcagtctaacaaataactaagaaaggtgaagggtgatgaagtccacagggatcacgctagggatgttccatgccttctctgaagctaagattccttggcagcgtttgacagtaaccatagtgggtacctactgagatcactataaagataaaatagggggaagcgtatttgtactgaactggaaaaacatacaaataaagagtaaatcatggaaaaaaaaaaaaaaaaaaa

SEQ ID NO: 17 - DNA, 3378 bp, Rattus norvegicus
atgccccgcgctcctcgttgccccgccgtgcgctctctactgcgcagccgatatcgggaggtgtggccgctggcgacctttgtgcggcgcctggggcttgagggcagtcggcttgtgcaacccggggacccgaaggtcttccgcacgttggttgcccagtgcctagtgtgcgtgccctggggctcacagccgccacctgctgacctttccttccaccaggtgtcatccctgaaagagctggtgtccagggttgtgcagaaactttgcgagcgcggtgagaggaatgtgctggcttttggctttgcactgcttaacggggccagaggtgggcctcccatggccttcacgaccagcgtgcatagctacttgcccaactcggttactgagtccctgtgtgtcagtggtgcatggatgctactgttgagccgagtgggcgacgacctgctggtctacctgctgtcgcactgtgcgctctacctgctggtgccccccagctgtgcctaccaggtgtgcgggtcacccctgtaccaaatttgtgccaccacggatacctggtcctctgtgcccgctggttacaggcccactcgacccgtgggcgggaatttcactaaccttgggtccgcacaccagatcaaaaacagtggtcaccaggaagcaccaaaaccccaggccttgccatcacgaggtacgaagaggcttctgagtctcaccagtacaaacgtgccttcagctaagaaggccaggtttgaacctgccctgagagtggataagggaccccacaggcaggtggtaccaaccccatcaggcaaaacatgggcgccaagtcctgctgcgtcccccaaggtgcctcctgcagcgaaaaacttgtctttgaaaggaaaggcatctgacccgagtctctctgggtcggtgtgctgtaaacacaagcccagctcctcgtccctgctgtcatcaccaccccaagatgctgaaaagctcaggccattcactgagaccagacatttcctttactccaggggaggtggccaagaggagctaaatccctcattcctactcaacagcctcccgcctagcttgaccggggccaggagactggtggagatcatctttctgggctcaaggcctaggacatcaggaccattctgcaggacccgccgcctgccccgtcgatactggcagatgcgacccctattccagcagctgctcatgaaccacgcaaagtgccaatatgtcagattcctccggtcgcactgcagatttcgaacagcaaaccagcgggtgccggatgccatggacaccagcccatcccacctcacgagtttgctccggttacacagcagcccctggcaggtatacggctttcttcgggcctgcctccgcgagctggtgcctgccggtctctggggcaccaggcacaatgagcgccgcttcttaaagaacgtgaagaagttcatctcgttggggaagtacgccaagctatccctgcaggaactgatgtggagggtgaaagtggaggactgccactggctccgcagcagcccagagaaggacactgtccctgccgcagagcaccgtctgagggagaggatccttgccatgttcctgttctggctaatggacacatatgtggtacagctgctgaggtcattcttctacatcacagagaccacgttccagaagaaccgccttttcttctaccgtaagagtgtgtggagcaagctgcagagcattggaatcaggcaacagcttgagagagttcagctacgggaactgtcacaagaggaggtcaagcatcaccaggacacttggctggccatgcctatctgcagattgcgcttcatccccaagctcaatggtctccggcccattgtgaacatgagttatggcatggacaccagagcttttggcaaaaagaagcagacccagtgtttcactcagagtctcaagactttgttcagcgtgctcaactacgagcggaccaaacatcctaaccttatgggtgcttcagtactgggtacgagtgacagctacaggatctggcggaccttcgtgctgcgtgtgcgtgctctggaccagacacccaggatgtactttgttaaggcagatgtgacaggggcctatgatgccatcccccaggacaagctcgtggaaattgtcgccaatataatcaggcgctcagagagcatgtactgtatccgccagtatgcagtggttcagaaagatagccaaggccaagtccacaagtccttcaggagacaggtctccaccctctctgacctccagccatacatgggccagttcaccaagcatctgcaggactcagatgccagtgcactgaggaactctgttgtcatcgagcagagcatctccatgaatgagactggcagtagcctgctccacttcttcctgcgctttgtccgtcacagtgtcgtgaagatcgatggcaggttctatgtgcaatgccagggcatcccccagggctccagcctgtccaccctgctctgcagtctgtgtttcggagacatggagaacaagctgtttgccgaggtgcagcaggacggcttgcttttacgttttgtcgatgactttctgttggtgacacctcacctggcccatgcaaaagcctttctcagcaccctggtccatggcgtgcccgagtatggctgcatgataaacttgcagaagacagtggtgaacttccctgtggagaccggcgccctgggaggtgcagccccgcaccagctgcctgctcactgcctgtttccctggtgtggcttactgctggacactcggactttggaagtattctgtgactactcaggttacggacggacctcaattaagatgagcctcaccttccagggtgtctccagggccgggaagaccatgcggtacaagctcttgtcagtcttgcggttgaagtgtcatggtctgtttctagacttgcaggtgaacagcctgcagacagtctgcatcaatatatacaagatcttcctgcttcaggcctacaggttccatgcatgtgtgattcggcttccctttggccagcatgttaggaagaaccatgcattctttctgggcatcatctccaacctagcatcctgctgctacgccatcctgaaggtcaagaatccaggagtgtcactaagggccaagggtgcccctggctcctttccgcccgaggccacacgttggctctgctaccaagccttcctgctcaagctggctgctcattctgtcacctacaagtgtctcctgggacctcttaggacagcccaaaaacagctgtgccggaagctcccagaggcaacaatgaccctccttaagactgcagctgacccagccctaagcacagattttcagaccattttggactaa

SEQ ID NO: 18 - protein, 1132 aa, Artificial
  Synthetic
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGSVPAAEHRLREEILAKFLHWLMSVYVVELLRSFFYVTETTFQKNRLFFYRKSVWSKLQSIGIRQHLKRVQLRELSEAEVRQHQEARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVIIEQSSSLNEASSGLFDVFLRFVCRHAVRIRGKSYVQCQGIPQGSILSTLLCSLCYGDMENKLFAGIRRDGLLLRLVDDFLLVTPHLTHAKAFLRTLVRGVPEYGCVVNLRKTVVNFPVEDEALGGTAFVQLPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRIISDTASLCYSILKAKNAGMSLGAKGAAGPLPSEAMQWLCHQAFLLKLTRHRVTYVPLLGSLRTAQTQLSRKLPGTTLSALEAAANPALPSDFKTILD

SEQ ID NO: 19 - DNA, 4036 bp, Artificial
  Synthetic
ctcgcggcgcgagtttcaggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctaggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctctgttccggccgcagagcaccgtctgcgtgaggagatcctggccaagttcttgcactggctgatgagtgtgtacgttgtcgagctgctcaggtctttcttttatgtcacggagaccacgtttcagaagaacaggctctttttctaccggaagagtgtctggagcaagttgcaaagcattggaatcagacagcacttgaagagggtgcagctgcgggagctgtcggaagcagaggtcaggcagcatcaggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgctgtggtccagaaggccgcccatgggcacgtccgcaaggccttcaagagccacgtctctaccttgacagacctccagccgtacatgcgacagttcgtggctcacctgcaggagaccagcccactgagggatgccgtcatcatcgagcagagctcctccctgaatgaggccagcagtggcctcttcgacgtcttcctacgcttcgtgtgccgccacgccgtgcgcatcaggggcaagtcctacgtccagtgccaggggatcccgcagggctccatcctgtccacgctgctctgcagcctgtgctacggcgacatggagaacaagctgtttgcggggattcggcgggacgggctgctcctgcgtttggtggatgatttcttgttggtgacacctcacctcacccacgcgaaagccttcctcaggaccctggtccgaggtgtccctgagtatggctgcgtggtgaacttgcggaagacagtagtgaacttccctgtagaagatgaggccctgggtggcacggcttttgttcagctgccggcccacggcctattcccctggtgcggcctgctgctggacacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcatcatctctgacacggcctccctctgctactccatcctgaaagccaagaacgcagggatgtcgctgggggccaagggtgccgccggccctctgccctccgaggccatgcagtggctgtgccaccaagcattcctgctcaagctgactcgacaccgcgtcacctacgtgccactcctggggtcactcaggacagcccagacgcagctgagtcggaagctcccggggacgacgctgagtgccctggaggccgcagccaacccggcactgccctcagacttcaagaccatcctggactgatggccacccgcccacagccgggccgagagcagacaccagcagccctgtcacgccgggctctacgtcccagggagggaggggcggcccacacccagacccgcaccgctgggagtctgaggcctgagtgagtgtctggccaaggcctgcatgtccggctgaaggctgagtgtccagctgaggcctgagcgagtgtccagccaagggctgagtgtccagcacacctgccgtcttcacttccccacaggctggcgctcggctccaccccagggccagcttttcctcgccaggagcccggcttcccactccccacatgggaatagtccatccccagattcgccattgtccacccctcgccctgccctcctttgccttccacgcccaccatccagatggagaccctgagaaggaccctgggagctctgggaatttggagtgaccaaaggtgtgccctgtacacaggtgaggaccctgcacctggatgggggtccctgtgggtcaaattggggggggggtgctgtgggagtaaaatactgaatatatgagtttttcagttttgaaaaaaa

SEQ ID NO: 20 - protein, 1132 aa, Pan troglodytes; identical to SEQ ID NO: 18

SEQ ID NO: 21 - DNA, 3396 bp, Artificial
  Synthetic
atgccgcgcgcgccgcgctgccgcgcggtgcgcagcctgctgcgcagccattatcgcgaagtgctgccgctggcgacctttgtgcgccgcctgggcccgcagggctggcgcctggtgcagcgcggcgatccggcggcgtttcgcgcgctggtggcgcagtgcctggtgtgcgtgccgtgggatgcgcgcccgccgccggcggcgccgagctttcgccaggtgagctgcctgaaagaactggtggcgcgcgtgctgcagcgcctgtgcgaacgcggcgcgaaaaacgtgctggcgtttggctttgcgctgctggatggcgcgcgcggcggcccgccggaagcgtttaccaccagcgtgcgcagctatctgccgaacaccgtgaccgatgcgctgcgcggcagcggcgcgtggggcctgctgctgcgccgcgtgggcgatgatgtgctggtgcatctgctggcgcgctgcgcgctgtttgtgctggtggcgccgagctgcgcgtatcaggtgtgcggcccgccgctgtatcagctgggcgcggcgacccaggcgcgcccgccgccgcatgcgagcggcccgcgccgccgcctgggctgcgaacgcgcgtggaaccatagcgtgcgcgaagcgggcgtgccgctgggcctgccggcgccgggcgcgcgccgccgcggcggcagcgcgagccgcagcctgccgctgccgaaacgcccgcgccgcggcgcggcgccggaaccggaacgcaccccggtgggccagggcagctgggcgcatccgggccgcacccgcggcccgagcgatcgcggcttttgcgtggtgagcccggcgcgcccggcggaagaagcgaccagcctggaaggcgcgctgagcggcacccgccatagccatccgagcgtgggccgccagcatcatgcgggcccgccgagcaccagccgcccgccgcgcccgtgggataccccgtgcccgccggtgtatgcggaaaccaaacattttctgtatagcagcggcgataaagaacagctgcgcccgagctttctgctgagcagcctgcgcccgagcctgaccggcgcgcgccgcctggtggaaaccatttttctgggcagccgcccgtggatgccgggcaccccgcgccgcctgccgcgcctgccgcagcgctattggcagatgcgcccgctgtttctggaactgctgggcaaccatgcgcagtgcccgtatggcgtgctgctgaaaacccattgcccgctgcgcgcggcggtgaccccggcggcgggcgtgtgcgcgcgcgaaaaaccgcagggcagcgtggcggcgccggaagaagaagataccgatccgcgccgcctggtgcagctgctgcgccagcatagcagcccgtggcaggtgtatggctttgtgcgcgcgtgcctgcgccgcctggtgccgccgggcctgtggggcagccgccataacgaacgccgctttctgcgcaacaccaaaaaatttattagcctgggcaaacatgcgaaactgagcctgcaggaactgacctggaaaatgagcgtgcgcgattgcgcgtggctgcgccgcagcccgggcgtgggcagcgtgccggcggcggaacatcgcctgcgcgaagaaattctggcgaaatttctgcattggctgatgagcgtgtatgtggtggaactgctgcgcagctttttttatgtgaccgaaaccacctttcagaaaaaccgcctgtttttttatcgcaaaagcgtgtggagcaaactgcagagcattggcattcgccagcatctgaaacgcgtgcagctgcgcgaactgagcgaagcggaagtgcgccagcatcaggaagcgcgcccggcgctgctgaccagccgcctgcgctttattccgaaaccggatggcctgcgcccgattgtgaacatggattatgtggtgggcgcgcgcacctttcgccgcgaaaaacgcgcggaacgcctgaccagccgcgtgaaagcgctgtttagcgtgctgaactatgaacgcgcgcgccgcccgggcctgctgggcgcgagcgtgctgggcctggatgatattcatcgcgcgtggcgcacctttgtgctgcgcgtgcgcgcgcaggatccgccgccggaactgtattttgtgaaagtggatgtgaccggcgcgtatgataccattccgcaggatcgcctgaccgaagtgattgcgagcattattaaaccgcagaacacctattgcgtgcgccgctatgcggtggtgcagaaagcggcgcatggccatgtgcgcaaagcgtttaaaagccatgtgagcaccctgaccgatctgcagccgtatatgcgccagtttgtggcgcatctgcaggaaaccagcccgctgcgcgatgcggtgattattgaacagagcagcagcctgaacgaagcgagcagcggcctgtttgatgtgtttctgcgctttgtgtgccgccatgcggtgcgcattcgcggcaaaagctatgtgcagtgccagggcattccgcagggcagcattctgagcaccctgctgtgcagcctgtgctatggcgatatggaaaacaaactgtttgcgggcattcgccgcgatggcctgctgctgcgcctggtggatgattttctgctggtgaccccgcatctgacccatgcgaaagcgtttctgcgcaccctggtgcgcggcgtgccggaatatggctgcgtggtgaacctgcgcaaaaccgtggtgaactttccggtggaagatgaagcgctgggcggcaccgcgtttgtgcagctgccggcgcatggcctgtttccgtggtgcggcctgctgctggatacccgcaccctggaagtgcagagcgattatagcagctatgcgcgcaccagcattcgcgcgagcctgacctttaaccgcggctttaaagcgggccgcaacatgcgccgcaaactgtttggcgtgctgcgcctgaaatgccatagcctgtttctggatctgcaggtgaacagcctgcagaccgtgtgcaccaacatttataaaattctgctgctgcaggcgtatcgctttcatgcgtgcgtgctgcagctgccgtttcatcagcaggtgtggaaaaacccgaccttttttctgcgcattattagcgataccgcgagcctgtgctatagcattctgaaagcgaaaaacgcgggcatgagcctgggcgcgaaaggcgcggcgggcccgctgccgagcgaagcgatgcagtggctgtgccatcaggcgtttctgctgaaactgacccgccatcgcgtgacctatgtgccgctgctgggcagcctgcgcaccgcgcagacccagctgagccgcaaactgccgggcaccaccctgagcgcgctggaagcggcggcgaacccggcgctgccgagcgattttaaaaccattctggat

SEQ ID NO: 22 - protein, 807 aa, Pan troglodytes
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGSVPAAEHRLREEILAKFLHWLMSVYVVELLRSFFYVTETTFQKNRLFFYRKSVWSKLQSIGIRQHLKRVQLRELSEAEVRQHQEARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVLRPVPGDPAGLHPVHAALQPVLRRHGEQAVCGDSAGRAAPAFGG

SEQ ID NO: 23 - DNA, 2421 bp, Artificial
  Synthetic
atgccgcgcgcgccgcgctgccgcgcggtgcgcagcctgctgcgcagccattatcgcgaagtgctgccgctggcgacctttgtgcgccgcctgggcccgcagggctggcgcctggtgcagcgcggcgatccggcggcgtttcgcgcgctggtggcgcagtgcctggtgtgcgtgccgtgggatgcgcgcccgccgccggcggcgccgagctttcgccaggtgagctgcctgaaagaactggtggcgcgcgtgctgcagcgcctgtgcgaacgcggcgcgaaaaacgtgctggcgtttggctttgcgctgctggatggcgcgcgcggcggcccgccggaagcgtttaccaccagcgtgcgcagctatctgccgaacaccgtgaccgatgcgctgcgcggcagcggcgcgtggggcctgctgctgcgccgcgtgggcgatgatgtgctggtgcatctgctggcgcgctgcgcgctgtttgtgctggtggcgccgagctgcgcgtatcaggtgtgcggcccgccgctgtatcagctgggcgcggcgacccaggcgcgcccgccgccgcatgcgagcggcccgcgccgccgcctgggctgcgaacgcgcgtggaaccatagcgtgcgcgaagcgggcgtgccgctgggcctgccggcgccgggcgcgcgccgccgcggcggcagcgcgagccgcagcctgccgctgccgaaacgcccgcgccgcggcgcggcgccggaaccggaacgcaccccggtgggccagggcagctgggcgcatccgggccgcacccgcggcccgagcgatcgcggcttttgcgtggtgagcccggcgcgcccggcggaagaagcgaccagcctggaaggcgcgctgagcggcacccgccatagccatccgagcgtgggccgccagcatcatgcgggcccgccgagcaccagccgcccgccgcgcccgtgggataccccgtgcccgccggtgtatgcggaaaccaaacattttctgtatagcagcggcgataaagaacagctgcgcccgagctttctgctgagcagcctgcgcccgagcctgaccggcgcgcgccgcctggtggaaaccatttttctgggcagccgcccgtggatgccgggcaccccgcgccgcctgccgcgcctgccgcagcgctattggcagatgcgcccgctgtttctggaactgctgggcaaccatgcgcagtgcccgtatggcgtgctgctgaaaacccattgcccgctgcgcgcggcggtgaccccggcggcgggcgtgtgcgcgcgcgaaaaaccgcagggcagcgtggcggcgccggaagaagaagataccgatccgcgccgcctggtgcagctgctgcgccagcatagcagcccgtggcaggtgtatggctttgtgcgcgcgtgcctgcgccgcctggtgccgccgggcctgtggggcagccgccataacgaacgccgctttctgcgcaacaccaaaaaatttattagcctgggcaaacatgcgaaactgagcctgcaggaactgacctggaaaatgagcgtgcgcgattgcgcgtggctgcgccgcagcccgggcgtgggcagcgtgccggcggcggaacatcgcctgcgcgaagaaattctggcgaaatttctgcattggctgatgagcgtgtatgtggtggaactgctgcgcagctttttttatgtgaccgaaaccacctttcagaaaaaccgcctgtttttttatcgcaaaagcgtgtggagcaaactgcagagcattggcattcgccagcatctgaaacgcgtgcagctgcgcgaactgagcgaagcggaagtgcgccagcatcaggaagcgcgcccggcgctgctgaccagccgcctgcgctttattccgaaaccggatggcctgcgcccgattgtgaacatggattatgtggtgggcgcgcgcacctttcgccgcgaaaaacgcgcggaacgcctgaccagccgcgtgaaagcgctgtttagcgtgctgaactatgaacgcgcgcgccgcccgggcctgctgggcgcgagcgtgctgggcctggatgatattcatcgcgcgtggcgcacctttgtgctgcgcgtgcgcgcgcaggatccgccgccggaactgtattttgtgaaagtggatgtgaccggcgcgtatgataccattccgcaggatcgcctgaccgaagtgattgcgagcattattaaaccgcagaacacctattgcgtgcgccgctatgcggtggtgcagaaagcggcgcatggccatgtgcgcaaagcgtttaaaagccatgtgctgcgcccggtgccgggcgatccggcgggcctgcatccggtgcatgcggcgctgcagccggtgctgcgccgccatggcgaacaggcggtgtgcggcgatagcgcgggccgcgcggcgccggcgtttggcggc

SEQ ID NO: 24 - protein, 1069 aa, Pan troglodytes
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGSVPAAEHRLREEILAKFLHWLMSVYVVELLRSFFYVTETTFQKNRLFFYRKSVWSKLQSIGIRQHLKRVQLRELSEAEVRQHQEARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVIIEQSSSLNEASSGLFDVFLRFVCRHAVRIRGKSYVQCQGIPQGSILSTLLCSLCYGDMENKLFAGIRRDGLLLRLVDDFLLVTPHLTHAKAFLSYARTSIRASLTFNRGFKAGRNMRRKLFGVLRLKCHSLFLDLQVNSLQTVCTNIYKILLLQAYRFHACVLQLPFHQQVWKNPTFFLRIISDTASLCYSILKAKNAGMSLGAKGAAGPLPSEAMQWLCHQAFLLKLTRHRVTYVPLLGSLRTAQTQLSRKLPGTTLSALEAAANPALPSDFKTILD

SEQ ID NO: 25 - DNA, 3207 bp, Artificial
  Synthetic
atgccgcgcgcgccgcgctgccgcgcggtgcgcagcctgctgcgcagccattatcgcgaagtgctgccgctggcgacctttgtgcgccgcctgggcccgcagggctggcgcctggtgcagcgcggcgatccggcggcgtttcgcgcgctggtggcgcagtgcctggtgtgcgtgccgtgggatgcgcgcccgccgccggcggcgccgagctttcgccaggtgagctgcctgaaagaactggtggcgcgcgtgctgcagcgcctgtgcgaacgcggcgcgaaaaacgtgctggcgtttggctttgcgctgctggatggcgcgcgcggcggcccgccggaagcgtttaccaccagcgtgcgcagctatctgccgaacaccgtgaccgatgcgctgcgcggcagcggcgcgtggggcctgctgctgcgccgcgtgggcgatgatgtgctggtgcatctgctggcgcgctgcgcgctgtttgtgctggtggcgccgagctgcgcgtatcaggtgtgcggcccgccgctgtatcagctgggcgcggcgacccaggcgcgcccgccgccgcatgcgagcggcccgcgccgccgcctgggctgcgaacgcgcgtggaaccatagcgtgcgcgaagcgggcgtgccgctgggcctgccggcgccgggcgcgcgccgccgcggcggcagcgcgagccgcagcctgccgctgccgaaacgcccgcgccgcggcgcggcgccggaaccggaacgcaccccggtgggccagggcagctgggcgcatccgggccgcacccgcggcccgagcgatcgcggcttttgcgtggtgagcccggcgcgcccggcggaagaagcgaccagcctggaaggcgcgctgagcggcacccgccatagccatccgagcgtgggccgccagcatcatgcgggcccgccgagcaccagccgcccgccgcgcccgtgggataccccgtgcccgccggtgtatgcggaaaccaaacattttctgtatagcagcggcgataaagaacagctgcgcccgagctttctgctgagcagcctgcgcccgagcctgaccggcgcgcgccgcctggtggaaaccatttttctgggcagccgcccgtggatgccgggcaccccgcgccgcctgccgcgcctgccgcagcgctattggcagatgcgcccgctgtttctggaactgctgggcaaccatgcgcagtgcccgtatggcgtgctgctgaaaacccattgcccgctgcgcgcggcggtgaccccggcggcgggcgtgtgcgcgcgcgaaaaaccgcagggcagcgtggcggcgccggaagaagaagataccgatccgcgccgcctggtgcagctgctgcgccagcatagcagcccgtggcaggtgtatggctttgtgcgcgcgtgcctgcgccgcctggtgccgccgggcctgtggggcagccgccataacgaacgccgctttctgcgcaacaccaaaaaatttattagcctgggcaaacatgcgaaactgagcctgcaggaactgacctggaaaatgagcgtgcgcgattgcgcgtggctgcgccgcagcccgggcgtgggcagcgtgccggcggcggaacatcgcctgcgcgaagaaattctggcgaaatttctgcattggctgatgagcgtgtatgtggtggaactgctgcgcagctttttttatgtgaccgaaaccacctttcagaaaaaccgcctgtttttttatcgcaaaagcgtgtggagcaaactgcagagcattggcattcgccagcatctgaaacgcgtgcagctgcgcgaactgagcgaagcggaagtgcgccagcatcaggaagcgcgcccggcgctgctgaccagccgcctgcgctttattccgaaaccggatggcctgcgcccgattgtgaacatggattatgtggtgggcgcgcgcacctttcgccgcgaaaaacgcgcggaacgcctgaccagccgcgtgaaagcgctgtttagcgtgctgaactatgaacgcgcgcgccgcccgggcctgctgggcgcgagcgtgctgggcctggatgatattcatcgcgcgtggcgcacctttgtgctgcgcgtgcgcgcgcaggatccgccgccggaactgtattttgtgaaagtggatgtgaccggcgcgtatgataccattccgcaggatcgcctgaccgaagtgattgcgagcattattaaaccgcagaacacctattgcgtgcgccgctatgcggtggtgcagaaagcggcgcatggccatgtgcgcaaagcgtttaaaagccatgtgagcaccctgaccgatctgcagccgtatatgcgccagtttgtggcgcatctgcaggaaaccagcccgctgcgcgatgcggtgattattgaacagagcagcagcctgaacgaagcgagcagcggcctgtttgatgtgtttctgcgctttgtgtgccgccatgcggtgcgcattcgcggcaaaagctatgtgcagtgccagggcattccgcagggcagcattctgagcaccctgctgtgcagcctgtgctatggcgatatggaaaacaaactgtttgcgggcattcgccgcgatggcctgctgctgcgcctggtggatgattttctgctggtgaccccgcatctgacccatgcgaaagcgtttctgagctatgcgcgcaccagcattcgcgcgagcctgacctttaaccgcggctttaaagcgggccgcaacatgcgccgcaaactgtttggcgtgctgcgcctgaaatgccatagcctgtttctggatctgcaggtgaacagcctgcagaccgtgtgcaccaacatttataaaattctgctgctgcaggcgtatcgctttcatgcgtgcgtgctgcagctgccgtttcatcagcaggtgtggaaaaacccgaccttttttctgcgcattattagcgataccgcgagcctgtgctatagcattctgaaagcgaaaaacgcgggcatgagcctgggcgcgaaaggcgcggcgggcccgctgccgagcgaagcgatgcagtggctgtgccatcaggcgtttctgctgaaactgacccgccatcgcgtgacctatgtgccgctgctgggcagcctgcgcaccgcgcagacccagctgagccgcaaactgccgggcaccaccctgagcgcgctggaagcggcggcgaacccggcgctgccgagcgattttaaaaccattctggat

SEQ ID NO: 26 - protein, 795 aa, Pan troglodytes
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGSVPAAEHRLREEILAKFLHWLMSVYVVELLRSFFYVTETTFQKNRLFFYRKSVWSKLQSIGIRQHLKRVQLRELSEAEVRQHQEARPALLTSRLRFIPKPDGLRPIVNMDYVVGARTFRREKRAERLTSRVKALFSVLNYERARRPGLLGASVLGLDDIHRAWRTFVLRVRAQDPPPELYFVKDRLTEVIASIIKPQNTYCVRRYAVVQKAAHGHVRKAFKSHVLRPVPGDPAGLHPVHAALQPVLRRHGEQAVCGDSAGRAAPAFGG

SEQ ID NO: 27 - DNA, 2385 bp, Artificial
  Synthetic
atgccgcgcgcgccgcgctgccgcgcggtgcgcagcctgctgcgcagccattatcgcgaagtgctgccgctggcgacctttgtgcgccgcctgggcccgcagggctggcgcctggtgcagcgcggcgatccggcggcgtttcgcgcgctggtggcgcagtgcctggtgtgcgtgccgtgggatgcgcgcccgccgccggcggcgccgagctttcgccaggtgagctgcctgaaagaactggtggcgcgcgtgctgcagcgcctgtgcgaacgcggcgcgaaaaacgtgctggcgtttggctttgcgctgctggatggcgcgcgcggcggcccgccggaagcgtttaccaccagcgtgcgcagctatctgccgaacaccgtgaccgatgcgctgcgcggcagcggcgcgtggggcctgctgctgcgccgcgtgggcgatgatgtgctggtgcatctgctggcgcgctgcgcgctgtttgtgctggtggcgccgagctgcgcgtatcaggtgtgcggcccgccgctgtatcagctgggcgcggcgacccaggcgcgcccgccgccgcatgcgagcggcccgcgccgccgcctgggctgcgaacgcgcgtggaaccatagcgtgcgcgaagcgggcgtgccgctgggcctgccggcgccgggcgcgcgccgccgcggcggcagcgcgagccgcagcctgccgctgccgaaacgcccgcgccgcggcgcggcgccggaaccggaacgcaccccggtgggccagggcagctgggcgcatccgggccgcacccgcggcccgagcgatcgcggcttttgcgtggtgagcccggcgcgcccggcggaagaagcgaccagcctggaaggcgcgctgagcggcacccgccatagccatccgagcgtgggccgccagcatcatgcgggcccgccgagcaccagccgcccgccgcgcccgtgggataccccgtgcccgccggtgtatgcggaaaccaaacattttctgtatagcagcggcgataaagaacagctgcgcccgagctttctgctgagcagcctgcgcccgagcctgaccggcgcgcgccgcctggtggaaaccatttttctgggcagccgcccgtggatgccgggcaccccgcgccgcctgccgcgcctgccgcagcgctattggcagatgcgcccgctgtttctggaactgctgggcaaccatgcgcagtgcccgtatggcgtgctgctgaaaacccattgcccgctgcgcgcggcggtgaccccggcggcgggcgtgtgcgcgcgcgaaaaaccgcagggcagcgtggcggcgccggaagaagaagataccgatccgcgccgcctggtgcagctgctgcgccagcatagcagcccgtggcaggtgtatggctttgtgcgcgcgtgcctgcgccgcctggtgccgccgggcctgtggggcagccgccataacgaacgccgctttctgcgcaacaccaaaaaatttattagcctgggcaaacatgcgaaactgagcctgcaggaactgacctggaaaatgagcgtgcgcgattgcgcgtggctgcgccgcagcccgggcgtgggcagcgtgccggcggcggaacatcgcctgcgcgaagaaattctggcgaaatttctgcattggctgatgagcgtgtatgtggtggaactgctgcgcagctttttttatgtgaccgaaaccacctttcagaaaaaccgcctgtttttttatcgcaaaagcgtgtggagcaaactgcagagcattggcattcgccagcatctgaaacgcgtgcagctgcgcgaactgagcgaagcggaagtgcgccagcatcaggaagcgcgcccggcgctgctgaccagccgcctgcgctttattccgaaaccggatggcctgcgcccgattgtgaacatggattatgtggtgggcgcgcgcacctttcgccgcgaaaaacgcgcggaacgcctgaccagccgcgtgaaagcgctgtttagcgtgctgaactatgaacgcgcgcgccgcccgggcctgctgggcgcgagcgtgctgggcctggatgatattcatcgcgcgtggcgcacctttgtgctgcgcgtgcgcgcgcaggatccgccgccggaactgtattttgtgaaagatcgcctgaccgaagtgattgcgagcattattaaaccgcagaacacctattgcgtgcgccgctatgcggtggtgcagaaagcggcgcatggccatgtgcgcaaagcgtttaaaagccatgtgctgcgcccggtgccgggcgatccggcgggcctgcatccggtgcatgcggcgctgcagccggtgctgcgccgccatggcgaacaggcggtgtgcggcgatagcgcgggccgcgcggcgccggcgtttggcggc

SEQ ID NO: 28 - protein, 1086 aa, Pan troglodytes
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGSVPAAEHRLREEILAKFLHWLMSVYVVELLRSFFYVTETTFQKNRLFFYRKSVWSKLQSIGIRQHLKRVQLRELSEAEVRQHQEARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVIIEQSSSLNEASSGLFDVFLRFVCRHAVRIRGKSYVQCQGIPQGSILSTLLCSLCYGDMENKLFAGIRRDGLLLRLVDDFLLVTPHLTHAKAFLRTLVRGVPEYGCVVNLRKTVVNFPVEDEALGGTAFVQLPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRIISDTASLCYSILKAKNAAQTQLSRKLPGTTLSALEAAANPALPSDFKTILD

SEQ ID NO: 29 - DNA, 3258 bp, Artificial
  Synthetic
atgccgcgcgcgccgcgctgccgcgcggtgcgcagcctgctgcgcagccattatcgcgaagtgctgccgctggcgacctttgtgcgccgcctgggcccgcagggctggcgcctggtgcagcgcggcgatccggcggcgtttcgcgcgctggtggcgcagtgcctggtgtgcgtgccgtgggatgcgcgcccgccgccggcggcgccgagctttcgccaggtgagctgcctgaaagaactggtggcgcgcgtgctgcagcgcctgtgcgaacgcggcgcgaaaaacgtgctggcgtttggctttgcgctgctggatggcgcgcgcggcggcccgccggaagcgtttaccaccagcgtgcgcagctatctgccgaacaccgtgaccgatgcgctgcgcggcagcggcgcgtggggcctgctgctgcgccgcgtgggcgatgatgtgctggtgcatctgctggcgcgctgcgcgctgtttgtgctggtggcgccgagctgcgcgtatcaggtgtgcggcccgccgctgtatcagctgggcgcggcgacccaggcgcgcccgccgccgcatgcgagcggcccgcgccgccgcctgggctgcgaacgcgcgtggaaccatagcgtgcgcgaagcgggcgtgccgctgggcctgccggcgccgggcgcgcgccgccgcggcggcagcgcgagccgcagcctgccgctgccgaaacgcccgcgccgcggcgcggcgccggaaccggaacgcaccccggtgggccagggcagctgggcgcatccgggccgcacccgcggcccgagcgatcgcggcttttgcgtggtgagcccggcgcgcccggcggaagaagcgaccagcctggaaggcgcgctgagcggcacccgccatagccatccgagcgtgggccgccagcatcatgcgggcccgccgagcaccagccgcccgccgcgcccgtgggataccccgtgcccgccggtgtatgcggaaaccaaacattttctgtatagcagcggcgataaagaacagctgcgcccgagctttctgctgagcagcctgcgcccgagcctgaccggcgcgcgccgcctggtggaaaccatttttctgggcagccgcccgtggatgccgggcaccccgcgccgcctgccgcgcctgccgcagcgctattggcagatgcgcccgctgtttctggaactgctgggcaaccatgcgcagtgcccgtatggcgtgctgctgaaaacccattgcccgctgcgcgcggcggtgaccccggcggcgggcgtgtgcgcgcgcgaaaaaccgcagggcagcgtggcggcgccggaagaagaagataccgatccgcgccgcctggtgcagctgctgcgccagcatagcagcccgtggcaggtgtatggctttgtgcgcgcgtgcctgcgccgcctggtgccgccgggcctgtggggcagccgccataacgaacgccgctttctgcgcaacaccaaaaaatttattagcctgggcaaacatgcgaaactgagcctgcaggaactgacctggaaaatgagcgtgcgcgattgcgcgtggctgcgccgcagcccgggcgtgggcagcgtgccggcggcggaacatcgcctgcgcgaagaaattctggcgaaatttctgcattggctgatgagcgtgtatgtggtggaactgctgcgcagctttttttatgtgaccgaaaccacctttcagaaaaaccgcctgtttttttatcgcaaaagcgtgtggagcaaactgcagagcattggcattcgccagcatctgaaacgcgtgcagctgcgcgaactgagcgaagcggaagtgcgccagcatcaggaagcgcgcccggcgctgctgaccagccgcctgcgctttattccgaaaccggatggcctgcgcccgattgtgaacatggattatgtggtgggcgcgcgcacctttcgccgcgaaaaacgcgcggaacgcctgaccagccgcgtgaaagcgctgtttagcgtgctgaactatgaacgcgcgcgccgcccgggcctgctgggcgcgagcgtgctgggcctggatgatattcatcgcgcgtggcgcacctttgtgctgcgcgtgcgcgcgcaggatccgccgccggaactgtattttgtgaaagtggatgtgaccggcgcgtatgataccattccgcaggatcgcctgaccgaagtgattgcgagcattattaaaccgcagaacacctattgcgtgcgccgctatgcggtggtgcagaaagcggcgcatggccatgtgcgcaaagcgtttaaaagccatgtgagcaccctgaccgatctgcagccgtatatgcgccagtttgtggcgcatctgcaggaaaccagcccgctgcgcgatgcggtgattattgaacagagcagcagcctgaacgaagcgagcagcggcctgtttgatgtgtttctgcgctttgtgtgccgccatgcggtgcgcattcgcggcaaaagctatgtgcagtgccagggcattccgcagggcagcattctgagcaccctgctgtgcagcctgtgctatggcgatatggaaaacaaactgtttgcgggcattcgccgcgatggcctgctgctgcgcctggtggatgattttctgctggtgaccccgcatctgacccatgcgaaagcgtttctgcgcaccctggtgcgcggcgtgccggaatatggctgcgtggtgaacctgcgcaaaaccgtggtgaactttccggtggaagatgaagcgctgggcggcaccgcgtttgtgcagctgccggcgcatggcctgtttccgtggtgcggcctgctgctggatacccgcaccctggaagtgcagagcgattatagcagctatgcgcgcaccagcattcgcgcgagcctgacctttaaccgcggctttaaagcgggccgcaacatgcgccgcaaactgtttggcgtgctgcgcctgaaatgccatagcctgtttctggatctgcaggtgaacagcctgcagaccgtgtgcaccaacatttataaaattctgctgctgcaggcgtatcgctttcatgcgtgcgtgctgcagctgccgtttcatcagcaggtgtggaaaaacccgaccttttttctgcgcattattagcgataccgcgagcctgtgctatagcattctgaaagcgaaaaacgcggcgcagacccagctgagccgcaaactgccgggcaccaccctgagcgcgctggaagcggcggcgaacccggcgctgccgagcgattttaaaaccattctggat

SEQ ID NO: 30 - DNA, 3399 bp, Artificial
  Synthetic
atgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactga

SEQ ID NO: 31 - DNA, 3369 bp, Artificial
  Synthetic
atgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggtgtgtgggtctcccctgtaccaaatttgtgccaccacggatatctggccctctgtgtccgctagttacaggcccacccgacccgtgggcaggaatttcactaaccttaggttcttacaacagatcaagagcagtagtcgccaggaagcaccgaaacccctggccttgccatctcgaggtacaaagaggcatctgagtctcaccagtacaagtgtgccttcagctaagaaggccagatgctatcctgtcccgagagtggaggagggaccccacaggcaggtgctaccaaccccatcaggcaaatcatgggtgccaagtcctgctcggtcccccgaggtgcctactgcagagaaagatttgtcttctaaaggaaaggtgtctgacctgagtctctctgggtcggtgtgctgtaaacacaagcccagctccacatctctgctgtcaccaccccgccaaaatgcctttcagctcaggccatttattgagaccagacatttcctttactccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccggggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaa

SEQ ID NO: 32 - DNA, 102 bp, Artificial
  Synthetic
ttaagctgccttctgcggggcttgccttctggccatgcccttcttctctcccttgcacctgtacctcttggtctttgaataaagcctgagtaggaagtctag

SEQ ID NO: 33 - DNA, 144 bp, Artificial
  Synthetic
caattggctcgctttcttgctgtccaatttctattaaaggttcctttgttccctaagtccaactactaaactgggggatattatgaagggccttgagcatctggattctgcctaataaaaaacatttattttcattgcgaattc

SEQ ID NO: 34 - DNA, 145 bp, Artificial
  Synthetic
caattggctcgctttcttgctgtccaatttctattaaaggttccttttgttccctaagtccaactactaaactgggggatattatgaagggccttgagcatctggattctgcctaataaaaaacatttcttttcattgcgaattc

SEQ ID NO: 35 - DNA, 47 bp, Artificial
  Synthetic
aggaaataagagagaaaagaagagtaagaagaaatataagagccacc

SEQ ID NO: 36 - DNA, 57 bp, Artificial
  Synthetic
ggacatttgcttctgacacaactgtgttcactagcaacctcaaacaactagtacacc

SEQ ID NO: 37 - DNA, 3723 bp, Artificial
  Synthetic
gggacatttgcttctgacacaactgtgttcactagcaacctcaaacaaccggtacaccatgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggtgtgtgggtctcccctgtaccaaatttgtgccaccacggatatctggccctctgtgtccgctagttacaggcccacccgacccgtgggcaggaatttcactaaccttaggttcttacaacagatcaagagcagtagtcgccaggaagcaccgaaacccctggccttgccatctcgaggtacaaagaggcatctgagtctcaccagtacaagtgtgccttcagctaagaaggccagatgctatcctgtcccgagagtggaggagggaccccacaggcaggtgctaccaaccccatcaggcaaatcatgggtgccaagtcctgctcggtcccccgaggtgcctactgcagagaaagatttgtcttctaaaggaaaggtgtctgacctgagtctctctgggtcggtgtgctgtaaacacaagcccagctccacatctctgctgtcaccaccccgccaaaatgcctttcagctcaggccatttattgagaccagacatttcctttactccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccggggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaacaattggctcgctttcttgctgtccaatttctattaaaggttccttttgttccctaagtccaactactaaactgggggatattatgaagggccttgagcatctggattctgcctaataaaaaacatttcttttcattgcgaattcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 38 - DNA, 3601 bp, Artificial
  Synthetic
aggaaataagagagaaaagaagagtaagaagaaatataagagccaccatgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggtgtgtgggtctcccctgtaccaaatttgtgccaccacggatatctggccctctgtgtccgctagttacaggcccacccgacccgtgggcaggaatttcactaaccttaggttcttacaacagatcaagagcagtagtcgccaggaagcaccgaaacccctggccttgccatctcgaggtacaaagaggcatctgagtctcaccagtacaagtgtgccttcagctaagaaggccagatgctatcctgtcccgagagtggaggagggaccccacaggcaggtgctaccaaccccatcaggcaaatcatgggtgccaagtcctgctcggtcccccgaggtgcctactgcagagaaagatttgtcttctaaaggaaaggtgtctgacctgagtctctctgggtcggtgtgctgtaaacacaagcccagctccacatctctgctgtcaccaccccgccaaaatgcctttcagctcaggccatttattgagaccagacatttcctttactccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccggggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaataattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 39 - DNA, 3753 bp, Artificial
  Synthetic
gggacatttgcttctgacacaactgtgttcactagcaacctcaaacaactagtacaccatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgacaattggctcgctttcttgctgtccaatttctattaaaggttccttttgttccctaagtccaactactaaactgggggatattatgaagggccttgagcatctggattctgcctaataaaaaacatttcttttcattgcgaattcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 40 - DNA, 3665 bp, Artificial
  Synthetic
aggaaataagagagaaaagaagagtaagaagaaatataagagccaccatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgataattaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2025/0263678 A1
(43) Pub. Date: Aug. 21, 2025

(54) COMPOSITIONS AND METHODS FOR DELIVERY OF RNA

(71) Applicant: REJUVENATION TECHNOLOGIES, INC., Mountain View, CA (US)

(72) Inventors: John RAMUNAS, Mountain View, CA (US); Glenn Jeremy MARKOV, Mountain View, CA (US); William Gillis OLSEN, Mountain View, CA (US)

(73) Assignee: REJUVENATION TECHNOLOGIES, INC., Mountain View, CA (US)

(21) Appl. No.: 18/270,179

(22) PCT Filed: Dec. 28, 2021
(86) PCT No.: PCT/US2021/065386

§ 371 Date: Jun. 28, 2023

Related U.S. Application Data
(60) Provisional application No. 63/131,528, filed on Dec. 29, 2020.

(51) Int. Cl. C12N 9/12; A61K 9/51; A61K 38/00; A61K 47/54; A61K 47/69; A61K 48/00; A61P 1/16

(52) U.S. Cl. CPC C12N 9/1276; A61K 9/5123; A61K 47/543; A61K 47/6929; A61K 48/0033; A61P 1/16; A61K 38/00; C12Y 207/07049

(57) ABSTRACT

The disclosure relates to compositions and methods for the treatment of fibrotic diseases and disorders and/or liver diseases and disorders, with one or more synthetic messenger ribonucleic acids (mRNAs) encoding telomerase reverse transcriptase (TERT).

### CROSS-REFERENCE TO RELATED APPLICATIONS

This application claims priority to and the benefit of U.S. Provisional Patent Application No. 63/131,528 entitled “COMPOSITIONS AND METHODS FOR DELIVERY OF RNA ENCODING TERT USING LIPIDS,” filed Dec. 29, 2020, the disclosure of which is hereby incorporated by reference in its entirety.

### INCORPORATION OF THE SEQUENCE LISTING

The contents of the text file named “REJU-002_03WO_SeqList_ST25.txt,” which was created on Dec. 28, 2021 and is 205 KB in size, are hereby incorporated by reference in their entirety.

### FIELD OF THE DISCLOSURE

The present disclosure relates generally to telomerase reverse transcriptase (TERT) messenger ribonucleic acid (mRNA) therapies for the treatment of fibrotic diseases and liver diseases.

### BACKGROUND

Drug treatment of fibrotic diseases and liver diseases remains elusive as evidenced by the high mortality rates of these diseases. Currently, cessation of the damaging activity or disease is the primary method for treating fibrosis, e.g., of the liver, and liver disease. Therefore, a need exists for pharmaceutical therapies to treat fibrotic diseases and liver diseases.

### SUMMARY

The disclosure relates to telomerase reverse transcriptase (TERT) messenger ribonucleic acid (mRNA) therapies for the treatment of fibrotic diseases and conditions, e.g. of the liver, and liver diseases and conditions. Treatment with compositions comprising TERT mRNA may prevent, reverse or treat fibrosis and other pathological features of fibrotic disease and/or liver disease leading to improvements in overall organ function and subject health. Accordingly, in some embodiments, provided herein are compositions comprising one or more synthetic messenger ribonucleic acids (mRNAs) encoding telomerase reverse transcriptase (TERT).

In some embodiments, the composition comprises: (i) a ribonucleic acid (RNA) encoding telomerase reverse transcriptase (TERT) and (ii) a delivery vehicle, wherein the RNA of (i) comprises one or more modified nucleotides and wherein the delivery vehicle of (ii) is operably-linked to the RNA of (i).

In some embodiments of the compositions of the disclosure, the delivery vehicle comprises one or more of a nanoparticle, a liposome, a cationic lipid, an exosome, an extracellular vesicle, a lipid nanoparticle, a natural lipoprotein particle, and an artificial lipoprotein particle.

In some embodiments of the compositions of the disclosure, the delivery vehicle comprises a lipid nanoparticle (LNP). In some embodiments, the delivery vehicle comprises an ionizable and/or cationic lipid.

In some embodiments, the delivery vehicle comprises a targeting moiety. In some embodiments, the targeting moiety results in the delivery vehicle specifically or selectively interacting with a liver cell. In some embodiments, the targeting moiety comprises cholesterol. In some embodiments, the targeting moiety is a lipid, a peptide, and/or an antibody. In some embodiments, the the LNP comprises an ionizable lipid, a phospholipid, a cholesterol, and/or a PEGylated lipid. In some embodiments, the LNP comprises a molar ratio of about 30-70 moles of an ionizable lipid, to about 0.1 to about 20 moles of a phospholipid, about 20 to about 60 moles of cholesterol, and about 0.1 to about 5.5 moles of PEGylated lipid.

In some embodiments of the compositions of the disclosure, including those in which the delivery vehicle is a lipid nanoparticle (LNP), the delivery vehicle comprises a compound of Formula I:

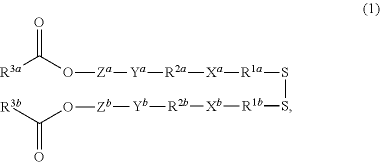

wherein R^{1a} and R^{1b} each independently represents an alkylene group having 1 to 6 carbon atoms, wherein X^{a} and X^{b} are each independently an acyclic alkyl tertiary amino group having 1 to 6 carbon atoms and 1 tertiary amino group, or 2 to 5 carbon atoms, and A cyclic alkylene tertiary amino group having 1 to 2 tertiary amino groups, wherein R^{2a} and R^{2b} each independently represent an alkylene group having 8 or less carbon atoms or an oxydialkylene group, wherein Y^{a} and Y^{b} each independently represent an ester bond, an amide bond, a carbamate bond, an ether bond or a urea bond; wherein Z^{a} and Z^{b} are each independently a divalent group derived from an aromatic compound having 3 to 16 carbon atoms, having at least one aromatic ring, and optionally having a hetero atom, and wherein R^{3a} and R^{3b} each independently represent a residue derived from a reaction product of a fat-soluble vitamin having a hydroxyl group and succinic anhydride or glutaric anhydride, or a sterol derivative having a hydroxyl group and succinic anhydride or a residue derived from a reaction product with glutaric anhydride or an aliphatic hydrocarbon group having 12 to 22 carbon atoms.

In some embodiments of the compositions of the disclosure, including those in which the delivery vehicle is a lipid nanoparticle (LNP), the compound of Formula I is:

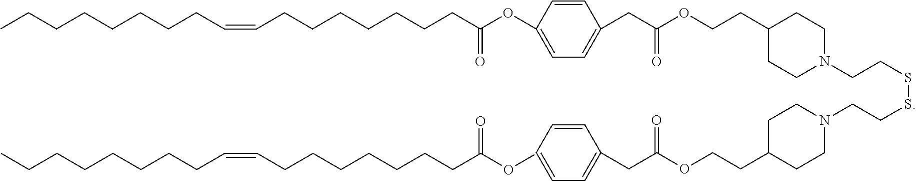

In some embodiments of the compositions of the disclosure, including those in which the delivery vehicle is a lipid nanoparticle (LNP), the compound of Formula I is:

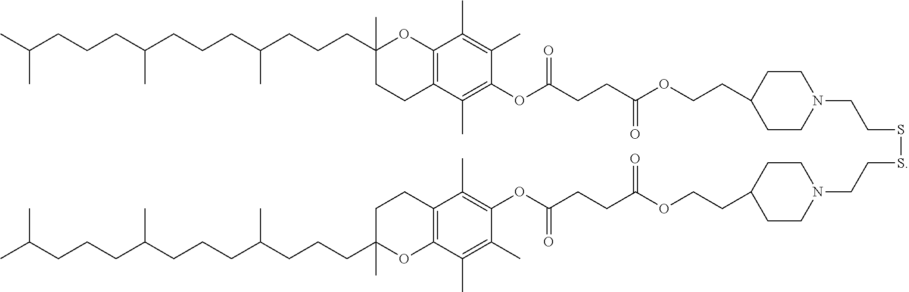 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

In some embodiments of the compositions of the disclosure, including those in which the delivery vehicle is a lipid nanoparticle (LNP), the compound of Formula I is:

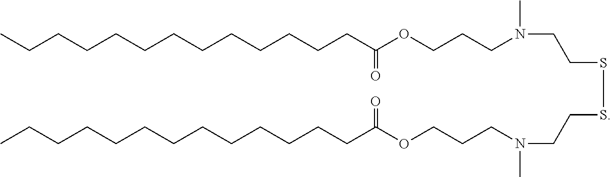 [SMILES: CCCCCCCCCCCCCC(=O)OCCCN(C)CCSSCCN(C)CCCOC(=O)CCCCCCCCCCCCC]

In some embodiments of the compositions of the disclosure, including those in which the delivery vehicle is a lipid nanoparticle (LNP), the compound of Formula I is:

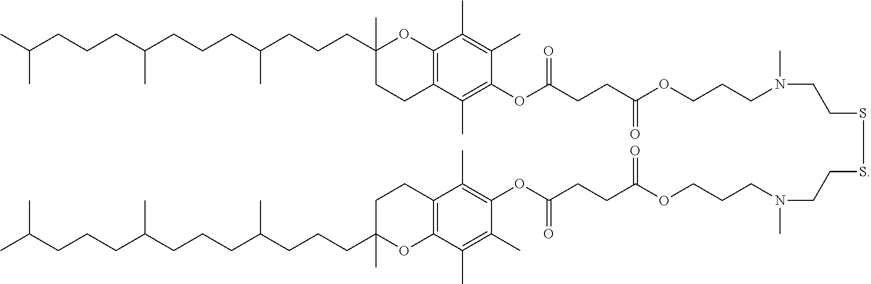 [SMILES: Cc1c(C)c2c(c(C)c1OC(=O)CCC(=O)OCCCN(C)CCSSCCN(C)CCCOC(=O)CCC(=O)Oc1c(C)c(C)c3c(c1C)CCC(C)(CCCC(C)CCCC(C)CCCC(C)C)O3)CCC(C)(CCCC(C)CCCC(C)CCCC(C)C)O2]

In some embodiments of the compositions of the disclosure, including those in which the delivery vehicle is a lipid nanoparticle (LNP), the compound of Formula I is:

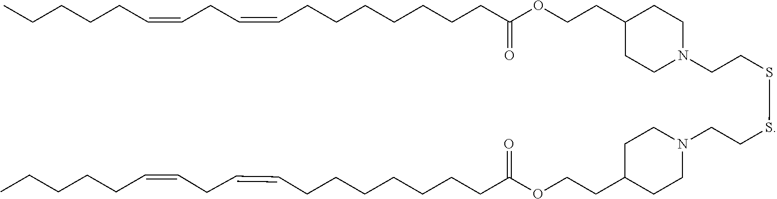 [SMILES: CCCCC/C=C\C/C=C\CCCCCCCC(=O)OCCC1CCN(CCSSCCN2CCC(CCOC(=O)CCCCCCC/C=C\C/C=C\CCCCC)CC2)CC1]

In some embodiments of the compositions of the disclosure, including those in which the delivery vehicle is a lipid nanoparticle (LNP), the compound of Formula I is:

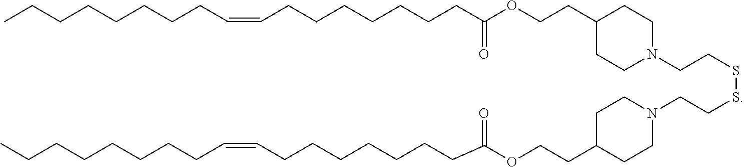 [SMILES: CCCCCCCC/C=C\CCCCCCCC(=O)OCCC1CCN(CCSSCCN2CCC(CCOC(=O)CCCCCCC/C=C\CCCCCCCC)CC2)CC1]

In some embodiments, the RNA comprises a sequence at least 70%, at least 75%, at least 80%, at least 85%, at least 90%, at least 95%, at least 97%, at least 99%, or 100% identical to any one of SEQ ID NOS: 1-5, 30-31, or 37-40.

In some embodiments of the compositions of the disclosure, including those in which the delivery vehicle is a lipid nanoparticle (LNP), the RNA comprises a 5′ cap. In some embodiments, the 5′cap comprises an anti-reverse cap analog (ARCA). In some embodiments, the ARCA comprises an 3′-O-Me-m7G(5′)ppp(5′)G structure. In some embodiments, the 5′ cap comprises m7G(5′)ppp(5′)(2′OMeA)pG. In some embodiments, the 5′ cap comprises m7(3′OMeG)(5′)ppp(5′)(2′OMeA)pG.

In some embodiments of the compositions of the disclosure, including those in which the delivery vehicle is a lipid nanoparticle (LNP), the RNA further comprises at least one untranslated region (UTR). The UTR may comprise a sequence at least 70%, at least 75%, at least 80%, at least 85%, at least 90%, at least 95%, at least 97%, at least 99%, or 100% identical to any one of SEQ ID NOs: 32-36. In some embodiments, the at least one UTR is positioned 5′ to (i) the RNA encoding TERT. In some embodiments, the at least one UTR is positioned 3′ to the RNA of (i). In some embodiments, the UTR comprises a human sequence. In some embodiments, the UTR comprises a non-human or synthetic sequence. In some embodiments, the UTR comprises a chimeric sequence. In some embodiments, the UTR increases stability, increases half-life, increases a transcription rate or decreases a time until initiation of transcription of the RNA of (i). In some embodiments, the UTR comprises a sequence having at least 70% identity to a UTR sequence isolated or derived from one or more of α-globin, β-globin, c-fos, and a tobacco etch virus.

In some embodiments of the compositions of the disclosure, including those in which the delivery vehicle is a lipid nanoparticle (LNP), the one or more modified nucleotides of the RNA of (i) comprise one or more of a modified adenine or analog thereof, a modified cytidine or analog thereof, a modified guanosine or analog thereof, and a modified uridine or analog thereof. In some embodiments, the one or more modified nucleotides of the RNA of (i) comprise one or more of 1-methylpseudouridine also known as N1-Methylpseudouridine, pseudouridine (N1m), 2-thiouridine, and 5-methylcytidine. In some embodiments, the one or more modified nucleotides of the RNA of (i) comprise 5-methoxyuridine (5-moU). In some embodiments, the one or more modified nucleotides of the RNA of (i) comprise one or more of m1A 1-methyladenosine, m6A N6-methyladenosine, Am 2′-O-methyladenosine, i6A N6-isopentenyladenosine, io6A N6-(cis-hydroxyisopentenyl)adenosine, ms2io6A 2-methylthio-N6-(cis-hydroxyisopentenyl) adenosine, g6A N6-glycinylcarbamoyladenosine, t6A N6-threonylcarbamoyladenosine, ms2t6A 2-methylthio-N6-threonyl carbamoyladenosine, Ar(p) 2′-O-ribosyladenosine (phosphate), m6 2A N6,N6-dimethyladenosine, m6Am N6,2′-O-dimethyladenosine, m6 2Am N6,N6,2′-O-trimethyladenosine, m1Am 1,2′-O-dimethyladenosine, m3C 3-methylcytidine, m5C 5-methylcytidine, Cm 2′-O-methylcytidine, ac4C N4-acetylcytidine, f5C 5-formylcytidine, m4C N4-methylcytidine, hm5C 5-hydroxymethylcytidine, f5Cm 5-formyl-2′-O-methylcytidine, m1G 1-methylguanosine, m2G N2-methylguanosine, m7G 7-methylguanosine, Gm 2′-O-methylguanosine, m2 2G N2,N2-dimethylguanosine, Gr(p) 2′-O-ribosylguanosine (phosphate), yW wybutosine, o2yW peroxywybutosine, OHyW hydroxywybutosine, OHyW* undermodified hydroxywybutosine, imG wyosine, m2,7G N2,7-dimethylguanosine, m2,2,7G N2,N2,7-trimethylguanosine I inosine, m1I 1-methylinosine, Im 2′-O-methylinosine, Q queuosine, galQ galactosyl-queuosine, manQ mannosyl-queuosine, Ψ pseudouridine, D dihydrouridine, m5U 5-methyluridine, Um 2′-O-methyluridine, m5Um 5,2′-O-dimethyluridine, m1Ψ 1-methylpseudouridine, Ψm 2′-O-methylpseudouridine, s2U 2-thiouridine, ho5U 5-hydroxyuridine, chm5U 5-(carboxyhydroxymethyl)uridine, mchm5U 5-(carboxyhydroxymethyl)uridine, methyl ester mcm5U 5-methoxycarbonylmethyluridine, mcm5Um 5-methoxycarbonylmethyl-2′-O-methyluridine, mcm5s2U 5-methoxycarbonylmethyl-2-thiouridine, ncm5U 5-carbamoylmethyluridine, ncm5Um 5-carbamoylmethyl-2′-O-methyluridine, cmnm5U 5-carboxymethylaminomethyluridine, m3U 3-methyluridine, m1acp3Ψ 1-methyl-3-(3-amino-3-carboxypropyl) pseudouridine, cm5U 5-carboxymethyluridine, m3Um 3,2′-O-dimethyluridine, m5D 5-methyldihydrouridine,™5U 5-taurinomethyluridine, τm5s2U 5-taurinomethyl-2-thiouridine, 2-Aminoadenosine, 2-Amino-6-chloropurineriboside, 8-Azaadenosine, 6-Chloropurineriboside, 5-Iodocytidine, 5-Iodouridine, Inosine, 2′-O-Methylinosine, Xanthosine, 4-Thiouridine, 06-Methylguanosine, 5,6-Dihydrouridine, 2-Thiocytidine, 6-Azacytidine, 6-Azauridine, 2′-O-Methyl-2-aminoadenosine, 2′-O-Methylpseudouridine, N1-Methyladenosine, 2′-O-Methyl-5-methyluridine, 7-Deazaguanosine, 8-Azidoadenosine, 5-Bromocytidine, 5-Bromouridine, 7-Deazaadenosine, 5-Aminoallyluridine, 5-Aminoallylcytidine, 8-Oxoguanosine, 2-Aminopurine-riboside, Pseudoisocytidine, N1-Methylpseudouridine, 5,6-Dihydro-5-Methyluridine, N6-Methyl-2-Aminoadenosine, 5-Carboxycytidine, 5-Hydroxymethyluridine, Thienoguanosine, 5-Hydroxycytidine, 5-Formyluridine, 5-Carboxyuridine, 5-Methoxyuridine, 5-Methoxycytidine, Thienouridine, 5-Carboxymethylesteruridine, Thienocytidine, 8-Oxoadenoosine, Isoguanosine, N1-Ethylpseudouridine, N1-Methyl-2′-O-Methylpseudouridine, N1-Methoxymethylpseudouridine, N1-Propylpseudouridine, 2′-O-Methyl-N6-Methyladenosine, 2-Amino-6-Cl-purine-2′-deoxyriboside, 2-Amino-2′-deoxyadenosine, 2-Aminopurine-2′-deoxyriboside, 5-Bromo-2′-deoxycytidine, 5-Bromo-2′-deoxyuridine, 6-Chloropurine-2′-deoxyriboside, 7-Deaza-2′-deoxyadenosine, 7-Deaza-2′-deoxyguanosine, 2′-Deoxyinosine, 5-Propynyl-2′-deoxycytidine, 5-Propynyl-2′-deoxyuridine, 5-Fluoro-2′-deoxyuridine, 5-Iodo-2′-deoxycytidine, 5-Iodo-2′-deoxyuridine, N6-Methyl-2′-deoxyadenosine, 5-Methyl-2′-deoxycytidine, 06-Methyl-2′-deoxyguanosine, N2-Methyl-2′-deoxyguanosine, 8-Oxo-2′-deoxyadenosine, 8-Oxo-2′-deoxyguanosine, 2-Thiothymidine, 2′-Deoxy-P-nucleoside, 5-Hydroxy-2′-deoxycytidine, 4-Thiothymidine, 2-Thio-2′-deoxycytidine, 6-Aza-2′-deoxyuridine, 6-Thio-2′-deoxyguanosine, 8-Chloro-2′-deoxyadenosine, 5-Aminoallyl-2′-deoxycytidine, 5-Aminoallyl-2′-deoxyuridine, N4-Methyl-2′-deoxycytidine, 2′-Deoxyzebularine, 5-Hydroxymethyl-2′-deoxyuridine, 5-Hydroxymethyl-2′-deoxycytidine, 5-Propargylamino-2′-deoxycytidine, 5-Propargylamino-2′-deoxyuridine, 5-Carboxy-2′-deoxycytidine, 5-Formyl-2′-deoxycytidine, 5-[(3-Indolyl)propionamide-N-allyl]-2′-deoxyuridine, 5-Carboxy-2′-deoxyuridine, 5-Formyl-2′-deoxyuridine, 7-Deaza-7-Propargylamino-2′-deoxyadenosine, 7-Deaza-7-Propargylamino-2′-deoxyguanosine, Biotin-16-Aminoallyl-2′-dUTP, Biotin-16-Aminoallyl-2′-dCTP, Biotin-16-Aminoallylcytidine, N4-Biotin-OBEA-2′-deoxycytidine, Biotin-16-Aminoallyluridine, Dabcyl-5-3-Aminoallyl-2′-dUTP, Desthiobiotin-6-Aminoallyl-2′-deoxycytidine, Desthiobiotin-16-Aminoallyl-Uridine, Biotin-16-7-Deaza-7-Propargylamino-2′-deoxyguanosine, Cyanine 3-5-Propargylamino-2′-deoxycytidine, Cyanine 3-6-Propargylamino-2′-deoxyuridine, Cyanine 5-6-Propargylamino-2′-deoxycytidine, Cyanine 5-6-Propargylamino-2′-deoxyuridine, Cyanine 3-Aminoallylcytidine, Cyanine 3-Aminoallyluridine, Cyanine 5-Aminoallylcytidine, Cyanine 5-Aminoallyluridine, Cyanine 7-Aminoallyluridine, 2′-Fluoro-2′-deoxyadenosine, 2′-Fluoro-2′-deoxycytidine, 2′-Fluoro-2′-deoxyguanosine, 2′-Fluoro-2′-deoxyuridine, 2′-O-Methyladenosine, 2′-O-Methylcytidine, 2′-O-Methylguanosine, 2′-O-Methyluridine, Puromycin, 2′-Amino-2′-deoxycytidine, 2′-Amino-2′-deoxyuridine, 2′-Azido-2′-deoxycytidine, 2′-Azido-2′-deoxyuridine, Aracytidine, Arauridine, 2′-Azido-2′-deoxyadenosine, 2′-Amino-2′-deoxyadenosine, Araadenosine, 2′-Fluoro-thymidine, 3′-O-Methyladenosine, 3′-O-Methylcytidine, 3′-O-Methylguanosine, 3′-O-Methyluridine, 2′-Azido-2′-deoxyguanosine, Araguanosine, 2′-Deoxyuridine, 3′-O-(2-nitrobenzyl)-2′-Deoxyadenosine, 3′-O-(2-nitrobenzyl)-2′-Deoxyinosine, 3′-Deoxyadenosine, 3′-Deoxyguanosine, 3′-Deoxycytidine, 3′-Deoxy-5-Methyluridine, 3′-Deoxyuridine, 2′,3′-Dideoxyadenosine, 2′,3′-Dideoxyguanosine, 2′,3′-Dideoxyuridine, 2′,3′-Dideoxythymidine, 2′,3′-Dideoxycytidine, 3′-Azido-2′,3′-dideoxyadenosine, 3′-Azido-2′,3′-dideoxythymidine, 3′-Amino-2′,3′-dideoxyadenosine, 3′-Amino-2′,3′-dideoxycytidine, 3′-Amino-2′,3′-dideoxyguanosine, 3′-Amino-2′,3′-dideoxythymidine, 3′-Azido-2′,3′-dideoxycytidine, 3′-Azido-2′,3′-dideoxyuridine, 5-Bromo-2′,3′-dideoxyuridine, 2′,3′-Dideoxyinosine, 2′-Deoxyadenosine-5′-O-(1-Thiophosphate), 2′-Deoxycytidine-5′-O-(1-Thiophosphate), 2′-Deoxyguanosine-5′-O-(1-Thiotriphosphate), 2′-Deoxythymidine-5′-O-(1-Thiophosphate), Adenosine-5′-O-(1-Thiophosphate), Cytidine-5′-O-(1-Thiophosphate), Guanosine-5′-O-(1-Thiophosphate), Uridine-5′-O-(1-Thiophosphate), 2′,3′-Dideoxyadenosine-5′-O-(1-Thiophosphate), 2′,3′-Dideoxycytidine-5′-O-(1-Thiophosphate), 2′,3′-Dideoxyguanosine-5′-O-(1-Thiophosphate), 3′-Deoxythymidine-5′-O-(1-Thiophosphate), 3′-Azido-2′,3′-dideoxythymidine-5′-O-(1-Thiophosphate), 2′,3′-Dideoxyuridine-5′-O-(1-Thiophosphate), 2′-Deoxyadenosine-5′-O-(1-Boranophosphate), 2′-Deoxycytidine-5′-O-(1-Boranophosphate), 2′-Deoxyguanosine-5′-O-(1-Boranophosphate), and 2′-Deoxythymidine-5′-O-(1-Boranophosphate).

In some embodiments of the compositions of the disclosure, including those in which the delivery vehicle is a lipid nanoparticle (LNP), the delivery vehicle comprises the RNA encoding TERT. In some embodiments, one or more of a surface, a layer or a volume of the delivery vehicle comprises the RNA encoding TERT. In some embodiments, the surface comprises an outer surface or an inner surface. In some embodiments, the layer comprises a lipid monolayer or lipid bi-layer. In some embodiments, the volume comprises an internal volume.

In some embodiments, the disclosure provides a composition comprising a (i) a ribonucleic acid (RNA) encoding telomerase reverse transcriptase (TERT) and (ii) a delivery vehicle, wherein the RNA of (i) comprises one or more modified nucleotides and wherein the delivery vehicle of (ii) is operably-linked to the RNA of (i).

In some embodiments of the compositions of the disclosure, including those in which the delivery vehicle is a lipid nanoparticle (LNP), the composition further comprises a ribonucleic acid (RNA) encoding TElomerase RNA Component (TERC). In some embodiments, the delivery vehicle is operably-linked to a ribonucleic acid (RNA) encoding TElomerase RNA Component (TERC). In some embodiments, the delivery vehicle comprises the RNA encoding TERC. In some embodiments, one or more of a surface, a layer or a volume of the delivery vehicle comprises the RNA encoding TERC. In some embodiments, the surface comprises an outer surface or an inner surface. In some embodiments, the layer comprises a lipid monolayer or lipid bi-layer. In some embodiments, the volume comprises an internal volume.

In some embodiments the RNA encoding TERT comprises a sequence with at least 70%, at least 75%, at least 80%, at least 85%, at least 90%, at least 95%, at least 97%, at least 99%, or 100% sequence identity to any one of SEQ ID NOS: 1-5, 7, 9, 14-17, 19, 21, 23, 25, 27, 29-31, 37-40. In some embodiments, the RNA encoding TERT comprises a UTR sequence, optionally a UTR sequence with at least 70%, at least 75%, at least 80%, at least 85%, at least 90%, at least 95%, at least 97%, at least 99%, or 100% sequence identity to any one of SEQ ID NOS: 32-34, 35, and 36.

In some embodiments, the RNA comprises a self-replicating RNA. In some embodiments, the RNA comprises a circular RNA.

The disclosure provides a method of increasing telomerase activity in a cell, the method comprising contacting the cell and the composition of the disclosure. In some embodiments, the cell is in vivo, ex vivo or in vitro.

The disclosure provides a method of extending telomeres in a cell, the method comprising contacting the cell and the composition of the disclosure. In some embodiments, the cell is in vivo, ex vivo or in vitro.

The disclosure provides a cell comprising the composition of the disclosure.

The disclosure provides a formulation comprising the cell of the disclosure, which comprises a composition of the disclosure. In some embodiments of the formulation, a plurality of cells comprises a cell of the disclosure, which comprises a composition of the disclosure. In some embodiments of the formulation, each cell of the plurality is a cell of the disclosure, which comprises a composition of the disclosure.

The disclosure provides a method of treating a disease or disorder comprising administering to a subject an effective amount of a composition of the disclosure.

The disclosure provides a method of treating a disease or disorder comprising administering to a subject an effective amount of a cell of the disclosure, which comprises a composition of the disclosure.

The disclosure provides a method of treating a disease or disorder comprising administering to a subject an effective amount of a formulation of the disclosure.

The disclosure provides a method of delaying the onset of a disease comprising administering to a subject an effective amount of a composition of the disclosure.

The disclosure provides a method of delaying the onset of a disease comprising administering to a subject an effective amount of a cell of the disclosure, which comprises a composition of the disclosure.

The disclosure provides a method of delaying the onset of a disease comprising administering to a subject an effective amount of a formulation of the disclosure.

In some embodiments, the disclosure provides a method of treating a fibrotic disease in a subject in need thereof, comprising: administering to the subject an effective amount of a composition comprising one or more synthetic messenger ribonucleic acids (mRNAs) encoding telomerase reverse transcriptase (TERT).

In some embodiments of the method, the composition comprises a delivery vehicle, optionally wherein the delivery vehicle is a nanoparticle, optionally a lipid nanoparticle (LNP). In some embodiments, the LNP comprises an ionizable lipid, a phospholipid, a cholesterol, and/or a PEGylated lipid.

In some embodiments, the LNP comprises a molar ratio of about 50 to about 60 moles of an ionizable lipid, to about 4 to about 6 moles of a phospholipid, about 35 to about 45 moles of cholesterol, and about 1.0 to about 2.0 moles of PEGylated lipid.

In some embodiments, the LNP comprises a molar ratio of about 30 to 40 moles of an ionizable lipid, to about 14 to about 18 moles of a phospholipid, about 40 to about 50 moles of a cholesterol, and about 2.0 to about 3.0 moles of a PEGylated lipid.

In some embodiments, the LNP comprises a molar ratio of about 55 moles of an ionizable lipid, to about 5 moles of a phospholipid, about 40 moles of a cholesterol, and about 1.5 moles of a PEGylated lipid.

In some embodiments, the LNP comprises a molar ratio of about 52.5 moles of an ionizable lipid, to about 7.5 moles of a phospholipid, about 40 moles of a cholesterol, and about 1.5 moles of a PEGylated lipid.

In some embodiments, the TERT synthetic mRNA comprises at least one modified nucleoside from the list in Table 1B. In some embodiments, the modified nucleoside is pseudouridine or a pseudouridine analog, optionally wherein the pseudouridine analog is N-1-methylpseudouridine. In some embodiments, the modified nucleoside is 5-methoxyuridine.

In some embodiments, the TERT synthetic mRNA comprises an untranslated region (UTR). In some embodiments the UTR comprises a sequence at least 70%, at least 75%, at least 80%, at least 85%, at least 90%, at least 95%, at least 97%, at least 99%, or 100% identical to any one of SEQ ID NOS: 32-36.

In some embodiments, the TERT synthetic mRNA comprises a 5′ cap structure, wherein the 5′ cap structure is IRES, Cap0, Cap1, ARCA, inosine, N1-methyl-guanosine, 2′fluoro-guanosine, 7-deaza-guanosine, CleanCap™, m7(3′OMeG)(5′)ppp(5′)(2′OMeA)pG, 8-oxo-guanosine, 2-amino-guanosine, LNA-guanosine, 2-azido-guanosine, Cap2, Cap4, CAP-003, or CAP-225.

In some embodiments, the TERT synthetic mRNA comprises a poly-adenosine (poly-A) nucleotide sequence 3′ to the encoding region.

In some embodiments, the TERT synthetic mRNA comprises a chain terminating nucleotide, wherein the nucleotide is 3′-deoxyadenosine (cordycepin), 3′-deoxyuridine, 3′-deoxycytosine, 3′-deoxyguanosine, 3′-deoxythymine, 2′,3′-dideoxynucleosides, 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or —O— methylnucleoside.

In some embodiments, the TERT synthetic mRNA is codon optimized. In some embodiments, the TERT synthetic mRNA comprises at least 70%, at least 75%, at least 80%, at least 85%, at least 90%, at least 95%, at least 97%, at least 99%, or 100% identical to any one of SEQ ID NOS: 1, 2, 7, 9, 30, 39, or 40.

In some embodiments, the delivery vehicle is a liposome, an ionizable lipid, an extracellular vesicle, or an exosome. In some embodiments the delivery vehicle is an extracellular vesicle of an exosome, optionally wherein the extracellular vesicle or exosome comprises a targeting moiety of one or more of a lipid, a peptide, or an antibody

In some embodiments, the method reduces fibrosis.

In some embodiments, the subject is human.

In some embodiments, the disclosure describes a composition for use. In some embodiments the composition for use is a pharmaceutical composition comprising one or more pharmaceutically acceptable solvents or excipients.

In some embodiments, the disclosure provides a kit for treating a fibrotic disease in a subject, comprising a composition and instructions for use thereof.

In some embodiments, the disclosure provides a method of treating a liver disease in a subject in need thereof, comprising administering to the subject a composition comprising one or more synthetic messenger ribonucleic acids (mRNAs) encoding telomerase reverse transcriptase (TERT).

In some embodiments, the method reduces liver fibrosis. In some embodiments, the liver disease is non-alcoholic steatohepatitis (NASH) or non-alcoholic fatty liver disease (NAFLD).

In some embodiments the liver disease is alcoholic hepatitis, liver cirrhosis, liver fibrosis, compensated cirrhosis, decompensated cirrhosis, acute-on-chronic liver failure, fibrotic stage F4 Non-alcoholic steatohepatitis (NASH), biliary atresia, primary biliary cirrhosis, primary sclerosing cholangitis, and/or chronic liver disease, hemochromatosis, Wilson's disease, or ischemic hepatitis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a schematic illustrating long-term therapeutic benefit from transient, rapid telomere extension via telomerase reverse transcriptase (TERT) mRNA. In particular, the speed of telomere extension made possible by TERT mRNA treatment enables telomere maintenance by very infrequent TERT mRNA dosing. The telomerase activity resulting from TERT mRNA delivery rapidly extends telomeres in a brief period, before the mRNA is turned over, thus allowing the protective anti-cancer mechanism of telomere-shortening to function most of the time. Between treatments, normal telomerase activity and telomere shortening is present, and therefore the anti-cancer safety mechanism of telomere shortening to prevent out-of-control proliferation remains intact, while the risk of short telomere-related disease remains low. In contrast, the best existing small molecule treatment for extending telomeres requires chronic delivery, and thus presents a chronic cancer risk, and even then has a small, inconsistent effect on telomere length, with no detectable effect on telomere length at all in about half of patients.

FIG. 2 is a series of graphs showing that TERT mRNA LNPs exhibit low toxicity by liver panel. Mice were dosed intravenously with reporter mRNA encapsulated in a lipid nanoparticle employing either LNP1 or LNP2, as shown in Table 5 (N=1-4 per condition). Mice were sacrificed and blood was collected at the time points indicated (12, 24, and 72 hours). Mice receiving saline (N=4) and carbon tetrachloride (CCl4, N=4) served as negative and positive controls, respectively. Error bars display standard error of the mean.

FIG. 3 is a series of photographs showing that TERT mRNA LNPs cause normal histology. Cre mRNA was encapsulated into LNP 1 (Table 5) and delivered intravenously (i.v.) into tdTomato fl/fl mice. Organs were harvested 72 hours later, fixed, paraffin embedded, and sectioned. Organs from an untreated tdTomato fl/fl mouse are shown for reference.

FIG. 4 is a series of photographs showing that TERT mRNA LNPs transfect hepatocytes with high efficiency. Cre mRNA was encapsulated into LNP 1 (Table 5) and delivered intravenously (i.v.) into tdTomato fl/fl mice. Organs were harvested 72 hours later, fixed, paraffin embedded, and sectioned. Photographs depict immunohistochemistry (IHC) with anti-tdTomato. Organs from an untreated tdTomato fl/fl mouse are shown for reference.

FIG. 5 is a series of photographs showing that TERT mRNA LNPs also target some cells in spleen. Cre mRNA was encapsulated into LNP 1 (Table 5) and delivered intravenously (i.v.) into tdTomato fl/fl mice. Organs were harvested 72 hours later, fixed, paraffin embedded, and sectioned. Photographs depict immunohistochemistry (IHC) with anti-tdTomato. Organs from an untreated tdTomato fl/fl mouse are shown for reference.

FIG. 6 is a pair of graphs showing that TERT mRNA LNPs cause high telomerase activity in liver. Tert mRNA was formulated with LNP1 or LNP2 (Table 5) and delivered to i.v into TERT KO mice. 20 hours later, the livers were harvested for telomerase repeat amplification protocol (TRAP). Wild-type C57B16/J and untreated TERT KO mouse livers were used as positive and negative controls, respectively.

FIG. 7 is a photograph depicting the results of an assaying demonstrating that luciferase mRNA LNPs cause high bioluminescence signal in liver. Various LNPs designated as Lipid Nanoparticle 1 (LNP1), Lipid Nanoparticle 2 (LNP2), or Lipid Nanoparticle 3 (LNP3) (Table 5). Empty LNP shown as a negative control (ctrl). Luciferase mRNA was formulated with LNP 1, 2, and 3 and delivered via IV injection into C57B16/J mice. 20 hours later, these mice were shaved and imaged after injection with luciferin using the IVIS BLI system.

FIG. 8 is a graph and a series of photographs of a first study demonstrating that TERT LNPs reduce fibrosis in thioacetamide (TAA) drinking water model. The addition of thioacetamide (TAA) to drinking water represents an art-recognized model for the induction of experimental liver fibrosis in rodents (Wallace M C, Hamesch K, Lunova M, et al. Standard operating procedures in experimental liver research: thioacetamide model in mice and rats. Lab Anim 2015; 49:21-9). In this experiment, TERT KO mice received 0.3 g/L TAA in their drinking water for 9.5 weeks. Mice were treated with LNPs carrying either Tert mRNA or Luciferase (LUC) mRNA once weekly. Liver sections were stained with Picrosirius red (PSR), and a quantification of showed a 24% mean reduction in PSR stained tissue in mice treated with TERT LNPs compared to those treated with LUC LNPs. Scale bar on photographs equals 500 μm.

FIGS. 9A AND 9B are graphs and photographs of a study demonstrating that TERT LNPs Reduce Fibrosis in a Thioacetamide (TAA) Drinking Water Model. TERT KO mice received 0.3 g/L TAA in their drinking water for 9.4 weeks and were treated with TERT or LUC mRNA-LNPs once weekly. By picrosirius red (PSR) staining, there was an 18% mean reduction in fibrosis in female mice and a 37% mean reduction was observed in males treated with TERT mRNA-LNPs, representing a significant (p=0.041) reduction in fibrosis. The scale bar on photographs is 500 μm. Additionally, using the 0 through 4 scoring system developed by the Pathology Committee of the NASH Clinical Research Network (Kleiner et al Hepatology 2005), animals treated with TERT mRNA LNPs had a significant reduction in fibrosis compared to control animals treated with LUC (luciferase) mRNA LNPs (p=0.032) as seen in FIG. 9B. For all scoring, liver fibrosis was scored independently for each of 3 lobes per mouse (right, median, and left) in a blinded manner. The scores were averaged together to get a score per mouse, which were then plotted in the graphs (FIGS. 8, 9A, and 9B).

FIGS. 10A AND 10B are graphs demonstrating that TERT mRNA improves survival. Survival plotted as fraction of mice alive as a function of days post first dose of either TERT or a Luciferase (Luc) negative control. Same experimental procedure was followed as described in FIG. 9, except that the mice were 4^{th} generation (G4) TERT KOs aged to over 30 weeks at the start of the study. These mice were dosed once weekly with TERT or LUC, and survival was recorded after the first dose. TERT treated mice showed a 42% increase in median survival and a 58% increase in maximal survival. FIG. 10B shows the survival of a group of aged mice that did not receive thioacetamide (TAA) in drinking water (no TAA).

FIGS. 11A AND 11B show the effects of TERT mRNA on pathological liver fibrosis in mice. FIG. 11A shows the assessment of pathological fibrosis in liver sections from TERT knockout mice with TAA-induced liver fibrosis, as described in FIG. 9, by a certified pathologist based on the Non-alcoholic Fatty Liver Disease (NAFLD) Activity Score (NAS). TERT knockout mice without TAA-induced liver fibrosis (saline IV) exhibited a mild inflammation score of 1 (<2 foci per 200× field of view). Untreated control mice (C57Bl/6 strain) exhibited no detectable inflammation. FIG. 11B shows inflammation was significantly reduced in TERT knockout mice treated with TERT mRNA compared to the control (LUC): TERT mRNA treatment resulted in a 60% reduction in the number of animals with a score of >1.

FIGS. 12A AND 12B show the transfection efficiency of mRNA in liver. FIG. 12A shows the quantification of percent (%) positive hepatocytes after different doses of Cre mRNA encapsulated in lipid nanoparticle using ionizable lipid 1 (LNP1) delivered intravenously to tdTomato fl/fl (HTT flox/flox) mice. Hepatocytes were identified in liver tissue sections using nuclear size and circularity by QuPath software. The experimental design was the same as for FIGS. 3-5. FIG. 12B is representative images of immunohistochemistry (IHC) using an anti-tdTomato antibody in liver sections.

FIG. 13 shows levels of liver damage makers following TERT mRNA delivery. TERT mRNA was formulated with LNP1 or D-Lin-MC3-DMA (MC3) and delivered intravenously into C57B16 mice at 0.6 mg/kg. 24 hours later, the plasma was taken for measurement of alanine aminotransferase (ALT) and aspartate aminotransferase (AST). TERT-LNP1 had equivalent or lower levels of ALT and AST compared to MC3. These liver enzymes serve as markers of acute toxicity.

FIGS. 14 AND 14B show the transfection efficiency in fibrotic liver. FIG. 14 A shows the quantification of percent (%) positive hepatocytes following delivery of Cre mRNA encapsulated in lipid nanoparticle using LNP1 to tdTomato fl/fl mice after 16 weeks of treatment with thioacetamide (TAA) in drinking water at 0.3 g/L. Hepatocytes were identified using nuclear size and circularity by QuPath software. The experimental design was the same as for FIGS. 3-5. FIG. 12B: is representative immunohistochemical (IHC) images using an anti-tdTomato antibody in liver sections.

FIGS. 15A AND 15B show telomere extension in liver. The experimental design is as follows: 3 doses of either TERT mRNA (SEQ ID NO: 37) or luciferase (LUC) mRNA formulated with LNP1 were delivered to TERT KO mice intravenously once weekly at 0.5 mg/kg. The mRNA-LNP dosing was preceded two days prior by a dose of thioacetamide intraperitoneally (i.p.) at 50 mg/kg. Mice were harvested 1 week after the final dose of mRNA-LNP. Telomere length was quantified in hepatocytes using Q-FISH. Liver tissue was fixed, sectioned, and stained with TelC fluorescent probe that labels the telomeres. Individual telomere fluorescence was quantified on a per cell basis and the average was taken for each mouse. The median fluorescence is shown in FIG. 15A and 10th percentile fluorescence is shown in FIG. 15B. Each point represents a single mouse. Hepatocytes in mice treated with TERT mRNA had significantly longer telomeres than LUC mRNA treated control animals. At least 300 cells were analyzed per mouse per treatment group.

FIGS. 16A AND 16B show telomerase activity in human hepatocytes. Human hepatocytes from a 51-year-old donor cultured were transfected with green fluorescent protein (GFP) mRNA or TERT mRNA using Messenger Max from Thermo Scientific at 1 μg/ml. Cells were harvested for each time point indicated in FIG. 16A for the TRAP assay to measure telomerase activity. Telomerase activity produces a ladder pattern that was detected using an Agilent Bioanalyzer. Telomerase activity returned to baseline by day 14.

FIGS. 17A AND 17B show that telomere length was quantified from human hepatocytes from a 51-year-old donor. In the experimental design human hepatocytes were cultured on glass coverslips. Cells were transfected once with TERT mRNA at 1 μg/ml using Messenger Max from Thermo Scientific or left untreated (UT). Cells were fixed and stained with TelC fluorescent probe using the Q-FISH protocol. Individual telomere fluorescence was quantified on a per cell level. The mean fluorescence is shown in FIG. 17A and the 10th percentile fluorescence is shown in FIG. 17B. At least 150 cells were analyzed per treatment group.

FIGS. 18A AND 18B show TERT mRNA (SEQ ID NO: 40) formulated with LNP1 and imaged at high resolution using the Thermo Scientific Talos Glacios Cryo transmission electron microscope (TEM) at 34,000 magnification and 200 kv voltage. A representative image is shown in FIG. 18A; the TEM copper grid is the dark region on the right. The particle size was characterized using dynamic light scattering (DLS) using a Brookhaven 90Plus Particle Analyzer as shown in FIG. 18B.

FIG. 19 shows results of the telomerase activity assay “telomerase repeat amplification protocol” (TRAP) in human fibroblasts treated for 24 hours with 1 μg/ml TERT mRNAs of from left to right, untreated cells, SEQ ID NOS:39, 40, 1, 2, 31, 3, 5, and 4 respectively, and a GFP mRNA control. Telomerase activity is indicated by a characteristic ladder pattern as shown by the transfection of TERT mRNAs of SEQ ID NOS: 39, 40, 1, 2, 31, 3, 5, and 4 to varying degrees. The samples transfected with human TERT mRNA showed higher levels of telomerase activity than the samples transfected with mouse TERT mRNA. Untreated and GFP mRNA samples did exhibit telomerase activity.

FIG. 20 shows in vivo delivery of mRNA in a photograph depicting the results demonstrating that luciferase mRNA LNPs cause high bioluminescence signal in liver. Luciferase mRNA was formulated with SS-OP using the lipid ratios for LNP1, as shown in Table 5. The lipid:mRNA ratios (wt/wt) were varied. The formulated mRNA LNPs were delivered via IV injection into C57B16/J mice at 0.6 mg/kg. As a negative control, a mouse was injected with saline. 24 hours later, these mice were shaved and imaged after injection with luciferin using the Lago instrument from Spectral Instruments Imaging. Depicted is an BLI image from mice dosed with lipid:mRNA ratios of 175, 42, and 25. The signal was highest in the mice receiving LNPs with a lipid:mRNA ratio (wt/wt) of 175 and 42. The other data presented here using LNP1 uses a wt/wt ratio of 42.

FIG. 21 shows in vivo delivery of mRNA in a photograph depicting the results demonstrating that luciferase mRNA LNPs causing high bioluminescence signal in liver. LNPs designated as Lipid Nanoparticle 4 (LNP4) or Lipid Nanoparticle 5 (LNP5) were formulated using the recipe in Table 5 with luciferase mRNA. These LNPs were delivered via IV injection into C57B16/J mice at 0.6 mg/kg. As a negative control, a mouse was injected with saline. 20 hours later, these mice were shaved and imaged after injection with luciferin using the Lago instrument from Spectral Instruments Imaging. LNP4 consisted of the formula for LNP2, but with SS-OP substituted for cKK-E12. LNP5 consisted of the formula for LNP1, but with cKK-E12 substituted for SS-OP. Bioluminescent imaging indicates that both of these LNPs had successful delivery to the liver.

FIG. 22 shows in vivo delivery of mRNA in a photograph depicting the results demonstrating that luciferase mRNA LNPs causing high bioluminescence signal in liver. Luciferase mRNA was formulated with lipids per the recipe for LNP1 in Table 5. The ingredient that was varied was the molar ratio of DMG-PEG2000. As shown in FIG. 22, DMG-PEG2000 was added as either 1, 1.5, 2, or 3 parts relative to the molar sum of all lipids, while the molar ratio for the other 3 lipids is held constant. This corresponds to a molar percentage for DMG-PEG2000 of approximately 1.0%, 1.5%, 2.0%, and 2.9%, 20 hours after intravenous delivery at 0.6 mg/kg, the C57BL/6J mice were shaved and imaged following luciferin injection using the Lago instrument from Spectral Instruments Imaging. The signal was strong from all of the mice receiving active Luciferase mRNA LNPs, and the best signal was seen when DMG-PEG2000 was added in a molar ratio of 1.5:101.5 for a total of (˜1.5%). The other data presented here uses LNP1 with this molar ratio of DMG-PEG2000.

FIG. 23 is a capillary electrophoresis gel image showing that TERT mRNA LNPs cause high telomerase activity in liver. Tert mRNA (mTert SEQ 37) was formulated with LNP3, a lipid nanoparticle containing DLin-MC3-DMA (Table 5) and delivered i.v into TERT KO mice at 0.6 mg/kg. 16 hours or 8 days later (as indicated in the image), the livers were harvested for telomerase repeat amplification protocol (TRAP). The negative control was a TRAP performed on a liver from a TERT KO mouse that was injected with saline. Livers from mice treated with TERT mRNA LNP3 exhibit elevated telomerase activity which returns to baseline levels, indicating the increase in telomerase activity was transient.

### DETAILED DESCRIPTION

Provided herein are compositions and methods that may be used for preventing or treating fibrotic diseases and liver diseases or disorders. The present disclosure describes the surprising result that compositions comprising an mRNA encoding telomerase reverse transcriptase (TERT) reduce liver fibrosis. TERT mRNA therapies as described herein may be delivered in lipid nanoparticles (LNPs), or by other delivery vehicles. Diseases that may be treated include, without limitation, fibrotic diseases, e.g. of the liver, and other liver diseases.

Telomerase reverse transcriptase (TERT) is an enzyme known to maintain and extend chromosomal ends (telomeres). The TERT enzyme is a catalytic subunit of the ribonucleoprotein telomerase. TERT adds simple sequence repeats to telomeres by copying a template sequence 5′-GGTTAG-3′ within the RNA component of telomerase. This addition of repetitive deoxyribonucleic acid (DNA) sequences helps slow telomere shortening, which occurs over time, e.g., due to incomplete DNA replication during mitosis.

TERT translocates between the nucleus and cytoplasm and has been shown to be a critical factor in a number of other biological processes, including cell proliferation and cancer metastasis. Thus, the level of TERT in the nucleus may be a critical step in regulating cell and organismal health.

Telomerase reverse transcriptase (TERT) is also known in the art as TRT, cutaneous malignant melanoma 9 (CMM9), dyskeratosis congenita autosomal dominant 2 (DKCA2), autosomal recessive dyskeratosis congenita-4 (DKCB4), human ever shorter telomeres 2 (HEST2), pulmonary fibrosis/bone marrow failure telomere related 1 (PFBMFT1), telomerase catalytic subunit (TCS1), and telomerase associated protein 2 (TP2).

In some embodiments, the treatments described herein may stop, slow, or reverse progression of a fibrotic disease, e.g., a liver disease, or other liver diseases.

TERT mRNA is transient and only requires a few hours to extend telomeres in human cells before being degraded. Therefore, TERT mRNA leaves the protective anti-cancer telomere shortening mechanism intact. The present disclosure provides compositions and methods for delivery of TERT mRNA and treatment of fibrotic diseases and liver diseases.

During normal aging, telomeres shorten by approximately 30-100 base pairs per year due to oxidation and incomplete DNA replication during S phase of the cell cycle (Kurenova E V, et al. Telomere functions. A review. Biochemistry (Mosc) 1997; 62:1242-53). Telomerase, consisting of the TERT protein and a polynucleotide template (TERC), extends telomeres, but in humans, it is inactive in most somatic cell types and is only active at low levels that are insufficient to prevent net telomere shortening in many progenitor cell types. The exception is the spermatogenic lineage, in which telomerase is active enough to maintain telomere length over the human lifespan (Takubo K, Aida J, Izumiyama-Shimomura N, et al. Changes of telomere length with aging. Geriatric Gerontology Int 2010; 10 Suppl 1:S197-206). As the TERC component is present at high levels in all cell types, typically over 10,000 copies per cell, TERT is the limiting component. Because short telomeres limit the proliferative and regenerative capacities of cells, they are associated with aging, early death, and a vast number of diseases and conditions.

Telomeres comprise repetitive DNA sequences at the ends of linear chromosomes that, when sufficiently long, can allow each chromosome end to form a loop that protects the ends from acting as double-stranded or single-stranded DNA breaks. Telomeres can shorten over time, due in part to oxidative damage and incomplete DNA replication, eventually leading to critically short telomeres unable to form the protective loop, exposure of the chromosome ends, chromosome-chromosome fusions, DNA damage responses, and cellular senescence, apoptosis, or malignancy.

Telomere length maintenance can play a role in preventing cellular senescence and apoptosis and resulting cellular and organ dysfunction. In many diseases, the need for cell replication to replace cells damaged or killed by the underlying disease mechanism shortens telomeres more rapidly than normal, exhausting the replicative capacity of cells, and leading to tissue dysfunction, exacerbated or additional symptoms, disability, or death. Further, genetic mutations of telomerase enzyme (TERT) can be linked to fatal inherited diseases of inadequate telomere maintenance.

The prospect of preventing, delaying, or treating dysfunction, conditions, and diseases by telomere extension motivates a need for safe and effective treatments to extend telomeres in animal cells in vivo and/or in vitro, and safe and effective compositions and methods for delivering therapies to the animal cells to extend telomeres. Further, there is a need to safely and rapidly extend telomeres in cells for use in cell therapy, cell and tissue engineering, and regenerative medicine. At the same time, however, there can be a danger in the constitutive, as opposed to transient, activation of telomerase activity. Indeed, for cell therapy applications, there is a need to avoid cell immortalization. To this end, transient, rather than constitutive, telomerase activity can be advantageous for safety, e.g., if the elevated telomerase activity is not only brief but extends telomeres rapidly enough that the treatment does not need to be repeated continuously.

Thus, there is need for therapies that safely extend telomeres to potentially prevent, delay, ameliorate, or treat these and other conditions and diseases, to do the same for the gradual decline in physical form and function and mental function that accompanies chronological aging, and to enable cell therapies and regenerative medicine. Furthermore, there is need for improved methods of delivering these therapies, e.g., nucleic acid molecules encoding telomerase, to cells.

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

It must be noted that, as used herein and in the appended claims, the singular forms “a,” “and,” and “the” include plural referents unless the context clearly dictates otherwise. Thus, for example, reference to “a drug candidate” refers to one or mixtures of such candidates, and reference to “the method” includes reference to equivalent steps and methods known to those skilled in the art, and so forth.

As used herein, the term “approximately” or “about,” as applied to one or more values of interest, refers to a value that is similar in magnitude and/or within a similar range to a stated reference value. In certain embodiments, the term “approximately” or “about” may refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

“G,” “C,” “A,” “T” and “U” generally stand for the bases, guanine, cytosine, adenine, thymidine and uracil, respectively. Nucleobases can form nucleosides by the addition of a five carbon sugar. If the sugar is ribose then the nucleoside is a ribonucleoside. Nucleosides can in turn form nucleotides by the addition of one or more linker groups such as phosphate groups. Nucleotides can in turn form polymers (polynucleotides) which include short polymers (oligonucleotides). However, it will be understood that the terms “base”, “nucleobase”, “nucleoside”, “ribonucleoside”, “nucleotide”, “ribonucleotide” can also refer to a modified base, nucleobase, nucleoside, ribonucleoside, nucleotide, or ribonucleotide, as further detailed below, or a surrogate replacement moiety (see, e.g., Table 1B and elsewhere herein). The skilled person is well aware that guanine, cytosine, adenine, thymidine, uracil can be replaced by other moieties without substantially impairing one or more of certain properties (such as base pairing properties, translatability, or protein binding properties) of an oligonucleotide or polynucleotide comprising a nucleotide bearing such replacement moiety. Sequences containing such replacement moieties are suitable for the compositions and methods featured in the disclosure. Similarly, the skilled person is well aware that ribose can be replaced with other moieties without impairing certain properties (such as base pairing properties, translatability, or protein binding properties) of an oligonucleotide or polynucleotide comprising a nucleotide bearing such replacement moiety. Sequences containing such replacement moieties are suitable for the compositions and methods featured in the disclosure. Similarly, the skilled person is well aware that phosphate can be replaced with other moieties without impairing certain properties (such as base pairing properties, translatability, or protein binding properties) of an oligonucleotide or polynucleotide comprising a nucleotide bearing such replacement moiety. Sequences containing such replacement moieties are suitable for the compositions and methods featured in the disclosure.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

As used herein, the terms “identity” and “identical” refer, with respect to a polypeptide or polynucleotide sequence-of-interest, to the percentage of exact matching residues in an alignment of that the sequence-of-interest to a reference sequence, such as an alignment generated by the BLAST algorithm. Identity is calculated, unless specified otherwise, across the full length of the reference sequence. Thus a sequence-of-interest “shares at least x % identity to” a reference sequence if, when the reference sequence is aligned (as a query sequence) is aligned to the sequence-of-interest (as subject sequence), at least x % (rounded down) of the residues in the subject sequence are aligned as an exact match to a corresponding residue in the query sequence, the denominator being the full length of the reference sequence plus the lengths of any gaps inserted into the reference sequence by alignment of the reference sequence to the sequence-of-interest. Where the subject sequence has variable positions (e.g., residues denoted X), an alignment to any residue in the query sequence is counted as a match. Sequence alignments may be performed using the NCBI Blast service (BLAST+ version 2.12.0) or another program giving the same results.

The term “native” or “wild-type” as used herein refers to a nucleotide sequence, e.g. gene, or gene product, e.g. RNA or polypeptide, that is present in a wild-type cell, tissue, organ or organism. The term “variant” as used herein refers to a mutant of a reference polynucleotide or polypeptide sequence, for example a native polynucleotide or polypeptide sequence, i.e., having less than 100% sequence identity with the reference polynucleotide or polypeptide sequence. Put another way, a variant comprises at least one nucleotide difference (e.g., nucleotide substitution, nucleotide insertion, nucleotide deletion) or one amino acid difference (e.g., amino acid substitution, amino acid insertion, amino acid deletion) relative to a reference polynucleotide sequence, e.g. a native polynucleotide or polypeptide sequence. For example, a variant may be a polynucleotide having a sequence identity of 50% or more, 60% or more, or 70% or more with a full length native polynucleotide sequence, e.g. an identity of 75% or 80% or more, such as 85%, 90%, or 95% or more, for example, 98% or 99% identity with the full length native polynucleotide sequence. As another example, a variant may be a polypeptide having a sequence identity of 70% or more with a full length native polypeptide sequence, e.g. an identity of 75% or 80% or more, such as 85%, 90%, or 95% or more, for example, 98% or 99% identity with the full length native polypeptide sequence. Variants may also include variant fragments of a reference, e.g. native, sequence sharing a sequence identity of 70% or more with a fragment of the reference, e.g. native, sequence, e.g. an identity of 75% or 80% or more, such as 85%, 90%, or 95% or more, for example, 98% or 99% identity with the native sequence.

As used herein, the term “codon optimized” refers to any process used to improve gene expression and increase the translational efficiency of a gene of interest by accommodating the codon bias of the host organism, and/or to reduce the immunogenicity of the polynucleotide.

The terms “treating” or “treatment” are used herein to generally mean obtaining a desired pharmacologic and/or physiologic effect with a therapeutic agent. The effect may be prophylactic in terms of completely or partially preventing a disease or symptom thereof, e.g. reducing the likelihood that the disease or symptom thereof occurs in the subject, and/or may be therapeutic in terms of completely or partially reducing a symptom, or a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting or slowing the onset or development of the disease; or (c) relieving the disease, e.g., causing regression of the disease or symptoms associated with the disease. The therapeutic agent may be administered before, during or after the onset of disease. The treatment of ongoing disease, where the treatment stabilizes or reduces the undesirable clinical symptoms of the patient, may be of particular interest. In some embodiments, treatment is performed prior to complete loss of function in the affected tissues. In some embodiments, the subject therapy will be administered before the symptomatic stage of the disease; and, in some embodiments, during the symptomatic stage of the disease; and, in some embodiments, after the symptomatic stage of the disease.

In some embodiments, therapies as described herein treat fibrotic diseases or liver diseases, including but not limited to fibrotic liver diseases. The fibrotic or liver diseases may be associated with a TERT mutation, mutation in other genes, or non-genetic causes. Diseases that may be treated using the composition and methods of the present disclosure include non-alcoholic fatty liver disease (NAFLD), non-alcoholic steatohepatitis (NASH), e.g., stage F4 NASH, alcoholic hepatitis, alcoholic liver disease, liver cirrhosis, e.g. compensated and non-compensated cirrhosis, liver fibrosis, hemochromatosis, biliary atresia, primary biliary cirrhosis, primary sclerosing cholangitis, chronic liver disease, acute-on-chronic liver failure (ACLF), Wilson's disease, or ischemic hepatitis.

The terms “individual,” “subject,” and “patient” are used interchangeably herein and refer to any subject for whom treatment or therapy is desired. The subject may be a mammalian subject. Mammalian subjects include, e.g., humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion or service animal (e.g. cats or dogs).

A subject “in need thereof,” as used herein, refers to any subject suffering from or identified to be at risk of developing a fibrotic disease or liver disease.

It is to be understood that this disclosure is not limited to the particular methodology, products, apparatus and factors described, as such methods, apparatus and formulations may vary. It is also to be understood that the terminology used herein is for the purpose of describing particular embodiments only, and it is not intended to limit the scope of the present disclosure which will be limited only by appended claims.

I. Synthetic mRNAs

A synthetic mRNA as used herein may refer to any sequence comprising a mutation (point or deletion) or additional nucleotides not found in the wild type sequence. For example, a synthetic TERT mRNA may refer to a wild type sequence encoding a human TERT sequence, flanked by the addition of 1, 2, 3, 10, 100 or more nucleotides. Similarly, the nucleotides themselves may encode amino acids distinct from the wild type, or be modified to reduce immunogenicity in the cell or tissue. An mRNA sequence in some embodiments may comprise any of the following modifications, including but not limited to an untranslated region (UTR), a 5′ cap, and a poly-adenosine tail. In some embodiments, the RNA may be circular and/or self-replicating. Illustrative methods of making circular mRNAs are provided in Chen et al. Science. 1995 Apr. 21; 268(5209):415-7; Perriman R. (2002) Circular mRNA Encoding for Monomeric and Polymeric Green Fluorescent Protein. In: Hicks B. W. (eds) Green Fluorescent Protein. Methods in Molecular Biology, vol 183. Humana Press; Wang et al. RNA. 2015 Feb; 21(2):172-9. doi: 10.1261/rna.048272.114. Epub 2014 Dec 1; Wesselhoeft et al. Nat Commun. 2018 Jul. 6; 9(1):2629; and Wesselhoeft et al. Mol Cell. 2019 May 2; 74(3):508-520.e4. Illustrative methods of making self-replicating mRNAs are provided in Tews B. A., Meyers G. (2017) Self-Replicating RNA. In: Kramps T., Elbers K. (eds) RNA Vaccines. Methods in Molecular Biology, vol 1499. Humana Press; Leyman et al. Mol Pharm. 2018 Feb. 5; 15(2):377-384; and Huysmans et al. Mol Ther Nucleic Acids. 2019 Sep. 6; 17:388-395.

TERT mRNAs

In some embodiments, a composition may comprise a reverse transcriptase telomerase (TERT) mRNA sequence to treat one or more phenotypes or symptoms associated with a fibrotic disease or liver disease. In some embodiments, a TERT mRNA refers to an mRNA encoding any full length, functional fragment or portion of a TERT protein, including wild type sequences or variants thereof.

In some embodiments, a TERT mRNA may comprise a codon-optimized sequence. In some embodiments, a TERT mRNA may comprise a uridine depleted human TERT sequence. In some embodiments, the codon-optimized sequence may comprise a sequence at least 70%, at least 75%, at least 80%, at least 85%, at least 90%, at least 95%, at least 97%, at least 99%, or 100% identical to SEQ ID NO 1:

ATGCCCAGAGCCCCCAGATGCAGAGCCGTGAGAAGCCTGCTGAGAAGCCA
CTACAGAGAGGTGCTGCCCCTGGCCACCTTCGTGAGAAGACTGGGCCCCC
AGGGCTGGAGACTGGTGCAGAGAGGCGACCCCGCCGCCTTCAGAGCCCTG
GTGGCCCAGTGCCTGGTGTGCGTGCCCTGGGACGCCAGACCCCCTCCCGC
CGCCCCCAGCTTCAGACAGGTGAGCTGCCTGAAGGAGCTGGTGGCCAGAG
TGCTGCAGAGACTGTGCGAGAGAGGCGCCAAGAACGTGCTGGCCTTCGGC
TTCGCCCTGCTGGACGGCGCCAGAGGCGGCCCTCCCGAGGCCTTCACCAC
CAGCGTGAGAAGCTACCTGCCCAACACCGTGACCGACGCCCTGAGAGGCA
GCGGCGCCTGGGGCCTGCTGCTGAGAAGAGTGGGCGACGACGTGCTGGTG
CACCTGCTGGCCAGATGCGCCCTGTTCGTGCTGGTGGCCCCCAGCTGCGC
CTACCAGGTGTGCGGCCCTCCCCTGTACCAGCTGGGCGCCGCCACCCAGG
CCAGACCCCCTCCCCACGCCAGCGGCCCCAGAAGAAGACTGGGCTGCGAG
AGAGCCTGGAACCACAGCGTGAGAGAGGCCGGCGTGCCCCTGGGCCTGCC
CGCCCCCGGCGCCAGAAGAAGAGGCGGCAGCGCCAGCAGAAGCCTGCCCC
TGCCCAAGAGACCCAGAAGAGGCGCCGCCCCCGAGCCCGAGAGAACCCCC
GTGGGCCAGGGCAGCTGGGCCCACCCCGGCAGAACCAGAGGCCCCAGCGA
CAGAGGCTTCTGCGTGGTGAGCCCCGCCAGACCCGCCGAGGAGGCCACCA
GCCTGGAGGGCGCCCTGAGCGGCACCAGACACAGCCACCCCAGCGTGGGC
AGACAGCACCACGCCGGCCCTCCCAGCACCAGCAGACCTCCCAGACCCTG
GGACACCCCCTGCCCTCCCGTGTACGCCGAGACCAAGCACTTCCTGTACA
GCAGCGGCGACAAGGAGCAGCTGAGACCCAGCTTCCTGCTGAGCAGCCTG
AGACCCAGCCTGACCGGCGCCAGAAGACTGGTGGAGACCATCTTCCTGGG
CAGCAGACCCTGGATGCCCGGCACCCCCAGAAGACTGCCCAGACTGCCCC
AGAGATACTGGCAGATGAGACCCCTGTTCCTGGAGCTGCTGGGCAACCAC
GCCCAGTGCCCCTACGGCGTGCTGCTGAAGACCCACTGCCCCCTGAGAGC
CGCCGTGACCCCCGCCGCCGGCGTGTGCGCCAGAGAGAAGCCCCAGGGCA
GCGTGGCCGCCCCCGAGGAGGAGGACACCGACCCCAGAAGACTGGTGCAG
CTGCTGAGACAGCACAGCAGCCCCTGGCAGGTGTACGGCTTCGTGAGAGC
CTGCCTGAGAAGACTGGTGCCTCCCGGCCTGTGGGGCAGCAGACACAACG
AGAGAAGATTCCTGAGAAACACCAAGAAGTTCATCAGCCTGGGCAAGCAC
GCCAAGCTGAGCCTGCAGGAGCTGACCTGGAAGATGAGCGTGAGAGACTG
CGCCTGGCTGAGAAGAAGCCCCGGCGTGGGCTGCGTGCCCGCCGCCGAGC
ACAGACTGAGAGAGGAGATCCTGGCCAAGTTCCTGCACTGGCTGATGAGC
GTGTACGTGGTGGAGCTGCTGAGAAGCTTCTTCTACGTGACCGAGACCAC
CTTCCAGAAGAACAGACTGTTCTTCTACAGAAAGAGCGTGTGGAGCAAGC
TGCAGAGCATCGGCATCAGACAGCACCTGAAGAGAGTGCAGCTGAGAGAG
CTGAGCGAGGCCGAGGTGAGACAGCACAGAGAGGCCAGACCCGCCCTGCT
GACCAGCAGACTGAGATTCATCCCCAAGCCCGACGGCCTGAGACCCATCG
TGAACATGGACTACGTGGTGGGCGCCAGAACCTTCAGAAGAGAGAAGAGA
GCCGAGAGACTGACCAGCAGAGTGAAGGCCCTGTTCAGCGTGCTGAACTA
CGAGAGAGCCAGAAGACCCGGCCTGCTGGGCGCCAGCGTGCTGGGCCTGG
ACGACATCCACAGAGCCTGGAGAACCTTCGTGCTGAGAGTGAGAGCCCAG
GATCCCCCTCCCGAGCTGTACTTCGTGAAGGTGGACGTGACCGGCGCCTA
CGACACCATCCCCCAGGACAGACTGACCGAGGTGATCGCCAGCATCATCA
AGCCCCAGAACACCTACTGCGTGAGAAGATACGCCGTGGTGCAGAAGGCC
GCCCACGGCCACGTGAGAAAGGCCTTCAAGAGCCACGTGAGCACCCTGAC
CGACCTGCAGCCCTACATGAGACAGTTCGTGGCCCACCTGCAGGAGACCA
GCCCCCTGAGAGACGCCGTGGTGATCGAGCAGAGCAGCAGCCTGAACGAG
GCCAGCAGCGGCCTGTTCGACGTGTTCCTGAGATTCATGTGCCACCACGC
CGTGAGAATCAGAGGCAAGAGCTACGTGCAGTGCCAGGGCATCCCCCAGG
GCAGCATCCTGAGCACCCTGCTGTGCAGCCTGTGCTACGGCGACATGGAG
AACAAGCTGTTCGCCGGCATCAGAAGAGACGGCCTGCTGCTGAGACTGGT
GGACGACTTCCTGCTGGTGACACCCCACCTGACCCACGCCAAGACCTTCC
TGAGAACCCTGGTGAGAGGCGTGCCCGAGTACGGCTGCGTGGTGAACCTG
AGAAAGACCGTGGTGAACTTCCCCGTGGAGGACGAGGCCCTGGGCGGCAC
CGCCTTCGTGCAGATGCCCGCCCACGGCCTGTTCCCCTGGTGCGGCCTGC
TGCTGGACACCAGAACCCTGGAGGTGCAGAGCGACTACAGCAGCTACGCC
AGAACCAGCATCAGAGCCAGCCTGACCTTCAACAGAGGCTTCAAGGCCGG
CAGAAACATGAGAAGAAAGCTGTTCGGCGTGCTGAGACTGAAGTGCCACA
GCCTGTTCCTGGACCTGCAGGTGAACAGCCTGCAGACCGTGTGCACCAAC
ATCTACAAGATCCTGCTGCTGCAGGCCTACAGATTCCACGCCTGCGTGCT
GCAGCTGCCCTTCCACCAGCAGGTGTGGAAGAACCCCACCTTCTTCCTGA
GAGTGATCAGCGACACCGCCAGCCTGTGCTACAGCATCCTGAAGGCCAAG
AACGCCGGCATGAGCCTGGGCGCCAAGGGCGCCGCCGGCCCCCTGCCCAG
CGAGGCCGTGCAGTGGCTGTGCCACCAGGCCTTCCTGCTGAAGCTGACCA
GACACAGAGTGACCTACGTGCCCCTGCTGGGCAGCCTGAGAACCGCCCAG
ACCCAGCTGAGCAGAAAGCTGCCCGGCACCACCCTGACCGCCCTGGAGGC
CGCCGCCAACCCCGCCCTGCCCAGCGACTTCAAGACCATCCTGGACTGA

In some embodiments, a TERT mRNA may comprise a mutant human TERT sequence. In some embodiments, the mutant human TERT mRNA may encode a Y707F mutation in the resulting peptide sequence. In some embodiments a mutation in the TERT mRNA sequence encodes a mutation in the nuclear export signal which may result in nuclear retention of the TERT peptide. In some embodiments, the mutant TERT mRNA sequence may comprise a sequence at least 70%, at least 75%, at least 80%, at least 85%, at least 90%, at least 95%, at least 97%, at least 99%, or 100% identical to SEQ ID NO 2:

ATGCCGCGCGCTCCCCGCTGCCGAGCCGTGCGCTCCCTGCTGCGCAGCCA
CTACCGCGAGGTGCTGCCGCTGGCCACGTTCGTGCGGCGCCTGGGGCCCC
AGGGCTGGCGGCTGGTGCAGCGCGGGGACCCGGCGGCTTTCCGCGCGCTG
GTGGCCCAGTGCCTGGTGTGCGTGCCCTGGGACGCACGGCCGCCCCCCGC
CGCCCCCTCCTTCCGCCAGGTGTCCTGCCTGAAGGAGCTGGTGGCCCGAG
TGCTGCAGAGGCTGTGCGAGCGCGGCGCGAAGAACGTGCTGGCCTTCGGC
TTCGCGCTGCTGGACGGGGCCCGCGGGGGCCCCCCCGAGGCCTTCACCAC
CAGCGTGCGCAGCTACCTGCCCAACACGGTGACCGACGCACTGCGGGGGA
GCGGGGCGTGGGGGCTGCTGCTGCGCCGCGTGGGCGACGACGTGCTGGTT
CACCTGCTGGCACGCTGCGCGCTCTTTGTGCTGGTGGCTCCCAGCTGCGC
CTACCAGGTGTGCGGGCCGCCGCTGTACCAGCTCGGCGCTGCCACTCAGG
CCCGGCCCCCGCCACACGCTAGTGGACCCCGAAGGCGTCTGGGATGCGAA
CGGGCCTGGAACCATAGCGTCAGGGAGGCCGGGGTCCCCCTGGGCCTGCC
AGCCCCGGGTGCGAGGAGGCGCGGGGGCAGTGCCAGCCGAAGTCTGCCGT
TGCCCAAGAGGCCCAGGCGTGGCGCTGCCCCTGAGCCGGAGCGGACGCCC
GTTGGGCAGGGGTCCTGGGCCCACCCGGGCAGGACGCGTGGACCGAGTGA
CCGTGGTTTCTGTGTGGTGTCACCTGCCAGACCCGCCGAAGAAGCCACCT
CTTTGGAGGGTGCGCTCTCTGGCACGCGCCACTCCCACCCATCCGTGGGC
CGCCAGCACCACGCGGGCCCCCCATCCACATCGCGGCCACCACGTCCCTG
GGACACGCCTTGTCCCCCGGTGTACGCCGAGACCAAGCACTTCCTCTACT
CCTCAGGCGACAAGGAGCAGCTGCGGCCCTCCTTCCTACTCAGCTCTCTG
AGGCCCAGCCTGACTGGCGCTCGGAGGCTCGTGGAGACCATCTTTCTGGG
TTCCAGGCCCTGGATGCCAGGGACTCCCCGCAGGTTGCCCCGCCTGCCCC
AGCGCTACTGGCAAATGCGGCCCCTGTTTCTGGAGCTGCTTGGGAACCAC
GCGCAGTGCCCCTACGGGGTGCTCCTCAAGACGCACTGCCCGCTGCGAGC
TGCGGTCACCCCAGCAGCCGGTGTCTGTGCCCGGGAGAAGCCCCAGGGCT
CTGTGGCGGCCCCCGAGGAGGAGGACACAGACCCCCGTCGCCTGGTGCAG
CTGCTCCGCCAGCACAGCAGCCCCTGGCAGGTGTACGGCTTCGTGCGGGC
CTGCCTGCGCCGGCTGGTGCCCCCAGGCCTCTGGGGCTCCAGGCACAACG
AACGCCGCTTCCTCAGGAACACCAAGAAGTTCATCTCCCTGGGGAAGCAT
GCCAAGCTCTCGCTGCAGGAGCTGACGTGGAAGATGAGCGTGCGGGACTG
CGCTTGGCTGCGCAGGAGCCCAGGGGTTGGCTGTGTTCCGGCCGCAGAGC
ACCGTCTGCGTGAGGAGATCCTGGCCAAGTTCCTGCACTGGCTGATGAGT
GTGTACGTCGTCGAGCTGCTCAGGTCTTTCTTTTATGTCACGGAGACCAC
GTTTCAAAAGAACAGGCTCTTTTTCTACCGGAAGAGTGTCTGGAGCAAGT
TGCAAAGCATTGGAATCAGACAGCACTTGAAGAGGGTGCAGCTGCGGGAG
CTGTCGGAAGCAGAGGTCAGGCAGCATCGGGAAGCCAGGCCCGCCCTGCT
GACGTCCAGACTCCGCTTCATCCCCAAGCCTGACGGGCTGCGGCCGATTG
TGAACATGGACTACGTCGTGGGAGCCAGAACGTTCCGCAGAGAAAAGAGG
GCCGAGCGTCTCACCTCGAGGGTGAAGGCACTGTTCAGCGTGCTCAACTA
CGAGCGGGCGCGGCGCCCCGGCCTCCTGGGCGCCTCTGTGCTGGGCCTGG
ACGATATCCACAGGGCCTGGCGCACCTTCGTGCTGCGTGTGCGGGCCCAG
GACCCGCCGCCTGAGCTGTTCTTTGTCAAGGTGGATGTGACGGGCGCGTA
CGACACCATCCCCCAGGACAGGCTCACGGAGGTCATCGCCAGCATCATCA
AACCCCAGAACACGTACTGCGTGCGTCGGTATGCCGTGGTCCAGAAGGCC
GCCCATGGGCACGTCCGCAAGGCCTTCAAGAGCCACGTCTCTACCTTGAC
AGACCTCCAGCCGTACATGCGACAGTTCGTGGCTCACCTGCAGGAGACCA
GCCCGCTGAGGGATGCCGTCGTCATCGAGCAGAGCTCCTCCCTGAATGAG
GCCAGCAGTGGCCTCTTCGACGTCTTCCTACGCTTCATGTGCCACCACGC
CGTGCGCATCAGGGGCAAGTCCTACGTCCAGTGCCAGGGGATCCCGCAGG
GCTCCATCCTCTCCACGCTGCTCTGCAGCCTGTGCTACGGCGACATGGAG
AACAAGCTGTTTGCGGGGATTCGGCGGGACGGGCTGCTCCTGCGTTTGGT
GGATGATTTCTTGTTGGTGACACCTCACCTCACCCACGCGAAAACCTTCC
TCAGGACCCTGGTCCGAGGTGTCCCTGAGTATGGCTGCGTGGTGAACTTG
CGGAAGACAGTGGTGAACTTCCCTGTAGAAGACGAGGCCCTGGGTGGCAC
GGCTTTTGTTCAGATGCCGGCCCACGGCCTATTCCCCTGGTGCGGCCTGC
TGCTGGATACCCGGACCCTGGAGGTGCAGAGCGACTACTCCAGCTATGCC
CGGACCTCCATCAGAGCCAGTCTCACCTTCAACCGCGGCTTCAAGGCTGG
GAGGAACATGCGTCGCAAACTCTTTGGGGTCTTGCGGCTGAAGTGTCACA
GCCTGTTTCTGGATTTGCAGGTGAACAGCCTCCAGACGGTGTGCACCAAC
ATCTACAAGATCCTCCTGCTGCAGGCGTACAGGTTTCACGCATGTGTGCT
GCAGCTCCCATTTCATCAGCAAGTTTGGAAGAACCCCACATTTTTCCTGC
GCGTCATCTCTGACACGGCCTCCCTCTGCTACTCCATCCTGAAAGCCAAG
AACGCAGGGATGTCGCTGGGGGCCAAGGGCGCCGCCGGCCCTCTGCCCTC
CGAGGCCGTGCAGTGGCTGTGCCACCAAGCATTCCTGCTCAAGCTGACTC
GACACCGTGTCACCTACGTGCCACTCCTGGGGTCACTCAGGACAGCCCAG
ACGCAGCTGAGTCGGAAGCTCCCGGGGACGACGCTGACTGCCCTGGAGGC
CGCAGCCAACCCGGCACTGCCCTCAGACTTCAAGACCATCCTGGACTGA

In some embodiments, a mouse TERT mRNA may comprise a codon-optimized sequence. In some embodiments, a TERT mRNA may comprise a uridine depleted mouse TERT sequence. In some embodiments, the codon-optimized sequence may comprise a sequence at least 70%, at least 75%, at least 80%, at least 85%, at least 90%, at least 95%, at least 97%, at least 99%, or 100% identical to SEQ ID NO 3:

ATGACCAGGGCCCCTAGGTGCCCTGCCGTGAGGAGCCTGCTGAGGAGCAG
GTACAGGGAGGTGTGGCCTCTGGCCACCTTCGTGAGGAGGCTGGGCCCTG
AGGGCAGGAGGCTGGTGCAGCCTGGCGACCCTAAGATCTACAGGACCCTG
GTGGCCCAGTGCCTGGTGTGCATGCACTGGGGCAGCCAGCCTCCTCCTGC
CGACCTGAGCTTCCACCAGGTGAGCAGCCTGAAGGAGCTGGTGGCCAGGG
TGGTGCAGAGGCTGTGCGAGAGGAACGAGAGGAACGTGCTGGCCTTCGGC
TTCGAGCTGCTGAACGAGGCCAGGGGCGGCCCTCCTATGGCCTTCACCAG
CAGCGTGAGGAGCTACCTGCCTAACACCGTGATCGAGACCCTGAGGGTGA
GCGGCGCCTGGATGCTGCTGCTGAGCAGGGTGGGCGACGACCTGCTGGTG
TACCTGCTGGCCCACTGCGCCCTGTACCTGCTGGTGCCTCCTAGCTGCGC
CTACCAGGTGTGCGGCAGCCCTCTGTACCAGATCTGCGCCACCACCGACA
TCTGGCCTAGCGTGAGCGCCAGCTACAGGCCTACCAGGCCTGTGGGCAGG
AACTTCACCAACCTGAGGTTCCTGCAGCAGATCAAGAGCAGCAGCAGGCA
GGAGGCCCCTAAGCCTCTGGCCCTGCCTAGCAGGGGCACCAAGAGGCACC
TGAGCCTGACCAGCACCAGCGTGCCTAGCGCCAAGAAGGCCAGGTGCTAC
CCTGTGCCTAGGGTGGAGGAGGGCCCTCACAGGCAGGTGCTGCCTACCCC
TAGCGGCAAGAGCTGGGTGCCTAGCCCTGCCAGGAGCCCTGAGGTGCCTA
CCGCCGAGAAGGACCTGAGCAGCAAGGGCAAGGTGAGCGACCTGAGCCTG
AGCGGCAGCGTGTGCTGCAAGCACAAGCCTAGCAGCACCAGCCTGCTGAG
CCCTCCTAGGCAGAACGCCTTCCAGCTGAGGCCTTTCATCGAGACCAGGC
ACTTCCTGTACAGCAGGGGCGACGGCCAGGAGAGGCTGAACCCTAGCTTC
CTGCTGAGCAACCTGCAGCCTAACCTGACCGGCGCCAGGAGGCTGGTGGA
GATCATCTTCCTGGGCAGCAGGCCTAGGACCAGCGGCCCTCTGTGCAGGA
CCCACAGGCTGAGCAGGAGGTACTGGCAGATGAGGCCTCTGTTCCAGCAG
CTGCTGGTGAACCACGCCGAGTGCCAGTACGTGAGGCTGCTGAGGAGCCA
CTGCAGGTTCAGGACCGCCAACCAGCAGGTGACCGACGCCCTGAACACCA
GCCCTCCTCACCTGATGGACCTGCTGAGGCTGCACAGCAGCCCTTGGCAG
GTGTACGGCTTCCTGAGGGCCTGCCTGTGCAAGGTGGTGAGCGCCAGCCT
GTGGGGCACCAGGCACAACGAGAGGAGGTTCTTCAAGAACCTGAAGAAGT
TCATCAGCCTGGGCAAGTACGGCAAGCTGAGCCTGCAGGAGCTGATGTGG
AAGATGAAGGTGGAGGACTGCCACTGGCTGAGGAGCAGCCCTGGCAAGGA
CAGGGTGCCTGCCGCCGAGCACAGGCTGAGGGAGAGGATCCTGGCCACCT
TCCTGTTCTGGCTGATGGACACCTACGTGGTGCAGCTGCTGAGGAGCTTC
TTCTACATCACCGAGAGCACCTTCCAGAAGAACAGGCTGTTCTTCTACAG
GAAGAGCGTGTGGAGCAAGCTGCAGAGCATCGGCGTGAGGCAGCACCTGG
AGAGGGTGAGGCTGAGGGAGCTGAGCCAGGAGGAGGTGAGGCACCACCAG
GACACCTGGCTGGCCATGCCTATCTGCAGGCTGAGGTTCATCCCTAAGCC
TAACGGCCTGAGGCCTATCGTGAACATGAGCTACAGCATGGGCACCAGGG
CCCTGGGCAGGAGGAAGCAGGCCCAGCACTTCACCCAGAGGCTGAAGACC
CTGTTCAGCATGCTGAACTACGAGAGGACCAAGCACCCTCACCTGATGGG
CAGCAGCGTGCTGGGCATGAACGACATCTACAGGACCTGGAGGGCCTTCG
TGCTGAGGGTGAGGGCCCTGGACCAGACCCCTAGGATGTACTTCGTGAAG
GCCGACGTGACCGGCGCCTACGACGCCATCCCTCAGGGCAAGCTGGTGGA
GGTGGTGGCCAACATGATCAGGCACAGCGAGAGCACCTACTGCATCAGGC
AGTACGCCGTGGTGAGGAGGGACAGCCAGGGCCAGGTGCACAAGAGCTTC
AGGAGGCAGGTGACCACCCTGAGCGACCTGCAGCCTTACATGGGCCAGTT
CCTGAAGCACCTGCAGGACAGCGACGCCAGCGCCCTGAGGAACAGCGTGG
TGATCGAGCAGAGCATCAGCATGAACGAGAGCAGCAGCAGCCTGTTCGAC
TTCTTCCTGCACTTCCTGAGGCACAGCGTGGTGAAGATCGGCGACAGGTG
CTACACCCAGTGCCAGGGCATCCCTCAGGGCAGCAGCCTGAGCACCCTGC
TGTGCAGCCTGTGCTTCGGCGACATGGAGAACAAGCTGTTCGCCGAGGTG
CAGAGGGACGGCCTGCTGCTGAGGTTCGTGGACGACTTCCTGCTGGTGAC
CCCTCACCTGGACCAGGCCAAGACCTTCCTGAGCACCCTGGTGCACGGCG
TGCCTGAGTACGGCTGCATGATCAACCTGCAGAAGACCGTGGTGAACTTC
CCTGTGGAGCCTGGCACCCTGGGCGGCGCCGCCCCTTACCAGCTGCCTGC
CCACTGCCTGTTCCCTTGGTGCGGCCTGCTGCTGGACACCCAGACCCTGG
AGGTGTTCTGCGACTACAGCGGCTACGCCCAGACCAGCATCAAGACCAGC
CTGACCTTCCAGAGCGTGTTCAAGGCCGGCAAGACCATGAGGAACAAGCT
GCTGAGCGTGCTGAGGCTGAAGTGCCACGGCCTGTTCCTGGACCTGCAGG
TGAACAGCCTGCAGACCGTGTGCATCAACATCTACAAGATCTTCCTGCTG
CAGGCCTACAGGTTCCACGCCTGCGTGATCCAGCTGCCTTTCGACCAGAG
GGTGAGGAAGAACCTGACCTTCTTCCTGGGCATCATCAGCAGCCAGGCCA
GCTGCTGCTACGCCATCCTGAAGGTGAAGAACCCTGGCATGACCCTGAAG
GCCAGCGGCAGCTTCCCTCCTGAGGCCGCCCACTGGCTGTGCTACCAGGC
CTTCCTGCTGAAGCTGGCCGCCCACAGCGTGATCTACAAGTGCCTGCTGG
GCCCTCTGAGGACCGCCCAGAAGCTGCTGTGCAGGAAGCTGCCTGAGGCC
ACCATGACCATCCTGAAGGCCGCCGCCGACCCTGCCCTGAGCACCGACTT
CCAGACCATCCTGGACTGA

In some embodiments, a mouse TERT mRNA may comprise a mutant mouse TERT sequence. In some embodiments, the mutant mouse TERT mRNA may encode a Y707F mutation in the resulting peptide sequence. In some embodiments a mutation in the TERT mRNA sequence encodes a mutation in the nuclear export signal which may result in nuclear retention of the TERT peptide. In some embodiments, the mutant mouse TERT mRNA sequence may comprise a sequence at least 70%, at least 75%, at least 80%, at least 85%, at least 90%, at least 95%, at least 97%, at least 99%, or 100% identical to SEQ ID NO 4:

ATGACCCGCGCTCCTCGTTGCCCCGCGGTGCGCTCTCTGCTGCGCAGCCG
ATACCGGGAGGTGTGGCCGCTGGCAACCTTTGTGCGGCGCCTGGGGCCCG
AGGGCAGGCGGCTTGTGCAACCCGGGGACCCGAAGATCTACCGCACTTTG
GTTGCCCAATGCCTAGTGTGCATGCACTGGGGCTCACAGCCTCCACCTGC
CGACCTTTCCTTCCACCAGGTGTCATCCCTGAAAGAGCTGGTGGCCAGGG
TTGTGCAGAGACTCTGCGAGCGCAACGAGAGAAACGTGCTGGCTTTTGGC
TTTGAGCTGCTTAACGAGGCCAGAGGCGGGCCTCCCATGGCCTTCACTAG
TAGCGTGCGTAGCTACTTGCCCAACACTGTTATTGAGACCCTGCGTGTCA
GTGGTGCATGGATGCTACTGTTGAGCCGAGTGGGCGACGACCTGCTGGTC
TACCTGCTGGCACACTGTGCTCTTTATCTTCTGGTGCCCCCCAGCTGTGC
CTACCAGGTGTGTGGGTCTCCCCTGTACCAAATTTGTGCCACCACGGATA
TCTGGCCCTCTGTGTCCGCTAGTTACAGGCCCACCCGACCCGTGGGCAGG
AATTTCACTAACCTTAGGTTCTTACAACAGATCAAGAGCAGTAGTCGCCA
GGAAGCACCGAAACCCCTGGCCTTGCCATCTCGAGGTACAAAGAGGCATC
TGAGTCTCACCAGTACAAGTGTGCCTTCAGCTAAGAAGGCCAGATGCTAT
CCTGTCCCGAGAGTGGAGGAGGGACCCCACAGGCAGGTGCTACCAACCCC
ATCAGGCAAATCATGGGTGCCAAGTCCTGCTCGGTCCCCCGAGGTGCCTA
CTGCAGAGAAAGATTTGTCTTCTAAAGGAAAGGTGTCTGACCTGAGTCTC
TCTGGGTCGGTGTGCTGTAAACACAAGCCCAGCTCCACATCTCTGCTGTC
ACCACCCCGCCAAAATGCCTTTCAGCTCAGGCCATTTATTGAGACCAGAC
ATTTCCTTTACTCCAGGGGAGATGGCCAAGAGCGTCTAAACCCCTCATTC
CTACTCAGCAACCTCCAGCCTAACTTGACTGGGGCCAGGAGACTGGTGGA
GATCATCTTTCTGGGCTCAAGGCCTAGGACATCAGGACCACTCTGCAGGA
CACACCGTCTATCGCGTCGATACTGGCAGATGCGGCCCCTGTTCCAACAG
CTGCTGGTGAACCATGCAGAGTGCCAATATGTCAGACTCCTCAGGTCACA
TTGCAGGTTTCGAACAGCAAACCAACAGGTGACAGATGCCTTGAACACCA
GCCCACCGCACCTCATGGATTTGCTCCGCCTGCACAGCAGTCCCTGGCAG
GTATATGGTTTTCTTCGGGCCTGTCTCTGCAAGGTGGTGTCTGCTAGTCT
CTGGGGTACCAGGCACAATGAGCGCCGCTTCTTTAAGAACTTAAAGAAGT
TCATCTCGTTGGGGAAATACGGCAAGCTATCACTGCAGGAACTGATGTGG
AAGATGAAAGTAGAGGATTGCCACTGGCTCCGCAGCAGCCCGGGGAAGGA
CCGTGTCCCCGCTGCAGAGCACCGTCTGAGGGAGAGGATCCTGGCTACGT
TCCTGTTCTGGCTGATGGACACATACGTGGTACAGCTGCTTAGGTCATTC
TTTTACATCACAGAGAGCACATTCCAGAAGAACAGGCTCTTCTTCTACCG
TAAGAGTGTGTGGAGCAAGCTGCAGAGCATTGGAGTCAGGCAACACCTTG
AGAGAGTGCGGCTACGGGAGCTGTCACAAGAGGAGGTCAGGCATCACCAG
GACACCTGGCTAGCCATGCCCATCTGCAGACTGCGCTTCATCCCCAAGCC
CAACGGCCTGCGGCCCATTGTGAACATGAGTTATAGCATGGGTACCAGAG
CTTTGGGCAGAAGGAAGCAGGCCCAGCATTTCACCCAGCGTCTCAAGACT
CTCTTCAGCATGCTCAACTATGAGCGGACAAAACATCCTCACCTTATGGG
GTCTTCTGTACTGGGTATGAATGACATCTACAGGACCTGGCGGGCCTTTG
TGCTGCGTGTGCGTGCTCTGGACCAGACACCCAGGATGTACTTTGTTAAG
GCAGATGTGACCGGGGCCTITGATGCCATCCCCCAGGGTAAGCTGGTGGA
GGTTGTTGCCAATATGATCAGGCACTCGGAGAGCACGTACTGTATCCGCC
AGTATGCAGTGGTCCGGAGAGATAGCCAAGGCCAAGTCCACAAGTCCTTT
AGGAGACAGGTCACCACCCTCTCTGACCTCCAGCCATACATGGGCCAGTT
CCTTAAGCATCTGCAGGATTCAGATGCCAGTGCACTGAGGAACTCCGTTG
TCATCGAGCAGAGCATCTCTATGAATGAGAGCAGCAGCAGCCTGTTTGAC
TTCTTCCTGCACTTCCTGCGTCACAGTGTCGTAAAGATTGGTGACAGGTG
CTATACGCAGTGCCAGGGCATCCCCCAGGGCTCCAGCCTATCCACCCTGC
TCTGCAGTCTGTGTTTCGGAGACATGGAGAACAAGCTGTTTGCTGAGGTG
CAGCGGGATGGGTTGCTTTTACGTTTTGTTGATGACTTTCTGTTGGTGAC
GCCTCACTTGGACCAAGCAAAAACCTTCCTCAGCACCCTGGTCCATGGCG
TTCCTGAGTATGGGTGCATGATAAACTTGCAGAAGACAGTGGTGAACTTC
CCTGTGGAGCCTGGTACCCTGGGTGGTGCAGCTCCATACCAGCTGCCTGC
TCACTGCCTGTTTCCCTGGTGTGGCTTGCTGCTGGACACTCAGACTTTGG
AGGTGTTCTGTGACTACTCAGGTTATGCCCAGACCTCAATTAAGACGAGC
CTCACCTTCCAGAGTGTCTTCAAAGCTGGGAAGACCATGCGGAACAAGCT
CCTGTCGGTCTTGCGGTTGAAGTGTCACGGTCTATTTCTAGACTTGCAGG
TGAACAGCCTCCAGACAGTCTGCATCAATATATACAAGATCTTCCTGCTT
CAGGCCTACAGGTTCCATGCATGTGTGATTCAGCTTCCCTTTGACCAGCG
TGTTAGGAAGAACCTCACATTCTTTCTGGGCATCATCTCCAGCCAAGCAT
CCTGCTGCTATGCTATCCTGAAGGTCAAGAATCCAGGAATGACACTAAAG
GCCTCTGGCTCCTTTCCTCCTGAAGCCGCACATTGGCTCTGCTACCAGGC
CTTCCTGCTCAAGCTGGCTGCTCATTCTGTCATCTACAAATGTCTCCTGG
GACCTCTGAGGACAGCCCAAAAACTGCTGTGCCGGAAGCTCCCAGAGGCG
ACAATGACCATCCTTAAAGCTGCAGCTGACCCAGCCCTAAGCACAGACTT
TCAGACCATTTTGGACTAA

In some embodiments, a mouse TERT mRNA may comprise a mutant mouse TERT sequence. In some embodiments, the mutant mouse TERT mRNA may encode a Y697F mutation in the resulting peptide sequence. In some embodiments a mutation in the TERT mRNA sequence encodes a mutation in the nuclear export signal which may result in nuclear retention of the TERT peptide. In some embodiments, the mutant mouse TERT mRNA sequence may comprise a sequence at least 70%, at least 75%, at least 80%, at least 85%, at least 90%, at least 95%, at least 97%, at least 99%, or 100% identical to SEQ ID NO 5:

ATGACCCGCGCTCCTCGTTGCCCCGCGGTGCGCTCTCTGCTGCGCAGCCG
ATACCGGGAGGTGTGGCCGCTGGCAACCTTTGTGCGGCGCCTGGGGCCCG
AGGGCAGGCGGCTTGTGCAACCCGGGGACCCGAAGATCTACCGCACTTTG
GTTGCCCAATGCCTAGTGTGCATGCACTGGGGCTCACAGCCTCCACCTGC
CGACCTTTCCTTCCACCAGGTGTCATCCCTGAAAGAGCTGGTGGCCAGGG
TTGTGCAGAGACTCTGCGAGCGCAACGAGAGAAACGTGCTGGCTTTTGGC
TTTGAGCTGCTTAACGAGGCCAGAGGCGGGCCTCCCATGGCCTTCACTAG
TAGCGTGCGTAGCTACTTGCCCAACACTGTTATTGAGACCCTGCGTGTCA
GTGGTGCATGGATGCTACTGTTGAGCCGAGTGGGCGACGACCTGCTGGTC
TACCTGCTGGCACACTGTGCTCTTTATCTTCTGGTGCCCCCCAGCTGTGC
CTACCAGGTGTGTGGGTCTCCCCTGTACCAAATTTGTGCCACCACGGATA
TCTGGCCCTCTGTGTCCGCTAGTTACAGGCCCACCCGACCCGTGGGCAGG
AATTTCACTAACCTTAGGTTCTTACAACAGATCAAGAGCAGTAGTCGCCA
GGAAGCACCGAAACCCCTGGCCTTGCCATCTCGAGGTACAAAGAGGCATC
TGAGTCTCACCAGTACAAGTGTGCCTTCAGCTAAGAAGGCCAGATGCTAT
CCTGTCCCGAGAGTGGAGGAGGGACCCCACAGGCAGGTGCTACCAACCCC
ATCAGGCAAATCATGGGTGCCAAGTCCTGCTCGGTCCCCCGAGGTGCCTA
CTGCAGAGAAAGATTTGTCTTCTAAAGGAAAGGTGTCTGACCTGAGTCTC
TCTGGGTCGGTGTGCTGTAAACACAAGCCCAGCTCCACATCTCTGCTGTC
ACCACCCCGCCAAAATGCCTTTCAGCTCAGGCCATTTATTGAGACCAGAC
ATTTCCTTTACTCCAGGGGAGATGGCCAAGAGCGTCTAAACCCCTCATTC
CTACTCAGCAACCTCCAGCCTAACTTGACTGGGGCCAGGAGACTGGTGGA
GATCATCTTTCTGGGCTCAAGGCCTAGGACATCAGGACCACTCTGCAGGA
CACACCGTCTATCGCGTCGATACTGGCAGATGCGGCCCCTGTTCCAACAG
CTGCTGGTGAACCATGCAGAGTGCCAATATGTCAGACTCCTCAGGTCACA
TTGCAGGTTTCGAACAGCAAACCAACAGGTGACAGATGCCTTGAACACCA
GCCCACCGCACCTCATGGATTTGCTCCGCCTGCACAGCAGTCCCTGGCAG
GTATATGGTTTTCTTCGGGCCTGTCTCTGCAAGGTGGTGTCTGCTAGTCT
CTGGGGTACCAGGCACAATGAGCGCCGCTTCTTTAAGAACTTAAAGAAGT
TCATCTCGTTGGGGAAATACGGCAAGCTATCACTGCAGGAACTGATGTGG
AAGATGAAAGTAGAGGATTGCCACTGGCTCCGCAGCAGCCCGGGGAAGGA
CCGTGTCCCCGCTGCAGAGCACCGTCTGAGGGAGAGGATCCTGGCTACGT
TCCTGTTCTGGCTGATGGACACATACGTGGTACAGCTGCTTAGGTCATTC
TTTTACATCACAGAGAGCACATTCCAGAAGAACAGGCTCTTCTTCTACCG
TAAGAGTGTGTGGAGCAAGCTGCAGAGCATTGGAGTCAGGCAACACCTTG
AGAGAGTGCGGCTACGGGAGCTGTCACAAGAGGAGGTCAGGCATCACCAG
GACACCTGGCTAGCCATGCCCATCTGCAGACTGCGCTTCATCCCCAAGCC
CAACGGCCTGCGGCCCATTGTGAACATGAGTTATAGCATGGGTACCAGAG
CTTTGGGCAGAAGGAAGCAGGCCCAGCATTTCACCCAGCGTCTCAAGACT
CTCTTCAGCATGCTCAACTATGAGCGGACAAAACATCCTCACCTTATGGG
GTCTTCTGTACTGGGTATGAATGACATCTACAGGACCTGGCGGGCCTTTG
TGCTGCGTGTGCGTGCTCTGGACCAGACACCCAGGATGTTCTTTGTTAAG
GCAGATGTGACCGGGGCCTATGATGCCATCCCCCAGGGTAAGCTGGTGGA
GGTTGTTGCCAATATGATCAGGCACTCGGAGAGCACGTACTGTATCCGCC
AGTATGCAGTGGTCCGGAGAGATAGCCAAGGCCAAGTCCACAAGTCCTTT
AGGAGACAGGTCACCACCCTCTCTGACCTCCAGCCATACATGGGCCAGTT
CCTTAAGCATCTGCAGGATTCAGATGCCAGTGCACTGAGGAACTCCGTTG
TCATCGAGCAGAGCATCTCTATGAATGAGAGCAGCAGCAGCCTGTTTGAC
TTCTTCCTGCACTTCCTGCGTCACAGTGTCGTAAAGATTGGTGACAGGTG
CTATACGCAGTGCCAGGGCATCCCCCAGGGCTCCAGCCTATCCACCCTGC
TCTGCAGTCTGTGTTTCGGAGACATGGAGAACAAGCTGTTTGCTGAGGTG
CAGCGGGATGGGTTGCTTTTACGTTTTGTTGATGACTTTCTGTTGGTGAC
GCCTCACTTGGACCAAGCAAAAACCTTCCTCAGCACCCTGGTCCATGGCG
TTCCTGAGTATGGGTGCATGATAAACTTGCAGAAGACAGTGGTGAACTTC
CCTGTGGAGCCTGGTACCCTGGGTGGTGCAGCTCCATACCAGCTGCCTGC
TCACTGCCTGTTTCCCTGGTGTGGCTTGCTGCTGGACACTCAGACTTTGG
AGGTGTTCTGTGACTACTCAGGTTATGCCCAGACCTCAATTAAGACGAGC
CTCACCTTCCAGAGTGTCTTCAAAGCTGGGAAGACCATGCGGAACAAGCT
CCTGTCGGTCTTGCGGTTGAAGTGTCACGGTCTATTTCTAGACTTGCAGG
TGAACAGCCTCCAGACAGTCTGCATCAATATATACAAGATCTTCCTGCTT
CAGGCCTACAGGTTCCATGCATGTGTGATTCAGCTTCCCTTTGACCAGCG
TGTTAGGAAGAACCTCACATTCTTTCTGGGCATCATCTCCAGCCAAGCAT
CCTGCTGCTATGCTATCCTGAAGGTCAAGAATCCAGGAATGACACTAAAG
GCCTCTGGCTCCTTTCCTCCTGAAGCCGCACATTGGCTCTGCTACCAGGC
CTTCCTGCTCAAGCTGGCTGCTCATTCTGTCATCTACAAATGTCTCCTGG
GACCTCTGAGGACAGCCCAAAAACTGCTGTGCCGGAAGCTCCCAGAGGCG
ACAATGACCATCCTTAAAGCTGCAGCTGACCCAGCCCTAAGCACAGACTT
TCAGACCATTTTGGACTAA

The compositions comprise a ribonucleic acid, e.g., a synthetic ribonucleic acid coding for a telomerase reverse transcriptase (TERT), wherein telomeres are extended within a cell treated with the compound. The ribonucleic acids used in the transient expression of TERT can comprise a ribonucleic acid coding for a TERT protein. The ribonucleic acids can further comprise one or more sequences that affect the expression and/or stability of the ribonucleic acid in a cell. For example, the ribonucleic acids can contain a 5′ cap and untranslated region (UTR) to the 5′ and/or 3′ side of the coding sequence. The ribonucleic acids may further contain a 3′ tail, such as a poly-A tail. The poly-A tail can, for example, increase the stability of the ribonucleic acid. In some embodiments, the poly-A tail comprises at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides. In some embodiments, the poly-A tail comprises between 1 and 25 nucleotides, between 25 and 50 nucleotides, between 50 and 75 nucleotides, between 75 and 100 nucleotides, between 100 and 125 nucleotides, between 125 and 150 nucleotides, between 150 and 175 nucleotides, between 175 and 200 nucleotides, between 200 and 225 nucleotides, or between 225 and 250 nucleotides, inclusive of the endpoints for each range. In some embodiments, the poly-A tail comprises between 100 and 200 nucleotides, inclusive of the endpoints.

In some embodiments, the 5′ cap of the ribonucleic acid is a non-immunogenic cap. In some embodiments, the 5′ cap may increase the translation of the ribonucleic acid. In some embodiments, the 5′ cap may be treated with phosphatase to modulate the innate immunogenicity of the ribonucleic acid. In some embodiments, the 5′ cap is an anti-reverse cap analog (“ARCA”), such as a 3′-O-Me-m7G(5′)ppp(5′)G RNA cap structure analog. In some embodiments, the 5′ cap is m7G(5′)ppp(5′)(2′OmeA)pG (also known as CleanCap© AG). In some embodiments, the 5′ cap is m7(3′OmeG)(5′)ppp(5′)(2′OmeA)pG (also known as CleanCap® AG (3′ Ome)).

The above features, or others, may increase translation of the TERT protein encoded by the ribonucleic acid, may increase or decrease the stability of the ribonucleic acid itself in a cell type-specific or cell type-independent manner, or may do both. In some embodiments, the 5′ UTR and/or the 3′ UTR are from a gene that has a very stable mRNA and/or an mRNA that is rapidly translated, for example, α-globin or β-globin, c-fos, or tobacco etch virus. In some embodiments, the 5′ UTR and 3′ UTR are from different genes or are from different species than the species into which the compositions are being delivered. The UTRs may also be assemblies of parts of UTRs from the mRNAs of different genes, where the parts are selected to achieve a certain combination of stability and efficiency of translation. The UTRs may also comprise designed sequences that confer properties to the RNA such as cell type-specific stability or cell type-independent stability.

The ribonucleic acids of the present disclosure may comprise one or more modified nucleosides, and/or comprise primary sequences of nucleosides, that modulate translation, stability, or immunogenicity of the RNA (“mRNA”). Most mature RNA molecules in eukaryotic cells contain nucleosides that are modified versions of the canonical unmodified RNA nucleosides, adenine, cytidine, guanosine, and uridine. For example, the 5′ cap of mature RNA comprises a modified nucleoside, and other modified nucleosides often occur elsewhere in the RNA. Those modifications may prevent the RNA from being recognized as a foreign RNA. Synthetic RNA molecules made using certain nucleosides are much less immunogenic than unmodified RNA. The immunogenicity can be reduced even further by purifying the synthetic mRNA, for example by using high performance liquid chromatography (HPLC). The modified nucleosides may be, for example, chosen from the nucleosides listed below. The nucleosides are, in some embodiments, pseudouridine, 1-methylpseudouridine, 2-thiouridine, 5-methoxyuridine, or 5-methylcytidine. The primary sequence may be modified in ways that increase or decrease immunogenicity. Under some circumstances, it may be desirable for the modified RNA to retain some immunogenicity.

Accordingly, in some embodiments, the ribonucleic acids of the instant compositions comprise a 1-methylpseudouridine, pseudouridine, a 5-methoxyuridine (5-moU), a 2-thiouridine, a 5-methylcytidine, or another modified nucleoside. Modified nucleosides found in eukaryotic cells include m1A 1-methyladenosine, m6A N6-methyladenosine, Am 2′-O-methyladenosine, i6A N6-isopentenyladenosine, io6A N6-(cis-hydroxyisopentenyl)adenosine, ms2io6A 2-methylthio-N6-(cis-hydroxyisopentenyl) adenosine, g6A N6-glycinylcarbamoyladenosine, t6A N6-threonylcarbamoyladenosine, ms2t6A 2-methylthio-N6-threonyl carbamoyladenosine, Ar(p) 2′-O-ribosyladenosine (phosphate), m6 2A N6,N6-dimethyladenosine, m6Am N6,2′-O-dimethyladenosine, m6 2Am N6,N6,2′-O-trimethyladenosine, m1Am 1,2′-O-dimethyladenosine, m3C 3-methylcytidine, m5C 5-methylcytidine, Cm 2′-O-methylcytidine, ac4C N4-acetylcytidine, f5C 5-formylcytidine, m4C N4-methylcytidine, hm5C 5-hydroxymethylcytidine, f5Cm 5-formyl-2′-O-methylcytidine, m1G 1-methylguanosine, m2G N2-methylguanosine, m7G 7-methylguanosine, Gm 2′-O-methylguanosine, m2 2G N2,N2-dimethylguanosine, Gr(p) 2′-O-ribosylguanosine (phosphate), yW wybutosine, o2yW peroxywybutosine, OhyW hydroxywybutosine, OhyW* undermodified hydroxywybutosine, imG wyosine, m2,7G N2,7-dimethylguanosine, m2,2,7G N2,N2,7-trimethylguanosine I inosine, m1I 1-methylinosine, Im 2′-O-methylinosine, Q queuosine, galQ galactosyl-queuosine, manQ mannosyl-queuosine, Ψ pseudouridine, D dihydrouridine, m5U 5-methyluridine, Um 2′-O-methyluridine, m5Um 5,2′-O-dimethyluridine, m1Ψ 1-methylpseudouridine, Ψm 2′-O-methylpseudouridine, s2U 2-thiouridine, ho5U 5-hydroxyuridine, chm5U 5-(carboxyhydroxymethyl)uridine, mchm5U 5-(carboxyhydroxymethyl)uridine, methyl ester mcm5U 5-methoxycarbonylmethyluridine, mcm5Um 5-methoxycarbonylmethyl-2′-O-methyluridine, mcm5s2U 5-methoxycarbonylmethyl-2-thiouridine, ncm5U 5-carbamoylmethyluridine, ncm5Um 5-carbamoylmethyl-2′-O-methyluridine, cmnm5U 5-carboxymethylaminomethyluridine, m3U 3-methyluridine, m1acp3Ψ 1-methyl-3-(3-amino-3-carboxypropyl) pseudouridine, cm5U 5-carboxymethyluridine, m3Um 3,2′-O-dimethyluridine, m5D 5-methyldihydrouridine, τm5U 5-taurinomethyluridine, τm5s2U 5-taurinomethyl-2-thiouridine, 2-Aminoadenosine, 2-Amino-6-chloropurineriboside, 8-Azaadenosine, 6-Chloropurineriboside, 5-Iodocytidine, 5-Iodouridine, Inosine, 2′-O-Methylinosine, Xanthosine, 4-Thiouridine, 06-Methylguanosine, 5,6-Dihydrouridine, 2-Thiocytidine, 6-Azacytidine, 6-Azauridine, 2′-O-Methyl-2-aminoadenosine, 2′-O-Methylpseudouridine, N1-Methyladenosine, 2′-O-Methyl-5-methyluridine, 7-Deazaguanosine, 8-Azidoadenosine, 5-Bromocytidine, 5-Bromouridine, 7-Deazaadenosine, 5-Aminoallyluridine, 5-Aminoallylcytidine, 8-Oxoguanosine, 2-Aminopurine-riboside, Pseudoisocytidine, N1-Methylpseudouridine, 5,6-Dihydro-5-Methyluridine, N6-Methyl-2-Aminoadenosine, 5-Carboxycytidine, 5-Hydroxymethyluridine, Thienoguanosine, 5-Hydroxycytidine, 5-Formyluridine, 5-Carboxyuridine, 5-Methoxyuridine, 5-Methoxycytidine, Thienouridine, 5-Carboxymethylesteruridine, Thienocytidine, 8-Oxoadenoosine, Isoguanosine, N1-Ethylpseudouridine, N1-Methyl-2′-O-Methylpseudouridine, N1-Methoxymethylpseudouridine, N1-Propylpseudouridine, 2′-O-Methyl-N6-Methyladenosine, 2-Amino-6-Cl-purine-2′-deoxyriboside, 2-Amino-2′-deoxyadenosine, 2-Aminopurine-2′-deoxyriboside, 5-Bromo-2′-deoxycytidine, 5-Bromo-2′-deoxyuridine, 6-Chloropurine-2′-deoxyriboside, 7-Deaza-2′-deoxyadenosine, 7-Deaza-2′-deoxyguanosine, 2′-Deoxyinosine, 5-Propynyl-2′-deoxycytidine, 5-Propynyl-2′-deoxyuridine, 5-Fluoro-2′-deoxyuridine, 5-Iodo-2′-deoxycytidine, 5-Iodo-2′-deoxyuridine, N6-Methyl-2′-deoxyadenosine, 5-Methyl-2′-deoxycytidine, 06-Methyl-2′-deoxyguanosine, N2-Methyl-2′-deoxyguanosine, 8-Oxo-2′-deoxyadenosine, 8-Oxo-2′-deoxyguanosine, 2-Thiothymidine, 2′-Deoxy-P-nucleoside, 5-Hydroxy-2′-deoxycytidine, 4-Thiothymidine, 2-Thio-2′-deoxycytidine, 6-Aza-2′-deoxyuridine, 6-Thio-2′-deoxyguanosine, 8-Chloro-2′-deoxyadenosine, 5-Aminoallyl-2′-deoxycytidine, 5-Aminoallyl-2′-deoxyuridine, N4-Methyl-2′-deoxycytidine, 2′-Deoxyzebularine, 5-Hydroxymethyl-2′-deoxyuridine, 5-Hydroxymethyl-2′-deoxycytidine, 5-Propargylamino-2′-deoxycytidine, 5-Propargylamino-2′-deoxyuridine, 5-Carboxy-2′-deoxycytidine, 5-Formyl-2′-deoxycytidine, 5-[(3-Indolyl)propionamide-N-allyl]-2′-deoxyuridine, 5-Carboxy-2′-deoxyuridine, 5-Formyl-2′-deoxyuridine, 7-Deaza-7-Propargylamino-2′-deoxyadenosine, 7-Deaza-7-Propargylamino-2′-deoxyguanosine, Biotin-16-Aminoallyl-2′-dUTP, Biotin-16-Aminoallyl-2′-dCTP, Biotin-16-Aminoallylcytidine, N4-Biotin-OBEA-2′-deoxycytidine, Biotin-16-Aminoallyluridine, Dabcyl-5-3-Aminoallyl-2′-dUTP, Desthiobiotin-6-Aminoallyl-2′-deoxycytidine, Desthiobiotin-16-Aminoallyl-Uridine, Biotin-16-7-Deaza-7-Propargylamino-2′-deoxyguanosine, Cyanine 3-5-Propargylamino-2′-deoxycytidine, Cyanine 3-6-Propargylamino-2′-deoxyuridine, Cyanine 5-6-Propargylamino-2′-deoxycytidine, Cyanine 5-6-Propargylamino-2′-deoxyuridine, Cyanine 3-Aminoallylcytidine, Cyanine 3-Aminoallyluridine, Cyanine 5-Aminoallylcytidine, Cyanine 5-Aminoallyluridine, Cyanine 7-Aminoallyluridine, 2′-Fluoro-2′-deoxyadenosine, 2′-Fluoro-2′-deoxycytidine, 2′-Fluoro-2′-deoxyguanosine, 2′-Fluoro-2′-deoxyuridine, 2′-O-Methyladenosine, 2′-O-Methylcytidine, 2′-O-Methylguanosine, 2′-O-Methyluridine, Puromycin, 2′-Amino-2′-deoxycytidine, 2′-Amino-2′-deoxyuridine, 2′-Azido-2′-deoxycytidine, 2′-Azido-2′-deoxyuridine, Aracytidine, Arauridine, 2′-Azido-2′-deoxyadenosine, 2′-Amino-2′-deoxyadenosine, Araadenosine, 2′-Fluoro-thymidine, 3′-O-Methyladenosine, 3′-O-Methylcytidine, 3′-O-Methylguanosine, 3′-O-Methyluridine, 2′-Azido-2′-deoxyguanosine, Araguanosine, 2′-Deoxyuridine, 3′-O-(2-nitrobenzyl)-2′-Deoxyadenosine, 3′-O-(2-nitrobenzyl)-2′-Deoxyinosine, 3′-Deoxyadenosine, 3′-Deoxyguanosine, 3′-Deoxycytidine, 3′-Deoxy-5-Methyluridine, 3′-Deoxyuridine, 2′,3′-Dideoxyadenosine, 2′,3′-Dideoxyguanosine, 2′,3′-Dideoxyuridine, 2′,3′-Dideoxythymidine, 2′,3′-Dideoxycytidine, 3′-Azido-2′,3′-dideoxyadenosine, 3′-Azido-2′,3′-dideoxythymidine, 3′-Amino-2′,3′-dideoxyadenosine, 3′-Amino-2′,3′-dideoxycytidine, 3′-Amino-2′,3′-dideoxyguanosine, 3′-Amino-2′,3′-dideoxythymidine, 3′-Azido-2′,3′-dideoxycytidine, 3′-Azido-2′,3′-dideoxyuridine, 5-Bromo-2′,3′-dideoxyuridine, 2′,3′-Dideoxyinosine, 2′-Deoxyadenosine-5′-O-(1-Thiophosphate), 2′-Deoxycytidine-5′-O-(1-Thiophosphate), 2′-Deoxyguanosine-5′-O-(1-Thiophosphate), 2′-Deoxythymidine-5′-O-(1-Thiophosphate), Adenosine-5′-O-(1-Thiophosphate), Cytidine-5′-O-(1-Thiophosphate), Guanosine-5′-O-(1-Thiophosphate), Uridine-5′-O-(1-Thiophosphate), 2′,3′-Dideoxyadenosine-5′-O-(1-Thiophosphate), 2′,3′-Dideoxycytidine-5′-O-(1-Thiophosphate), 2′,3′-Dideoxyguanosine-5′-O-(1-Thiophosphate), 3′-Deoxythymidine-5′-O-(1-Thiophosphate), 3′-Azido-2′,3′-dideoxythymidine-5′-O-(1-Thiophosphate), 2′,3′-Dideoxyuridine-5′-O-(1-Thiophosphate), 2′-Deoxyadenosine-5′-O-(1-Boranophosphate), 2′-Deoxycytidine-5′-O-(1-Boranophosphate), 2′-Deoxyguanosine-5′-O-(1-Boranophosphate), and 2′-Deoxythymidine-5′-O-(1-Boranophosphate).

Without intending to be bound by theory, the presence of the modified nucleosides, and/or sequences of nucleosides that alter secondary structure of the RNA and/or binding of RNA to RNA binding proteins or microRNA, may enable mRNA to avoid activation of an immune response mediated by various receptors, including the Toll-like receptors and RIG-1. Non-immunogenic mRNA has been used as a therapeutic agent in mice via topical delivery. Kormann et al. (2011) Nature Biotechnology 29:154-157. In some embodiments, the ribonucleic acids comprise more than one of the above nucleosides or combination of the above nucleosides. In some embodiments, the ribonucleic acids comprise 1-methylpseudouridine, 5-methoxyuridine, or pseudouridine and 5-methylcytidine.

In some embodiments, an immune response to the mRNA may be desired, and the RNA may be modified to induce an optimal level of innate immunity. In other embodiments, an immune response to the mRNA may not be desired, and the RNA may be modified in order to minimize such a reaction. The RNA can be modified for either situation.

The ribonucleic acid molecules can be synthetic ribonucleic acids. The term “synthetic”, as used herein, can mean that the ribonucleic acids are in some embodiments prepared using the tools of molecular biology under the direction of a human, for example as described below. The synthetic ribonucleic acids may, for example, be prepared by in vitro synthesis using cellular extracts or purified enzymes and nucleic acid templates. The synthetic ribonucleic acids may in some embodiments be prepared by chemical synthesis, either partially or completely. Alternatively, or in addition, the synthetic ribonucleic acids may in some embodiments be prepared by engineered expression in a cell, followed by disruption of the cell and at least partial purification of the ribonucleic acid.

The ribonucleic acids of the present disclosure may be prepared using a variety of techniques, as would be understood by one of ordinary skill in the art. In some embodiments, the ribonucleic acids may be prepared by in vitro synthesis. In some embodiments, the ribonucleic acids may be prepared by chemical synthesis. In some embodiments, the ribonucleic acids may be prepared by a combination of in vitro synthesis and chemical synthesis. As described above, the term “synthetic” should be understood to include ribonucleic acids that are prepared either by chemical synthesis, by in vitro synthesis, by expression in vivo and at least partial purification, or by a combination of such, or other, chemical or molecular biological methods.

The ribonucleic acids may, in some embodiments, be purified. As noted above, purification may reduce immunogenicity of the ribonucleic acids and may be advantageous in some circumstances. In some embodiments, the ribonucleic acids are purified by one or more of HPLC, DNAse treatment, protease treatment, or by affinity capture and elution.

The protein structure of TERT can include at least three distinct domains: a long extension at the amino-terminus (the N-terminal extension, NTE) that contains conserved domains and an unstructured linker region; a catalytic reverse-transcriptase domain in the middle of the primary sequence that includes seven conserved reverse transcriptase (RT) motifs; and a short extension at the carboxyl-terminus. In some embodiments, the ribonucleic acid codes for a full-length TERT. In some embodiments, the ribonucleic acid codes for a catalytic reverse transcriptase domain of TERT. In some embodiments, the ribonucleic acid codes for a polypeptide having TERT activity. TERT activity may be measured using known methods including the telomerase repeat amplification protocol (TRAP).

The TERT encoded by the ribonucleic acids of the instant disclosure may be a mammalian, avian, reptilian, or fish TERT. In some embodiments, the TERT is a mammalian TERT, such as human TERT. Meyerson et al. (1997) Cell 90:785-795; Nakamura et al. (1997) Science 277:955-959; Wick et al. (1999) Gene 232:97-106.

The amino acid sequence of two human TERT isoforms are available as NCBI Reference Sequences: NP_937983.2 and NP_001180305.1.

| The amino acid sequence of human TERT isoform 1 may comprise or consist of the | |
|---|---|
| sequence of SEQ ID NO: 6 (also described at GenBank Accession No. NP_937983.2): | |
| 1 | mprparcrav rsllrshyre vlplatfvrr lgpqgwrlvq rgdpaafral vaqclvcvpw |
| 61 | darpppaaps frqvsclkel varvlqrlce rgaknvlafg falldgargg ppeafttsvr |
| 121 | sylpntvtda lrgsgawgll lrrvgddvlv hllarcalfv lvapscayqv cgpplyqlga |
| 181 | atqarpppha sgprrrlgce rawnhsvrea gvplglpapg arrrggsasr slplpkrprr |
| 241 | gaapepertp vgqgswahpg rtrgpsdrgf cvvsparpae eatslegals gtrhshpsvg |
| 301 | rqhhagppst srpprpwdtp cppvyaethk flyssgdkeq lrpsfllssl rpsltgarrl |
| 361 | vetiflgsrp wmpgtprrlp rlpqrywqmr plflellgnh aqcpygvllk thcplraavt |
| 421 | paagvcarek pqgsvaapee edtdprrlvq llrqhsspwq vygfvraclr rlvppglwgs |
| 481 | rhnerrflrn tkkfislgkh aklslqeltw kmsvrdcawl rrspgvgcvp aaehrlreei |
| 541 | lakflhwlms vyvvellrsf fyvtettfqk nrlffyrksv wsklqsigir qhlkrvqlre |
| 601 | lseaevrqhr earpalltsr lrfipkpdgl rpivnmdyvv gartfrrekr aerltsrvka |
| 661 | lfsvlnyera rrpgllgasv lglddihraw rtfvlrvraq dpppelyfvk vdvtgaydti |
| 721 | pqdrltevia siikpqntyc vrryavvqka ahghvrkafk shvstltdlq pymrqfvahl |
| 781 | qetsplrdav vieqssslne assglfdvfl rfmchhavri rgksyvqcqg ipqgsilstl |
| 841 | lcslcygdme nklgagirrd glllrlvddf llvtphltha ktflrtlvrg vpeygcvvnl |
| 901 | rktvvnfpve dealggtafv qmpahglfpw cgllldtrtl evqsdyssya rtsirasltf |
| 961 | nrgfkagrnm rrklfgvlrl kchslfldlq vnslqtvctn iykilllqay rfhacvlqlp |
| 1021 | fhqqvwknpt fflrvisdta slcysilkak nagmslgakg aagplpseav qwlchqafll |
| 1081 | kltrhrvtyv pllgslrtaq tqlsrklpgt tltaleaaan palpsdfkti ld. |
| The nucleic acid sequence of human TERT isoform 1 may comprise or consist of the | |
| sequence SEQ ID NO: 7 (also described at GenBank Accession No. NM_198253.3): | |
| 1 | ctctcctcgc ggcgcgagtt tcaggcagcg ctgcgtcctg ctgcgcacgt gggaagccct |
| 61 | ggccccggcc acccccgcga tgccgcgcgc tccccgctgc cgagccgtgc gctccctgct |
| 121 | gcgcagccac taccgcgagg tgctgccgct ggccacgttc gtgcggcgcc tggggcccca |
| 181 | gggctggcgg ctggtgcagc gcggggaccc ggcggctttc cgcgcgctgg tggcccagtg |
| 241 | cctggtgtgc gtgccctggg acgcacggcc gccccccgcc gccccctcct tccgccaggt |
| 301 | gtcctgcctg aaggagctgg tggcccgagt gctgcagagg ctgtgcgagc gcggcgcgaa |
| 361 | gaacgtgctg gccttcggct tcgcgctgct ggacggggcc cgcgggggcc cccccgaggc |
| 421 | cttcaccacc agcgtgcgca gctacctgcc caacacggtg accgacgcac tgcgggggag |
| 481 | cggggcgtgg gggctgctgc tgcgccgcgt gggcgacgac gtgctggttc acctgctggc |
| 541 | acgctgcgcg ctctttgtgc tggtggctcc cagctgcgcc taccaggtgt gcgggccgcc |
| 601 | gctgtaccag ctcggcgctg ccactcaggc ccggcccccg ccacacgcta gtggaccccg |
| 661 | aaggcgtctg ggatgcgaac gggcctggaa ccatagcgtc agggaggccg gggtccccct |
| 721 | gggcctgcca gccccgggtg cgaggaggcg cgggggcagt gccagccgaa gtctgccgtt |
| 781 | gcccaagagg cccaggcgtg gcgctgcccc tgagccggag cggacgcccg ttgggcaggg |
| 841 | gtcctggccc cacccgggca ggacgcgtgg accgagtgac cgtggtttct gtgtggtgtc |
| 901 | acctgccaga cccgccgaag aagccacctc tttggagggt gcgctctctg gcacgcgcca |
| 961 | ctcccaccca tccgtggccc gccagcacca cgcgggcccc ccatccacat cgcggccacc |
| 1021 | acgtccctgg gacacgcctt gtcccccggt gtacgccgag accaagcact tcctctactc |
| 1081 | ctcaggcgac aaggagcagc tgcggccctc cttcctactc agctctctga ggcccagcct |
| 1141 | gactggcgct cggaggctcg tggagaccat ctttctgggt tccaggccct ggatgccagg |
| 1201 | gactccccgc aggttgcccc gcctgcccca gcgctactgg caaatgcggc ccctgtttct |
| 1261 | ggagctgctt gggaaccacg cgcagtgccc ctacggggtg ctcctcaaga cgcactgccc |
| 1321 | gctgcgagct gcggtcaccc cagcagccgg tgtctgtgcc cgggagaagc cccagggctc |
| 1381 | tgtggcggcc cccgaggagg aggacacaga cccccgtcgc ctggtgcagc tgctccgcca |
| 1441 | gcacagcagc ccctggcagg tgtacggctt cgtgcgggcc tgcctgcgcc ggctggtgcc |
| 1501 | cccaggcctc tggggctcca ggcacaacga acgccgcttc ctcaggaaca ccaagaagtt |
| 1561 | catctccctg gggaagcatg ccaagctctc gctgcaggag ctgacgtgga agatgagcgt |
| 1621 | gcgggactgc gcttggctgc gcaggagccc aggggttggc tgtgttccgg ccgcagagca |
| 1681 | ccgtctgcgt gaggagatcc tggccaagtt cctgcactgg ctgatgagtg tgtacgtcgt |
| 1741 | cgagctgctc aggtctttct tttatgtcac ggagaccacg tttcaaaaga acaggctctt |
| 1801 | tttctaccgg aagagtgtct ggagcaagtt gcaaagcatt ggaatcagac agcacttgaa |
| 1861 | gagggtgcag ctgcgggagc tgtcggaagc agaggtcagg cagcatcggg aagccaggcc |
| 1921 | cgccctgctg acgtccagac tccgcttcat ccccaagcct gacgggctgc ggccgattgt |
| 1981 | gaacatggac tacgtcgtgg gagccagaac gttccgcaga gaaaagaggg ccgagcgtct |
| 2041 | cacctcgagg gtgaaggcac tgttcagcgt gctcaactac gagcgggcgc ggcgccccgg |
| 2101 | cctcctgggc gcctctgtgc tgggcctgga cgatatccac agggcctggc gcaccttcgt |
| 2161 | gctgcgtgtg cgggcccagg acccgccgcc tgagctgtac tttgtcaagg tggatgtgac |
| 2221 | gggcgcgtac gacaccatcc cccaggacag gctcacggag gtcatcgcca gcatcatcaa |
| 2281 | accccagaac acgtactgcg tgcgtcggta tgccgtggtc cagaaggccg cccatgggca |
| 2341 | cgtccgcaag gccttcaaga gccacgtctc taccttgaca gacctccagc cgtacatgcg |
| 2401 | acagttcgtg gctcacctgc aggagaccag cccgctgagg gatgccgtcg tcatcgagca |
| 2461 | gagctcctcc ctgaatgagg ccagcagtgg cctcttcgac gtcttcctac gcttcatgtg |
| 2521 | ccaccacgcc gtgcgcatca ggggcaagtc ctacgtccag tgccagggga tcccgcaggg |
| 2581 | ctccatcctc tccacgctgc tctgcagcct gtgctacggc gacatggaga acaagctgtt |
| 2641 | tgcggggatt cggcgggacg ggctgctcct gcgtttggtg gatgatttct tgttggtgac |
| 2701 | acctcacctc acccacgcga aaaccttcct caggaccctg gtccgaggtg tccctgagta |
| 2761 | tggctgcgtg gtgaacttgc ggaagacagt ggtgaacttc cctgtagaag acgaggccct |
| 2821 | gggtggcacg gcttttgttc agatgccggc ccacggccta ttcccctggt gcggcctgct |
| 2881 | gctggatacc cggaccctgg aggtgcagag cgactactcc agctatgccc ggacctccat |
| 2941 | cagagccagt ctcaccttca accgcggctt caaggctggg aggaacatgc gtcgcaaact |
| 3001 | ctttggggtc ttgcggctga agtgtcacag cctgtttctg gatttgcagg tgaacagcct |
| 3061 | ccagacggtg tgcaccaaca tctacaagat cctcctgctg caggcgtaca ggtttcacgc |
| 3121 | atgtgtgctg cagctcccat ttcatcagca agtttggaag aaccccacat ttttcctgcg |
| 3181 | cgtcatctct gacacggcct ccctctgcta ctccatcctg aaagccaaga acgcagggat |
| 3241 | gtcgctgggg gccaagggcg ccgccggccc tctgccctcc gaggccgtgc agtggctgtg |
| 3301 | ccaccaagca ttcctgctca agctgactcg acaccgtgtc acctacgtgc cactcctggg |
| 3361 | gtcactcagg acagcccaga cgcagctgag tcggaagctc ccggggacga cgctgactgc |
| 3421 | cctggaggcc gcagccaacc cggcactgcc ctcagacttc aagaccatcc togactgatg |
| 3481 | gccacccgcc cacagccagg ccgagagcag acaccagcag ccctgtcacg ccgggctcta |
| 3541 | cgtcccaggg agggaggggc ggcccacacc caggcccgca ccgctgggag tctgaggcct |
| 3601 | gagtgagtgt ttggccgagg cctgcatgtc cggctgaagg ctgagtgtcc ggctgaggcc |
| 3661 | tgagcgagtg tccagccaag ggctgagtgt ccagcacace tgccgtcttc acttccccac |
| 3721 | aggctggcgc tcggctccac cccagggcca gcttttcctc accaggagcc cggcttccac |
| 3781 | tccccacata ggaatagtcc atccccagat tcgccattgt tcacccctcg ccctgccctc |
| 3841 | ctttgccttc cacccccacc atccaggtgg agaccctgag aaggaccctg ggagctctgg |
| 3901 | gaatttggag tgaccaaagg tgtgccctgt acacaggcga ggaccctgca cctggatggg |
| 3961 | ggtccctgtg ggtcaaattg gggggaggtg ctgtgggagt aaaatactga atatatgagt |
| 4021 | ttttcagttt tgaaaaaaa. |
| The amino acid sequence of human TERT isoform 2 may comprise or consist of the | |
| sequence of SEQ ID NO: 8 (also described at GenBank Accession No. NP_001180305.1): | |
| 1 | mpraprcrav rsllrshyre vlplatfvrr lgpqgwrlvq rgdpaafral vaqclvcvpw |
| 61 | darpppaaps frqvsclkel varvlqrlve rgaknvlafg falldgargg ppeafttsvr |
| 121 | sylpntvtda lrgsgawgll lrrvgddvlv hllarcalfv lvapscayqv cgpplyqlga |
| 181 | atqarpppha sgprrrlgce rawnhsvrea gvplglpapg arrrggsasr slplpkrprr |
| 241 | gaapepertp vgqgswahpg rtrgpsdrgf cvvsparpae eatslegals gtrhshpsvg |
| 301 | rqhhagppst srpprpwdtp cppvyaetkh flyssgdkeq lrpsfllssl rpsltgarrl |
| 361 | vetiflgsrp wmpgtprrlp rlpqrywqmr plflellgnh aqcpygvllk thcplraavt |
| 421 | paagvcarek pqgsvaapee edtdprrlvq llrqhsspwq vygfvraclr rlvppglwgs |
| 481 | rhnerrflrn tkkfislgkh aklslqeltw kmsvrdcawl rrspgvgcvp aaerhlreei |
| 541 | lakflhwlms vyvvellrsf fyvtettfqk nrlffyrksv wsklqsigir qhlkrvqlre |
| 601 | lseaevrqhr earpalltsr lrfipkpdgl rpivnmdyvv gartfrrekr aerltsrvka |
| 661 | lfsvlnyera rrpgllgasv lglddihraw rtfvlrvraq dpppelyfvk vdvtgaydti |
| 721 | pqdrltevia siikpqntyc vrryavvqka ahghvrkafk shvstltdlq pymrqfvahl |
| 781 | qetsplrdav vieqssslne assglfdvfl rfmchhavri rgksyvqcqg ipqgsilstl |
| 841 | lcslcygdme nklfagirrd glllrlvddf llvtphltha ktflsyarts irasltfnrg |
| 901 | fkagrnmrrk lfgvlrlkch slfldlqvns lqtvctniyk illlqayrfh acvlqlpfhq |
| 961 | qvwknptffl rvisdtaslc ysilkaknag mslgakgaag plpseavqwl chqafllklt |
| 1021 | rhrvtyvpll gslrtaqtql srklpgttlt aleaaanpal psdfktild. |
| The amino acid sequence of human TERT isoform 2 may comprise or consist of the | |
| sequence of SEQ ID NO: 9 (also described at GenBank Accession No. NP_001193376.3): | |
| 1 | ctctcctcgc ggcgcgagtt tcaggcagcg ctgcgtcctg ctgcgcacgt gggaagccct |
| 61 | ggccccggcc acccccgcga tgccgcgcgc tccccgctgc cgagccgtgc gctccctgct |
| 121 | gcgcagccac taccgcgagg tgctgccgct ggccacgttc gtgcggcgcc tggggcccca |
| 181 | gggctggcgg ctggtgcagc gcggggaccc ggcggctttc cgcgcgctgg tggcccagtg |
| 241 | cctggtgtgc gtgccctggg acgcacggcc gccccccgcc gccccctect tccgccaggt |
| 301 | gtcctgcctg aaggagctgg tggcccgagt gctgcagagg ctgtgcgagc gcggcgcgaa |
| 361 | gaacgtgctg gccttcggct tcgcgctgct ggacggggcc cgcgggggcc cccccgaggc |
| 421 | cttcaccacc agcgtgcgca gctacctgcc caacacggtg accgacgcac tgcgggggag |
| 481 | cggggcgtgg gggctgctgc tgcgccgcgt gggcgacgac gtgctggttc acctgctggc |
| 541 | acgctgcgcg ctctttgtgc tggtggctcc cagctgcgcc taccaggtgt gcgggccgcc |
| 601 | gctgtaccag ctcggcgctg ccactcaggc ccggcccccg ccacacgcta gtggaccccg |
| 661 | aaggcgtctg ggatgcgaac gggcctggaa ccatagcgtc agggaggccg gggtccccct |
| 721 | gggcctgcca gccccgggtg cgaggaggcg cgggggcagt gccagccgaa gtctgccgtt |
| 781 | gcccaagagg cccaggcgtg gcgctgcccc tgagccggag cggacgcccg ttgggcaggg |
| 841 | gtcctgggcc cacccgggca ggacgcgtgg accgagtgac cgtggtttct gtgtggtgtc |
| 901 | acctgccaga cccgccgaag aagccacctc tttggagggt gcgctctctg gcacgcgcca |
| 961 | ctcccaccca tccgtgggcc gccagcacca cgcgggcccc ccatccacat cgcggccacc |
| 1021 | acgtccctgg gacacgcctt gtcccccggt gtacgccgag accaagcact tcctctactc |
| 1081 | ctcaggcgac aaggagcagc tgcggccctc cttcctactc agctctctga ggcccagcct |
| 1141 | gactggcgct cggaggctcg tggagaccat ctttctgggt tccaggccct ggatgccagg |
| 1201 | gactccccgc aggttgcccc gcctgcccca gcgctactgg caaatgcggc ccctgtttct |
| 1261 | ggagctgctt gggaaccacg cgcagtgccc ctacggggtg ctcctcaaga cgcactgccc |
| 1321 | gctgcgagct gcggtcaccc cagcagccgg tgtctgtgcc cgggagaagc cccagggctc |
| 1381 | tgtggcggcc cccgaggagg aggacacaga cccccgtcgc ctggtgcagc tgctccgcca |
| 1441 | gcacagcagc ccctggcagg tgtacggctt cgtgcgggcc tgcctgcgcc ggctggtgcc |
| 1501 | cccaggcctc tggggctcca ggcacaacga acgccgcttc ctcaggaaca ccaagaagtt |
| 1561 | catctccctg gggaagcatg ccaagctctc gctgcaggag ctgacgtgga agatgagcgt |
| 1621 | gcgggactgc gcttggctgc gcaggagccc aggggttggc tgtgttccgg ccgcagagca |
| 1681 | ccgtctgcgt gaggagatcc tggccaagtt cctgcactgg ctgatgagtg tgtacgtcgt |
| 1741 | cgagctgctc aggtctttct tttatgtcac ggagaccacg tttcaaaaga acaggctctt |
| 1801 | tttctaccgg aagagtgtct ggagcaagtt gcaaagcatt ggaatcagac agcacttgaa |
| 1861 | gagggtgcag ctgcgggagc tgtcggaagc agaggtcagg cagcatcggg aagccaggcc |
| 1921 | cgccctgctg acgtccagac tccgcttcat ccccaagcct gacgggctgc ggccgattgt |
| 1981 | gaacatggac tacgtcgtgg gagccagaac gttccgcaga gaaaagaggg ccgagcgtct |
| 2041 | cacctcgagg gtgaaggcac tgttcagcgt gctcaactac gagcgggcgc ggcgccccgg |
| 2101 | cctcctgggc gcctctgtgc tgggcctgga cgatatccac agggcctggc gcaccttcgt |
| 2161 | gctgcgtgtg cgggcccagg acccgccgcc tgagctgtac tttgtcaagg tggatgtgac |
| 2221 | gggcgcgtac gacaccatcc cccaggacag gctcacggag gtcatcgcca gcatcatcaa |
| 2281 | accccagaac acgtactgcg tgcgtcggta tgccgtggtc cagaaggccg cccatgggca |
| 2341 | cgtccgcaag gccttcaaga gccacgtctc taccttgaca gacctccagc cgtacatgcg |
| 2401 | acagttcgtg gctcacctgc aggagaccag cccgctgagg gatgccgtcg tcatcgagca |
| 2461 | gagctcctcc ctgaatgagg ccagcagtgg cctcttcgac gtcttcctac gcttcatgtg |
| 2521 | ccaccacgcc gtgcgcatca ggggcaagtc ctacgtccag toccagggga tcccgcaggg |
| 2581 | ctccatcctc tccacgctgc tctgcagcct gtgctacggc gacatggaga acaagctgtt |
| 2641 | tgcggggatt cggcgggacg ggctgctcct gcgtttggtg gatgatttct tgttggtgac |
| 2701 | acctcacctc acccacgcga aaaccttcct cagctatgcc cggacctcca tcagagccag |
| 2761 | tctcaccttc aaccgcggct tcaaggctgg gaggaacatg cgtcgcaaac tctttggggt |
| 2821 | cttgcggctg aagtgtcaca gcctgtttct ggatttgcag gtgaacagcc tccagacggt |
| 2881 | gtgcaccaac atctacaaga tcctcctgct gcaggcgtac aggtttcacg catgtgtgct |
| 2941 | gcagctccca tttcatcage aagtttggaa gaaccccaca tttttcctgc gcgtcatctc |
| 3001 | tgacacggcc tccctctgct actccatcct gaaagccaag aacgcaggga tgtcgctggg |
| 3061 | ggccaagggc gccgccggcc ctctgccctc cgaggccgtg cagtggctgt gccaccaagc |
| 3121 | attcctgctc aagctgactc gacaccgtgt cacctacgtg ccactcctgg ggtcactcag |
| 3181 | gacagcccag acgcagctga gtcggaagct cccggggacg acgctgactg ccctggaggc |
| 3241 | cgcagccaac ccggcactgc cctcagactt caagaccatc ctggactgat ggccacccgc |
| 3301 | ccacagccag gccgagagca gacaccagca gccctgtcac gccgggctct acgtcccagg |
| 3361 | gagggagggg cggcccacac ccaggcccgc accgctggga gtctgaggcc tgagtgagtg |
| 3421 | tttggccgag gcctgcatgt ccggctgaag gctgagtgtc cggctgaggc ctgagcgagt |
| 3481 | gtccagccaa gggctgagtg tccagcacac ctgccgtctt cacttcccca caggctggcg |
| 3541 | ctcggctcca ccccagggcc agcttttcct caccaggagc ccggcttcca ctccccacat |
| 3601 | aggaatagtc catccccaga ttcgccattg ttcacccctc gccctgccct cctttgcctt |
| 3661 | ccacccccac catccaggtg gagaccctga gaaggaccct gggagctctg ggaatttgga |
| 3721 | gtgaccaaag gtgtgccctg tacacaggcg aggaccctgc acctggatgg gggtccctgt |
| 3781 | gggtcaaatt ggggggaggt gctgtgggag taaaatactg aatatatgag tttttcagtt |
| 3841 | ttgaaaaaaa. |

In some embodiments, a human TERT mRNA may comprise a wild type TERT sequence. In some embodiments, the wild type TERT sequence may comprise a sequence at least 70%, at least 75%, at least 80%, at least 85%, at least 90%, at least 95%, at least 97%, at least 99%, or 100% identical to SEQ ID NO 30:

ATGCCGCGCGCTCCCCGCTGCCGAGCCGTGCGCTCCCTGCTGCGC
AGCCACTACCGCGAGGTGCTGCCGCTGGCCACGTTCGTGCGGCGC
CTGGGGCCCCAGGGCTGGCGGCTGGTGCAGCGCGGGGACCCGGCG
GCTTTCCGCGCGCTGGTGGCCCAGTGCCTGGTGTGCGTGCCCTGG
GACGCACGGCCGCCCCCCGCCGCCCCCTCCTTCCGCCAGGTGTCC
TGCCTGAAGGAGCTGGTGGCCCGAGTGCTGCAGAGGCTGTGCGAG
CGCGGCGCGAAGAACGTGCTGGCCTTCGGCTTCGCGCTGCTGGAC
GGGGCCCGCGGGGGCCCCCCCGAGGCCTTCACCACCAGCGTGCGC
AGCTACCTGCCCAACACGGTGACCGACGCACTGCGGGGGAGCGGG
GCGTGGGGGCTGCTGCTGCGCCGCGTGGGCGACGACGTGCTGGTT
CACCTGCTGGCACGCTGCGCGCTCTTTGTGCTGGTGGCTCCCAGC
TGCGCCTACCAGGTGTGCGGGCCGCCGCTGTACCAGCTCGGCGCT
GCCACTCAGGCCCGGCCCCCGCCACACGCTAGTGGACCCCGAAGG
CGTCTGGGATGCGAACGGGCCTGGAACCATAGCGTCAGGGAGGCC
GGGGTCCCCCTGGGCCTGCCAGCCCCGGGTGCGAGGAGGCGCGGG
GGCAGTGCCAGCCGAAGTCTGCCGTTGCCCAAGAGGCCCAGGCGT
GGCGCTGCCCCTGAGCCGGAGCGGACGCCCGTTGGGCAGGGGTCC
TGGGCCCACCCGGGCAGGACGCGTGGACCGAGTGACCGTGGTTTC
TGTGTGGTGTCACCTGCCAGACCCGCCGAAGAAGCCACCTCTTTG
GAGGGTGCGCTCTCTGGCACGCGCCACTCCCACCCATCCGTGGGC
CGCCAGCACCACGCGGGCCCCCCATCCACATCGCGGCCACCACGT
CCCTGGGACACGCCTTGTCCCCCGGTGTACGCCGAGACCAAGCAC
TTCCTCTACTCCTCAGGCGACAAGGAGCAGCTGCGGCCCTCCTTC
CTACTCAGCTCTCTGAGGCCCAGCCTGACTGGCGCTCGGAGGCTC
GTGGAGACCATCTTTCTGGGTTCCAGGCCCTGGATGCCAGGGACT
CCCCGCAGGTTGCCCCGCCTGCCCCAGCGCTACTGGCAAATGCGG
CCCCTGTTTCTGGAGCTGCTTGGGAACCACGCGCAGTGCCCCTAC
GGGGTGCTCCTCAAGACGCACTGCCCGCTGCGAGCTGCGGTCACC
CCAGCAGCCGGTGTCTGTGCCCGGGAGAAGCCCCAGGGCTCTGTG
GCGGCCCCCGAGGAGGAGGACACAGACCCCCGTCGCCTGGTGCAG
CTGCTCCGCCAGCACAGCAGCCCCTGGCAGGTGTACGGCTTCGTG
CGGGCCTGCCTGCGCCGGCTGGTGCCCCCAGGCCTCTGGGGCTCC
AGGCACAACGAACGCCGCTTCCTCAGGAACACCAAGAAGTTCATC
TCCCTGGGGAAGCATGCCAAGCTCTCGCTGCAGGAGCTGACGTGG
AAGATGAGCGTGCGGGACTGCGCTTGGCTGCGCAGGAGCCCAGGG
GTTGGCTGTGTTCCGGCCGCAGAGCACCGTCTGCGTGAGGAGATC
CTGGCCAAGTTCCTGCACTGGCTGATGAGTGTGTACGTCGTCGAG
CTGCTCAGGTCTTTCTTTTATGTCACGGAGACCACGTTTCAAAAG
AACAGGCTCTTTTTCTACCGGAAGAGTGTCTGGAGCAAGTTGCAA
AGCATTGGAATCAGACAGCACTTGAAGAGGGTGCAGCTGCGGGAG
CTGTCGGAAGCAGAGGTCAGGCAGCATCGGGAAGCCAGGCCCGCC
CTGCTGACGTCCAGACTCCGCTTCATCCCCAAGCCTGACGGGCTG
CGGCCGATTGTGAACATGGACTACGTCGTGGGAGCCAGAACGTTC
CGCAGAGAAAAGAGGGCCGAGCGTCTCACCTCGAGGGTGAAGGCA
CTGTTCAGCGTGCTCAACTACGAGCGGGCGCGGCGCCCCGGCCTC
CTGGGCGCCTCTGTGCTGGGCCTGGACGATATCCACAGGGCCTGG
CGCACCTTCGTGCTGCGTGTGCGGGCCCAGGACCCGCCGCCTGAG
CTGTACTTTGTCAAGGTGGATGTGACGGGCGCGTACGACACCATC
CCCCAGGACAGGCTCACGGAGGTCATCGCCAGCATCATCAAACCC
CAGAACACGTACTGCGTGCGTCGGTATGCCGTGGTCCAGAAGGCC
GCCCATGGGCACGTCCGCAAGGCCTTCAAGAGCCACGTCTCTACC
TTGACAGACCTCCAGCCGTACATGCGACAGTTCGTGGCTCACCTG
CAGGAGACCAGCCCGCTGAGGGATGCCGTCGTCATCGAGCAGAGC
TCCTCCCTGAATGAGGCCAGCAGTGGCCTCTTCGACGTCTTCCTA
CGCTTCATGTGCCACCACGCCGTGCGCATCAGGGGCAAGTCCTAC
GTCCAGTGCCAGGGGATCCCGCAGGGCTCCATCCTCTCCACGCTG
CTCTGCAGCCTGTGCTACGGCGACATGGAGAACAAGCTGTTTGCG
GGGATTCGGCGGGACGGGCTGCTCCTGCGTTTGGTGGATGATTTC
TTGTTGGTGACACCTCACCTCACCCACGCGAAAACCTTCCTCAGG
ACCCTGGTCCGAGGTGTCCCTGAGTATGGCTGCGTGGTGAACTTG
CGGAAGACAGTGGTGAACTTCCCTGTAGAAGACGAGGCCCTGGGT
GGCACGGCTTTTGTTCAGATGCCGGCCCACGGCCTATTCCCCTGG
TGCGGCCTGCTGCTGGATACCCGGACCCTGGAGGTGCAGAGCGAC
TACTCCAGCTATGCCCGGACCTCCATCAGAGCCAGTCTCACCTTC
AACCGCGGCTTCAAGGCTGGGAGGAACATGCGTCGCAAACTCTTT
GGGGTCTTGCGGCTGAAGTGTCACAGCCTGTTTCTGGATTTGCAG
GTGAACAGCCTCCAGACGGTGTGCACCAACATCTACAAGATCCTC
CTGCTGCAGGCGTACAGGTTTCACGCATGTGTGCTGCAGCTCCCA
TTTCATCAGCAAGTTTGGAAGAACCCCACATTTTTCCTGCGCGTC
ATCTCTGACACGGCCTCCCTCTGCTACTCCATCCTGAAAGCCAAG
AACGCAGGGATGTCGCTGGGGGCCAAGGGCGCCGCCGGCCCTCTG
CCCTCCGAGGCCGTGCAGTGGCTGTGCCACCAAGCATTCCTGCTC
AAGCTGACTCGACACCGTGTCACCTACGTGCCACTCCTGGGGTCA
CTCAGGACAGCCCAGACGCAGCTGAGTCGGAAGCTCCCGGGGACG
ACGCTGACTGCCCTGGAGGCCGCAGCCAACCCGGCACTGCCCTCA
GACTTCAAGACCATCCTGGACTGA

In some embodiments, a mouse TERT mRNA may comprise a wild type TERT sequence. In some embodiments, the wild type TERT sequence may comprise a sequence at least 70%, at least 75%, at least 80%, at least 85%, at least 90%, at least 95%, at least 97%, at least 99%, or 100% identical to SEQ ID NO 31:

ATGACCCGCGCTCCTCGTTGCCCCGCGGTGCGCTCTCTGCTGCGC
AGCCGATACCGGGAGGTGTGGCCGCTGGCAACCTTTGTGCGGCGC
CTGGGGCCCGAGGGCAGGCGGCTTGTGCAACCCGGGGACCCGAAG
ATCTACCGCACTTTGGTTGCCCAATGCCTAGTGTGCATGCACTGG
GGCTCACAGCCTCCACCTGCCGACCTTTCCTTCCACCAGGTGTCA
TCCCTGAAAGAGCTGGTGGCCAGGGTTGTGCAGAGACTCTGCGAG
CGCAACGAGAGAAACGTGCTGGCTTTTGGCTTTGAGCTGCTTAAC
GAGGCCAGAGGCGGGCCTCCCATGGCCTTCACTAGTAGCGTGCGT
AGCTACTTGCCCAACACTGTTATTGAGACCCTGCGTGTCAGTGGT
GCATGGATGCTACTGTTGAGCCGAGTGGGCGACGACCTGCTGGTC
TACCTGCTGGCACACTGTGCTCTTTATCTTCTGGTGCCCCCCAGC
TGTGCCTACCAGGTGTGTGGGTCTCCCCTGTACCAAATTTGTGCC
ACCACGGATATCTGGCCCTCTGTGTCCGCTAGTTACAGGCCCACC
CGACCCGTGGGCAGGAATTTCACTAACCTTAGGTTCTTACAACAG
ATCAAGAGCAGTAGTCGCCAGGAAGCACCGAAACCCCTGGCCTTG
CCATCTCGAGGTACAAAGAGGCATCTGAGTCTCACCAGTACAAGT
GTGCCTTCAGCTAAGAAGGCCAGATGCTATCCTGTCCCGAGAGTG
GAGGAGGGACCCCACAGGCAGGTGCTACCAACCCCATCAGGCAAA
TCATGGGTGCCAAGTCCTGCTCGGTCCCCCGAGGTGCCTACTGCA
GAGAAAGATTTGTCTTCTAAAGGAAAGGTGTCTGACCTGAGTCTC
TCTGGGTCGGTGTGCTGTAAACACAAGCCCAGCTCCACATCTCTG
CTGTCACCACCCCGCCAAAATGCCTTTCAGCTCAGGCCATTTATT
GAGACCAGACATTTCCTTTACTCCAGGGGAGATGGCCAAGAGCGT
CTAAACCCCTCATTCCTACTCAGCAACCTCCAGCCTAACTTGACT
GGGGCCAGGAGACTGGTGGAGATCATCTTTCTGGGCTCAAGGCCT
AGGACATCAGGACCACTCTGCAGGACACACCGTCTATCGCGTCGA
TACTGGCAGATGCGGCCCCTGTTCCAACAGCTGCTGGTGAACCAT
GCAGAGTGCCAATATGTCAGACTCCTCAGGTCACATTGCAGGTTT
CGAACAGCAAACCAACAGGTGACAGATGCCTTGAACACCAGCCCA
CCGCACCTCATGGATTTGCTCCGCCTGCACAGCAGTCCCTGGCAG
GTATATGGTTTTCTTCGGGCCTGTCTCTGCAAGGTGGTGTCTGCT
AGTCTCTGGGGTACCAGGCACAATGAGCGCCGCTTCTTTAAGAAC
TTAAAGAAGTTCATCTCGTTGGGGAAATACGGCAAGCTATCACTG
CAGGAACTGATGTGGAAGATGAAAGTAGAGGATTGCCACTGGCTC
CGCAGCAGCCCGGGGAAGGACCGTGTCCCCGCTGCAGAGCACCGT
CTGAGGGAGAGGATCCTGGCTACGTTCCTGTTCTGGCTGATGGAC
ACATACGTGGTACAGCTGCTTAGGTCATTCTTTTACATCACAGAG
AGCACATTCCAGAAGAACAGGCTCTTCTTCTACCGTAAGAGTGTG
TGGAGCAAGCTGCAGAGCATTGGAGTCAGGCAACACCTTGAGAGA
GTGCGGCTACGGGAGCTGTCACAAGAGGAGGTCAGGCATCACCAG
GACACCTGGCTAGCCATGCCCATCTGCAGACTGCGCTTCATCCCC
AAGCCCAACGGCCTGCGGCCCATTGTGAACATGAGTTATAGCATG
GGTACCAGAGCTTTGGGCAGAAGGAAGCAGGCCCAGCATTTCACC
CAGCGTCTCAAGACTCTCTTCAGCATGCTCAACTATGAGCGGACA
AAACATCCTCACCTTATGGGGTCTTCTGTACTGGGTATGAATGAC
ATCTACAGGACCTGGCGGGCCTTTGTGCTGCGTGTGCGTGCTCTG
GACCAGACACCCAGGATGTACTTTGTTAAGGCAGATGTGACCGGG
GCCTATGATGCCATCCCCCAGGGTAAGCTGGTGGAGGTTGTTGCC
AATATGATCAGGCACTCGGAGAGCACGTACTGTATCCGCCAGTAT
GCAGTGGTCCGGAGAGATAGCCAAGGCCAAGTCCACAAGTCCTTT
AGGAGACAGGTCACCACCCTCTCTGACCTCCAGCCATACATGGGC
CAGTTCCTTAAGCATCTGCAGGATTCAGATGCCAGTGCACTGAGG
AACTCCGTTGTCATCGAGCAGAGCATCTCTATGAATGAGAGCAGC
AGCAGCCTGTTTGACTTCTTCCTGCACTTCCTGCGTCACAGTGTC
GTAAAGATTGGTGACAGGTGCTATACGCAGTGCCAGGGCATCCCC
CAGGGCTCCAGCCTATCCACCCTGCTCTGCAGTCTGTGTTTCGGA
GACATGGAGAACAAGCTGTTTGCTGAGGTGCAGCGGGATGGGTTG
CTTTTACGTTTTGTTGATGACTTTCTGTTGGTGACGCCTCACTTG
GACCAAGCAAAAACCTTCCTCAGCACCCTGGTCCATGGCGTTCCT
GAGTATGGGTGCATGATAAACTTGCAGAAGACAGTGGTGAACTTC
CCTGTGGAGCCTGGTACCCTGGGTGGTGCAGCTCCATACCAGCTG
CCTGCTCACTGCCTGTTTCCCTGGTGTGGCTTGCTGCTGGACACT
CAGACTTTGGAGGTGTTCTGTGACTACTCAGGTTATGCCCAGACC
TCAATTAAGACGAGCCTCACCTTCCAGAGTGTCTTCAAAGCTGGG
AAGACCATGCGGAACAAGCTCCTGTCGGTCTTGCGGTTGAAGTGT
CACGGTCTATTTCTAGACTTGCAGGTGAACAGCCTCCAGACAGTC
TGCATCAATATATACAAGATCTTCCTGCTTCAGGCCTACAGGTTC
CATGCATGTGTGATTCAGCTTCCCTTTGACCAGCGTGTTAGGAAG
AACCTCACATTCTTTCTGGGCATCATCTCCAGCCAAGCATCCTGC
TGCTATGCTATCCTGAAGGTCAAGAATCCAGGAATGACACTAAAG
GCCTCTGGCTCCTTTCCTCCTGAAGCCGCACATTGGCTCTGCTAC
CAGGCCTTCCTGCTCAAGCTGGCTGCTCATTCTGTCATCTACAAA
TGTCTCCTGGGACCTCTGAGGACAGCCCAAAAACTGCTGTGCCGG
AAGCTCCCAGAGGCGACAATGACCATCCTTAAAGCTGCAGCTGAC
CCAGCCCTAAGCACAGACTTTCAGACCATTTTGGACTAA

In some embodiments, a TERT mRNA may comprise a nucleic acid sequence at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to any one of SEQ ID NOS: 1-5, 7, 9 or 30.

In some embodiments, a TERT mRNA may encode a modified TERT protein containing one or more amino acid substitutions, deletions, and/or insertions as compared to SEQ ID NOS: 6 or 8, while retaining substantial TERT activity. In some embodiments, a TERT mRNA may encode an amino acid sequence at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 6 or SEQ ID NO: 8.

In other embodiments, a TERT mRNA may encode an amino acid sequence with a mutation of L55Q, P65A, V70M, A202T, A279T, V299M, H412Y, a deletion of residue 441, R522K, K570N, R631Q, G682D, V694M, Y697F, P704S, Y707F, A716T, P721R, T726M, Y772C, P785L, V791I, R811C, L841F, R865H, V867M, R901W, K902N, P923L, S948R, R979W, V1025F, A1062T, V1090M, T1110M, and/or F1127L relative to the amino acid sequences of SEQ ID NO: 6. In some embodiments, the TERT mRNA may encode a TERT isoform in which the translated protein lacks amino acid residues 711-722, 764-807, 808-1132, or 885-947 relative to the amino acid sequences of SEQ ID NO: 6. In some embodiments about 1, about 5, about 10, about 20, or about 100 amino acids preceding or following the domain are also deleted from the amino acid sequence of SEQ ID NO: 6.

In some embodiments, the TERT mRNA may encode an amino acid sequence in which one or more of the protein regions are deleted or repeated relative to the amino acid sequences of SEQ ID NO: 6: residues 1-230 corresponding to the RNA-interacting domain 1, residues 58-197 corresponding to a “GQ” residue motif, residues 137-141 associated with the specificity of telomeric DNA and primer elongation, residues 210-320 corresponding to a disordered region, residues 231-324 associated with a linker sequence, residues 301-538 associated with oligomerization, residues 325-550 or 460-594 corresponding to an RNA-interacting domain, residues 376-521 corresponding to a “QFP” residue motif, residues 397-417 corresponding to a “CP” residue motif, residues 825-884 corresponding to a DNA repeat template, residues 618-729 corresponding to a reverse transcriptase like element, residues 914-928 associated with oligomerization, residues 930-934 associated with a primer grip sequence, and/or residues 936-1132 corresponding to the C-terminus. In some embodiments about 1, about 5, about 10, about 20, or about 100 amino acids preceding or following the domain are also deleted or repeated.

In some embodiments, a TERT mRNA may comprise or consist of a nucleotide sequence at least at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more identical to any of discloses nucleic acid sequences, or to any subsequence of a disclosed nucleic acid sequence, e.g., any 100 base pair (bp), 200 bp, 300 bp, 400 bp, 500 bp, or more of a disclosed nucleic acid sequence. In some embodiments, a TERT mRNA may encode an amino acid sequence at least at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more identical to any of one of the disclosed polypeptide sequences, or to any subsequence of a disclosed polypeptide sequence, e.g., any 50 amino acid (aa), 100 aa, 200 aa, 300 aa, 400 aa, 500 aa, or more of a disclosed polypeptide sequence.

Non-limiting TERT sequences of the disclosure, include TERT nucleic acid and amino acid sequences listed in Table 1A.

TABLE 1A

| | | | Example | Example |
|---|---|---|---|---|
| TERT | Amino Acid | Amino Acid | Nucleic Acid | Nucleic Acid |
| Species | SEQ ID NO: | Sequence | SEQ ID NO: | Sequence |
| Cat | | ASO67359.1 | | KX620456.1 |
| Dog | | NP_001026800.1 | | NM_001031630.1 |
| Mouse | | AAI27069.1 | | BC127068.1 |
| Mouse, | 10 | NP_033380.1 | 14 | NM_009354.2 |
| isoform 1 | | | | |
| Mouse, | 11 | NP_001349316.1 | 15 | NM_001362387.1 |
| isoform 2 | | | | |
| Mouse, | 12 | NP_001349317.1 | 16 | NM_001362388.1 |
| isoform 3 | | | | |
| Mouse | | EDL37055.1 | | Machine reverse |
| | | | | translation of |
| | | | | EDL37055.1 |
| Cow | | NP_001039707.1 | | NM_001046242.1 |
| Sheep, | | XP_027835754.1 | | XM_027979953.1 |
| isoform 1 | | | | |
| Sheep, | | XP_027835755.1 | | XM_027979954.1 |
| isoform 2 | | | | |
| Pig | | NP_001231229.1 | | NM_001244300.1 |
| African | | XP_023401395.1 | | XM_023545627.1 |
| Elephant | | | | |
| Chicken | | NP_001026178.1 | | NM_001031007.1 |
| Rat | 13 | NP_445875.1 | 17 | NM_053423.1 |
| Zebrafish | | NP_001077335.1 | | NM_001083866.1 |
| Japanese | | NP_001098286.1 | | NM_001104816.1 |
| medaka | | | | |
| Horse, | | XP_023481649.1 | | XM_023625881.1 |
| isoform 1 | | | | |
| Horse, | | XP_023481650.1 | | XM_023625882.1 |
| isoform 2 | | | | |
| Horse, | | XP_023481651.1 | | XM_023625883.1 |
| isoform 3 | | | | |

In some embodiments of the compositions and methods of the disclosure, an amino acid sequence of TERT may comprise or consist of a sequence having at least 70%, 75%, 80%, 85%, 90%, 95%, 97%, 99%, 100% or any percentage in between of identity to one or more of SEQ ID NOS: 6-8 or 10-13. In some embodiments of the compositions and methods of the disclosure, an amino acid sequence of a portion of TERT, functional or non-functional, may comprise or consist of a sequence having at least 70%, 75%, 80%, 85%, 90%, 95%, 97%, 99%, 100% or any percentage in between of identity to one or more of SEQ ID Nos: 6-8 or 10-13.

In some embodiments of the compositions and methods of the disclosure, a nucleic acid sequence of TERT may comprise or consist of a sequence having at least 70%, 75%, 80%, 85%, 90%, 95%, 97%, 99%, 100% or any percentage in between of identity to one or more of SEQ ID Nos: 1-5, 7, 9, 14-17, 30 or 31. In some embodiments of the compositions and methods of the disclosure, a nucleic acid sequence of a portion of non-human TERT, functional or non-functional, may comprise or consist of a sequence having at least 70%, 75%, 80%, 85%, 90%, 95%, 97%, 99%, 100% or any percentage in between of identity to one or more of SEQ ID Nos: 1-5, 7, 9, 14-17, 30 or 31.

```
The amino acid sequence of non-human primate TERT isoform 1 may comprise or
consist of the sequence of SEQ ID NO: 18 (also described at GenBank Accession No.
XP_016808391.2):
      1 mpraprcrav rsllrshyre vlplatfvrr lgpqgwrlvq rgdpaafral vaqclvcvpw
    61 darpppaaps frqvsclkel varvlqrlce rgaknvlafg falldgargg ppeafttsvr
  121 sylpntvtda lrgsgawgll lrrvgddvlv hllarcalfv lvapscayqv cgpplyqlga
  181 atqarpppha sgprrrlgce rawnhsvrea gvplglpapg arrrggsasr slplpkrprr
  241 gaapepertp vgqgswahpg rtrgpsdrgf cvvsparpae eatslegals gtrhshpsvg
  301 rqhhagppst srpprpwdtp cppvyaetkh flyssgdkeq lrpsfllssl rpsltgarrl
  361 vetiflgsrp wmpgtprrlp rlpqrywqmr plflellgnh aqcpygvllk thcplraavt
  421 paagvcarek pqgsvaapee edtdprrlvq llrqhsspwq vygfvraclr rlvppglwgs
  481 rhnerrflrn tkkfislgkh aklslqeltw kmsvrdcawl rrspgvgsvp aaehrlreei
  541 lakflhwlms vyvvellrsf fyvtettfqk nrlffyrksv wsklqsigir qhlkrvqlre
  601 lseaevrqhq earpalltsr lrfipkpdgl rpivnmdyvv gartfrrekr aerltsrvka
  661 lfsvlnyera rrpgllgasv lglddihraw rtfvlrvraq dpppelyfvk vdvtgaydti
  721 pqdrltevia siikpqntyc vrryavvqka ahghvrkafk shvstltdlq pymrqfvahl
  781 qetsplrdav iieqssslne assglfdvfl rfvcrhavri rgksyvqcqg ipqgsilstl
  841 lcslcygdme nklfagirrd glllrlvddf llvtphltha kaflrtlvrg vpeygcvvnl
  901 rktvvnfpve dealggtafv qlpahglfpw cgllldtrtl evqsdyssya rtsirasltf
  961 nrgfkagrnm rrklfgvlrl kchslfldlq vnslqtvctn iykilllqay rfhacvlqlp
1021 fhqqvwknpt fflriisdta slcysilkak nagmslgakg aagplpseam qwlchqafll
1081 kltrhrvtyv pllgslrtaq tqlsrklpgt tlsaleaaan palpsdfkti ld.
The nucleic acid sequence of non-human primate TERT isoform 1 may comprise or
consist of the sequence of SEQ ID NO: 19 (also described at GenBank Accession No.
XM_016952902.2):
      1 ctcgcggcgc gagtttcagg cagcgctgcg tcctgctgcg cacgtgggaa gccctggccc
    61 cggccacccc cgcgatgccg cgcgctcccc gctgccgagc cgtgcgctcc ctgctgcgca
  121 gccactaccg cgaggtgctg ccgctggcca cgttcgtgcg gcgcctgggg ccccagggct
  181 ggcggctggt gcagcgcggg gacccggcgg ctttccgcgc gctggtggcc cagtgcctgg
  241 tgtgcgtgcc ctgggacgca cggccgcccc ccgccgcccc ctccttccgc caggtgtcct
  301 gcctgaagga gctggtggcc cgagtgctgc agaggctgtg cgagcgcggc gcgaagaacg
  361 tgctggcctt cggcttcgcg ctgctggacg gggcccgcgg gggccccccc gaggccttca
  421 ccaccagcgt gcgcagctac ctgcccaaca cggtgaccga cgcactgcgg gggagcgggg
  481 cgtgggggct gctgctgcgc cgcgtgggcg acgacgtgct ggttcacctg ctggcacgct
  541 gcgcgctctt tgtgctggtg gctcccagct gcgcctacca ggtgtgcggg ccgccgctgt
  601 accagctcgg cgctgccact caggcccggc ccccgccaca cgctagtgga ccccgaaggc
  661 gtctaggatg cgaacgggcc tggaaccata gcgtcaggga ggccggggtc cccctgggcc
  721 tgccagcccc gggtgcgagg aggcgcgggg gcagtgccag ccgaagtctg ccgttgccca
  781 agaggcccag gcgtggcgct gcccctgage cggagcggac gcccgttggg caggggtcct
  841 gggcccaccc gggcaggacg cgtggaccga gtgaccgtgg tttctgtgtg gtgtcacctg
  901 ccagacccgc cgaagaagcc acctctttgg agggtgcgct ctctggcacg cgccactccc
  961 acccatccgt gggccgccag caccacgcgg gccccccatc cacatcgcgg ccaccacgtc
1021 cctgggacac gccttgtccc ccggtgtacg ccgagaccaa gcacttcctc tactcctcag
1081 gcgacaagga gcagctgcgg ccctccttcc tactcagctc tctgaggccc agcctgactg
1141 gcgctcggag gctcgtggag accatctttc tgggttccag gccctggatg ccagggactc
1201 cccgcaggtt gccccgcctg ccccagcgct actggcaaat gcggcccctg tttctggagc
1261 tgcttgggaa ccacgcgcag tgcccctacg gggtgctcct caagacgcac tgcccgctgc
1321 gagctgcggt caccccagca gccggtgtct gtgcccggga gaagccccag ggctctgtgg
1381 cggcccccga ggaggaggac acagaccccc gtcgcctggt gcagctgctc cgccagcaca
1441 gcagcccctg gcaggtgtac ggcttcgtgc gggcctgcct gcgccggctg gtgcccccag
1501 gcctctgggg ctccaggcac aacgaacgcc gcttcctcag gaacaccaag aagttcatct
1561 ccctggggaa gcatgccaag ctctcgctgc aggagctgac gtggaagatg agcgtgcggg
1621 actgcgcttg gctgcgcagg agcccagggg ttggctctgt tccggccgca gagcaccgtc
1681 tgcgtgagga gatcctggcc aagttcttgc actggctgat gagtgtgtac gttgtcgagc
1741 tgctcaggtc tttcttttat gtcacggaga ccacgtttca gaagaacagg ctctttttct
1801 accggaagag tgtctggagc aagttgcaaa gcattggaat cagacagcac ttgaagaggg
1861 tgcagctgcg ggagctgtcg gaagcagagg tcaggcagca tcaggaagcc aggcccgccc
1921 tgctgacgtc cagactccgc ttcatcccca agcctgacgg gctgcggccg attgtgaaca
1981 tggactacgt cgtgggagcc agaacgttcc gcagagaaaa gagggccgag cgtctcacct
2041 cgagggtgaa ggcactgttc agcgtgctca actacgagcg ggcgcggcgc cccggcctcc
2101 tgggcgcctc tgtgctgggc ctggacgata tccacagggc ctggcgcacc ttcgtgctgc
2161 gtgtgcgggc ccaggacccg ccgcctgagc tgtactttgt caaggtggat gtgacgggcg
2221 cgtacgacac catcccccag gacaggctca cggaggtcat cgccagcatc atcaaacccc
2281 agaacacgta ctgcgtgcgt cggtatgctg tggtccagaa ggccgcccat gggcacgtcc
2341 gcaaggcctt caagagccac gtctctacct tgacagacct ccagccgtac atgcgacagt
2401 tcgtggctca cctgcaggag accagcccac tgagggatgc cgtcatcatc gagcagagct
2461 cctccctgaa tgaggccagc agtggcctct tcgacgtctt cctacgcttc gtgtgccgcc
2521 acgccgtgcg catcaggggc aagtcctacg tccagtgcca ggggatcccg cagggctcca
2581 tcctgtccac gctgctctgc agcctgtgct acggcgacat ggagaacaag ctgtttgcgg
2641 ggattcggcg ggacgggctg ctcctgcgtt tggtggatga tttcttgttg gtgacacctc
2701 acctcaccca cgcgaaagcc ttcctcagga ccctggtccg aggtgtccct gagtatggct
2761 gcgtggtgaa cttgcggaag acagtagtga acttccctgt agaagatgag gccctgggtg
2821 gcacggcttt tgttcagctg ccggcccacg gcctattccc ctggtgcggc ctgctgctgg
2881 acacccggac cctggaggtg cagagcgact actccagcta tgcccggacc tccatcagag
2941 ccagtctcac cttcaaccgc ggcttcaagg ctgggaggaa catgcgtcgc aaactctttg
3001 gggtcttgcg gctgaagtgt cacagcctgt ttctggattt gcaggtgaac agcctccaga
3061 cggtgtgcac caacatctac aagatcctcc tgctgcaggc gtacaggttt cacgcatgtg
3121 tgctgcagct cccatttcat cagcaagttt ggaagaaccc cacatttttc ctgcgcatca
3181 tctctgacac ggcctccctc tgctactcca tectgaaagc caagaacgca gggatgtcgc
3241 tgggggccaa gggtgccgcc ggccctctgc cctccgaggc catgcagtgg ctgtgccacc
3301 aagcattcct gctcaagctg actcgacacc gcgtcaccta cgtgccacte ctggggtcac
3361 tcaggacage ccagacgcag ctgagtcgga agctcccggg gacgacgctg agtgccctgg
3421 aggccgcagc caacccggca ctgccctcag acttcaagac catcctggac tgatggccac
3481 ccgcccacag ccgggccgag agcagacacc agcagccctg tcacgccggg ctctacgtcc
3541 cagggaggga ggggcggccc acacccagac ccgcaccgct gggagtctga ggcctgagtg
3601 agtgtctggc caaggcctgc atgtccggct gaaggctgag tgtccagctg aggcctgagc
3661 gagtgtccag ccaagggctg agtgtccagc acacctgccg tcttcacttc cccacaggct
3721 ggcgctcggc tccaccccag ggccagcttt tcctcgccag gagcccggct tcccactccc
3781 cacatgggaa tagtccatcc ccagattcgc cattgtccac ccctcgccct gccctccttt
3841 gccttccacg cccaccatcc agatggagac cctgagaagg accctgggag ctctgggaat
3901 ttggagtgac caaaggtgtg ccctgtacac aggtgaggac cctgcacctg gatgggggtc
3961 cctgtgggtc aaattggggg gggggtgctg tgggagtaaa atactgaata tatgagtttt
4021 tcagttttga aaaaaa.
The amino acid sequence of non-human primate TERT isoform 2 may comprise or
consist of the sequence of SEQ ID NO: 20, GenBank Accession No. PNI27662.1:
      1 mpraprcrav rsllrshyre vlplatfvrr lgpqgwrlvq rgdpaafral vaqclvcvpw
    61 darpppaaps frqvsclkel varvlqrlce rgaknvlafg falldgargg ppeafttsvr
  121 sylpntvtda lrgsgawgll lrrvgddvlv hllarcalfv lvapscayqv cgpplyqlga
  181 atqarpppha sgprrrlgce rawnhsvrea gvplglpapg arrrggsasr slplpkrprr
  241 gaapepertp vgqgswahpg rtrgpsdrgf cvvsparpae eatslegals gtrhshpsvg
  301 rqhhagppst srpprpwdtp cppvyaetkh flyssgdkeq lrpsfllssl rpsltgarrl
  361 vetiflgsrp wmpgtprrlp rlpqrywqmr plflellgnh aqcpygvllk thcplraavt
  421 paagvcarek pqgsvaapee edtdprrlvq llrqhsspwq vygfvraclr rlvppglwgs
  481 rhnerrflrn tkkfislgkh aklslqeltw kmsvrdcawl rrspgvgsvp aaehrlreei
  541 lakflhwlms vyvvellrsf fyvtettfqk nrlffyrksv wsklqsigir qhlkrvqlre
  601 lseaevrqhq earpalltsr lrfipkpdgl rpivnmdyvv gartfrrekr aerltsrvka
  661 lfsvlnyera rrpgllgasv lglddihraw rtfvlrvraq dpppelyfvk vdvtgaydti
  721 pqdrltevia siikpqntyc vrryavvqka ahghvrkafk shvstltdlq pymrqfvahl
  781 qetsplrdav iieqssslne assglfdvfl rfvcrhavri rgksyvqcqg ipqgsilstl
  841 lcslcygdme nklfagirrd glllrlvddf llvtphltha kaflrtlvrg vpeygcvvnl
  901 rktvvnfpve dealggtafv qlpahglfpw cgllldtrtl evqsdyssya rtsirasltf
  961 nrgfkagrnm rrklfgvlrl kchslfldlq vnslqtvctn iykilllqay rfhacvlqlp
1021 fhqqvwknpt fflriisdta slcysilkak nagmslgakg aagplpseam qwlchqafll
1081 kltrhrvtyv pllgslrtaq tqlsrklpgt tlsaleaaan palpsdfkti ld.
The nucleic acid sequence of non-human primate TERT isoform 2 may comprise or
consist of the sequence of SEQ ID NO: 21 (reverse machine translation of
GenBank Accession No. PNI27662.1):
      1 atgccgcgcg cgccgcgctg ccgcgcggtg cgcagcctgc tgcgcagcca ttatcgcgaa
    61 gtgctgccgc tggcgacctt tgtgcgccgc ctgggcccgc agggctggcg cctggtgcag
  121 cgcggcgatc cggcggcgtt tcgcgcgctg gtggcgcagt gcctggtgtg cgtgccgtgg
  181 gatgcgcgcc cgccgccggc ggcgccgagc tttcgccagg tgagctgcct gaaagaactg
  241 gtggcgcgcg tgctgcagcg cctgtgcgaa cgcggcgcga aaaacgtgct ggcgtttggc
  301 tttgcgctgc tggatggcgc gcgcggcggc ccgccggaag cgtttaccac cagcgtgcgc
  361 agctatctgc cgaacaccgt gaccgatgcg ctgcgcggca gcggcgcgtg gggcctgctg
  421 ctgcgccgcg tgggcgatga tgtgctggtg catctgctgg cgcgctgcgc gctgtttgtg
  481 ctggtggcgc cgagctgcgc gtatcaggtg tgcggcccgc cgctgtatca gctgggcgcg
  541 gcgacccagg cgcgcccgcc gccgcatgcg agcggcccgc gccgccgcct gggctgcgaa
  601 cgcgcgtgga accatagcgt gcgcgaagcg ggcgtgccgc tgggcctgcc ggcgccgggc
  661 gcgcgccgcc gcggcggcag cgcgagccgc agcctgccgc tgccgaaacg cccgcgccgc
  721 ggcgcggcgc cggaaccgga acgcaccccg gtgggccagg gcagctgggc gcatccgggc
  781 cgcacccgcg gcccgagcga tcgcggcttt tgcgtggtga gcccggcgcg cccggcggaa
  841 gaagcgacca gcctggaagg cgcgctgagc ggcacccgcc atagccatcc gagcgtgggc
  901 cgccagcatc atgcgggccc gccgagcacc agccgcccgc cgcgcccgtg ggataccccg
  961 tgcccgccgg tgtatgcgga aaccaaacat tttctgtata gcagcggcga taaagaacag
1021 ctgcgcccga gctttctgct gagcagcctg cgcccgagcc tgaccggcgc gcgccgcctg
1081 gtggaaacca tttttctggg cagccgcccg tggatgccgg gcaccccgcg ccgcctgccg
1141 cgcctgccgc agcgctattg gcagatgcgc ccgctgtttc tggaactgct gggcaaccat
1201 gcgcagtgcc cgtatggcgt gctgctgaaa acccattgcc cgctgcgcgc ggcggtgacc
1261 ccggcggcgg gcgtgtgcgc gcgcgaaaaa ccgcagggca gcgtggcggc gccggaagaa
1321 gaagataccg atccgcgccg cctggtgcag ctgctgcgcc agcatagcag cccgtggcag
1381 gtgtatggct ttgtgcgcgc gtgcctgcgc cgcctggtgc cgccgggcct gtggggcagc
1441 cgccataacg aacgccgctt tctgcgcaac accaaaaaat ttattagcct gggcaaacat
1501 gcgaaactga gcctgcagga actgacctgg aaaatgagcg tgcgcgattg cgcgtggctg
1561 cgccgcagcc cgggcgtggg cagcgtgccg gcggcggaac atcgcctgcg cgaagaaatt
1621 ctggcgaaat ttctgcattg gctgatgagc gtgtatgtgg tggaactgct gcgcagcttt
1681 ttttatgtga ccgaaaccac ctttcagaaa aaccgcctgt ttttttatcg caaaagcgtg
1741 tggagcaaac tgcagagcat tggcattcgc cagcatctga aacgcgtgca gctgcgcgaa
1801 ctgagcgaag cggaagtgcg ccagcatcag gaagcgcgcc cggcgctgct gaccagccgc
1861 ctgcgcttta ttccgaaacc ggatggcctg cgcccgattg tgaacatgga ttatgtggtg
1921 ggcgcgcgca cctttcgccg cgaaaaacgc gcggaacgcc tgaccagccg cgtgaaagcg
1981 ctgtttagcg tgctgaacta tgaacgcgcg cgccgcccgg gcctgctggg cgcgagcgtg
2041 ctgggcctgg atgatattca tcgcgcgtgg cgcacctttg tgctgcgcgt gcgcgcgcag
2101 gatccgccgc cggaactgta ttttgtgaaa gtggatgtga ccggcgcgta tgataccatt
2161 ccgcaggatc gcctgaccga agtgattgcg agcattatta aaccgcagaa cacctattgc
2221 gtgcgccgct atgcggtggt gcagaaagcg gcgcatggcc atgtgcgcaa agcgtttaaa
2281 agccatgtga gcaccctgac cgatctgcag ccgtatatgc gccagtttgt ggcgcatctg
2341 caggaaacca gcccgctgcg cgatgcggtg attattgaac agagcagcag cctgaacgaa
2401 gcgagcagcg gcctgtttga tgtgtttctg cgctttgtgt gccgccatgc ggtgcgcatt
2461 cgcggcaaaa gctatgtgca gtgccagggc attccgcagg gcagcattct gagcaccctg
2521 ctgtgcagcc tgtgctatgg cgatatggaa aacaaactgt ttgcgggcat tcgccgcgat
2581 ggcctgctgc tgcgcctggt ggatgatttt ctgctggtga ccccgcatct gacccatgcg
2641 aaagcgtttc tgcgcaccct ggtgcgcggc gtgccggaat atggctgcgt ggtgaacctg
2701 cgcaaaaccg tggtgaactt tccggtggaa gatgaagcgc tgggcggcac cgcgtttgtg
2761 cagctgccgg cgcatggcct gtttccgtgg tgcggcctgc tgctggatac ccgcaccctg
2821 gaagtgcaga gcgattatag cagctatgcg cgcaccagca ttcgcgcgag cctgaccttt
2881 aaccgcggct ttaaagcggg ccgcaacatg cgccgcaaac tgtttggcgt gctgcgcctg
2941 aaatgccata gcctgtttct ggatctgcag gtgaacagcc tgcagaccgt gtgcaccaac
3001 atttataaaa ttctgctgct gcaggcgtat cgctttcatg cgtgcgtgct gcagctgccg
3061 tttcatcagc aggtgtggaa aaacccgacc ttttttctgc gcattattag cgataccgcg
3121 agcctgtgct atagcattct gaaagcgaaa aacgcgggca tgagcctggg cgcgaaaggc
3181 gcggcgggcc cgctgccgag cgaagcgatg cagtggctgt gccatcaggc gtttctgctg
3241 aaactgaccc gccatcgcgt gacctatgtg ccgctgctgg gcagcctgcg caccgcgcag
3301 acccagctga gccgcaaact gccgggcacc accctgagcg cgctggaagc ggcggcgaac
3361 ccggcgctgc cgagcgattt taaaaccatt ctggat,
The amino acid sequence of non-human primate TERT isoform 3 may comprise or
consist of the sequence of SEQ ID NO: 22 (also described at GenBank Accession No.
PNI27663.1):
      1 mpraprcrav rsllrshyre vlplatfvrr lgpqgwrlvq rgdpaafral vaqclvcvpw
    61 darpppaaps frqvsclkel varvlqrlce rgaknvlafg falldgargg ppeafttsvr
  121 sylpntvtda lrgsgawgll lrrvgddvlv hllarcalfv lvapscayqv cgpplyqlga
  181 atqarpppha sgprrrlgce rawnhsvrea gvplglpapg arrrggsasr slplpkrprr
  241 gaapepertp vgqgswahpg rtrgpsdrgf cvvsparpae eatslegals gtrhshpsvg
  301 rqhhagppst srpprpwdtp cppvyaetkh flyssgdkeq lrpsfllssl rpsltgarrl
  361 vetiflgsrp wmpgtprrlp rlpqrywqmr plflellgnh aqcpygvllk thcplraavt
  421 paagvcarek pqgsvaapee edtdprrlvq llrqhsspwq vygfvraclr rlvppglwgs
  481 rhnerrflrn tkkfislgkh aklslqeltw kmsvrdcawl rrspgvgsvp aaehrlreei
  541 lakflhwlms vyvvellrsf fyvtettfqk nrlffyrksv wsklqsigir qhlkrvqlre
  601 lseaevrqhq earpalltsr lrfipkpdgl rpivnmdyvv gartfrrekr aerltsrvka
  661 lfsvlnyera rrpgllgasv lglddihraw rtfvlrvraq dpppelyfvk vdvtgaydi
  721 pqdrltevia siikpqntyc vrryavvqka ahghvrkafk shvlrpvpgd paglhpvhaa
  781 lqpvlrrhge qavcgdsagr aapafgg.
The nucleic acid sequence of non-human primate TERT isoform 3 may comprise or
consist of the sequence of SEQ ID NO: 23 (reverse machine translation
of GenBank Accession No. PNI27663.1):
      1 atgccgcgcg cgccgcgctg ccgagcggtg cgcagcctgc tgcgcagcca ttatcgcgaa
    61 gtgctgccgc tggcgacctt tgtgcgccgc ctgggcccgc agggctggcg cctggtgcag
  121 cgcggcgatc cggcggcgtt tcgcgcgctg gtggcgcagt gcctggtgtg cgtgccgtgg
  181 gatgcgcgcc cgccgccggc ggcgccgagc tttcgccagg tgagctgcct gaaagaactg
  241 gtggcgcgcg tgctgcagcg cctgtgcgaa cgcggcgcga aaaacgtgct ggcgtttggc
  301 tttgcgctgc tggatggcgc gcgcggcggc ccgccggaag cgtttaccac cagcgtgcgc
  361 agctatctgc cgaacaccgt gaccgatgcg ctgcgcggca gcggcgcgtg gggcctgctg
  421 ctgcgccgcg tgggcgatga tgtgctggtg catctgctgg cgcgctgcgc gctgtttgtg
  481 ctggtggcgc cgagctgcgc gtatcaggtg tgcggcccgc cgctgtatca gctgggcgcg
  541 gcgacccagg cgcgcccgcc gccgcatgcg agcggcccgc gccgccgcct gggctgcgaa
  601 cgcgcgtgga accatagcgt gcgcgaagcg ggcgtgccgc tgggcctgcc ggcgccgggc
  661 gcgcgccgcc gcggcggcag cgcgagccgc agcctgccgc tgccgaaacg cccgcgccgc
  721 ggcgcggcgc cggaaccgga acgcaccccg gtgggccagg gcagctgggc gcatccgggc
  781 cgcacccgcg gcccgagcga tcgcggcttt tgcgtggtga gcccggcgcg cccggcggaa
  841 gaagcgacca gcctggaagg cgcgctgagc ggcacccgcc atagccatcc gagcgtgggc
  901 cgccagcatc atgcgggccc gccgagcacc agccgcccgc cgcgcccgtg ggataccccg
  961 tgcccgccgg tgtatgcgga aaccaaacat tttctgtata gcagcggcga taaagaacag
1021 ctgcgcccga gctttctgct gagcagcctg cgcccgagcc tgaccggcgc gcgccgcctg
1081 gtggaaacca tttttctggg cagccgcccg tggatgccgg gcaccccgcg ccgcctgccg
1141 cgcctgccgc agcgctattg gcagatgcgc ccgctgtttc tggaactgct gggcaaccat
1201 gcgcagtgcc cgtatggcgt gctgctgaaa acccattgcc cgctgcgcgc ggcggtgacc
1261 ccggcggcgg gcgtgtgcgc gcgcgaaaaa ccgcagggca gcgtggcggc gccggaagaa
1321 gaagataccg atccgcgccg cctggtgcag ctgctgcgcc agcatagcag cccgtggcag
1381 gtgtatggct ttgtgcgcgc gtgcctgcgc cgcctggtgc cgccgggcct gtggggcagc
1441 cgccataacg aacgccgctt tctgcgcaac accaaaaaat ttattagcct gggcaaacat
1501 gcgaaactga gcctgcagga actgacctgg aaaatgagcg tgcgcgattg cgcgtggctg
1561 cgccgcagcc cgggcgtggg cagcgtgccg gcggcggaac atcgcctgcg cgaagaaatt
1621 ctggcgaaat ttctgcattg gctgatgagc gtgtatgtgg tggaactgct gcgcagcttt
1681 ttttatgtga ccgaaaccac ctttcagaaa aaccgcctgt ttttttatcg caaaagcgtg
1741 tggagcaaac tgcagagcat tggcattcgc cagcatctga aacgcgtgca gctgcgcgaa
1801 ctgagcgaag cggaagtgcg ccagcatcag gaagcgcgcc cggcgctgct gaccagccgc
1861 ctgcgcttta ttccgaaacc ggatggcctg cgcccgattg tgaacatgga ttatgtggtg
1921 ggcgcgcgca cctttcgccg cgaaaaacgc gcggaacgcc tgaccagccg cgtgaaagcg
1981 ctgtttagcg tgctgaacta tgaacgcgcg cgccgcccgg gcctgctggg cgcgagcgtg
2041 ctgggcctgg atgatattca tcgcgcgtgg cgcacctttg tgctgcgcgt gcgcgcgcag
2101 gatccgccgc cggaactgta ttttgtgaaa gtggatgtga ccggcgcgta tgataccatt
2161 ccgcaggatc gcctgaccga agtgattgcg agcattatta aaccgcagaa cacctattgc
2221 gtgcgccgct atgcggtggt gcagaaagcg gcgcatggcc atgtgcgcaa agcgtttaaa
2281 agccatgtgc tgcgcccggt gccgggcgat ccggcgggcc tgcatccggt gcatgcggcg
2341 ctgcagccgg tgctgcgccg ccatggcgaa caggcggtgt gcggcgatag cgcgggccgc
2401 gcggcgccgg cgtttggcgg c.
The amino acid sequence of non-human primate TERT isoform 4 may comprise or
consist of the sequence of SEQ ID NO: 24 (also described at GenBank Accession No.
PNI27664.1):
      1 mpraprcrav rsllrshyre vlplatfvrr lgpqgwrlvq rgdpaafral vaqclvcvpw
    61 darpppaaps frqvsclkel varvlqrlce rgaknvlafg falldgargg ppeafttsvr
  121 sylpntvtda lrgsgawgll lrrvgddvlv hllarcalfv Ivapscayqv cgpplyqlga
  181 atqarpppha sgprrrlgce rawnhsvrea gvplglpapg arrrggsasr slplpkrprr
  241 gaapepertp vgqgswahpg rtrgpsdrgf cvvsparpae eatslegals gtrhshpsvg
  301 rqhhagppst srpprpwdtp cppvyaetkh flyssgdkeq lrpsfllssl rpsltgarrl
  361 vetiflgsrp wmpgtprrlp rlpqrywqmr plflellgnh aqcpygvllk thcplraavt
  421 paagvcarek pqgsvaapee edtdprrlvq llrqhsspwq vygfvraclr rlvppglwgs
  481 rhnerrflrn tkkfislgkh aklslqeltw kmsvrdcawl rrspgvgsvp aaehrlreei
  541 lakflhwlms vyvvellrsf fyvtettfqk nrlffyrksv wsklqsigir qhlkrvqlre
  601 lseaevrqhq earpalltsr lrfipkpdgl rpivnmdyvv gartfrrekr aerltsrvka
  661 lfsvlnyera rrpgllgasv lglddihraw rtfvlrvraq dpppelyfvk vdvtgaydti
  721 pqdrltevia siikpqntyc vrryavvqka ahghvrkafk shvstltdlq pymrqfvahl
  781 qetsplrdav iieqssslne assglfdvfl rfvcrhavri rgksyvqcqg ipqgsilstl
  841 lcslcygdme nklfagirrd glllrlvddf llvtphltha kaflsyarts irasltfnrg
  901 fkagrnmrrk lfgvlrlkch slfldlqvns lqtvctniyk illlqayrfh acvlqlpfhq
  961 qvwknptffl riisdtaslc ysilkaknag mslgakgaag plpseamqwl chqafllklt
1021 rhrvtyvpll gslrtaqtql srklpgttls aleaaanpal psdfktild.
The nucleic acid sequence of non-human primate TERT isoform 4 may comprise or
consist of the sequence of SEQ ID NO: 25 (reverse machine translation of
GenBank Accession No. PNI27664.1):
      1 atgccgcgcg cgccgcgctg ccgcgcggtg cgcagcctgc tgcgcagcca ttatcgcgaa
    61 gtgctgccgc tggcgacctt tgtgcgccgc ctgggcccgc agggctggcg cctggtgcag
  121 cgcggcgatc cggcggcgtt tcgcgcgctg gtggcgcagt gcctggtgtg cgtgccgtgg
  181 gatgcgcgcc cgccgccggc ggcgccgagc tttcgccagg tgagctgcct gaaagaactg
  241 gtggcgcgcg tgctgcagcg cctgtgcgaa cgcggcgcga aaaacgtgct ggcgtttggc
  301 tttgcgctgc tggatggcgc gcgcggcggc ccgccggaag cgtttaccac cagcgtgcgc
  361 agctatctgc cgaacaccgt gaccgatgcg ctgcgcggca gcggcgcgtg gggcctgctg
  421 ctgcgccgcg tgggcgatga tgtgctggtg catctgctgg cgcgctgcgc gctgtttgtg
  481 ctggtggcgc cgagctgcgc gtatcaggtg tgcggcccgc cgctgtatca gctgggcgcg
  541 gcgacccagg cgcgcccgcc gccgcatgcg agcggcccgc gccgccgcct gggctgcgaa
  601 cgcgcgtgga accatagcgt gcgcgaagcg ggcgtgccgc tgggcctgcc ggcgccgggc
  661 gcgcgccgcc gcggcggcag cgcgagccgc agcctgccgc tgccgaaacg cccgcgccgc
  721 ggcgcggcgc cggaaccgga acgcaccccg gtgggccagg gcagctgggc gcatccgggc
  781 cgcacccgcg gcccgagcga tcgcggcttt tgcgtggtga gcccggcgcg cccggcggaa
  841 gaagcgacca gcctggaagg cgcgctgagc ggcacccgcc atagccatcc gagcgtgggc
  901 cgccagcatc atgcgggccc gccgagcacc agccgcccgc cgcgcccgtg ggataccccg
  961 tgcccgccgg tgtatgcgga aaccaaacat tttctgtata gcagcggcga taaagaacag
1021 ctgcgcccga gctttctgct gagcagcctg cgcccgagcc tgaccggcgc gcgccgcctg
1081 gtggaaacca tttttctggg cagccgcccg tggatgccgg gcaccccgcg ccgcctgccg
1141 cgcctgccgc agcgctattg gcagatgcgc ccgctgtttc tggaactgct gggcaaccat
1201 gcgcagtgcc cgtatggcgt gctgctgaaa acccattgcc cgctgcgcgc ggcggtgacc
1261 ccggcggcgg gcgtgtgcgc gcgcgaaaaa ccgcagggca gcgtggcggc gccggaagaa
1321 gaagataccg atccgcgccg cctggtgcag ctgctgcgcc agcatagcag cccgtggcag
1381 gtgtatggct ttgtgcgcgc gtgcctgcgc cgcctggtgc cgccgggcct gtggggcagc
1441 cgccataacg aacgccgctt tctgcgcaac accaaaaaat ttattagcct gggcaaacat
1501 gcgaaactga gcctgcagga actgacctgg aaaatgagcg tgcgcgattg cgcgtggctg
1561 cgccgcagcc cgggcgtggg cagcgtgccg gcggcggaac atcgcctgcg cgaagaaatt
1621 ctggcgaaat ttctgcattg gctgatgagc gtgtatgtgg tggaactgct gcgcagcttt
1681 ttttatgtga ccgaaaccac ctttcagaaa aaccgcctgt ttttttatcg caaaagcgtg
1741 tggagcaaac tgcagagcat tggcattcgc cagcatctga aacgcgtgca gctgcgcgaa
1801 ctgagcgaag cggaagtgcg ccagcatcag gaagcgcgcc cggcgctgct gaccagccgc
1861 ctgcgcttta ttccgaaacc ggatggcctg cgcccgattg tgaacatgga ttatgtggtg
1921 ggcgcgcgca cctttcgccg cgaaaaacgc gcggaacgcc tgaccagccg cgtgaaagcg
1981 ctgtttagcg tgctgaacta tgaacgcgcg cgccgcccgg gcctgctggg cgcgagcgtg
2041 ctgggcctgg atgatattca tcgcgcgtgg cgcacctttg tgctgcgcgt gcgcgcgcag
2101 gatccgccgc cggaactgta ttttgtgaaa gtggatgtga ccggcgcgta tgataccatt
2161 ccgcaggatc gcctgaccga agtgattgcg agcattatta aaccgcagaa cacctattgc
2221 gtgcgccgct atgcggtggt gcagaaagcg gcgcatggcc atgtgcgcaa agcgtttaaa
2281 agccatgtga gcaccctgac cgatctgcag ccgtatatgc gccagtttgt ggcgcatctg
2341 caggaaacca gcccgctgcg cgatgcggtg attattgaac agagcagcag cctgaacgaa
2401 gcgagcagcg gcctgtttga tgtgtttctg cgctttgtgt gccgccatgc ggtgcgcatt
2461 cgcggcaaaa gctatgtgca gtgccagggc attccgcagg gcagcattct gagcaccctg
2521 ctgtgcagcc tgtgctatgg cgatatggaa aacaaactgt ttgcgggcat tcgccgcgat
2581 ggcctgctgc tgcgcctggt ggatgatttt ctgctggtga ccccgcatct gacccatgcg
2641 aaagcgtttc tgagctatgc gcgcaccagc attcgcgcga gcctgacctt taaccgcggc
2701 tttaaagcgg gccgcaacat gcgccgcaaa ctgtttggcg tgctgcgcct gaaatgccat
2761 agcctgtttc tggatctgca ggtgaacagc ctgcagaccg tgtgcaccaa catttataaa
2821 attctgctgc tgcaggcgta tcgctttcat gcgtgcgtgc tgcagctgcc gtttcatcag
2881 caggtgtgga aaaacccgac cttttttctg cgcattatta gcgataccgc gagcctgtgc
2941 tatagcattc tgaaagcgaa aaacgcgggc atgagcctgg gcgcgaaagg cgcggcgggc
3001 ccgctgccga gcgaagcgat gcagtggctg tgccatcagg cgtttctgct gaaactgacc
3061 cgccatcgcg tgacctatgt gccgctgctg ggcagcctgc gcaccgcgca gacccagctg
3121 agccgcaaac tgccgggcac caccctgagc gcgctggaag cggcggcgaa cccggcgctg
3181 ccgagcgatt ttaaaaccat tctggat.
The amino acid sequence of non-human primate TERT isoform 5 may comprise or
consist of the sequence of SEQ ID NO: 26 (also described at GenBank Accession No.
PNI27665.1):
      1 mpraprcrav rsllrshyre vlplatfvrr lgpqgwrlvq rgdpaafral vaqclvcvpw
    61 darpppaaps frqvsclkel varvlqrlce rgaknvlafg falldgargg ppeafttsvr
  121 sylpntvtda lrgsgawgll lrrvgddvlv hllarcalfv Ivapscayqv cgpplyqlga
  181 atqarpppha sgprrrlgce rawnhsvrea gvplglpapg arrrggsasr slplpkrprr
  241 gaapepertp vgqgswahpg rtrgpsdrgf cvvsparpae eatslegals gtrhshpsvg
  301 rqhhagppst srpprpwdtp cppvyaetkh flyssgdkeq lrpsfllssl rpsltgarrl
  361 vetiflgsrp wmpgtprrlp rlpqrywqmr plflellgnh aqcpygvllk thcplraavt
  421 paagvcarek pqgsvaapee edtdprrlvq llrqhsspwq vygfvraclr rlvppglwgs
  481 rhnerrflrn tkkfislgkh aklslqeltw kmsvrdcawl rrspgvgsvp aaehrlreei
  541 lakflhwlms vyvvellrsf fyvtettfqk nrlffyrksv wsklqsigir qhlkrvqlre
  601 lseaevrqhq earpalltsr lrfipkpdgl rpivnmdyvv gartfrrekr aerltsrvka
  661 lfsvlnyera rrpgllgasv lglddihraw rtfvlrvraq dpppelyfvk drlteviasi
  721 ikpqntycvr ryavvqkaah ghvrkafksh vlrpvpgdpa glhpvhaalq pvlrrhgeqa
  781 vcgdsagraa pafgg.
The nucleic acid sequence of non-human primate TERT isoform 5 may comprise or
consist of the sequence of SEQ ID NO: 27 (reverse machine translation of
GenBank Accession No. PNI27665.1):
      1 atgccgcgcg cgccgcgctg ccgcgcggtg cgcagcctgc tgcgcagcca ttatcgcgaa
    61 gtgctgccgc tggcgacctt tgtgcgccgc ctgggcccgc agggctggg cctggtgcag
  121 cgcggcgatc cggcggcgtt tcgcgcgctg gtggcgcagt gcctggtgtg cgtgccgtgg
  181 gatgcgcgcc cgccgccggc ggcgccgagc tttcgccagg tgagctgcct gaaagaactg
  241 gtggcgcgcg tgctgcagcg cctgtgcgaa cgcggcgcga aaaacgtgct ggcgtttggc
  301 tttgcgctgc tggatggcgc gcgcggcggc ccgccggaag cgtttaccac cagcgtgcgc
  361 agctatctgc cgaacaccgt gaccgatgcg ctgcgcggca gcggcgcgtg gggcctgctg
  421 ctgcgccgcg tgggcgatga tgtgctggtg catctgctgg cgcgctgcgc gctgtttgtg
  481 ctggtggcgc cgagctgcgc gtatcaggtg tgcggcccgc cgctgtatca gctgggcgcg
  541 gcgacccagg cgcgcccgcc gccgcatgcg agcggcccgc gccgccgcct gggctgcgaa
  601 cgcgcgtgga accatagcgt gcgcgaagcg ggcgtgccgc tgggcctgcc ggcgccgggc
  661 gcgcgccgcc gcggcggcag cgcgagccgc agcctgccgc tgccgaaacg cccgcgccgc
  721 ggcgcggcgc cggaaccgga acgcaccccg gtgggccagg gcagctgggc gcatccgggc
  781 cgcacccgcg gcccgagcga tcgcggcttt tgcgtggtga gcccggcgcg cccggcggaa
  841 gaagcgacca gcctggaagg cgcgctgagc ggcacccgcc atagccatcc gagcgtgggc
  901 cgccagcatc atgcgggccc gccgagcacc agccgcccgc cgcgcccgtg ggataccccg
  961 tgcccgccgg tgtatgcgga aaccaaacat tttctgtata gcagcggcga taaagaacag
1021 ctgcgcccga gctttctgct gagcagcctg cgcccgagcc tgaccggcgc gcgccgcctg
1081 gtggaaacca tttttctggg cagccgcccg tggatgccgg gcaccccgcg ccgcctgccg
1141 cgcctgccgc agcgctattg gcagatgcgc ccgctgtttc tggaactgct gggcaaccat
1201 gcgcagtgcc cgtatggcgt gctgctgaaa acccattgcc cgctgcgcgc ggcggtgacc
1261 ccggcggcgg gcgtgtgcgc gcgcgaaaaa ccgcagggca gcgtggcggc gccggaagaa
1321 gaagataccg atccgcgccg cctggtgcag ctgctgcgcc agcatagcag cccgtggcag
1381 gtgtatggct ttgtgcgcgc gtgcctgcgc cgcctggtgc cgccgggcct gtggggcagc
1441 cgccataacg aacgccgctt tctgcgcaac accaaaaaat ttattagcct gggcaaacat
1501 gcgaaactga gcctgcagga actgacctgg aaaatgagcg tgcgcgattg cgcgtggctg
1561 cgccgcagcc cgggcgtggg cagcgtgccg gcggcggaac atcgcctgcg cgaagaaatt
1621 ctggcgaaat ttctgcattg gctgatgagc gtgtatgtgg tggaactgct gcgcagcttt
1681 ttttatgtga ccgaaaccac ctttcagaaa aaccgcctgt ttttttatcg caaaagcgtg
1741 tggagcaaac tgcagagcat tggcattcgc cagcatctga aacgcgtgca gctgcgcgaa
1801 ctgagcgaag cggaagtgcg ccagcatcag gaagcgcgcc cggcgctgct gaccagccgc
1861 ctgcgcttta ttccgaaacc ggatggcctg cgcccgattg tgaacatgga ttatgtggtg
1921 ggcgcgcgca cctttcgccg cgaaaaacgc gcggaacgcc tgaccagccg cgtgaaagcg
1981 ctgtttagcg tgctgaacta tgaacgcgcg cgccgcccgg gcctgctggg cgcgagcgtg
2041 ctgggcctgg atgatattca tcgcgcgtgg cgcacctttg tgctgcgcgt gcgcgcgcag
2101 gatccgccgc cggaactgta ttttgtgaaa gatcgcctga ccgaagtgat tgcgagcatt
2161 attaaaccgc agaacaccta ttgcgtgcgc cgctatgcgg tggtgcagaa agcggcgcat
2221 ggccatgtgc gcaaagcgtt taaaagccat gtgctgcgcc cggtgccggg cgatccggcg
2281 ggcctgcatc cggtgcatgc ggcgctgcag ccggtgctgc gccgccatgg cgaacaggcg
2341 gtgtgcggcg atagcgcggg ccgcgcggcg ccggcgtttg gcggc.
The amino acid sequence of non-human primate TERT isoform 6 may comprise or
consist of the sequence of SEQ ID NO: 28 (also described at GenBank
Accession No. PNI27666.1):
      1 mpraprcrav rsllrshyre vlplatfvrr lgpqgwrlvq rgdpaafral vaqclvcvpw
    61 darpppaaps frqvsclkel varvlqrlce rgaknvlafg falldgargg ppeafttsvr
  121 sylpntvtda lrgsgawgll lrrvgddvlv hllarcalfv lvapscayqv cgpplyqlga
  181 atqarpppha sgprrrlgce rawnhsvrea gvplglpapg arrrggsasr slplpkrprr
  241 gaapepertp vgqgswahpg rtrgpsdrgf cvvsparpae eatslegals gtrhshpsvg
  301 rqhhagppst srpprpwdtp cppvyaetkh flyssgdkeq lrpsfllssl rpsltgarrl
  361 vetiflgsrp wmpgtprrlp rlpqrywqmr plflellgnh aqcpygvllk thcplraavt
  421 paagvcarek pqgsvaapee edtdprrlvq llrqhsspwq vygfvraclr rlvppglwgs
  481 rhnerrflrn tkkfislgkh aklslqeltw kmsvrdcawl rrspgvgsvp aaehrlreei
  541 lakflhwlms vyvvellrsf fyvtettfqk nrlffyrksv wsklqsigir qhlkrvqlre
  601 lseaevrqhq earpalltsr lrfipkpdgl rpivnmdyvv gartfrrekr aerltsrvka
  661 lfsvlnyera rrpgllgasv lglddihraw rtfvlrvraq dpppelyfvk vdvtgaydti
  721 pqdrltevia siikpqntyc vrryavvqka ahghvrkafk shvstltdlq pymrqfvahl
  781 qetsplrdav iieqssslne assglfdvfl rfvcrhavri rgksyvqcqg ipqgsilstl
  841 lcslcygdme nklfagirrd glllrlvddf llvtphltha kaflrtlvrg vpeygcvvnl
  901 rktvvnfpve dealggtafv qlpahglfpw cgllldtrtl evqsdyssya rtsirasltf
  961 nrgfkagrnm rrklfgvlrl kchslfldlq vnslqtvctn iykilllqay rfhacvlqlp
1021 fhqqvwknpt fflriisdta slcysilkak naaqtqlsrk lpgttlsale aaanpalpsd
1081 fktild.
The nucleic acid sequence of non-human primate TERT isoform 6 may comprise or
consist of the sequence of SEQ ID NO: 29 (reverse machine translation of
GenBank Accession No. PNI27666.1):
      1 atgccgcgcg cgccgcgctg ccgcgcggtg cgcagcctgc tgcgcagcca ttatcgcgaa
    61 gtgctgccgc tggcgacctt tgtgcgccgc ctgggcccgc agggctggcg cctggtgcag
  121 cgcggcgatc cggcggcgtt tcgcgcgctg gtggcgcagt gcctggtgtg cgtgccgtgg
  181 gatgcgcgcc cgccgccggc ggcgccgagc tttcgccagg tgagctgcct gaaagaactg
  241 gtggcgcgcg tgctgcagcg cctgtgcgaa cgcggcgcga aaaacgtgct ggcgtttggc
  301 tttgcgctgc tggatggcgc gcgcggcggc ccgccggaag cgtttaccac cagcgtgcgc
  361 agctatctgc cgaacaccgt gaccgatgcg ctgcgcggca gcggcgcgtg gggcctgctg
  421 ctgcgccgcg tgggcgatga tctgctggtg catctgctgg cgcgctgcgc gctgtttgtg
  481 ctggtggcgc cgagctgcgc gtatcaggtg tgcggcccgc cgctgtatca gctgggcgcg
  541 gcgacccagg cgcgcccgcc gccgcatgcg agcggcccgc gccgccgcct gggctgcgaa
  601 cgcgcgtgga accatagcgt gcgcgaagcg ggcgtgccgc tgggcctgcc ggcgccgggc
  661 gcgcgccgcc gcggcggcag cgcgagccgc agcctgccgc tgccgaaacg cccgcgccgc
  721 ggcgcggcgc cggaaccgga acgcaccccg gtgggccagg gcagctgggc gcatccgggc
  781 cgcacccgcg gcccgagcga tcgcggcttt tgcgtggtga gcccggcgcg cccggcggaa
  841 gaagcgacca gcctggaagg cgcgctgagc ggcacccgcc atagccatcc gagcgtgggc
  901 cgccagcatc atgcgggccc gccgagcacc agccgcccgc cgcgcccgtg ggataccccg
  961 tgcccgccgg tgtatgcgga aaccaaacat tttctgtata gcagcggcga taaagaacag
1021 ctgcgcccga gctttctgct gagcagcctg cgcccgagcc tgaccggcgc gcgccgcctg
1081 gtggaaacca tttttctggg cagccgcccg tggatgccgg gcaccccgcg ccgcctgccg
1141 cgcctgccgc agcgctattg gcagatgcgc ccgctgtttc tggaactgct gggcaaccat
1201 gcgcagtgcc cgtatggcgt gctgctgaaa acccattgcc cgctgcgcgc ggcggtgacc
1261 ccggcggcgg gcgtgtgcgc gcgcgaaaaa ccgcagggca gcgtggcggc gccggaagaa
1321 gaagataccg atccgcgccg cctggtgcag ctgctgcgcc agcatagcag cccgtggcag
1381 gtgtatggct ttgtgcgcgc gtgcctgcgc cgcctggtgc cgccgggcct gtggggcagc
1441 cgccataacg aacgccgctt tctgcgcaac accaaaaaat ttattagcct gggcaaacat
1501 gcgaaactga gcctgcagga actgacctgg aaaatgagcg tgcgcgattg cgcgtggctg
1561 cgccgcagcc cgggcgtggg cagcgtgccg gcggcggaac atcgcctgcg cgaagaaatt
1621 ctggcgaaat ttctgcattg gctgatgagc gtgtatgtgg tggaactgct gcgcagcttt
1681 ttttatgtga ccgaaaccac ctttcagaaa aaccgcctgt ttttttatcg caaaagcgtg
1741 tggagcaaac tgcagagcat tggcattcgc cagcatctga aacgcgtgca gctgcgcgaa
1801 ctgagcgaag cggaagtgcg ccagcatcag gaagcgcgcc cggcgctgct gaccagccgc
1861 ctgcgcttta ttccgaaacc ggatggcctg cgcccgattg tgaacatgga ttatgtggtg
1921 ggcgcgcgca cctttcgccg cgaaaaacgc gcggaacgcc tgaccagccg cgtgaaagcg
1981 ctgtttagcg tgctgaacta tgaacgcgcg cgccgcccgg gcctgctggg cgcgagcgtg
2041 ctgggcctgg atgatattca tcgcgcgtgg cgcacctttg tgctgcgcgt gcgcgcgcag
2101 gatccgccgc cggaactgta ttttgtgaaa gtggatgtga ccggcgcgta tgataccatt
2161 ccgcaggatc gcctgaccga agtgattgcg agcattatta aaccgcagaa cacctattgc
2221 gtgcgccgct atgcggtggt gcagaaagcg gcgcatggcc atgtgcgcaa agcgtttaaa
2281 agccatgtga gcaccctgac cgatctgcag ccgtatatgc gccagtttgt ggcgcatctg
2341 caggaaacca gcccgctgcg cgatgcggtg attattgaac agagcagcag cctgaacgaa
2401 gcgagcagcg gcctgtttga tgtgtttctg cgctttgtgt gccgccatgc ggtgcgcatt
2461 cgcggcaaaa gctatgtgca gtgccagggc attccgcagg gcagcattct gagcaccctg
2521 ctgtgcagcc tgtgctatgg cgatatggaa aacaaactgt ttgcgggcat tcgccgcgat
2581 ggcctgctgc tgcgcctggt ggatgatttt ctgctggtga ccccgcatct gacccatgcg
2641 aaagcgtttc tgcgcaccct ggtgcgcggc gtgccggaat atggctgcgt ggtgaacctg
2701 cgcaaaaccg tggtgaactt tccggtggaa gatgaagcgc tgggcggcac cgcgtttgtg
2761 cagctgccgg cgcatggcct gtttccgtgg tgcggcctgc tgctggatac ccgcaccctg
2821 gaagtgcaga gcgattatag cagctatgcg cgcaccagca ttcgcgcgag cctgaccttt
2881 aaccgcggct ttaaagcggg ccgcaacatg cgccgcaaac tgtttggcgt gctgcgcctg
2941 aaatgccata gcctgtttct ggatctgcag gtgaacagcc tgcagaccgt gtgcaccaac
3001 atttataaaa ttctgctgct gcaggcgtat cgctttcatg cgtgcgtgct gcagctgccg
3061 tttcatcagc aggtgtggaa aaacccgacc ttttttctgc gcattattag cgataccgcg
3121 agcctgtgct atagcattct gaaagcgaaa aacgcggcgc agacccagct gagccgcaaa
3181 ctgccgggca ccaccctgag cgcgctggaa gcggcggcga acccggcgct gccgagcgat
3241 tttaaaacca ttctggat.
```

In some embodiments of the compositions and methods of the disclosure, an amino acid sequence of TERT may comprise or consist of a sequence having at least 70%, 75%, 80%, 85%, 90%, 95%, 97%, 99%, 100% or any percentage in between of identity to one or more of SEQ ID NOS: 6, 8, 10-13, 18, 20, 22, 24, 26, or 28. In some embodiments of the compositions and methods of the disclosure, an amino acid sequence of a portion of TERT, functional or non-functional, may comprise or consist of a sequence having at least 70%, 75%, 80%, 85%, 90%, 95%, 97%, 99%, 100% or any percentage in between of identity to one or more of SEQ ID NOS: 6, 8, 10-13, 18, 20, 22, 24, 26, or 28.

In some embodiments of the compositions and methods of the disclosure, a nucleic acid sequence of TERT may comprise or consist of a sequence having at least 70%, 75%, 80%, 85%, 90%, 95%, 97%, 99%, 100% or any percentage in between of identity to one or more of SEQ ID Nos: 1-5, 7, 9, 14-17, 19, 21, 23, 25, 27, 29, 30, or 31. In some embodiments of the compositions and methods of the disclosure, a nucleic acid sequence of a portion of non-human primate TERT, functional or non-functional, may comprise or consist of a sequence having at least 70%, 75%, 80%, 85%, 90%, 95%, 97%, 99%, 100% or any percentage in between of identity to one or more of SEQ ID Nos: 1-5, 7, 9, 14-17, 19, 21, 23, 25, 27, 29, 30, or 31. In some embodiments, the instant ribonucleic acids may correspond to the native gene sequences coding for the above-listed TERT proteins or may correspond to variants that are made possible due to the redundancy of the genetic code, as would be understood by one of ordinary skill in the art. In some embodiments, the codon selection may be optimized to optimize protein expression and/or reduced or increased immunogenicity using algorithms and methods known by those of ordinary skill in the art.

In some embodiments, an mRNA sequence may be synthesized as an unmodified or modified mRNA. An mRNA may be modified to enhance stability and/or evade immune detection and degradation. A modified mRNA may include, for example, one or more of a nucleotide modification, a nucleoside modification, a backbone modification, a sugar modification, and/or a base modification. In some embodiments, the modified nucleoside is pseudouridine or a pseudouridine analog. In some embodiments, the pseudouridine analog is N-1-methylpseudouridine. In some embodiments, the modified nucleoside is 5-methoxyuridine. In some embodiments a modified nucleotide as used herein may comprise any of the moieties listed in Table 1B.

TABLE 1B
Common name

pseudouridine
N-1-methylpseudouridine
5-methoxyuridine
1,2′-O-dimethyladenosine
1-methyl-3-(3-amino-3-carboxypropyl) pseudouridine
1-methyladenosine
1-methylguanosine
1-methylinosine
1-methylpseudouridine
2,2-dimethyl-guanosine
2′,3′-dideoxyadenosine
2′,3′-Dideoxycytidine
2′,3′-Dideoxyguanosine
2′,3′-Dideoxyinosine
2′,3′-dideoxynucleosides
2′,3′-Dideoxythymidine
2′,3′-dideoxythymine
2′,3′-Dideoxyuridine
2,6-diaminopurine
2′-O-ribosyladenosine (phosphate)
2′-Amino-2′-deoxyadenosine
2-Amino-2′-deoxyadenosine
2′-Amino-2′-deoxyuridine
2-Amino-6-chloropurineriboside
2-Amino-6-Cl-purine-2′-deoxyriboside
2-aminoadenosine
2-Aminoadenosine
2-Aminopurine-2′-deoxyriboside
2-Aminopurine-riboside
2′-Azido-2′-deoxyadenosine
2′-Azido-2′-deoxycytidine
2′-Azido-2′-deoxyguanosine
2′-Azido-2′-deoxyuridine
2′-Deoxyinosine
2′-Deoxy-P-nucleoside
2′-Deoxyuridine
2′-Deoxyzebularine
2′-Fluoro-2′-deoxyadenosine
2′-Fluoro-2′-deoxycytidine
2′-Fluoro-2′-deoxyguanosine
2′-Fluoro-2′-deoxyuridine
2′-Fluoro-thymidine
2-methyl-adenosine
2-methyl-guanosine
2-methylthio-N⁶-(cis-hydroxyisopentenyl) adenosine
2-methylthio-N-6-isopentenyl-adenosine
2-methylthio-N⁶-threonylcarbamoyladenosine
2′-O-Methyl-2-aminoadenosine
2′-O-Methyl-5-methyluridine
2′-O-methyladenosine
2′-O-methylcytidine
2′-O-methylguanosine
2′-O-methylinosine
2′-O-Methyl-N6-Methyladenosine
2′-O-methylpseudouridine
2′-O-methyluridine
2′-O-ribosylguanosine (phosphate)
2-Thio-2′-deoxycytidine
2-Thiocytidine
2-Thiothymidine
2-thiouridine
3,2′ -O-dimethyluridine
3′-Amino-2′,3′-dideoxyadenosine
3′-Amino-2′,3′-dideoxycytidine
3′-Amino-2′,3′-dideoxyguanosine
3′-Amino-2′,3′-dideoxythymidine
3′-Azido-2′,3′-dideoxyadenosine
3′-Azido-2′,3′-dideoxycytidine
3′-Azido-2′,3′-dideoxythymidine
3′-Azido-2′,3′-dideoxyuridine
3′-Deoxy-5-Methyluridine
3′-Deoxyadenosine
3′-deoxyadenosine (cordycepin)
3′-Deoxycytidine
3′-Deoxyguanosine
3′-deoxythymine
3′-Deoxyuridine
3-methylcytidine
3-methyluridine
3′-O-(2-nitrobenzyl)-2′-Deoxyadenosine
3′-O-(2-nitrobenzyl)-2′-Deoxyinosine
3′-O-Methyladenosine
3′-O-Methylcytidine
3′-O-Methylguanosine
3′-O-Methyluridine
4-acetyl-cytidine
4-Thiothymidine
4-Thiouridine
5-(carboxyhydroxymethyl) uridine methyl ester
5-(carboxyhydroxymethyl)uridine
5,2′-O-dimethyluridine
5,6-Dihydro-5-Methyluridine
5,6-Dihydrouridine
5-[(3-Indolyl)propionamide-N-allyl]-2′-deoxyuridine
5-Aminoallyl-2′-deoxycytidine
5-Aminoallyl-2′-deoxyuridine
5-Aminoallylcytidine
5-Aminoallyluridine
5-Bromo-2′,3′-dideoxyuridine
5-Bromo-2′-deoxycytidine
5-Bromo-2′-deoxyuridine
5-Bromocytidine
5-Bromouridine
5-carbamoylmethy1-2′-O-methyluridine
5-carbamoylmethyluridine
5-Carboxy-2′-deoxycytidine
5-Carboxycytidine
5-carboxymethylaminomethyl-2-thio-uridine
5-carboxymethylaminomethyluridine
5-Carboxymethylesteruridine
5-carboxymethyluridine
5-Carboxyuridine
5-Fluoro-2′-deoxyuridine
5-fluoro-uridine
5-Formyl-2′-deoxycytidine
5-Formyl-2′-deoxyuridine
5-formyl-2′-O-methylcytidine
5-formylcytidine
5-Formyluridine
5-Hydroxy-2′ -deoxycytidine
5-Hydroxycytidine
5-Hydroxymethyl-2′-deoxycytidine
5-Hydroxymethyl-2′ -deoxyuridine
5-hydroxymethylcytidine
5-Hydroxymethyluridine
5-hydroxyuridine
5-Iodo-2′-deoxycytidine
5-Iodo-2′-deoxyuridine
5-Iodocytidine
5-Iodouridine
5-methoxyaminomethyl-2-thio-uridine
5-methoxycarbonylmethy 1-2′-O-methyluridine
5-methoxycarbonylmethyl-2-thiouridine
5′-methoxycarbonylmethyl-uridine
5-methoxycarbonylmethyluridine
5-Methoxycytidine
5-Methoxyuridine
5-methoxy-uridine
5-Methyl-2′-deoxycytidine
5-methyl-2-thio-uridine
5-methylaminomethyl-uridine
5-methylcytidine
5-methyldihydrouridine
5-methyluridine
5-Propargylamino-2′-deoxycytidine
5-Propargylamino-2′-deoxyuridine
5-Propynyl-2′-deoxycytidine
5-taurinomethyl-2-thiouridine
5-taurinomethyluridine
6-Aza-2′-deoxyuridine
6-Azacytidine
6-Azauridine
6-chloropurine riboside
6-Chloropurine-2′-deoxyriboside
6-O-methylguanosine
6-Thio-2′-deoxyguanosine
7-Deaza-2′-deoxyadenosine
7-Deaza-2′-deoxyguanosine
7-Deaza-7-Propargylamino-2′-deoxyadenosine
7-Deaza-7-Propargylamino-2′-deoxyguanosine
7-Deazaadenosine
7-Deazaguanosine
7-methylguanosine
7-methyl-guanosine
8-Azaadenosine
8-Azidoadenosine
8-Chloro-2′-deoxyadenosine
8-Oxo-2′-deoxyadenosine
8-Oxo-2′-deoxyguanosine
8-Oxoadenoosine
8-Oxoguanosine
a 2′-deoxynucleoside
ac4C N4-acetylcytidine
Am 2′-O-methyladenosine
an —O-methylnucleoside
Ar(p) 2′ —O-ribosyladenosine (phosphate)
Araadenosine
Aracytidine
Araguanosine
Arauridin
benzimidazole riboside
beta-D-mannosyl-queosine
Biotin-16-7-Deaza-7-Propargylamino-2′-deoxyguanosine
Biotin-16-Aminoallyl-2′-dCTP
Biotin-16-Aminoallyl-2′-dUTP
Biotin-16-Aminoallylcytidine
Biotin-16-Aminoallyluridine
chm5U 5-(carboxyhydroxymethyl)uridine
2′-O-methylcytidine
5-carboxymethyluridine
5-carboxymethylaminomethyluridine
Cyanine 3-5-Propargylamino-2′-deoxycytidine
Cyanine 3-6-Propargylamino-2′-deoxyuridine
Cyanine 3-Aminoallylcytidine
Cyanine 3-Aminoallyluridine
Cyanine 5-6-Propargylamino-2′-deoxycytidine
Cyanine 5-6-Propargylamino-2′-deoxyuridine
Cyanine 5-Aminoallylcytidine
Cyanine 5-Aminoallyluridine
Cyanine 7-Aminoallyluridine
dihydrouridine
Dabcyl-5-3-Aminoallyl-2′-dUTP
Desthiobiotin-16-Aminoallyl-Uridine
Desthiobiotin-6-Aminoallyl-2′-deoxycytidine
dihydrouridine
5-formylcytidine
5-formyl-2′-O-methylcytidine
N6-glycinylcarbamoyladenosine
galactosyl-queuosine
2′-O-methylguanosine
2′-O-ribosylguanosine (phosphate)
5-hydroxymethylcytidine
5-hydroxyuridine
hydroxywybutosine
N6-isopentenyladenosine
2′-O-methylinosine
wyosine
inosine
N6-(cis-hydroxyisopentenyl)adenosine
Isoguanosine
l-methylguanosine
1-methyladenosine
1-methyl-3-(3-amino-3-carboxypropyl) pseudouridine
1,2′-O-dimethyladenosine
1-methylguanosine
1-methylinosine
1-methylpseudouridine
N2,N2-dimethylguanosine
N2,N2,7-trimethylguanosine I inosine
N2,7-dimethylguanosine
N2-methylguanosine
3-methylcytidine
3-methyluridine
3,2′-O-dimethyluridine
N4-methylcytidine
5-methylcytidine
5-methyldihydrouridine
5-methyluridine
5,2′-O-dimethyluridine
N6,N6-dimethyladenosine
N6,N6,2′-O-trimethyladenosine
N6-methyladenosine
N6,2′-O-dimethyladenosine
7-methylguanosine
mannosyl-queuosine
5-(carboxyhydroxymethyl)uridine
5-methoxycarbonylmethyl-2-thiouridine
5-methoxycarbonylmethyl-2′-O-methyluridine
5-methoxycarbonylmethyluridine
2-methylthio-N6-(cis-hydroxyisopentenyl) adenosine
2-methylthio-N6-threonyl carbamoyladenosine
N1-Ethylpseudouridine
N1-Methoxymethylpseudouridine
N1-Methyl-2′-O-Methylpseudouridine
N1-Methyladenosine
N1-Propylpseudouridine
N²,7-dimethylguanosine
N²,N²,7-trimethylguanosine
N²,N²-dimethylguanosine
N2-Methyl-2′-deoxyguanosine
N²-methylguanosine
N⁴-acetylcytidine
N4-Biotin-OBEA-2′-deoxycytidine
N4-Methyl-2′-deoxycytidine
N⁴-methylcytidine
N⁶-(cis-hydroxyisopentenyl)-adenosine
N⁶,2′-O-dimethyladenosine
N⁶ ,N⁶,2′-O-trimethyladenosine
N⁶,N⁶-dimethyladenosine
N⁶-glycinylcarbamoyladenosine
N⁶-isopentenyladenosine
N6-isopentenyl-adenosine
N6-Methyl-2-Aminoadenosine
N6-Methyl-2′-deoxyadenosine
N⁶-methyladenosine
N6-methyl-adenosine
N⁶-threonylcarbamoyladenosine
ncm5U 5-carbamoylmethyluridine
ncm5Um 5-carbamoylmethyl-2′-O-methyluridine
Nl-methyladenosine
N-uridine-5-oxyaceticacidmethylester
peroxywybutosine
O6-Methyl-2′-deoxyguanosine
O6-Methylguanosine
hydroxywybutosine
undermodified hydroxywybutosine
O-Methylpseudouridine
peroxywybutosine
Pseudoisocytidine
Puromycin
queosine
2-thiouridine
N6-threonylcarbamoyladenosine
Thienocytidine
Thienoguanosine
Thienouridine
2′-O-methyluridine
undermodified hydroxywybutosine
uridine-5-oxyaceticacid(v)
uridine-5-oxyaceticacidmethylester
wybutosine
wybutoxosine
wyosine
Xanthosine
5-taurinomethyl-2-thiouridine
5-taurinomethyluridine
2′-O-methylpseudouridine

In some embodiments, an mRNA may be synthesized from naturally occurring bases and/or base analogs (modified bases) including, but not limited to, purines (adenine (A), guanine (G)) or pyrimidines (thymine (T), cytosine (C), uracil (U)), and analogues and derivatives thereof, e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), pseudouridine, N-1-methyl-pseudouridine, dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, beta-D-mannosyl-queosine, wybutoxosine, and phosphoramidates, phosphorothioates, peptide nucleotides, methylphosphonates, 7-deazaguanosine, 5-methylcytosine and inosine.

In some embodiments, an mRNA may be synthesized from naturally occurring nucleosides and/or nucleoside analogs (modified nucleosides) including, but not limited to, nucleosides comprising adenosine (A), guanosine (G)) or pyrimidines (thymine (T), cytidine (C), uridine (U)), and nucleoside comprising analogues and derivatives thereof, e.g., 3′-deoxyadenosine (cordycepin), 3′-deoxyuridine, 3′-deoxycytosine, 3′-deoxyguanosine, 3′-deoxythymine, 2′,3′-dideoxynucleosides, 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, —O— methylnucleoside, 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouridine, N-1-methyl-pseudouridine, dihydro-uracil, 2-thio-uracil, 4-thio-uridine, 5-carboxymethylaminomethyl-2-thio-uridine, 5-(carboxyhydroxymethyl)-uridine, 5-fluoro-uridine, 5-bromo-uridine, 5-carboxymethylaminomethyl-uridine, 5-methyl-2-thio-uridine, 5-methyl-uridine, N-uridine-5-oxyacetic acid methyl ester, 5-methylaminomethyl-uridine, 5-methoxyaminomethyl-2-thio-uridine, 5′-methoxycarbonylmethyl-uracil, 5-methoxy-uracil, uracil-5-oxyacetic acid methyl ester, uracil-5-oxyacetic acid (v), 1-methyl-pseudouridine, queosine, beta-D-mannosyl-queosine, wybutoxosine, 7-deazaguanosine, 5-methylcytosine, and inosine.

The preparation of such base, nucleoside, nucleotide, and backbone analogues, modifications, and derivatives is known to a person skilled in the art e.g. from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, all of which are incorporated by reference in their entirety.

In some embodiments, uracil nucleosides of the mRNA are about 80%, about 90%, 95%, 99%, or 100% depleted and replaced with a uracil nucleoside analog, e.g., pseudouridine, 5-methoxyuridine, or N-1-methyl-pseudouridine.

In some embodiments, an mRNA may contain an RNA backbone modification. Typically, a backbone modification is a modification in which the phosphates of the backbone of the nucleotides contained in the RNA are chemically modified. Exemplary backbone modifications may include, but are not limited to, modifications in which the phosphodiester linkage is replaced with a member from the group consisting of peptides, methylphosphonates, methylphosphoramidates, phosphoramidates, phosphorothioates (e.g., cytidine 5′-O-(1-thiophosphate)), boranophosphates, and/or positively charged guanidimum groups, or other means of replacing the phosphodiester linkage.

In some embodiments, an mRNA may contain sugar modifications. A sugar modification may include but is not limited to, 2′ O-methyl sugar modifications, 2′ fluoro sugar modifications (e.g. 2′-fluororibose), 3′ amino sugar modifications, 2′ thio sugar modifications, 2′-O-alkyl sugar modifications, 5-methylthioribose, and sugar modifications of 2′-deoxy-2′-fluoro-ribonucleotide (2′-fluoro-2′-deoxycytidine, 2′-fluoro-2′-deoxyuridine), 2′-deoxy-2′-deamine-ribonucleotide (2′-amino-2′-deoxycytidine, 2,-amino-2′-deoxyuridine), 2′-O-alkylribonucleotide, 2′-deoxy-2′-C-alkylribonucleotide (2′-O-methylcytidine, 2′-methyluridine), 2′-C-alkylribonucleotide, and isomers thereof (2′-aracytidine, 2′-arauridine), or azidophosphates (2′-azido-2′-deoxycytidine, 2′-azido-2′-deoxyuridine).

In some embodiments, an mRNA may be synthesized from one or more of the nucleotide triphosphates comprising any of the nucleosides and nucleotides disclosed herein, or any of the following nucleoside triphosphates: 2′-Deoxyadenosine-5′-O-(1-Thiotriphosphate), 2′-Deoxycytidine-5′-O-(1-Thiotriphosphate), 2′-Deoxyguanosine-5′-O-(1-Thiotriphosphate), 2′-Deoxythymidine-5′-O-(1-Thiotriphosphate), Adenosine-5′-O-(1-Thiotriphosphate), Cytidine-5′-O-(1-Thiotriphosphate), Guanosine-5′-O-(1-Thiotriphosphate), Uridine-5′-O-(1-Thiotriphosphate), 2′,3′-Dideoxyadenosine-5′-O-(1-Thiotriphosphate), 2′,3′-Dideoxycytidine-5′-O-(1-Thiotriphosphate), 2′,3′-Dideoxyguanosine-5′-O-(1-Thiotriphosphate), 3′-Deoxythymidine-5′-O-(1-Thiotriphosphate), 3′-Azido-2′,3′-dideoxythymidine-5′-O-(1-Thiotriphosphate), 2′,3′-Dideoxyuridine-5′-O-(1-Thiotriphosphate), 2′-Deoxyadenosine-5′-O-(1-Boranotriphosphate), 2′-Deoxycytidine-5′-O-(1-Boranotriphosphate), 2′-Deoxyguanosine-5′-O-(1-Boranotriphosphate), and 2′-Deoxythymidine-5′-O-(1-Boranotriphosphate).

In some embodiments, an mRNA may include the addition of a “cap” on the N-terminal (5′) end, and a “tail” on the C-terminal (3′) end. The presence of the cap may provide resistance to nucleases found in eukaryotic cells. The presence of a “tail” may protect the mRNA from exonuclease degradation.

### Cap Structure

In some embodiments, an mRNA may include a 5′ cap structure. A 5′ cap may comprise for example, a triphosphate linkage and a guanine nucleotide in which the 7-nitrogen is methylated. Examples of cap structures include, but are not limited to, m7G(5′)ppp (5′)A, G(5′)ppp(5′)A, and G(5′)ppp(5′)G. Naturally occurring cap structures comprise a 7-methyl guanosine that is linked via a triphosphate bridge to the 5′-end of the first transcribed nucleotide, resulting in a dinucleotide cap of m7G(5′)ppp(5′)N, where N is any nucleoside. In vivo, the cap is added in the nucleus by the enzyme guanylyl transferase immediately after initiation of transcription.

In some embodiments, a 5′cap may comprise an m7(3′OmeG)(5′)ppp(5′)(2′OmeA)pG or (CleanCap™ 3′ Ome) structure. In some embodiments, a 5′ cap may comprise a m7G(5′)ppp(5′)G. In some embodiments, the Anti-Reverse Cap Analog (“ARCA”) or modified ARCA, is a 5′ cap in which the 2′ or 3′ OH group is replaced with —OCH3. In some embodiments, the ARCA comprises an 3′-O-Me-m7G(5′)ppp(5′)G structure. In some embodiments, the 5′ cap comprises m7G(5′)ppp(5′)(2′OmeA)pG. Additional mRNA caps may include, but are not limited to, a chemical structures selected from the group consisting of m7GpppG, m7GpppA, m7GpppC; unmethylated caps (e.g., GpppG); a 71emethylated cap (e.g., m2′7GpppG), a trimethylated cap analog, or anti reverse cap analogs (e.g., ARCA; m7,2′0meGpppG, m72′dGpppG, m7′3′0meGpppG, m7,3 dGpppG and their tetraphosphate derivatives) (see, e.g., Jemielity, J. et al, ‘Wove anti-reverse cap analogs with superior translational properties”, RNA, 9: 1108-1122 (2003)).

In some embodiments, a suitable cap is a 7-methyl guanylate (“m7G”) linked via a triphosphate bridge to the 5′-end of the first transcribed nucleotide, resulting in m7G(5′)ppp(5′)N, where N is any nucleoside. A embodiment of a m7G cap utilized in embodiments of the disclosure is m7G(5′)ppp(5′)G. In some embodiments, the cap is a Cap0 structure. Cap0 structures lack a 2′-O-methyl residue of the ribose attached to bases 1 and 2. In some embodiments, the cap is a Cap1 structure. Cap1 structures have a 2′-O-methyl residue at base 2. In some embodiments, the cap is a Cap2 structure. Cap2 structures have a 2′-O-methyl residue attached to both bases 2 and 3.

A variety of m7G cap analogs are known in the art, many of which are commercially available. These include the m7 GpppG described above, as well as the ARCA 3′-OCH3 and 2′-OCH3 cap analogs (Jemielity, J. et al., RNA, 9: 1108-1122 (2003)). Additional cap analogs for use in embodiments of the disclosure include N7-benzylated dinucleoside tetraphosphate analogs (described in Grudzien, E. et at, RNA, 10: 1479-1487 (2004)), phosphorothioate cap analogs (described in Grudzien-Nogalska, E., et al, RNA, 13: 1745-1755 (2007)), and cap analogs (including biotinylated cap analogs) described in U.S. Pat. Nos. 8,093,367 and 8,304,529, incorporated by reference herein.

In some embodiments, the 5′ cap is inosine, N1-methyl-guanosine, 2′fluoro-guanosine, 7-deaza-guanosine, m7(3′OmeG)(5′)ppp(5′)(2′OmeA)pG, CleanCap™ m7(3′OmeG)(5′)ppp(5′)(2′OmeA)pG, 8-oxo-guanosine, 2-amino-guanosine, LNA-guanosine, 2-azido-guanosine, Cap2, Cap4, CAP-003, or CAP-225.

In some embodiments, the 5′ cap comprises or consists of an internal ribosome entry site (IRES). In some embodiments the IRES is within the 5′ UTR. In some embodiments, the 5′ cap comprises or consists of a 2A self-cleavage peptide, e.g, one or more of P2A, T2A, E2A and F2A.

### Tail Structure

The presence of a “tail” may serve to protect an mRNA from exonuclease degradation. The poly-A tail is thought to stabilize natural messengers and synthetic sense RNA. Therefore, in certain embodiments a long poly-A tail can be added to an mRNA molecule thus rendering the RNA more stable. Poly-A tails can be added using a variety of art-recognized techniques. For example, long poly-A tails can be added to synthetic or in vitro transcribed RNA using poly-A polymerase (Yokoe, et al. Nature Biotechnology. 1996; 14: 1252-1256). A transcription vector can also encode long poly-A tails. In addition, poly-A tails can be added by transcription directly from PCR products. Poly-A may also be ligated to the 3′ end of a sense RNA with RNA ligase (see, e.g., Molecular Cloning A Laboratory Manual, 2^{nd} Ed., ed. By Sambrook, Fritsch and Maniatis (Cold Spring Harbor Laboratory Press: 1991 edition)).

In some embodiments, an mRNA may include a 3′ poly(A) tail structure. The length of the poly-A tail may be at least about 10, 50, 100, 200, 300, 400 or at least about 500 nucleotides. In some embodiments, a poly-A tail on the 3′ terminus of an mRNA may include about 10 to 300 adenosine nucleotides (e.g., about 10 to 200 adenosine nucleotides, about 10 to 150 adenosine nucleotides, about 10 to 100 adenosine nucleotides, about 20 to 70 adenosine nucleotides, or about 20 to 60 adenosine nucleotides). In some embodiments, the poly A tail is 120 adenosine nucleotides.

In some embodiments, an mRNA may include a 3′ polyI tail structure. A poly-C tail on th’ 3′ terminus of mRNA may include about 10 to 200 cytosine nucleotides (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A tail or may substitute the poly-A tail. In some embodiments, the length of the poly-A or poly C tail is associated with the stability of a modified sense mRNA and, therefore, the transcription of the protein. For example, because the length of the poly-A tail may influence the half-life of a sense mRNA molecule, the length of the poly-A tail may be adjusted to modify the level of resistance of the mRNA to nucleases, thereby providing more control over the time course of polynucleotide expression and/or polypeptide production.’

5‘ an’ 3′ Untranslated Regions (UTRs)

In some embodiments, an mRNA may include 5′ untranslated region (UTR) and/or a 3′ UTR. In some embodiments, a 5′ UTR may include one or more elements that affect the stability or translation of an mRNA. In some embodiments, the 5′UTR for example, may include an iron responsive element. In some embodiments, 5′ UTR may be between about 50 to about 100, or from about 50 to about 500 nucleotides in length. In some embodiments, 3′ UTR includes one or more of a poly-A signal, a binding site for proteins that may affect mRNA stability or localization, or one or more binding sites for miRNAs. In some embodiments, 3′ UTR may be between about 0 and about 50 nucleotides, or about 50 to about 100 nucleotides in length.

Example 3‘ an’ 5′ UTR sequences may be derived from mRNAs with relatively long half-lives (e.g., globin, actin, GAPDH, tubulin, histone, or citric acid cycle enzymes) to increase the stability of the sense mRNA molecule. For example, 5′ UTR sequence may include a partial sequence of a cytomegalovirus (CMV) immediate-early 1 (IE1) gene, or a fragment thereof to improve the nuclease resistance and/or improve the half-life of the polynucleotide. Generally, these modifications improve the stability and/or pharmacokinetic properties (e.g., half-life) of the polynucleotide relative to their unmodified counterparts, and include, for example modifications made to improve such polynucleoti'es' resistance to in vivo nuclease digestion.

In some embodiments, a UTR may improve tissue specific expression, e.g., in the liver.

In some embodiments, the 3′ UTR is a mouse alpha-globin 3′ UTR. In some embodiments, the 3′ UTR comprises a sequence at least 70%, at least 75%, at least 80%, at least 85%, at least 90%, at least 95%, at least 97%, at least 99%, or 100% identical to SEQ ID NO: 32

TTAAGCTGCCTTCTGCGGGGCTTGCCTTCTGGCCATGCCCTTCTT
CTCTCCCTTGCACCTGTACCTCTTGGTCTTTGAATAAAGCCTGAG
TAGGAAGTCTAG

In some embodiments, the 3′ UTR is a wild type human beta-globin 3′ UTR. In some embodiments, the 3′ UTR comprises a sequence at least 70%, at least 75%, at least 80%, at least 85%, at least 90%, at least 95%, at least 97%, at least 99%, or 100% identical to SEQ ID NO: 33

CAATTGGCTCGCTTTCTTGCTGTCCAATTTCTATTAAAGGTTCCT
TTGTTCCCTAAGTCCAACTACTAAACTGGGGGATATTATGAAGGG
CCTTGAGCATCTGGATTCTGCCTAATAAAAAACATTTATTTTCAT
TGCGAATTC

In some embodiments, the 3′ UTR is a variant human beta-globin 3′ UTR. In some embodiments, the 3′ UTR comprises a sequence at least 70%, at least 75%, at least 80%, at least 85%, at least 90%, at least 95%, at least 97%, at least 99%, or 100% identical to SEQ ID NO: 34

CAATTGGCTCGCTTTCTTGCTGTCCAATTTCTATTAAAGGTTCCT
TTTGTTCCCTAAGTCCAACTACTAAACTGGGGGATATTATGAAGG
GCCTTGAGCATCTGGATTCTGCCTAATAAAAAACATTTCTTTTCA
TTGCGAATTC

In some embodiments, the 5′ UTR is a synthetic 5′ UTR. In some embodiments, the 5′ UTR comprises a sequence at least 70%, at least 75%, at least 80%, at least 85%, at least 90%, at least 95%, at least 97%, at least 99%, or 100% identical to SEQ ID NO: 35

AGGAAATAAGAGAGAAAAGAAGAGTAAGAAGAAATATAAGAGCCA
CC

In some embodiments, the 5′ UTR is a human beta-globin 5′ UTR. In some embodiments, the 5′ UTR comprises a sequence at least 70%, at least 75%, at least 80%, at least 85%, at least 90%, at least 95%, at least 97%, at least 99%, or 100% identical to SEQ ID NO: 36

GGACATTTGCTTCTGACACAACTGTGTTCACTAGCAACCTCAAAC
AACTAGTACACC

In some embodiments, the UTR may be any of, or functional variants of, those described in any of PCT Application No. WO2017053297A1 and U.S. patent Ser. No. 10/519,189B2, both of which are incorporated herein in their entirety.

Exemplary Therapeutic TERT mRNA Sequences

In some embodiments, a TERT mRNA may refer to the full length mRNA sequence, ie. coding and non-coding, delivered to the tissue, e.g. the liver. Example sequences include the sequences comprising mouse TERT of SEQ ID NOS: 37 and 38, and the sequences comprising human TERT of SEQ ID NOS: 39 and 40.

In some embodiments, the mouse TERT mRNA comprises a sequence at least 70%, at least 75%, at least 80%, at least 85%, at least 90%, at least 95%, at least 97%, at least 99%, or 100% identical to SEQ ID NO: 37

CAACCGGTACACCATGACCCGCGCTCCTCGTTGCCCCGCGGTGC
GCTCTCTGCTGCGCAGCCGATACCGGGAGGTGTGGCCGCTGGCAA
CCTTTGTGCGGCGCCTGGGGCCCGAGGGCAGGCGGCTTGTGCAAC
CCGGGGACCCGAAGATCTACCGCACTTTGGTTGCCCAATGCCTAG
TGTGCATGCACTGGGGCTCACAGCCTCCACCTGCCGACCTTTCCT
TCCACCAGGTGTCATCCCTGAAAGAGCTGGTGGCCAGGGTTGTGC
AGAGACTCTGCGAGCGCAACGAGAGAAACGTGCTGGCTTTTGGCT
TTGAGCTGCTTAACGAGGCCAGAGGCGGGCCTCCCATGGCCTTCA
CTAGTAGCGTGCGTAGCTACTTGCCCAACACTGTTATTGAGACCC
TGCGTGTCAGTGGTGCATGGATGCTACTGTTGAGCCGAGTGGGCG
ACGACCTGCTGGTCTACCTGCTGGCACACTGTGCTCTTTATCTTC
TGGTGCCCCCCAGCTGTGCCTACCAGGTGTGTGGGTCTCCCCTGT
ACCAAATTTGTGCCACCACGGATATCTGGCCCTCTGTGTCCGCTA
GTTACAGGCCCACCCGACCCGTGGGCAGGAATTTCACTAACCTTA
GGTTCTTACAACAGATCAAGAGCAGTAGTCGCCAGGAAGCACCGA
AACCCCTGGCCTTGCCATCTCGAGGTACAAAGAGGCATCTGAGTC
TCACCAGTACAAGTGTGCCTTCAGCTAAGAAGGCCAGATGCTATC
CTGTCCCGAGAGTGGAGGAGGGACCCCACAGGCAGGTGCTACCAA
CCCCATCAGGCAAATCATGGGTGCCAAGTCCTGCTCGGTCCCCCG
AGGTGCCTACTGCAGAGAAAGATTTGTCTTCTAAAGGAAAGGTGT
CTGACCTGAGTCTCTCTGGGTCGGTGTGCTGTAAACACAAGCCCA
GCTCCACATCTCTGCTGTCACCACCCCGCCAAAATGCCTTTCAGC
TCAGGCCATTTATTGAGACCAGACATTTCCTTTACTCCAGGGGAG
ATGGCCAAGAGCGTCTAAACCCCTCATTCCTACTCAGCAACCTCC
AGCCTAACTTGACTGGGGCCAGGAGACTGGTGGAGATCATCTTTC
TGGGCTCAAGGCCTAGGACATCAGGACCACTCTGCAGGACACACC
GTCTATCGCGTCGATACTGGCAGATGCGGCCCCTGTTCCAACAGC
TGCTGGTGAACCATGCAGAGTGCCAATATGTCAGACTCCTCAGGT
CACATTGCAGGTTTCGAACAGCAAACCAACAGGTGACAGATGCCT
TGAACACCAGCCCACCGCACCTCATGGATTTGCTCCGCCTGCACA
GCAGTCCCTGGCAGGTATATGGTTTTCTTCGGGCCTGTCTCTGCA
AGGTGGTGTCTGCTAGTCTCTGGGGTACCAGGCACAATGAGCGCC
GCTTCTTTAAGAACTTAAAGAAGTTCATCTCGTTGGGGAAATACG
GCAAGCTATCACTGCAGGAACTGATGTGGAAGATGAAAGTAGAGG
ATTGCCACTGGCTCCGCAGCAGCCCGGGGAAGGACCGTGTCCCCG
CTGCAGAGCACCGTCTGAGGGAGAGGATCCTGGCTACGTTCCTGT
TCTGGCTGATGGACACATACGTGGTACAGCTGCTTAGGTCATTCT
TTTACATCACAGAGAGCACATTCCAGAAGAACAGGCTCTTCTTCT
ACCGTAAGAGTGTGTGGAGCAAGCTGCAGAGCATTGGAGTCAGGC
AACACCTTGAGAGAGTGCGGCTACGGGAGCTGTCACAAGAGGAGG
TCAGGCATCACCAGGACACCTGGCTAGCCATGCCCATCTGCAGAC
TGCGCTTCATCCCCAAGCCCAACGGCCTGCGGCCCATTGTGAACA
TGAGTTATAGCATGGGTACCAGAGCTTTGGGCAGAAGGAAGCAGG
CCCAGCATTTCACCCAGCGTCTCAAGACTCTCTTCAGCATGCTCA
ACTATGAGCGGACAAAACATCCTCACCTTATGGGGTCTTCTGTAC
TGGGTATGAATGACATCTACAGGACCTGGCGGGCCTTTGTGCTGC
GTGTGCGTGCTCTGGACCAGACACCCAGGATGTACTTTGTTAAGG
CAGATGTGACCGGGGCCTATGATGCCATCCCCCAGGGTAAGCTGG
TGGAGGTTGTTGCCAATATGATCAGGCACTCGGAGAGCACGTACT
GTATCCGCCAGTATGCAGTGGTCCGGAGAGATAGCCAAGGCCAAG
TCCACAAGTCCTTTAGGAGACAGGTCACCACCCTCTCTGACCTCC
AGCCATACATGGGCCAGTTCCTTAAGCATCTGCAGGATTCAGATG
CCAGTGCACTGAGGAACTCCGTTGTCATCGAGCAGAGCATCTCTA
TGAATGAGAGCAGCAGCAGCCTGTTTGACTTCTTCCTGCACTTCC
TGCGTCACAGTGTCGTAAAGATTGGTGACAGGTGCTATACGCAGT
GCCAGGGCATCCCCCAGGGCTCCAGCCTATCCACCCTGCTCTGCA
GTCTGTGTTTCGGAGACATGGAGAACAAGCTGTTTGCTGAGGTGC
AGCGGGATGGGTTGCTTTTACGTTTTGTTGATGACTTTCTGTTGG
TGACGCCTCACTTGGACCAAGCAAAAACCTTCCTCAGCACCCTGG
TCCATGGCGTTCCTGAGTATGGGTGCATGATAAACTTGCAGAAGA
CAGTGGTGAACTTCCCTGTGGAGCCTGGTACCCTGGGTGGTGCAG
CTCCATACCAGCTGCCTGCTCACTGCCTGTTTCCCTGGTGTGGCT
TGCTGCTGGACACTCAGACTTTGGAGGTGTTCTGTGACTACTCAG
GTTATGCCCAGACCTCAATTAAGACGAGCCTCACCTTCCAGAGTG
TCTTCAAAGCTGGGAAGACCATGCGGAACAAGCTCCTGTCGGTCT
TGCGGTTGAAGTGTCACGGTCTATTTCTAGACTTGCAGGTGAACA
GCCTCCAGACAGTCTGCATCAATATATACAAGATCTTCCTGCTTC
AGGCCTACAGGTTCCATGCATGTGTGATTCAGCTTCCCTTTGACC
AGCGTGTTAGGAAGAACCTCACATTCTTTCTGGGCATCATCTCCA
GCCAAGCATCCTGCTGCTATGCTATCCTGAAGGTCAAGAATCCAG
GAATGACACTAAAGGCCTCTGGCTCCTTTCCTCCTGAAGCCGCAC
ATTGGCTCTGCTACCAGGCCTTCCTGCTCAAGCTGGCTGCTCATT
CTGTCATCTACAAATGTCTCCTGGGACCTCTGAGGACAGCCCAAA
AACTGCTGTGCCGGAAGCTCCCAGAGGCGACAATGACCATCCTTA
AAGCTGCAGCTGACCCAGCCCTAAGCACAGACTTTCAGACCATTT
TGGACTAACAATTGGCTCGCTTTCTTGCTGTCCAATTTCTATTAA
AGGTTCCTTTTGTTCCCTAAGTCCAACTACTAAACTGGGGGATAT
TATGAAGGGCCTTGAGCATCTGGATTCTGCCTAATAAAAAACATT
TCTTTTCATTGCGAATTCAAAAAAAAAAAAAAAAAAAAAAAAAAA
AAAAAAAAAAAAAAAAAAAAAAAAAAAAAAAAAAAAAAAAAAAAA
AAAAAAAAAAAAAAAAAAAAAAAAAAAAAAAAAAAAAAAAAAAAA
AAAAAAAAAAAAAAAAAAAAAAAAAAAAAAAAAA

In some embodiments, the mouse TERT mRNA comprises a sequence at least 70%, at least 75%, at least 80%, at least 85%, at least 90%, at least 95%, at least 97%, at least 99%, or 100% identical to SEQ ID NO: 38

AGGAAATAAGAGAGAAAAGAAGAGTAAGAAGAAATATAAGAGCCA
CCATGACCCGCGCTCCTCGTTGCCCCGCGGTGCGCTCTCTGCTGC
GCAGCCGATACCGGGAGGTGTGGCCGCTGGCAACCTTTGTGCGGC
GCCTGGGGCCCGAGGGCAGGCGGCTTGTGCAACCCGGGGACCCGA
AGATCTACCGCACTTTGGTTGCCCAATGCCTAGTGTGCATGCACT
GGGGCTCACAGCCTCCACCTGCCGACCTTTCCTTCCACCAGGTGT
CATCCCTGAAAGAGCTGGTGGCCAGGGTTGTGCAGAGACTCTGCG
AGCGCAACGAGAGAAACGTGCTGGCTTTTGGCTTTGAGCTGCTTA
ACGAGGCCAGAGGCGGGCCTCCCATGGCCTTCACTAGTAGCGTGC
GTAGCTACTTGCCCAACACTGTTATTGAGACCCTGCGTGTCAGTG
GTGCATGGATGCTACTGTTGAGCCGAGTGGGCGACGACCTGCTGG
TCTACCTGCTGGCACACTGTGCTCTTTATCTTCTGGTGCCCCCCA
GCTGTGCCTACCAGGTGTGTGGGTCTCCCCTGTACCAAATTTGTG
CCACCACGGATATCTGGCCCTCTGTGTCCGCTAGTTACAGGCCCA
CCCGACCCGTGGGCAGGAATTTCACTAACCTTAGGTTCTTACAAC
AGATCAAGAGCAGTAGTCGCCAGGAAGCACCGAAACCCCTGGCCT
TGCCATCTCGAGGTACAAAGAGGCATCTGAGTCTCACCAGTACAA
GTGTGCCTTCAGCTAAGAAGGCCAGATGCTATCCTGTCCCGAGAG
TGGAGGAGGGACCCCACAGGCAGGTGCTACCAACCCCATCAGGCA
AATCATGGGTGCCAAGTCCTGCTCGGTCCCCCGAGGTGCCTACTG
CAGAGAAAGATTTGTCTTCTAAAGGAAAGGTGTCTGACCTGAGTC
TCTCTGGGTCGGTGTGCTGTAAACACAAGCCCAGCTCCACATCTC
TGCTGTCACCACCCCGCCAAAATGCCTTTCAGCTCAGGCCATTTA
TTGAGACCAGACATTTCCTTTACTCCAGGGGAGATGGCCAAGAGC
GTCTAAACCCCTCATTCCTACTCAGCAACCTCCAGCCTAACTTGA
CTGGGGCCAGGAGACTGGTGGAGATCATCTTTCTGGGCTCAAGGC
CTAGGACATCAGGACCACTCTGCAGGACACACCGTCTATCGCGTC
GATACTGGCAGATGCGGCCCCTGTTCCAACAGCTGCTGGTGAACC
ATGCAGAGTGCCAATATGTCAGACTCCTCAGGTCACATTGCAGGT
TTCGAACAGCAAACCAACAGGTGACAGATGCCTTGAACACCAGCC
CACCGCACCTCATGGATTTGCTCCGCCTGCACAGCAGTCCCTGGC
AGGTATATGGTTTTCTTCGGGCCTGTCTCTGCAAGGTGGTGTCTG
CTAGTCTCTGGGGTACCAGGCACAATGAGCGCCGCTTCTTTAAGA
ACTTAAAGAAGTTCATCTCGTTGGGGAAATACGGCAAGCTATCAC
TGCAGGAACTGATGTGGAAGATGAAAGTAGAGGATTGCCACTGGC
TCCGCAGCAGCCCGGGGAAGGACCGTGTCCCCGCTGCAGAGCACC
GTCTGAGGGAGAGGATCCTGGCTACGTTCCTGTTCTGGCTGATGG
ACACATACGTGGTACAGCTGCTTAGGTCATTCTTTTACATCACAG
AGAGCACATTCCAGAAGAACAGGCTCTTCTTCTACCGTAAGAGTG
TGTGGAGCAAGCTGCAGAGCATTGGAGTCAGGCAACACCTTGAGA
GAGTGCGGCTACGGGAGCTGTCACAAGAGGAGGTCAGGCATCACC
AGGACACCTGGCTAGCCATGCCCATCTGCAGACTGCGCTTCATCC
CCAAGCCCAACGGCCTGCGGCCCATTGTGAACATGAGTTATAGCA
TGGGTACCAGAGCTTTGGGCAGAAGGAAGCAGGCCCAGCATTTCA
CCCAGCGTCTCAAGACTCTCTTCAGCATGCTCAACTATGAGCGGA
CAAAACATCCTCACCTTATGGGGTCTTCTGTACTGGGTATGAATG
ACATCTACAGGACCTGGCGGGCCTTTGTGCTGCGTGTGCGTGCTC
TGGACCAGACACCCAGGATGTACTTTGTTAAGGCAGATGTGACCG
GGGCCTATGATGCCATCCCCCAGGGTAAGCTGGTGGAGGTTGTTG
CCAATATGATCAGGCACTCGGAGAGCACGTACTGTATCCGCCAGT
ATGCAGTGGTCCGGAGAGATAGCCAAGGCCAAGTCCACAAGTCCT
TTAGGAGACAGGTCACCACCCTCTCTGACCTCCAGCCATACATGG
GCCAGTTCCTTAAGCATCTGCAGGATTCAGATGCCAGTGCACTGA
GGAACTCCGTTGTCATCGAGCAGAGCATCTCTATGAATGAGAGCA
GCAGCAGCCTGTTTGACTTCTTCCTGCACTTCCTGCGTCACAGTG
TCGTAAAGATTGGTGACAGGTGCTATACGCAGTGCCAGGGCATCC
CCCAGGGCTCCAGCCTATCCACCCTGCTCTGCAGTCTGTGTTTCG
GAGACATGGAGAACAAGCTGTTTGCTGAGGTGCAGCGGGATGGGT
TGCTTTTACGTTTTGTTGATGACTTTCTGTTGGTGACGCCTCACT
TGGACCAAGCAAAAACCTTCCTCAGCACCCTGGTCCATGGCGTTC
CTGAGTATGGGTGCATGATAAACTTGCAGAAGACAGTGGTGAACT
TCCCTGTGGAGCCTGGTACCCTGGGTGGTGCAGCTCCATACCAGC
TGCCTGCTCACTGCCTGTTTCCCTGGTGTGGCTTGCTGCTGGACA
CTCAGACTTTGGAGGTGTTCTGTGACTACTCAGGTTATGCCCAGA
CCTCAATTAAGACGAGCCTCACCTTCCAGAGTGTCTTCAAAGCTG
GGAAGACCATGCGGAACAAGCTCCTGTCGGTCTTGCGGTTGAAGT
GTCACGGTCTATTTCTAGACTTGCAGGTGAACAGCCTCCAGACAG
TCTGCATCAATATATACAAGATCTTCCTGCTTCAGGCCTACAGGT
TCCATGCATGTGTGATTCAGCTTCCCTTTGACCAGCGTGTTAGGA
AGAACCTCACATTCTTTCTGGGCATCATCTCCAGCCAAGCATCCT
GCTGCTATGCTATCCTGAAGGTCAAGAATCCAGGAATGACACTAA
AGGCCTCTGGCTCCTTTCCTCCTGAAGCCGCACATTGGCTCTGCT
ACCAGGCCTTCCTGCTCAAGCTGGCTGCTCATTCTGTCATCTACA
AATGTCTCCTGGGACCTCTGAGGACAGCCCAAAAACTGCTGTGCC
GGAAGCTCCCAGAGGCGACAATGACCATCCTTAAAGCTGCAGCTG
ACCCAGCCCTAAGCACAGACTTTCAGACCATTTTGGACTAATAAT
TAAGCTGCCTTCTGCGGGGCTTGCCTTCTGGCCATGCCCTTCTTC
TCTCCCTTGCACCTGTACCTCTTGGTCTTTGAATAAAGCCTGAGT
AGGAAGTCTAGAAAAAAAAAAAAAAAAAAAAAAAAAAAAAAAAAA
AAAAAAAAAAAAAAAAAAAAAAAAAAAAAAAAAAAAAAAAAAAAA
A

In some embodiments, the human TERT mRNA comprises a sequence at least 70%, at least 75%, at least 80%, at least 85%, at least 90%, at least 95%, at least 97%, at least 99%, or 100% identical to SEQ ID NO: 39

GGGACATTTGCTTCTGACACAACTGTGTTCACTAGCAACCTCAAA
CAACTAGTACACCATGCCGCGCGCTCCCCGCTGCCGAGCCGTGCG
CTCCCTGCTGCGCAGCCACTACCGCGAGGTGCTGCCGCTGGCCAC
GTTCGTGCGGCGCCTGGGGCCCCAGGGCTGGCGGCTGGTGCAGCG
CGGGGACCCGGCGGCTTTCCGCGCGCTGGTGGCCCAGTGCCTGGT
GTGCGTGCCCTGGGACGCACGGCCGCCCCCCGCCGCCCCCTCCTT
CCGCCAGGTGTCCTGCCTGAAGGAGCTGGTGGCCCGAGTGCTGCA
GAGGCTGTGCGAGCGCGGCGCGAAGAACGTGCTGGCCTTCGGCTT
CGCGCTGCTGGACGGGGCCCGCGGGGGCCCCCCCGAGGCCTTCAC
CACCAGCGTGCGCAGCTACCTGCCCAACACGGTGACCGACGCACT
GCGGGGGAGCGGGGCGTGGGGGCTGCTGCTGCGCCGCGTGGGCGA
CGACGTGCTGGTTCACCTGCTGGCACGCTGCGCGCTCTTTGTGCT
GGTGGCTCCCAGCTGCGCCTACCAGGTGTGCGGGCCGCCGCTGTA
CCAGCTCGGCGCTGCCACTCAGGCCCGGCCCCCGCCACACGCTAG
TGGACCCCGAAGGCGTCTGGGATGCGAACGGGCCTGGAACCATAG
CGTCAGGGAGGCCGGGGTCCCCCTGGGCCTGCCAGCCCCGGGTGC
GAGGAGGCGCGGGGGCAGTGCCAGCCGAAGTCTGCCGTTGCCCAA
GAGGCCCAGGCGTGGCGCTGCCCCTGAGCCGGAGCGGACGCCCGT
TGGGCAGGGGTCCTGGGCCCACCCGGGCAGGACGCGTGGACCGAG
TGACCGTGGTTTCTGTGTGGTGTCACCTGCCAGACCCGCCGAAGA
AGCCACCTCTTTGGAGGGTGCGCTCTCTGGCACGCGCCACTCCCA
CCCATCCGTGGGCCGCCAGCACCACGCGGGCCCCCCATCCACATC
GCGGCCACCACGTCCCTGGGACACGCCTTGTCCCCCGGTGTACGC
CGAGACCAAGCACTTCCTCTACTCCTCAGGCGACAAGGAGCAGCT
GCGGCCCTCCTTCCTACTCAGCTCTCTGAGGCCCAGCCTGACTGG
CGCTCGGAGGCTCGTGGAGACCATCTTTCTGGGTTCCAGGCCCTG
GATGCCAGGGACTCCCCGCAGGTTGCCCCGCCTGCCCCAGCGCTA
CTGGCAAATGCGGCCCCTGTTTCTGGAGCTGCTTGGGAACCACGC
GCAGTGCCCCTACGGGGTGCTCCTCAAGACGCACTGCCCGCTGCG
AGCTGCGGTCACCCCAGCAGCCGGTGTCTGTGCCCGGGAGAAGCC
CCAGGGCTCTGTGGCGGCCCCCGAGGAGGAGGACACAGACCCCCG
TCGCCTGGTGCAGCTGCTCCGCCAGCACAGCAGCCCCTGGCAGGT
GTACGGCTTCGTGCGGGCCTGCCTGCGCCGGCTGGTGCCCCCAGG
CCTCTGGGGCTCCAGGCACAACGAACGCCGCTTCCTCAGGAACAC
CAAGAAGTTCATCTCCCTGGGGAAGCATGCCAAGCTCTCGCTGCA
GGAGCTGACGTGGAAGATGAGCGTGCGGGACTGCGCTTGGCTGCG
CAGGAGCCCAGGGGTTGGCTGTGTTCCGGCCGCAGAGCACCGTCT
GCGTGAGGAGATCCTGGCCAAGTTCCTGCACTGGCTGATGAGTGT
GTACGTCGTCGAGCTGCTCAGGTCTTTCTTTTATGTCACGGAGAC
CACGTTTCAAAAGAACAGGCTCTTTTTCTACCGGAAGAGTGTCTG
GAGCAAGTTGCAAAGCATTGGAATCAGACAGCACTTGAAGAGGGT
GCAGCTGCGGGAGCTGTCGGAAGCAGAGGTCAGGCAGCATCGGGA
AGCCAGGCCCGCCCTGCTGACGTCCAGACTCCGCTTCATCCCCAA
GCCTGACGGGCTGCGGCCGATTGTGAACATGGACTACGTCGTGGG
AGCCAGAACGTTCCGCAGAGAAAAGAGGGCCGAGCGTCTCACCTC
GAGGGTGAAGGCACTGTTCAGCGTGCTCAACTACGAGCGGGCGCG
GCGCCCCGGCCTCCTGGGCGCCTCTGTGCTGGGCCTGGACGATAT
CCACAGGGCCTGGCGCACCTTCGTGCTGCGTGTGCGGGCCCAGGA
CCCGCCGCCTGAGCTGTACTTTGTCAAGGTGGATGTGACGGGCGC
GTACGACACCATCCCCCAGGACAGGCTCACGGAGGTCATCGCCAG
CATCATCAAACCCCAGAACACGTACTGCGTGCGTCGGTATGCCGT
GGTCCAGAAGGCCGCCCATGGGCACGTCCGCAAGGCCTTCAAGAG
CCACGTCTCTACCTTGACAGACCTCCAGCCGTACATGCGACAGTT
CGTGGCTCACCTGCAGGAGACCAGCCCGCTGAGGGATGCCGTCGT
CATCGAGCAGAGCTCCTCCCTGAATGAGGCCAGCAGTGGCCTCTT
CGACGTCTTCCTACGCTTCATGTGCCACCACGCCGTGCGCATCAG
GGGCAAGTCCTACGTCCAGTGCCAGGGGATCCCGCAGGGCTCCAT
CCTCTCCACGCTGCTCTGCAGCCTGTGCTACGGCGACATGGAGAA
CAAGCTGTTTGCGGGGATTCGGCGGGACGGGCTGCTCCTGCGTTT
GGTGGATGATTTCTTGTTGGTGACACCTCACCTCACCCACGCGAA
AACCTTCCTCAGGACCCTGGTCCGAGGTGTCCCTGAGTATGGCTG
CGTGGTGAACTTGCGGAAGACAGTGGTGAACTTCCCTGTAGAAGA
CGAGGCCCTGGGTGGCACGGCTTTTGTTCAGATGCCGGCCCACGG
CCTATTCCCCTGGTGCGGCCTGCTGCTGGATACCCGGACCCTGGA
GGTGCAGAGCGACTACTCCAGCTATGCCCGGACCTCCATCAGAGC
CAGTCTCACCTTCAACCGCGGCTTCAAGGCTGGGAGGAACATGCG
TCGCAAACTCTTTGGGGTCTTGCGGCTGAAGTGTCACAGCCTGTT
TCTGGATTTGCAGGTGAACAGCCTCCAGACGGTGTGCACCAACAT
CTACAAGATCCTCCTGCTGCAGGCGTACAGGTTTCACGCATGTGT
GCTGCAGCTCCCATTTCATCAGCAAGTTTGGAAGAACCCCACATT
TTTCCTGCGCGTCATCTCTGACACGGCCTCCCTCTGCTACTCCAT
CCTGAAAGCCAAGAACGCAGGGATGTCGCTGGGGGCCAAGGGCGC
CGCCGGCCCTCTGCCCTCCGAGGCCGTGCAGTGGCTGTGCCACCA
AGCATTCCTGCTCAAGCTGACTCGACACCGTGTCACCTACGTGCC
ACTCCTGGGGTCACTCAGGACAGCCCAGACGCAGCTGAGTCGGAA
GCTCCCGGGGACGACGCTGACTGCCCTGGAGGCCGCAGCCAACCC
GGCACTGCCCTCAGACTTCAAGACCATCCTGGACTGACAATTGGC
TCGCTTTCTTGCTGTCCAATTTCTATTAAAGGTTCCTTTTGTTCC
CTAAGTCCAACTACTAAACTGGGGGATATTATGAAGGGCCTTGAG
CATCTGGATTCTGCCTAATAAAAAACATTTCTTTTCATTGCGAAT
TCAAAAAAAAAAAAAAAAAAAAAAAAAAAAAAAAAAAAAAAAAAA
AAAAAAAAAAAAAAAAAAAAAAAAAAAAAAAAAAAAAAAAAAAAA
AAAAAAAAAAAAAAAAAAAAAAAAAAAAAAAAAAAAAAAAAAAAA
AAAAAAAAAAAAAAAAAA

In some embodiments, the human TERT mRNA comprises a sequence at least 70%, at least 75%, at least 80%, at least 85%, at least 90%, at least 95%, at least 97%, at least 99%, or 100% identical to SEQ ID NO: 40

AGGAAATAAGAGAGAAAAGAAGAGTAAGAAGAAATATAAGAGCCA
CCATGCCGCGCGCTCCCCGCTGCCGAGCCGTGCGCTCCCTGCTGC
GCAGCCACTACCGCGAGGTGCTGCCGCTGGCCACGTTCGTGCGGC
GCCTGGGGCCCCAGGGCTGGCGGCTGGTGCAGCGCGGGGACCCGG
CGGCTTTCCGCGCGCTGGTGGCCCAGTGCCTGGTGTGCGTGCCCT
GGGACGCACGGCCGCCCCCCGCCGCCCCCTCCTTCCGCCAGGTGT
CCTGCCTGAAGGAGCTGGTGGCCCGAGTGCTGCAGAGGCTGTGCG
AGCGCGGCGCGAAGAACGTGCTGGCCTTCGGCTTCGCGCTGCTGG
ACGGGGCCCGCGGGGGCCCCCCCGAGGCCTTCACCACCAGCGTGC
GCAGCTACCTGCCCAACACGGTGACCGACGCACTGCGGGGGAGCG
GGGCGTGGGGGCTGCTGCTGCGCCGCGTGGGCGACGACGTGCTGG
TTCACCTGCTGGCACGCTGCGCGCTCTTTGTGCTGGTGGCTCCCA
GCTGCGCCTACCAGGTGTGCGGGCCGCCGCTGTACCAGCTCGGCG
CTGCCACTCAGGCCCGGCCCCCGCCACACGCTAGTGGACCCCGAA
GGCGTCTGGGATGCGAACGGGCCTGGAACCATAGCGTCAGGGAGG
CCGGGGTCCCCCTGGGCCTGCCAGCCCCGGGTGCGAGGAGGCGCG
GGGGCAGTGCCAGCCGAAGTCTGCCGTTGCCCAAGAGGCCCAGGC
GTGGCGCTGCCCCTGAGCCGGAGCGGACGCCCGTTGGGCAGGGGT
CCTGGGCCCACCCGGGCAGGACGCGTGGACCGAGTGACCGTGGTT
TCTGTGTGGTGTCACCTGCCAGACCCGCCGAAGAAGCCACCTCTT
TGGAGGGTGCGCTCTCTGGCACGCGCCACTCCCACCCATCCGTGG
GCCGCCAGCACCACGCGGGCCCCCCATCCACATCGCGGCCACCAC
GTCCCTGGGACACGCCTTGTCCCCCGGTGTACGCCGAGACCAAGC
ACTTCCTCTACTCCTCAGGCGACAAGGAGCAGCTGCGGCCCTCCT
TCCTACTCAGCTCTCTGAGGCCCAGCCTGACTGGCGCTCGGAGGC
TCGTGGAGACCATCTTTCTGGGTTCCAGGCCCTGGATGCCAGGGA
CTCCCCGCAGGTTGCCCCGCCTGCCCCAGCGCTACTGGCAAATGC
GGCCCCTGTTTCTGGAGCTGCTTGGGAACCACGCGCAGTGCCCCT
ACGGGGTGCTCCTCAAGACGCACTGCCCGCTGCGAGCTGCGGTCA
CCCCAGCAGCCGGTGTCTGTGCCCGGGAGAAGCCCCAGGGCTCTG
TGGCGGCCCCCGAGGAGGAGGACACAGACCCCCGTCGCCTGGTGC
AGCTGCTCCGCCAGCACAGCAGCCCCTGGCAGGTGTACGGCTTCG
TGCGGGCCTGCCTGCGCCGGCTGGTGCCCCCAGGCCTCTGGGGCT
CCAGGCACAACGAACGCCGCTTCCTCAGGAACACCAAGAAGTTCA
TCTCCCTGGGGAAGCATGCCAAGCTCTCGCTGCAGGAGCTGACGT
GGAAGATGAGCGTGCGGGACTGCGCTTGGCTGCGCAGGAGCCCAG
GGGTTGGCTGTGTTCCGGCCGCAGAGCACCGTCTGCGTGAGGAGA
TCCTGGCCAAGTTCCTGCACTGGCTGATGAGTGTGTACGTCGTCG
AGCTGCTCAGGTCTTTCTTTTATGTCACGGAGACCACGTTTCAAA
AGAACAGGCTCTTTTTCTACCGGAAGAGTGTCTGGAGCAAGTTGC
AAAGCATTGGAATCAGACAGCACTTGAAGAGGGTGCAGCTGCGGG
AGCTGTCGGAAGCAGAGGTCAGGCAGCATCGGGAAGCCAGGCCCG
CCCTGCTGACGTCCAGACTCCGCTTCATCCCCAAGCCTGACGGGC
TGCGGCCGATTGTGAACATGGACTACGTCGTGGGAGCCAGAACGT
TCCGCAGAGAAAAGAGGGCCGAGCGTCTCACCTCGAGGGTGAAGG
CACTGTTCAGCGTGCTCAACTACGAGCGGGCGCGGCGCCCCGGCC
TCCTGGGCGCCTCTGTGCTGGGCCTGGACGATATCCACAGGGCCT
GGCGCACCTTCGTGCTGCGTGTGCGGGCCCAGGACCCGCCGCCTG
AGCTGTACTTTGTCAAGGTGGATGTGACGGGCGCGTACGACACCA
TCCCCCAGGACAGGCTCACGGAGGTCATCGCCAGCATCATCAAAC
CCCAGAACACGTACTGCGTGCGTCGGTATGCCGTGGTCCAGAAGG
CCGCCCATGGGCACGTCCGCAAGGCCTTCAAGAGCCACGTCTCTA
CCTTGACAGACCTCCAGCCGTACATGCGACAGTTCGTGGCTCACC
TGCAGGAGACCAGCCCGCTGAGGGATGCCGTCGTCATCGAGCAGA
GCTCCTCCCTGAATGAGGCCAGCAGTGGCCTCTTCGACGTCTTCC
TACGCTTCATGTGCCACCACGCCGTGCGCATCAGGGGCAAGTCCT
ACGTCCAGTGCCAGGGGATCCCGCAGGGCTCCATCCTCTCCACGC
TGCTCTGCAGCCTGTGCTACGGCGACATGGAGAACAAGCTGTTTG
CGGGGATTCGGCGGGACGGGCTGCTCCTGCGTTTGGTGGATGATT
TCTTGTTGGTGACACCTCACCTCACCCACGCGAAAACCTTCCTCA
GGACCCTGGTCCGAGGTGTCCCTGAGTATGGCTGCGTGGTGAACT
TGCGGAAGACAGTGGTGAACTTCCCTGTAGAAGACGAGGCCCTGG
GTGGCACGGCTTTTGTTCAGATGCCGGCCCACGGCCTATTCCCCT
GGTGCGGCCTGCTGCTGGATACCCGGACCCTGGAGGTGCAGAGCG
ACTACTCCAGCTATGCCCGGACCTCCATCAGAGCCAGTCTCACCT
TCAACCGCGGCTTCAAGGCTGGGAGGAACATGCGTCGCAAACTCT
TTGGGGTCTTGCGGCTGAAGTGTCACAGCCTGTTTCTGGATTTGC
AGGTGAACAGCCTCCAGACGGTGTGCACCAACATCTACAAGATCC
TCCTGCTGCAGGCGTACAGGTTTCACGCATGTGTGCTGCAGCTCC
CATTTCATCAGCAAGTTTGGAAGAACCCCACATTTTTCCTGCGCG
TCATCTCTGACACGGCCTCCCTCTGCTACTCCATCCTGAAAGCCA
AGAACGCAGGGATGTCGCTGGGGGCCAAGGGCGCCGCCGGCCCTC
TGCCCTCCGAGGCCGTGCAGTGGCTGTGCCACCAAGCATTCCTGC
TCAAGCTGACTCGACACCGTGTCACCTACGTGCCACTCCTGGGGT
CACTCAGGACAGCCCAGACGCAGCTGAGTCGGAAGCTCCCGGGGA
CGACGCTGACTGCCCTGGAGGCCGCAGCCAACCCGGCACTGCCCT
CAGACTTCAAGACCATCCTGGACTGATAATTAAGCTGCCTTCTGC
GGGGCTTGCCTTCTGGCCATGCCCTTCTTCTCTCCCTTGCACCTG
TACCTCTTGGTCTTTGAATAAAGCCTGAGTAGGAAGAAAAAAAAA
AAAAAAAAAAAAAAAAAAAAAAAAAAAAAAAAAAAAAAAAAAAAA
AAAAAAAAAAAAAAAAAAAAAAAAAAAAAAAAAAAAAAAAAAAAA
AAAAAAAAAAAAAAAAAAAA

In some embodiments, a TERT mRNA may comprise a nucleic acid sequence at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to any one of SEQ ID NOS: 38-40.

The disclosure provides compositions for the extension of telomeres in a cell, the compositions comprising a compound of the present disclosure, as described above, and a further component. In some embodiments, the further component comprises a telomerase RNA component (TERC). In some embodiments, the compositions further comprise a telomerase RNA component (TERC). In some embodiments, the compositions further comprise one or more additional components that may facilitate delivery of the RNA to cells in vitro and/or in vivo. In some embodiments, the one or more additional components comprise a nanoparticle. In some embodiments, the nanoparticle comprises a lipid. In some embodiments, the nanoparticle or the lipid comprise a coatsome-like lipid or a compound of the disclosure. In some embodiments, the nanoparticle or the lipid comprise a compound of the disclosure according to Formula I.

### II. Delivery Vehicles

In some embodiments, one or more mRNAs may be delivered to a cell or tissue via delivery vehicles. In some embodiments a delivery vehicle may be a nanoparticle. In some embodiments, the delivery vehicle is a lipid nanoparticle (LNP) including but not limited to a nanoparticle comprising lipids and/or polymers, a liposome, a liposomal nanoparticle, a cationic lipid, or an exosome. As used herein, liposomal nanoparticles may be characterized as microscopic vesicles having an interior aqueous space sequestered from an outer medium by a membrane of one or more bilayers.

In some embodiments, the nanoparticle is a polymeric nanoparticle. In some embodiments, the nanoparticle is a metal nanoparticle. In other embodiments, the delivery vehicle comprises or consists of a recombinant virus or virus-like particle, e.g., an adenovirus, adeno-associated virus (AAV), herpesvirus, or retrovirus, e.g., lentivirus. In some embodiments, the delivery vehicle comprises or consists of a modified viral vector, e.g., an adenovirus dodecahedron or recombinant adenovirus conglomerate. In other embodiments, the delivery vehicle may comprise or consist of calcium phosphate nucleotides, aptamers, cell-penetrating peptides or other vectorial tags.

### A. Liposomal Delivery Vehicles

In some embodiments, a suitable delivery vehicle is a lipid nanoparticle (LNP), Exemplary LNPs may comprise one or more different lipids and/or polymers. In some embodiments, an LNP comprises one or more of ionizable lipids, neutral lipids, cholesterols, and/or stabilizing lipids (e.g., PEGylated lipids).

### Ionizable Lipids

In some embodiments, an LNP may comprise an ionizable lipid. An ionizable lipid may refer to any of a number of lipid species that have a net positive charge at a selected pH, such as a physiological pH. An ionizable lipid may also, for example, refer to a lipid in an ionized state, e.g., a cationic lipid. In some embodiments, an LNP may comprise an ionizable lipid as disclosed in either of WO 2010/053572 or WO 2012/170930, or variations thereof, both of which are incorporated herein by reference in their entirety.

In some embodiments, an LNP for liver delivery of a TERT mRNA may comprise one or more of MC3 (((6Z,9Z,28Z,31Z)-heptatriaconta-6,9,28,31-tetraen-19-yl 4-(dimethylamino)butanoate), 1,2-dilineoyl-3-dimethylammonium-propane (DLinDAP), DLin-MC3-DMA 4-(dimethylamino)-butanoic acid, (10Z,13Z)-1-(9Z,12Z)-9,12-octadecadien-1-yl-10,13-nonadecadien-1-yl ester and/or cKK-E12 3,6-Bis(4-(bis(2-hydroxydodecyl)amino)butyl)piperazine-2,5-dione. In some embodiments the LNP comprises 2,2-dilinoleyl-4-dimethylaminoethyl-[1,3]-dioxolane (Dlin-KC2-DMA, 1) and/or (6Z,9Z,28Z,31 Z)-Heptatriaconta-6,9,28,31-tetraen-19-yl 4-(dimethylamino)butanoate. In some embodiments, the ionizable lipid may have a pKa range of 6.1-6.7, optionally a pKa range of 6.2-6.5.

In some embodiments, an LNP comprises 1,2-dioleoyl-3-trimethylammonium-propane (DOTAP), N,N-distearyl-N,N-dimethylarnmonium bromide (DABB), or 1,2-dimyristoyl-sn-glycero-3-ethylphosphocholine (EPC). In some embodiments, an LNP comprises a ionizable lipid wherein the ionizable lipid is one or more of N-[1-(2,3-dioleyloxy)propyl]-N,N,N-trimethylammonium chloride (DOTMA), 5-carboxyspermylglycinedioctadecylamide (DOGS), 2,3-dioleyloxy-N-[2(spermine-carboxamido)ethyl]-N,N-dimethyl-1-propanaminium (DOSPA), 1,2-Dioleoyl-3-Dimethylammonium-Propane (DODAP), and/or 1,2-Dioleoyl-3-Trimethylammonium-Propane (DOTAP), or variations thereof. An LNP may also comprise one or more of 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane or (DLenDMA), 4-(dimethylamino)-butanoic acid, (10Z,13Z)-1-(9Z,12Z)-9,12-octadecadien-1-yl-10,13-nonadecadien-1-yl ester (DLin-MC3-DMA), N-dioleyl-N,N-dimethylammonium chloride (DODAC), or variations thereof. In other embodiments, an LNP may comprise a ionizable lipid of XTC (2,2-Dilinoleyl-4-dimethylaminoethyl-[1,3]-dioxolane), MC3 (((6Z,9Z,28Z,31Z)-heptatriaconta-6,9,28,31-tetraen-19-yl 4-(dimethylamino)butanoate), ALNY-100 ((3aR,5s,6aS)-N,N-dimethyl-2,2-di((9Z,12Z)-octadeca-9,12-dienyl)tetrahydro-3aH-cyclopenta[d][1,3]dioxol-5-amine)), NC98-5 (4,7,13-tris(3-oxo-3-(undecylamino)propyl)-N1,N16-diundecyl-4,7,10,13-tetraazahexadecane-1,16-diamide), or variations thereof.

In some embodiments, an LNP may comprise an ionizable lipid, e.g., one or more of (15Z,18Z)-N,N-dimethyl-6-(9Z,12Z)-octadeca-9,12-dien-1-yl) tetracosa-15,18-dien-1-amine, (15Z,18Z)-N,N-dimethyl-6-((9Z,12Z)-octadeca-9,12-dien-1-yl) tetracosa-4,15,18-trien-1-amine, and (15Z,18Z)-N,N-dimethyl-6-((9Z,12Z)-octadeca-9, and 12-dien-1-yl) tetracosa-5, 15,18-trien-1-amine (HGT5002).

In some embodiments, an LNP may comprise a cleavable ionizable lipid comprising a disulfide bond, e.g., COATSOME™ SS-OP, i.e. SS-OP™, COATSOME™ SS-M, COATSOME™ SS-E, COATSOME™ SS-EC, COATSOME™ SS-LC, COATSOME™ SS-OC and variations thereof. In some embodiments, an LNP may comprise about 10%, about 20%, about 30%, about 40%, about 50%, about 60%, about 70%, about 80%, or about 90% ionizable lipids relative to the other lipids.

### Other “Helper” Phospholipids

In some embodiments, an LNP may comprise additional lipids selected from one more of: distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or variants thereof. In some embodiments, an LNP may include one or more phosphatidyl lipids, for example, the phosphatidyl compounds (e.g., phosphatidylglycerol, phosphatidylcholine, phosphatidylserine and phosphatidylethanolamine). In some embodiments, an LNP may comprise sphingolipids, for example but not limited to, sphingosine, ceramide, sphingomyelin, cerebroside and ganglioside. In some embodiments, the aforementioned “helper” lipids contribute to the stability and/or specificity of the LNP composition.

### Cholesterol-Based Lipids

In some embodiments, an LNP may comprise one or more cholesterol-based lipids. A cholesterol-based lipid may include but is not limited to: PEGylated cholesterol, DC-Choi (N,N-dimethyl-N-ethylcarboxamidocholesterol), 1,4-bis(3-N-oleylamino-propyl)piperazine. In some embodiments, an LNP may comprise about 2% to about 30%, or about 5% to about 20% of cholesterol relative to the total lipid present.

### PEGylated Lipids

Without wishing to be bound by theory, it is contemplated that the addition of a lipid modified with an insulating molecule such as a protein or other polymer such as polyethylene-glycol (PEG), also known as a PEGylated lipid, may prevent complex aggregation and increase circulation lifetime to facilitate the delivery of the liposome encapsulated mRNA to the target cell. In some embodiments, the addition of a PEGylated lipid protects the LNP from immune targeting. In some embodiments, the PEGylated lipid forms a hydrophilic barrier around the hydrophobic LNP, preventing opsonization of plasma proteins and bypassing macrophage uptake. In some embodiments, lipids modified with other hydrophilic molecules may be substituted for PEGylated lipids in an LNP delivery vehicle.

In some embodiments of the disclosure, an LNP may comprise one or more PEGylated lipids. For example, the use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000](C8 PEG-2000 ceramide) is contemplated by the present disclosure in combination with one or more of the ionizable and/or other lipids. In some embodiments, PEGylated lipids comprise PEG-ceramides having shorter acyl chains (e.g., C14 or C18). In some embodiments, the PEGylated lipid DSPE-PEG-Maleimide-Lectin may be used. Other contemplated PEG-modified lipids include, but are not limited to, a polyethylene glycol chain of up to 5 kDa in length covalently attached to a lipid with alkyl chain(s) of C6-C2o length. Without wishing to be bound by a particular theory, it is contemplated that the addition of PEGylated lipids may prevent complex aggregation and increase circulation lifetime to facilitate the delivery of the liposome encapsulated mRNA to the target cell.

In some embodiments, PEGylated lipids may comprise about 0% to about 20%, about 0% to about 15%, about 0% to about 10%, about 1% to about 10%, about 1% to about 8%, 1% to about 6%, 1% to about 5%, about 2% to about 10%, about 4% to about 10%, of the total lipids present in the liposome by molar ratio. In some embodiments, the percentage of PEGylated lipids may be less than about 20%, about 15%, about 10%, about 9%, about 8%, about 7%, about 6%, about 5%, about 4%, about 3%, about 2%, or about 1% of the total lipids present in the liposome by molar ratio. In some embodiments, the percentage of PEGylated lipids may be greater than about 1%, about 2%, about 3%, about 4%, about 5%, about 6%, about 7%, about 8%, about 9%, about 10%, about 15%, or about 20% of the total lipids present in the liposome by molar ratio.

In some embodiments, a lipid nanoparticle formulation may comprise, consist essentially of or consist of any of those described in U.S. Pat. Nos. 11,185,595; 9,868,693; 10,195,156; 9,877,919; 9,738,593; 10,399,937; 10,106,490; 9,738,593; 10,821,186; or 8,058,069, each of which is incorporated by reference herein in its entirety; or described in U.S. Patent Application Publication Nos. US20180085474A1, US20210259980A1, US20200206362A1, US20210267895A1, US20200283372A1, or US20200163878A1, each of which is incorporated by reference herein in its entirety.

### Lipid Nanoparticle (LNP) Compositions

The following example LNP formulations are not intended to be limiting.

In some embodiments, an LNP may comprise a molar ratio of about 35, about 40, about 45, about 50, about 55, about 60, about 65, about 70, or about 75 moles of an ionizable lipid. In some embodiments, an LNP may comprise a molar ratio of about 0.1, about 1.0, about 2.0, about 3.0, about 4.0, about 5.0, about 6.0, about 7.0, about 8.0, about 10, about 12, about 14, about 16, about 18, about 20, about 25, about 30, about 40, or about 50 moles of another phospholipid. In some embodiments, an LNP may comprise a molar ratio of about 10, about 15, about 20, about 25, about 30, about 35, about 40, about 45, about 50, about 55, about 60, about 65, or about 70 moles of cholesterol. In some embodiments, an LNP may comprise a molar ratio of about 0.1, about 0.25, about 0.5, about 0.75, about 1.0, about 1.25, about 1.5, about 1.75, about 2.0, about 2.5, about 3.0, about 3.5, about 4.0, about 4.5 or about 5.0 moles of a PEGylated lipid.

In some embodiments, an LNP comprises a molar ratio of about 40-70 moles of an ionizable lipid to about 0.1 to about 20 moles of another phospholipid, about 20 to about 60 moles of cholesterol, and about 0.1 to about 5 moles of PEGylated lipid. In some embodiments, the LNP delivery vehicle comprises a molar ratio of about 50-60 moles of an ionizable lipid to about 4-18 moles of another phospholipid, about 35-50 moles of cholesterol, and about 1-3 moles of PEGylated lipid.

In some embodiments, an LNP may comprise a molar ratio of about 50 to about 60 moles of an ionizable lipid, about 4 to about 6 moles of a phospholipid, about 35 to about 45 moles of cholesterol, and about 1 to about 2 moles of PEGylated lipid.

In some embodiments, an LNP may comprise a molar ratio of about 30 to 40 moles of an ionizable lipid, about 14 to about 18 moles of a phospholipid, about 40 to about 50 moles of a cholesterol, and about 2.0 to about 3.0 moles of a PEGylated lipid.

In some embodiments, an LNP may comprise the ionizable lipid SS-OP™, the phospholipid DOPC, a cholesterol lipid, and the PEGylated lipid DMG-PEG2000. In some embodiments, an LNP may comprise a molar ratio of 55 moles of SS-OP™, to 5 moles of DOPC, 40 moles of a cholesterol lipid, and 1.5 moles of PEGylated lipid DMG-PEG2000. In some embodiments, an LNP may comprise a molar ratio of 52.5 moles of SS-OP™, to 7.5 moles of DOPC, 40 moles of a cholesterol lipid, and 1.5 moles of PEGylated lipid DMG-PEG2000.

In some embodiments, an LNP may comprise the ionizable lipid cKK-E12, the phospholipid DOPE, a cholesterol lipid, and the PEGylated lipid 14:0 PEG2000 PE. In some embodiments, an LNP may comprise a molar ratio of about 35 moles of cKK-E12, to about 16 moles of DOPE, about 46.5 moles of a cholesterol lipid, and about 2.5 moles of PEGylated lipid 14:0 PEG2000 PE.

In some embodiments, an LNP may comprise the ionizable lipid DLin-MC3-DMA, the phospholipid DSPC, a cholesterol lipid, and the PEGylated lipid DMG-PEG2000. In some embodiments, an LNP may comprise a molar ratio of about 50 moles of DLin-MC3-DMA, about 10 moles of the phospholipid DSPC, about 40 moles of a cholesterol lipid, and about 1.5 moles of the PEGylated lipid DMG-PEG2000.

In some embodiments, an LNP may comprise the ionizable lipid SS-OP™, the phospholipid DOPE, a cholesterol lipid, and the PEGylated lipid 14:0 PEG2000 PE. In some embodiments, an LNP may comprise a molar ratio of about 35 moles of SS-OP™, to about 16 moles of DOPE, about 46.5 moles of a cholesterol lipid, and about 2.5 moles of PEGylated lipid 14:0 PEG2000 PE.

In some embodiments, an LNP may comprise the ionizable lipid cKK-E12, the phospholipid DOPC, a cholesterol lipid, and the PEGylated lipid DMG-PEG2000. In some embodiments, an LNP may comprise a molar ratio of about 55 moles of cKK-E12, to about 5 moles of DOPC, about 40 moles of a cholesterol lipid, and about 1.5 moles of PEGylated lipid DMG-PEG2000.

### B. Polymer Nanoparticles

In some embodiments, a suitable delivery vehicle is formulated using a polymer as a carrier, alone or in combination with other carriers including various lipids described herein. Thus, in some embodiments, liposomal delivery vehicles, as used herein, also encompass polymer containing nanoparticles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, polyethylene glycol (PEG)-modified (PEGylated) protamine, poly-D-lysine (PLL), PEGylated PLL and polyethylenimine (PEI). When PEI is present, it may be linear or branched PEI of a molecular weight ranging from 10 to 40 kDA, e.g., 25 kDa branched PEI (Sigma #408727). In some embodiments the PEGylated lipid is 14:0 PEG2000 PE and/or DMG-PEG2000.

### C. Delivery Vehicles Targeting Liver

In some embodiments, delivery vehicles disclosed herein preferentially target specific organs, e.g., the liver. In various embodiments, the delivery vehicles may delivery mRNA to liver cells 10, 10², 10³, 10⁴, 10⁵, 10⁶, 10⁷, 10⁸, 10⁹, or 10¹⁰-fold more effectively compared a reference cell type (e.g., lung cells). However, it will be understood that some level of delivery to non-target cells/organs may be tolerated without decreasing the effectiveness in the target organ/cell. In some embodiments, the lipid composition of a delivery vehicle enhances delivery to the liver relative to other lipid compositions known in the art. In other embodiments, the lipid composition of a delivery vehicle enhances delivery to the liver relative to other lipid compositions. In some embodiments, the presence or level of cholesterol enhances delivery of a delivery vehicle, e.g. an LNP or extracellular vesicle to the liver.

In some embodiments, a delivery vehicle, e.g. an LNP, targeting the liver may comprise a molar ratio of about 35, about 40, about 45, about 50, about 55, about 60, about 65, about 70, or about 75 moles of an ionizable lipid. In some embodiments, an LNP may comprise a molar ratio of about 0.1, about 1.0, about 2.0, about 3.0, about 4.0, about 5.0, about 6.0, about 7.0, about 8.0, about 10, about 12, about 14, about 16, about 18, about 20, about 25, about 30, about 40, or about 50 moles of another phospholipid. In some embodiments, an LNP may comprise a molar ratio of about 10, about 15, about 20, about 25, about 30, about 35, about 40, about 45, about 50, about 55, about 60, about 65, or about 70 moles of cholesterol. In some embodiments, an LNP may comprise a molar ratio of about 0.1, about 0.25, about 0.5, about 0.75, about 1.0, about 1.25, about 1.5, about 1.75, about 2.0, about 2.5, about 3.0, about 3.5, about 4.0, about 4.5 or about 5.0 moles of a PEGylated lipid.

In some embodiments, a delivery vehicle, e.g. an LNP, targeting the liver comprises a molar ratio of about 40-70 moles of an ionizable lipid to about 0.1 to about 20 moles of another phospholipid, about 20 to about 60 moles of cholesterol, and about 0.1 to about 5 moles of PEGylated lipid. In some embodiments, the LNP delivery vehicle comprises a molar ratio of about 50-60 moles of an ionizable lipid to about 4-18 moles of another phospholipid, about 35-50 moles of cholesterol, and about 1-3 moles of PEGylated lipid.

In some embodiments, a delivery vehicle, e.g. an LNP, targeting the liver may comprise a molar ratio of about 50 to about 60 moles of an ionizable lipid, about 4 to about 6 moles of a phospholipid, about 35 to about 45 moles of cholesterol, and about 1 to about 2 moles of PEGylated lipid.

In some embodiments, a delivery vehicle, e.g. an LNP, targeting the liver may comprise a molar ratio of about 30 to 40 moles of an ionizable lipid, about 14 to about 18 moles of a phospholipid, about 40 to about 50 moles of a cholesterol, and about 2.0 to about 3.0 moles of a PEGylated lipid.

In some embodiments, a delivery vehicle, e.g. an LNP, targeting the liver may comprise the ionizable lipid SS-OP™, the phospholipid DOPC, a cholesterol lipid, and the PEGylated lipid DMG-PEG2000. In some embodiments, an LNP may comprise a molar ratio of 55 moles of SS-OP™, to 5 moles of DOPC, 40 moles of a cholesterol lipid, and 1.5 moles of PEGylated lipid DMG-PEG2000.

In some embodiments, a delivery vehicle, e.g. an LNP, targeting the liver may comprise the ionizable lipid cKK-E12, the phospholipid DOPE, a cholesterol lipid, and the PEGylated lipid 14:0 PEG2000 PE. In some embodiments, an LNP may comprise a molar ratio of about 35 moles of cKK-E12, to about 16 moles of DOPE, about 46.5 moles of a cholesterol lipid, and about 2.5 moles of PEGylated lipid 14:0 PEG2000 PE.

In some embodiments, a delivery vehicle, e.g. an LNP, targeting the liver may comprise the ionizable lipid DLin-MC3-DMA, the phospholipid DSPC, a cholesterol lipid, and the PEGylated lipid DMG-PEG2000. In some embodiments, an LNP may comprise a molar ratio of about 50 moles of DLin-MC3-DMA, about 10 moles of the phospholipid DSPC, about 40 moles of a cholesterol lipid, and about 1.5 moles of the PEGylated lipid DMG-PEG2000.

In some embodiments, a delivery vehicle, e.g. an LNP, targeting the liver may comprise the ionizable lipid SS-OP™, the phospholipid DOPE, a cholesterol lipid, and the PEGylated lipid 14:0 PEG2000 PE. In some embodiments, an LNP may comprise a molar ratio of about 35 moles of SS-OP™, to about 16 moles of DOPE, about 46.5 moles of a cholesterol lipid, and about 2.5 moles of PEGylated lipid 14:0 PEG2000 PE.

In some embodiments, a delivery vehicle, e.g. an LNP, targeting the liver may comprise the ionizable lipid cKK-E12, the phospholipid DOPC, a cholesterol lipid, and the PEGylated lipid DMG-PEG2000. In some embodiments, an LNP may comprise a molar ratio of about 55 moles of cKK-E12, to about 5 moles of DOPC, about 40 moles of a cholesterol lipid, and about 1.5 moles of PEGylated lipid DMG-PEG2000.

In some embodiments, a delivery vehicle comprises an organ-specific targeting ligand to enhance delivery to a particular organ, e.g. the liver. Ligands may include but are not limited to proteins (e.g., human serum albumin HSA), low-density lipoprotein (LDL), or globulin); carbohydrates (e.g., a dextran, pullulan, chitin, chitosan, inulin, cyclodextrin, N-acetylgalactosamine, or hyaluronic acid); or lipids. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene maleic acid anhydride copolymer, poly(L-lactide-co-glycolide) copolymer, divinyl ether maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Examples of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide. Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic. Other examples of ligands include dyes, intercalating agents (e.g. acridines), cross linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, bomeol, menthol, 1,3-propanediol, heptadecyl group, palmitic acid, myristic acid,03-(oleoyl)lithocholic acid, 03-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

In some embodiments, the organ targeting ligands are proteins, e.g., glycoproteins, or peptides, e.g., molecules having a specific affinity for a co-ligand, or antibodies e.g., an antibody, that binds to a specified cell type, e.g., a liver cell. In some embodiments, the ligands may be hormones or hormone receptors. Ligands may also be non-peptidic species, such as lipids, lectins, carbohydrates, vitamins, cofactors, multivalent lactose, multivalent galactose, N-acetyl-galactosamine, N-acetyl glucosamine multivalent mannose, or multivalent fructose.

In some embodiments, a delivery vehicle to target the liver, e.g. an LNP, may comprise an apoE ligand and/or a ligand comprising a multivalent N-acetylgalactosamine (GalNAc)-cluster, which binds with high affinity to the asialoglycoprotein receptor (ASGPR) expressed on hepatocytes. In some embodiments, an LNP may comprise Retinol Binding protein (RBP) for targeting hepatic cells, which express the RBP receptor.

In some embodiments, a delivery vehicle may comprise an extracellular vesicle, e.g. an exosome, to target the liver. In some embodiments, an extracellular vesicle comprises one or more tissue targeting moieties, including but not limited to lipids, peptides or antibodies.

Compositions of the disclosure may comprise one or more components that may facilitate delivery of the RNA to cells. Collectively or in part, components of the composition may comprise a delivery vehicle. In some embodiments, the delivery vehicle facilitates targeting and uptake of the ribonucleic acid of a composition of the disclosure to a target cell. Exemplary delivery vehicles include, but are not limited to, nanoparticles, lipid nanoparticles (LNPs), liposomes, micelles, exosomes, cationic lipids and a natural or artificial lipoprotein particle.

In some embodiments, a delivery vehicle comprises an ionizable lipid. An ionizable lipid may refer to any of a number of lipid species that have a net positive charge at a selected pH, such as a physiological pH. An ionizable lipid may also, for example, refer to a lipid in an ionized state, e.g., a cationic lipid.

In some embodiments, a cationic lipid formulation comprises a cationic lipid and a structural or matrix lipid. Cationic lipids may be composed of a cationic amine moiety and a lipid moiety, and the cationic amine moiety and a polyanion nucleic acid may interact to form a positively charged liposome or lipid membrane structure. In some embodiments, reference to a lipid “moiety” and a “lipid” may be equivalent. Thus, uptake into cells may be promoted and nucleic acids delivered into cells.

In some embodiments, the ionizable lipid may be a compound of Formula (1):

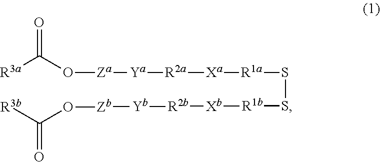

In the formula (1): R^{1a} and R^{1b} each independently represents an alkylene group having 1 to 6 carbon atoms, and may be linear or branched. The alkylene group may have 1 to 4 carbon atoms, or may have 1 to 2. Specific examples of the alkylene group having 1 to 6 carbon atoms include a methylene group, an ethylene group, a trimethylene group, an isopropylene group, a tetramethylene group, an isobutylene group, a pentamethylene group, and a neopentylene group. R^{1a} and R^{1b} may be each independently a methylene group, an ethylene group, a trimethylene group, an isopropylene group, or a tetramethylene group, and may be an ethylene group.

R^{1a} may be different or be the same as R^{1b}.

X^{a} and X^{b} are each independently an acyclic alkyl tertiary amino group having 1 to 6 carbon atoms and 1 tertiary amino group, or 2 to 5 carbon atoms, and a cyclic alkylene tertiary amino group having 1 to 2 tertiary amino groups, and/or each independently a cyclic alkylene having 2 to 5 carbon atoms and 1 to 2 tertiary amino groups and an alkylene tertiary amino group.

The alkyl group having 1 to 6 carbon atoms in the acyclic alkyl tertiary amino group having 1 to 6 carbon atoms and 1 tertiary amino group is branched even if it is linear. The alkyl group may be annular. The alkyl group may have 1 to 3 carbon atoms. Specific examples of the alkyl group having 1 to 6 carbon atoms include methyl group, ethyl group, propyl group, isopropyl group, n-butyl group, sec-butyl group, isobutyl group, tert-butyl group, pentyl group, and isopentyl group. Neopentyl group, t-pentyl group, 1,2-dimethylpropyl group, 2-methylbutyl group, 2-methylpentyl group, 3-methylpentyl group, 2,2-dimethylbutyl group, 2,3-dimethylbutyl group, A cyclohexyl group etc. can be mentioned.

A specific structure of an acyclic alkyl tertiary amino group having 1 to 6 carbon atoms and 1 tertiary amino group is represented by X¹.

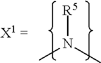

R⁵ of X¹ represents an alkyl group having 1 to 6 carbon atoms and may be linear, branched or cyclic. The alkyl group may have 1 to 3 carbon atoms. Specific examples of the alkyl group having 1 to 6 carbon atoms include methyl group, ethyl group, propyl group, isopropyl group, n-butyl group, sec-butyl group, isobutyl group, tert-butyl group, pentyl group, and isopentyl group. Neopentyl group, t-pentyl group, 1,2-dimethylpropyl group, 2-methylbutyl group, 2-methylpentyl group, 3-methylpentyl group, 2,2-dimethylbutyl group, 2,3-dimethylbutyl group, A cyclohexyl group etc. can be mentioned.

The number of carbon atoms in the cyclic alkylene tertiary amino group having 2 to 5 carbon atoms and 1 to 2 tertiary amino groups may be 4 to 5. Specific examples of the cyclic alkylene tertiary amino group having 2 to 5 carbon atoms and 1 to 2 tertiary amino groups include aziridylene group, azetidylene group, pyrrolidylene group, piperidylene group, imidazolidylene group, a piperazylene group, optionally a pyrrolidylene group, a piperidylene group or a piperazylene group.

Number is 2 to 5 carbon atoms, and specific structure of alkylene tertiary amino groups containing 1 annular tertiary amino group represented by X².

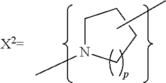

P of X² is 1 or 2. When p is 1, X² is a pyrrolidylene group, and when p is 2, X² is a piperidylene group.

A specific structure of a cyclic alkylene tertiary amino group having 2 to 5 carbon atoms and 2 tertiary amino groups is represented by X³.

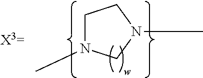

W of X³ is 1 or 2. When w is 1, X³ is an imidazolidylene group, and when w is 2, X³ is a piperazylene group.

X^{a} may be different be identical to X^{b}.

R^{2a} and R^{2b} each independently represent an alkylene group or an oxydialkylene group having 8 or less carbon atoms, optionally each independently an alkylene group having 8 or less carbon atoms.

The alkylene group having 8 or less carbon atoms may be linear or branched but is optionally linear. The number of carbon atoms contained in the alkylene group is optionally 6 or less, and optionally 4 or less. Specific examples of the alkylene group having 8 or less carbon atoms include methylene group, ethylene group, propylene group, isopropylene group, tetramethylene group, isobutylene group, pentamethylene group, hexamethylene group, heptamethylene group, octamethylene group, and the like. In some embodiments included are a methylene group, an ethylene group, a propylene group, and a tetramethylene group.

The oxydialkylene group having 8 or less carbon atoms refers to an alkylene group (alkylene-O-alkylene) via an ether bond, and the total number of carbon atoms of two alkylene groups is 8 or less. Here, the two alkylenes may be the same or different, but are optionally the same. Specific examples of the oxydialkylene group having 8 or less carbon atoms include an oxydimethylene group, an oxydiethylene group, an oxydipropylene group, and an oxydibutylene group.

R^{2a} may be same or different and R^{2b}.

Y^{a} and Y^{b} are each independently an ester bond, an amide bond, a carbamate bond, an ether bond or a urea bond, optionally each independently an ester bond, an amide bond or a carbamate bond. While Y binding orientation of Y^{a} and Y^{b} are not limited, if Y^{a} and Y^{b} is an ester bond, optionally, —Z^{a}—CO—R^{2a}— and —Z^{b}—CO—O—R^{2b}-Structure.

Y^{a} may be different or identical to Y^{b}.

Z^{a} and Z^{b} are each independently a divalent group derived from an aromatic compound having 3 to 16 carbon atoms, having at least one aromatic ring, and optionally having a hetero atom. Represents. The number of carbon atoms contained in the aromatic compound is optionally 6 to 12, or 6 to 7. Moreover, the number of aromatic rings contained in the aromatic compound is optionally one.

As the types of aromatic rings contained in the aromatic compound having 3 to 16 carbon atoms, as for aromatic hydrocarbon rings, benzene ring, naphthalene ring, anthracene ring, and aromatic heterocycles as imidazole ring, pyrazole ring, oxazole ring, Isoxazole ring, thiazole ring, isothiazole ring, triazine ring, pyrrole ring, furanthiophene ring, pyrimidine ring, pyridazine ring, pyrazine ring, pyridine ring, purine ring, pteridine ring, benzimidazole ring, indole ring, benzofuran ring, quinazoline ring, phthalazine ring, quinoline ring, isoquinoline ring, coumarin ring, chromone ring, benzodiazepine ring, phenoxazine ring, phenothiazine ring, acridine ring, etc., optionally benzene ring, naphthalene ring, anthracene ring. The aromatic ring may have a substituent. Examples of the substituent include an acyl group having 2 to 4 carbon atoms, an alkoxycarbonyl group having 2 to 4 carbon atoms, a carbamoyl group having 2 to 4 carbon atoms, and 2 to 2 carbon atoms. 4 acyloxy groups, acylamino groups having 2 to 4 carbon atoms, alkoxycarbonylamino groups having 2 to 4 carbon atoms, fluorine atoms, chlorine atoms, bromine atoms, iodine atoms, alkylsulfanyl groups having 1 to 4 carbon atoms, 1 carbon atom Alkylsulfonyl group having 4 to 4, arylsulfonyl group having 6 to 10 carbon atoms, nitro group, trifluoromethyl group, cyano group, alkyl group having 1 to 4 carbon atoms, ureido group having 1 to 4 carbon atoms, 1 to carbon atoms 4 alkoxy groups, aryl groups having 6 to 10 carbon atoms, aryloxy groups having 6 to 10 carbon atoms, and the like. Some examples include acetyl groups, methoxycarbonyl groups, methyl carbonate groups, and the like, moyl group, acetoxy group, acetamide group, methoxycarbonylamino group, fluorine atom, chlorine atom, bromine atom, iodine atom, methylsulfanyl group, phenylsulfonyl group, nitro group, trifluoromethyl group, cyano group, methyl group, ethyl group Propyl group, isopropyl group, t-butyl group, ureido group, methoxy group, ethoxy group, propoxy group, isopropoxy group, t-butoxy group, phenyl group and phenoxy group.

A specific structure of Z^{a} and Z^{b} includes Z¹.

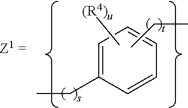

Wherein, s represents an integer of 0 to 3, t represents an integer of 0 to 3, u represents an integer of 0 to 4, represents a u-number of R 4 is independently a substituent.

S in Z¹ is optionally an integer of 0 to 1.

T in Z¹ is optionally an integer of 0 to 2.

U in Z¹ is optionally an integer of 0 to 2.

R 4 in Z¹ is a substituent of an aromatic ring (benzene ring) contained in an aromatic compound having 3 to 16 carbon atoms that does not inhibit the reaction in the process of synthesizing the ionizable lipid. Examples of the substituent include an acyl group having 2 to 4 carbon atoms, an alkoxycarbonyl group having 2 to 4 carbon atoms, a carbamoyl group having 2 to 4 carbon atoms, an acyloxy group having 2 to 4 carbon atoms, and an acylamino group having 2 to 4 carbon atoms, an alkoxycarbonylamino group having 2 to 4 carbon atoms, fluorine atom, chlorine atom, bromine atom, iodine atom, alkylsulfanyl group having 1 to 4 carbon atoms, alkylsulfonyl group having 1 to 4 carbon atoms, 6 to 10 carbon atoms Arylsulfonyl group, nitro group, trifluoromethyl group, cyano group, alkyl group having 1 to 4 carbon atoms, ureido group having 1 to 4 carbon atoms, alkoxy group having 1 to 4 carbon atoms, aryl group having 6 to 10 carbon atoms And aryloxy groups having 6 to 10 carbon atoms, and examples include acetyl, methoxycarbonyl, methylcarbamoyl, acetoxy, Mido group, methoxycarbonylamino group, fluorine atom, chlorine atom, bromine atom, iodine atom, methylsulfanyl group, phenylsulfonyl group, nitro group, trifluoromethyl group, cyano group, methyl group, ethyl group, propyl group, isopropyl group, T-butyl group, ureido group, methoxy group, ethoxy group, propoxy group, isopropoxy group, t-butoxy group, phenyl group and phenoxy group. When a plurality of R⁴ are present, each R⁴ may be the same or different.

Z^{a} may be different even identical to the Z^{b}.

R^{3a} and R^{3b} are each independently a residue derived from a reaction product of a fat-soluble vitamin having a hydroxyl group and succinic anhydride or glutaric anhydride, or a sterol derivative having a hydroxyl group and succinic anhydride or glutaric acid. Represents a residue derived from a reaction product with an anhydride, or an aliphatic hydrocarbon group having 12 to 22 carbon atoms, and optionally each independently a fat-soluble vitamin having a hydroxyl group and succinic anhydride or glutaric anhydride. Or a C 12-22 aliphatic hydrocarbon group, and optionally each independently an aliphatic hydrocarbon group having 12-22 carbon atoms.

Examples of the fat-soluble vitamin having a hydroxyl group include retinol, ergosterol, 7-dehydrocholesterol, calciferol, corcalciferol, dihydroergocalciferol, dihydrotaxolol, tocopherol, and tocotrienol. The fat-soluble vitamin having a hydroxyl group is optionally tocopherol.

Examples of the sterol derivative having a hydroxyl group include cholesterol, cholestanol, stigmasterol, P-sitosterol, lanosterol, ergosterol and the like, optionally cholesterol or cholestanol.

The aliphatic hydrocarbon group having 12 to 22 carbon atoms may be linear or branched. The aliphatic hydrocarbon group may be saturated or unsaturated. In the case of an unsaturated aliphatic hydrocarbon group, the number of unsaturated bonds contained in the aliphatic hydrocarbon group is usually 1 to 6, optionally 1 to 3, or 1 to 2. Unsaturated bonds include carbon-carbon double bonds and carbon-carbon triple bonds. The number of carbon atoms contained in the aliphatic hydrocarbon group is optionally 13 to 19, or 13 to 17. The aliphatic hydrocarbon group includes an alkyl group, an alkenyl group, an alkynyl group and the like, and optionally includes an alkyl group or an alkenyl group. Specific examples of the aliphatic hydrocarbon group having 12 to 22 carbon atoms include dodecyl, tridecyl, tetradecyl, pentadecyl, hexadecyl, heptadecyl, octadecyl, nonadecyl, icosyl, heicosyl, docosyl, Dodecenyl group, tridecenyl group, tetradecenyl group, pentadecenyl group, hexadecenyl group, heptadecenyl group, octadecenyl group, nonadecenyl group, icocenyl group, henicocenyl group, dococenyl group, dodecadienyl group, tridecadienyl group, tetradecadienyl group, pentadecadienyl group Group, hexadecadienyl group, heptadecadienyl group, octadecadienyl group, nonadecadienyl group, icosadenyl group, henicosadienyl group, docosadienyl group, octadecatrienyl group, icosatrienyl group, Cosatetraenyl group, icosapentaenyl group, docosahexaenyl group, isostearyl group, 1-hexylheptyl group, 1-hexylnonyl group, 1-octylnonyl group, 1-octylundecyl group, 1-decylundecyl group, etc. be able to. The aliphatic hydrocarbon group having 12 to 22 carbon atoms is optionally a tridecyl group, a pentadecyl group, a heptadecyl group, a nonadecyl group, a heptadecenyl group, a heptadecadienyl group, or a 1-hexylnonyl group, or a tridecyl group, A heptadecyl group, a heptadecenyl group, and a heptadecadienyl group.

In one embodiment of the present disclosure, the aliphatic hydrocarbon group having 12 to 22 carbon atoms represented by R^{3a} and R^{3b} is derived from a fatty acid. In this case, the carbonyl carbon derived from the fatty acid is contained in CO—O— in the formula (1). Specific examples of the aliphatic hydrocarbon group include a heptadecenyl group when linoleic acid is used as the fatty acid, and a heptadecenyl group when oleic acid is used as the fatty acid.

R^{3a} may be different be the same as R^{3b}.

In one embodiment of the present disclosure, R^{1a} is the same as R^{1b}, X^{a} is the same as X^{b}, R^{2a} is the same as R^{2b}, Y^{a} is the same as Y^{b}, and Z^{a} is identical to the Z^{b}, R^{3a} is the same as R^{3b}.

Preferable examples of the ionizable lipid represented by the formula (1) include the following ionizable lipids: Ionizable lipid (1-1); R^{1a} and R^{1b} are each independently an alkylene group having 1 to 6 carbon atoms (eg, methylene group, ethylene group); X a and X b are each independently an acyclic alkyl tertiary amino group having 1 to 6 carbon atoms and 1 tertiary amino group (eg, N(CH 3)), Or a cyclic alkylene tertiary amino group having 2 to 5 carbon atoms and 1 to 2 tertiary amino groups (eg, piperidylene group); R^{2a} and R^{2b} are each independently an alkylene group having 8 or less carbon atoms (eg, methylene group, ethylene group, propylene group); Y^{a} and Y^{b} are each independently an ester bond or an amide bond; Z^{a} and Z^{b} are each independently a divalent group derived from an aromatic compound having 3 to 16 carbon atoms, having at least one aromatic ring, and optionally having a hetero atom. (Eg, C 6 H 4 CH 2, CH 2 C 6 H 4 CH 2); R^{3a} and R^{3b} are each independently a residue derived from a reaction product of a fat-soluble vitamin having a hydroxyl group (eg, tocopherol) and succinic anhydride or glutaric anhydride, or an aliphatic group having 12 to 22 carbon atoms A hydrocarbon group (eg, heptadecenyl group, heptadecadienyl group, 1-hexylnonyl group);

Ionizable lipid (1-2); Ria and R^{1b} are each independently an alkylene group having 1 to 4 carbon atoms (eg, methylene group, ethylene group); X a and X b are each independently an acyclic alkyl tertiary amino group having 1 to 3 carbon atoms and 1 tertiary amino group (eg, —N(CH 3)), Or a cyclic alkylene tertiary amino group having 2 to 5 carbon atoms and 1 tertiary amino group (eg, piperidylene group); R^{2a} and R^{2b} are each independently an alkylene group having 6 or less carbon atoms (eg, methylene group, ethylene group, propylene group); Y^{a} and Y^{b} are each independently an ester bond or an amide bond; Z a and Z b are each independently a divalent group derived from an aromatic compound having 6 to 12 carbon atoms, one aromatic ring, and optionally having a hetero atom (Eg, C 6 H 4 CH 2, CH 2 C 6 H 4 CH 2); R^{3a} and R^{3b} are each independently a residue derived from a reaction product of a fat-soluble vitamin having a hydroxyl group (eg, tocopherol) and succinic anhydride, or an aliphatic hydrocarbon group having 13 to 19 carbon atoms (eg., Heptadecenyl group, heptadecadienyl group, 1-hexylnonyl group).

Ionizable lipid (1-3); R^{1a} and R^{1b} are each independently an alkylene group having 1 to 2 carbon atoms (eg, methylene group, ethylene group); X^{a} and X^{b} are each independently X¹:

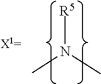

wherein R⁵ is an alkyl group having 1 to 3 carbon atoms (eg, a methyl group)), or X²:

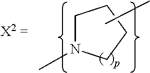

wherein p is 1 or 2), R^{2a} and R^{2b} are each independently an alkylene group having 4 or less carbon atoms (eg, methylene group, ethylene group, propylene group); Y^{a} and Y^{b} are each independently an ester bond or an amide bond; Z^{a} and Z^{b} are each independently Z¹:

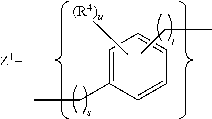

wherein s is an integer from 0 to 1, t is an integer from 0 to 2, u is an integer from 0 to 2 (optionally 0), and (R⁴)u are each independently represents a substituent. R^{3a} and R^{3b} are each independently a residue derived from a reaction product of a fat-soluble vitamin having a hydroxyl group (eg, tocopherol) and succinic anhydride, or an aliphatic hydrocarbon group having 13 to 17 carbon atoms (eg, Heptadecenyl group, heptadecadienyl group, 1-hexylnonyl group); Ionizable lipid (1).

Specific examples of the ionizable lipid (1) of the present disclosure include the following O-Ph-P3C1, O-Ph-P4C1, O-Ph-P4C2, O-Bn-P4C2, E-Ph-P4C2, L-Ph-P4C2, HD-Ph-P4C2, O-Ph-amide-P4C2, and O-Ph-C3M as seen in Tables 2, 3, and 4.

TABLE 2
Ionizable lipids

| | |
|---|---|
| O-Ph-P3C1 | 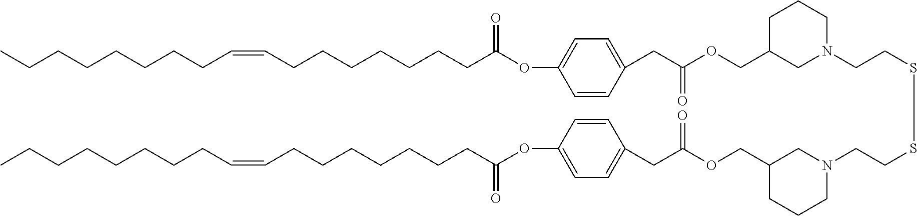 [SMILES: CCCCCCCC/C=C\CCCCCCCC(=O)Oc1ccc(CC(=O)OCC2CCCN(CCSSCCN3CCCC(COC(=O)Cc4ccc(OC(=O)CCCCCCC/C=C\CCCCCCCC)cc4)C3)C2)cc1] |
| O-Ph-P4C1 | 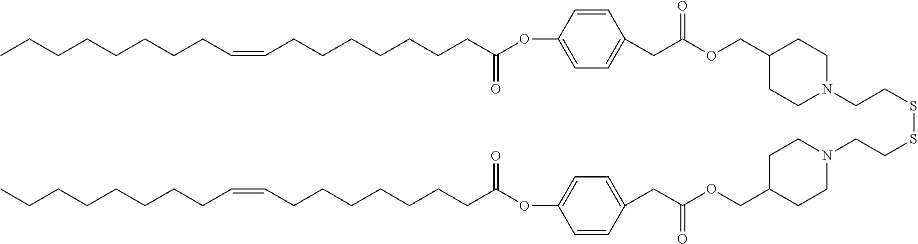 [SMILES: CCCCCCCC/C=C\CCCCCCCC(=O)Oc1ccc(CC(=O)OCC2CCN(CCSSCCN3CCC(COC(=O)Cc4ccc(OC(=O)CCCCCCC/C=C\CCCCCCCC)cc4)CC3)CC2)cc1] |
| O-Ph-P4C2 | 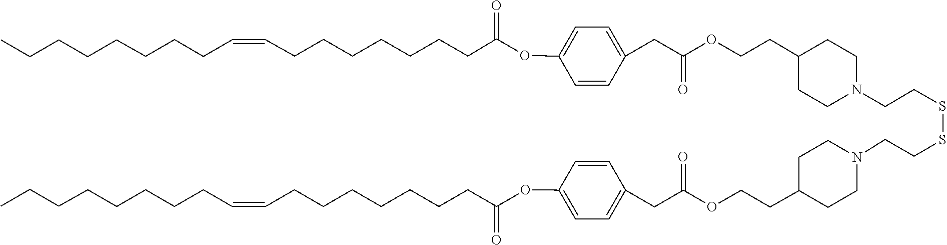 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1] |
| O-Bn-P4C2 | 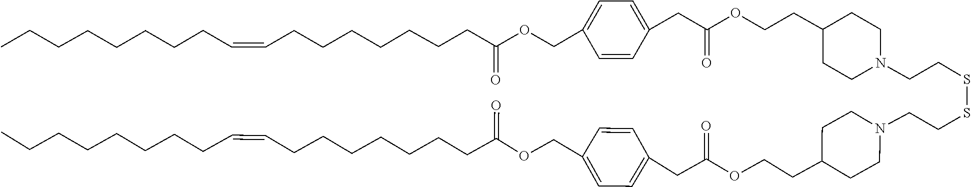 |
| E-Ph-P4C2 | 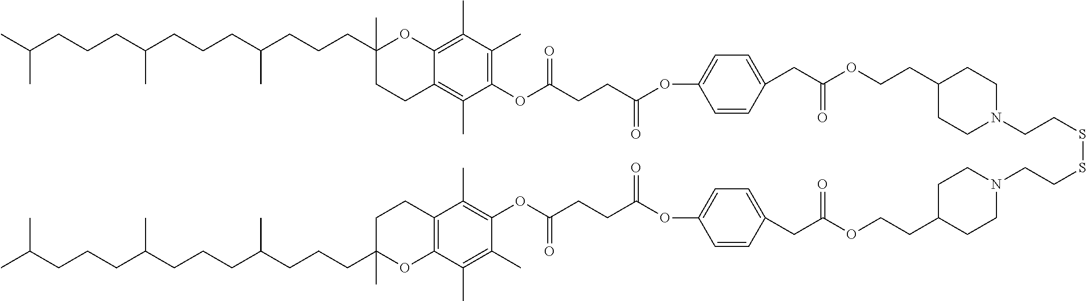 [SMILES: Cc1c(C)c2c(c(C)c1OC(=O)CCC(=O)Oc1ccc(CC(=O)OCCC3CCN(CCSSCCN4CCC(CCOC(=O)Cc5ccc(OC(=O)CCC(=O)Oc6c(C)c(C)c7c(c6C)CCC(C)(CCCC(C)CCCC(C)CCCC(C)C)O7)cc5)CC4)CC3)cc1)CCC(C)(CCCC(C)CCCC(C)CCCC(C)C)O2] |
| L-Ph-P4C2 | 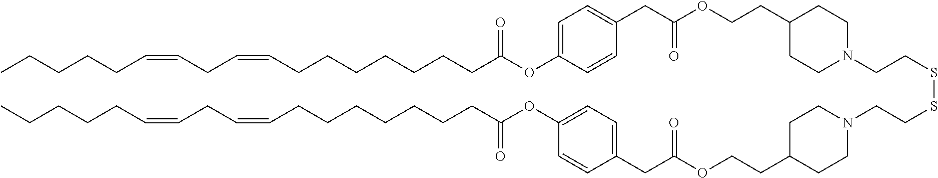 [SMILES: CCCCC/C=C\C/C=C\CCCCCCCC(=O)Oc1ccc(CC(=O)OCCC2CCN(CCSSCCN3CCC(CCOC(=O)Cc4ccc(OC(=O)CCCCCCC/C=C\C/C=C\CCCCC)cc4)CC3)CC2)cc1] |
| HD-Ph-P4C2 | 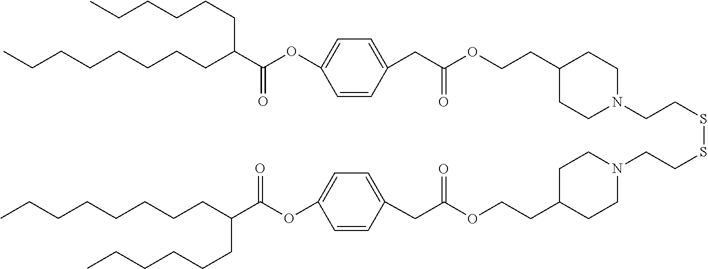 [SMILES: CCCCCCCCC(CCCCCC)C(=O)Oc1ccc(CC(=O)OCCC2CCN(CCSSCCN3CCC(CCOC(=O)Cc4ccc(OC(=O)C(CCCCCC)CCCCCCCC)cc4)CC3)CC2)cc1] |
| O-Ph- amide-P4C2 | 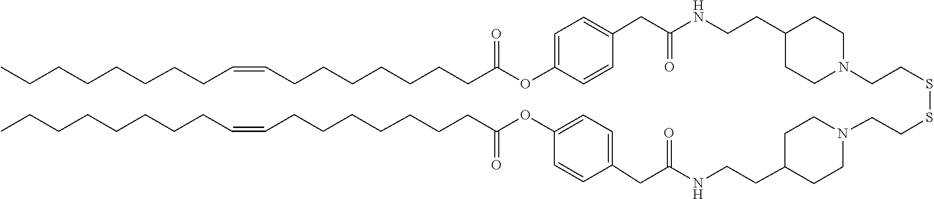 |
| O-Ph-C3M | 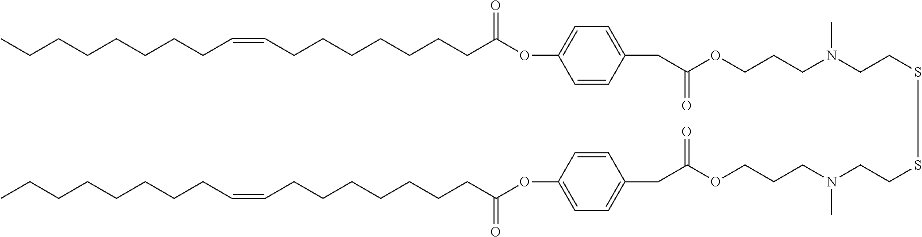 [SMILES: CCCCCCCC/C=C\CCCCCCCC(=O)Oc1ccc(CC(=O)OCCCN(C)CCSSCCN(C)CCCOC(=O)Cc2ccc(OC(=O)CCCCCCC/C=C\CCCCCCCC)cc2)cc1] |

TABLE 3
Ionizable lipids

| | |
|---|---|
| α-D- Toco- pherol- succin- oyl | 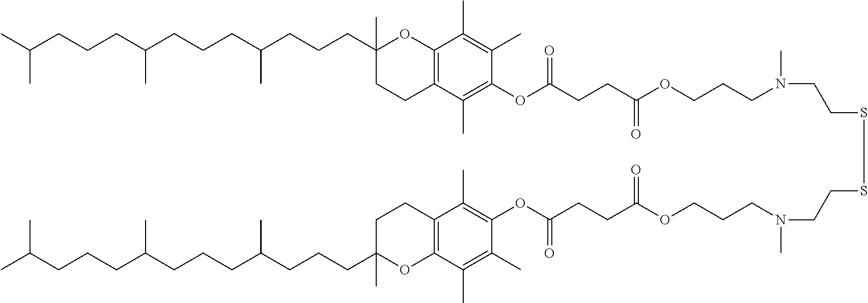 [SMILES: Cc1c(C)c2c(c(C)c1OC(=O)CCC(=O)OCCCN(C)CCSSCCN(C)CCCOC(=O)CCC(=O)Oc1c(C)c(C)c3c(c1C)CCC(C)(CCCC(C)CCCC(C)CCCC(C)C)O3)CCC(C)(CCCC(C)CCCC(C)CCCC(C)C)O2] |
| Lino- leoyl | 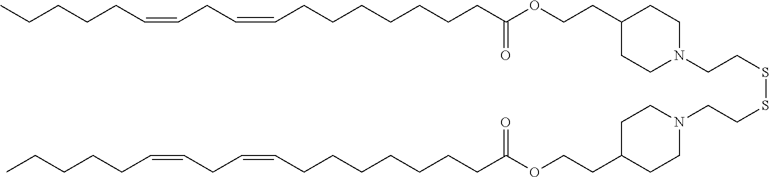 [SMILES: CCCCC/C=C\C/C=C\CCCCCCCC(=O)OCCC1CCN(CCSSCCN2CCC(CCOC(=O)CCCCCCC/C=C\C/C=C\CCCCC)CC2)CC1] |
| Ole- oyl | 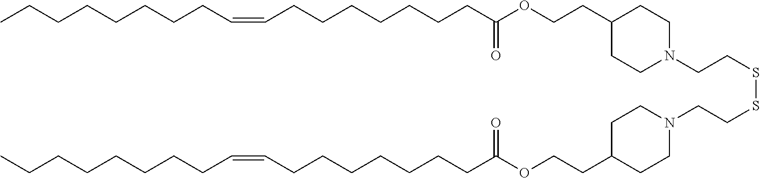 [SMILES: CCCCCCCC/C=C\CCCCCCCC(=O)OCCC1CCN(CCSSCCN2CCC(CCOC(=O)CCCCCCC/C=C\CCCCCCCC)CC2)CC1] |

In some embodiments, the delivery vehicle is an LNP capable of transfecting at least 50%, at least 60%, at least 70%, at least 80%, at least 90%, or at least 95% of a population of liver cells wherein the ionizable lipid is at least 10%, at least 20%, at least 30%, at least 40%, at least 50%, at least 60%, at least 70%, or at least 80% of the molar percentage of the LNP.

In some embodiments, the LNP comprises an ionizable lipid. In some embodiments, the ionizable lipid is no more than 20%, no more than 30%, no more than 40%, no more than 50%, no more than 60%, no more than 70%, or no more than 90% of the molar percentage of the LNP. Exemplary ionizable lipids include, but are not limited to: imidazole cholesterol ester (ICE), (15Z,18Z)-N,N-dimethyl-6-(9Z,12Z)-octadeca-9,12-dien-1-yl)tetracosa-15,18-dien-1-amine (HGT5000), (15Z,18Z) N,N-dimethyl-6-((9Z,12Z)-octadeca-9,12-dien-1-yl)tetracosa-4,15,18-trien-1-amine (HGT5001), and (15Z,18Z)-N,N-dimethyl-6-((9Z,12Z)-octadeca-9,12-dien-1-yl)tetracosa-5,15,18-trien-1-amine (HGT5002).

Lipids having the structure of Formula I are shown in Table 4 below. For example, SS-OP is also named 0-Ph-P4C2. The term “SS-OP analog” as used herein refers to a compound of Formula I.

TABLE 4
Nomenclature of Lipids

| Name | Structure |
|---|---|
| SS- M | 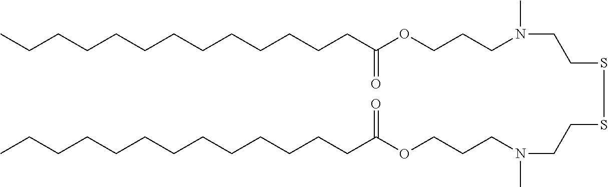 [SMILES: CCCCCCCCCCCCCC(=O)OCCCN(C)CCSSCCN(C)CCCOC(=O)CCCCCCCCCCCCC] |
| SS- E | 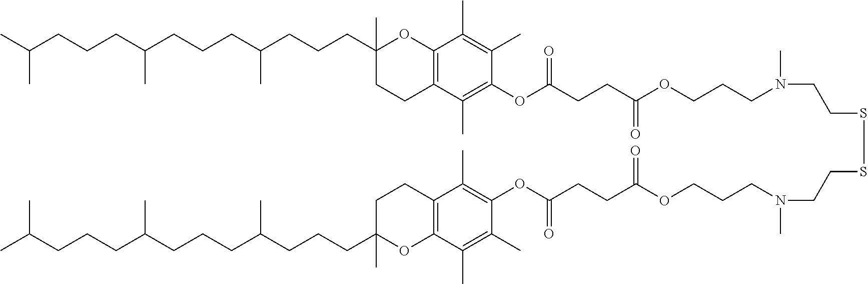 [SMILES: Cc1c(C)c2c(c(C)c1OC(=O)CCC(=O)OCCCN(C)CCSSCCN(C)CCCOC(=O)CCC(=O)Oc1c(C)c(C)c3c(c1C)CCC(C)(CCCC(C)CCCC(C)CCCC(C)C)O3)CCC(C)(CCCC(C)CCCC(C)CCCC(C)C)O2] |
| SS- EC | 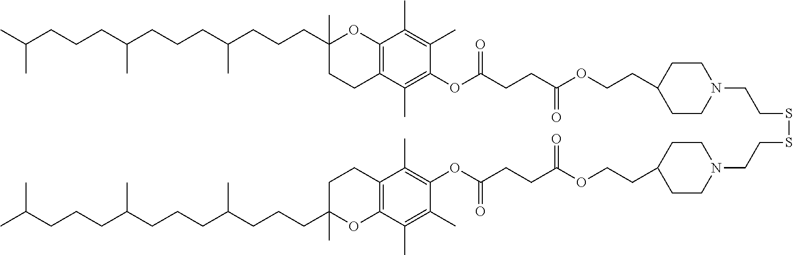 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2] |
| SS- LC | 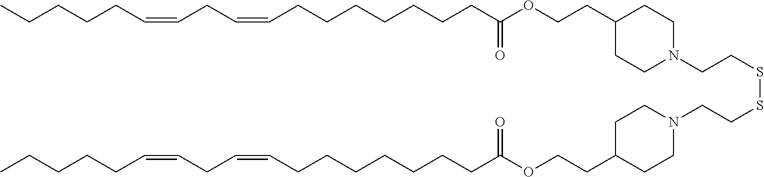 [SMILES: CCCCC/C=C\C/C=C\CCCCCCCC(=O)OCCC1CCN(CCSSCCN2CCC(CCOC(=O)CCCCCCC/C=C\C/C=C\CCCCC)CC2)CC1] |
| SS- OC | 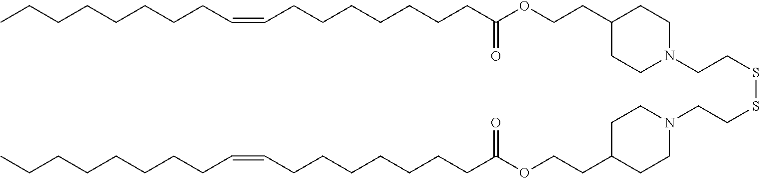 [SMILES: CCCCCCCC/C=C\CCCCCCCC(=O)OCCC1CCN(CCSSCCN2CCC(CCOC(=O)CCCCCCC/C=C\CCCCCCCC)CC2)CC1] |
| SS- OP | 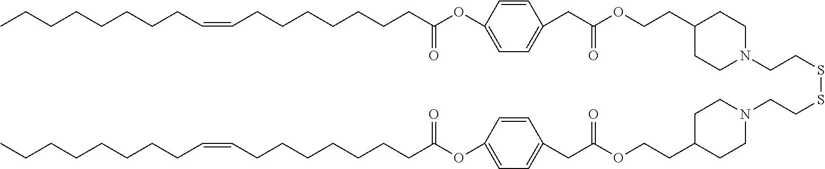 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1] |

Provided herein are compositions comprising i) a ribonucleic acid (RNA) coding for telomerase reverse transcriptase (TERT) and ii) a compound of Formula (I):

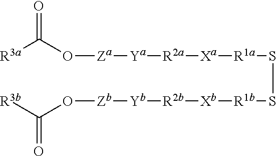

In the formula (I): R^{1a} and R^{1b} can each independently represent an alkylene group having 1 to 6 carbon atoms, and may be linear or branched, but is optionally linear. The alkylene group optionally has 1 to 4 carbon atoms, or 1 to 2. Specific examples of the alkylene group having 1 to 6 carbon atoms include a methylene group, an ethylene group, a trimethylene group, an isopropylene group, a tetramethylene group, an isobutylene group, a pentamethylene group, and a neopentylene group. R^{1a} and R^{1b} are optionally each independently a methylene group, an ethylene group, a trimethylene group, an isopropylene group, or a tetramethylene group, or an ethylene group.

R^{1a} may be different or be the same as R^{1b}.

X^{a} and X^{b} can each independently be an acyclic alkyl tertiary amino group having 1 to 6 carbon atoms and 1 tertiary amino group, or 2 to 5 carbon atoms, and a cyclic alkylene tertiary amino group having 1 to 2 tertiary amino groups, optionally each independently a cyclic alkylene having 2 to 5 carbon atoms and 1 to 2 tertiary amino groups and an alkylene tertiary amino group.

In some embodiments, the compound of Formula II is

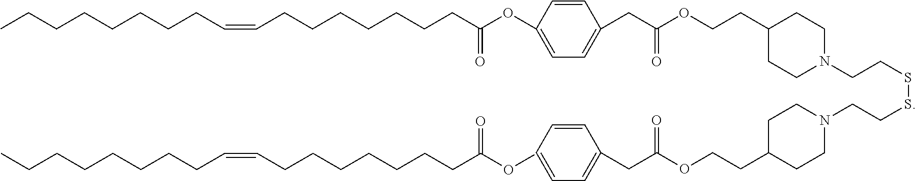

The RNA can be a synthetic RNA. The RNA can comprise at least one modified nucleoside. Also provided herein are methods for delivery of the compositions to a cell. The compound of Formula I can be used to aid in delivery of the RNA to a cell in vitro or in vivo. Once delivered to a cell, the synthetic RNA can transiently express exogenous telomerase in the cell, and telomeres within the cell treated with the synthetic RNA can be extended. Thus the compositions can be used to extend telomeres within a cell.

III. Formulation of mRNA and Nanoparticle Delivery Vehicle Compositions

The methods of synthesis of mRNA and lipid nanoparticles (LNPs) are well established. Synthetic mRNAs, e.g., comprising a 5′ cap, 5′ and 3′ UTRs coding sequence, and a poly-A tail, may be synthesized from modified and unmodified nucleotides by in vitro transcription of a DNA template using an RNA polymerase, for example T7 RNA polymerase. The DNA template may be generated, for example, by PCR or plasmid amplification and restriction digest, followed by purification.

Lipid nanoparticles (LNPs), liposomes, or polymer nanoparticle delivery vehicles carrying mRNA may be produced, for example, by mixing the lipids or polymers in an organic solvent, e.g., ethanol, with one or more mRNAs in an aqueous buffer, and then subject to buffer exchange and concentration. In some embodiments, the LNP, liposome, or polymer nanoparticle delivery vehicle may be produced using a microfluidic device to rapidly mix reagents and form monodisperse particles of controlled size. For example, the microfluidic mixer could be a staggered herringbone mixer (SHM). For example, the microfluidic mixer could be produce by the NanoAsssemblr made by Precision Nanosystems (PNI). In other embodiments, the LNP, liposome, or polymer nanoparticle delivery vehicle may be produced by a T-mixer. In some embodiments, the LNP, liposome, or polymer nanoparticle may encapsulate an mRNA and/or associate with one or more mRNAs through electrostatic interactions. The buffer exchange and concentration of the LNP, liposome, or polymer nanoparticle may be performed by tangential flow filtration. In other embodiments, the buffer exchange and concentration of the LNP, liposome, or polymer nanoparticle may be performed by centrifugal ultrafiltration using a membrane with a nominal molecule weight cutoff of <=500,000 Da, for example 100,000 Da.

In some embodiments, the lipid nanoparticle particles (LNP) formulations provided herein are capable of transfecting at least 50%, at least 60%, at least 70%, at least 80%, at least 90%, or at least 95% of a population of liver cells.

The form of the lipid membrane structure of the present disclosure is not particularly limited. For example, as a form 1 which the ionizable lipid of the present disclosure is dispersed in an aqueous solvent, liposomes (for example, monolayer liposomes, multilamellar liposomes, etc.), spherical micelles, string micelles, lipid nanoparticles (LNPs) or unspecified layered structures.

The lipid membrane structure of the present disclosure may further contain other components in addition to the ionizable lipid of the present disclosure. Examples of the other components include lipids (phospholipids (such as phosphatidylinositol, phosphatidylethanolamine, phosphatidylserine, phosphatidic acid, phosphatidylglycerol, phosphatidylcholine), glycolipids, peptide lipids, cholesterol, ionizable lipids other than cationic lipids, PEG lipids, etc), surfactants (eg 3-[(3-cholamidopropyl) dimethylammonio]propane sulfonate, cholic acid sodium salt, octyl glycoside, ND-gluco-N-methylalkanamides), polyethylene glycol, proteins and the like. The content of the other constituents in the lipid membrane structure of the present disclosure is usually 5 to 95 mol %, optionally 10 to 90 mol %, or 30 to 80 mol %.

The content of the ionizable lipid of the present disclosure contained in the lipid membrane structure of the present disclosure is not particularly limited.

The lipid membrane structure of the present disclosure is prepared by dispersing the ionizable lipid of the present disclosure and other components (lipids, etc.) in a suitable solvent or dispersion medium, for example, an aqueous solvent or an alcoholic solvent, and if necessary, tissue It can be prepared by performing an operation that induces crystallization.

Examples of the “operation for inducing organization” include an ethanol dilution method using a microchannel or a vortex, a simple hydration method, an ultrasonic treatment, a heating, a vortex, an ether injection method, a French press method, and a cholic acid method. Examples thereof include, but are not limited to, methods known per se such as Ca 2+ fusion method, freeze-thaw method, and reverse phase evaporation method.

The nucleic acid can be introduced into the cell in vivo and/or in vitro by encapsulating the nucleic acid in the lipid membrane structure containing the ionizable lipid of the present disclosure and bringing it into contact with the cell. Therefore, the present disclosure provides a nucleic acid introduction agent comprising the ionizable lipid or lipid membrane structure of the present disclosure.

The nucleic acid introduction agent of the present disclosure can introduce any nucleic acid into cells, Examples of the nucleic acid include, but are not limited to, DNA, RNA, RNA chimeric nucleic acid, DNA/RNA hybrid, and the like. The nucleic acid can be any one of 1 to 3 strands, but is optionally single strand or double strand, Nucleic acids may be other types of nucleotides that are N-glycosides of purine or pyrimidine bases, or other oligomers having a non-nucleotide backbone (e.g., commercially available peptide nucleic acids (PNA), etc.) or other oligomers with special linkages. The oligomer may contain nucleotides having a configuration that allows base pairing or base attachment as found in DNA or RNA. In addition, the nucleic acid may be substituted with, for example, a known modified nucleic acid, a labeled nucleic acid, a capped nucleic acid, a methylated nucleic acid, or one or more natural nucleotides known in the art, intramolecular nucleotide modified nucleic acids, nucleic acids with uncharged bonds (e.g., methyl sulfonate, phosphotriester, phosphoramidate, carbamate, etc.), charged bonds or sulfur containing bonds (eg phosphorothioate), side chain groups such as proteins (e.g., nucleases, nuclease inhibitors, toxins, antibodies, signal peptides, poly-L-lysine, etc.) and sugars (eg, monosaccharides), nucleic acids and nucleic acids with intercurrent compounds (eg, acridine, psoralen, etc.), nucleic acids containing chelate compounds (eg, metals, radioactive metals, boron, oxidizing metals, etc.), nucleic acids containing alkylating agents, and nucleic acids with modified bonds (eg, alpha anomeric nucleic acids, etc.)

The type of DNA that can be used in the present disclosure is not particularly limited, and can be appropriately selected depending on the purpose of use. Examples include plasmid DNA, cDNA, antisense DNA, chromosomal DNA, PAC, BAC, and CpG oligo, optionally plasmid DNA, cDNA, and antisense DNA, or plasmid DNA. Circular DNA such as plasmid DNA can be appropriately digested with a restriction enzyme or the like and used as linear DNA.

The type of RNA that can be used in the present disclosure is not particularly limited, and can be appropriately selected depending on the purpose of use. For example, siRNA, miRNA, shRNA, antisense RNA, messenger RNA (mRNA), single-stranded RNA genome, double-stranded RNA genome, RNA replicon, transfer RNA, ribosomal RNA, etc., optionally siRNA, miRNA, shRNA, miRNA, antisense RNA, RNA replicon.

The nucleic acid used in the present disclosure is optionally purified by a method commonly used by those skilled in the art.

The nucleic acid-introducing agent of the present disclosure encapsulating nucleic acid can be administered in vivo for the purpose of, for example, prevention and/or treatment of diseases. Accordingly, the nucleic acid used in the present disclosure is optionally a nucleic acid having preventive and/or therapeutic activity against a given disease (prophylactic/therapeutic nucleic acid). Examples of such nucleic acids include nucleic acids used for so-called gene therapy.

In order to introduce a nucleic acid into a cell using the nucleic acid introduction agent of the present disclosure, the nucleic acid was encapsulated by coexisting the target nucleic acid when forming the lipid membrane structure of the present disclosure. The lipid membrane structure of the present disclosure is formed. For example, when liposomes are formed by the ethanol dilution method, the aqueous solution of nucleic acid and the ethanol solution of the components of the lipid membrane structure of the present disclosure (lipids, etc.) are vigorously mixed by vortex or microchannel, etc, is diluted with an appropriate buffer. When liposomes are formed by the simple hydration method, the components (lipids, etc.) of the lipid membrane structure of the present disclosure are dissolved in an appropriate organic solvent, the solution is placed in a glass container, and the solvent is retained by drying under reduced pressure and left to obtain a lipid film. Here, an aqueous solution of nucleic acid is added and hydrated, followed by sonication with a sonicator. The present disclosure also provides the above lipid membrane structure in which such a nucleic acid is encapsulated.

An example of a lipid membrane structure in which a nucleic acid is encapsulated is LNP encapsulated in a nucleic acid by forming an electrostatic complex between the nucleic acid and a ionizable lipid. This LNP can be used as a drug delivery system for selectively delivering a nucleic acid or the like into a specific cell. For example, a DNA vaccine by introducing an antigen gene into a dendritic cell, a gene therapy drug for a tumor, RNA It is useful for nucleic acid drugs that suppress the expression of target genes using interference.

The particle diameter of the lipid membrane structure of the present disclosure encapsulating nucleic acid is not particularly limited, but is optionally 10 nm to 500 nm, or 30 nm to 300 nm. The particle diameter can be measured using a particle size distribution measuring apparatus such as Zetasizer Nano (Malvern). The particle diameter of the lipid membrane structure can be appropriately adjusted according to the method for preparing the lipid membrane structure.

The surface potential (zeta potential) of the lipid membrane structure of the present disclosure encapsulating nucleic acid is not particularly limited, but may be −60 to +60 mV, −45 to 45 mV, −30 to +30 mV, −15 to +15 mV, or −10 to −10 mV. In conventional gene transfer, particles having a positive surface potential have been mainly used. While this is useful as a method to positive electrostatic interaction with negatively charged cell surface heparin sulfate and promote cellular uptake, positive surface charge is delivered intracellularly. There is a possibility that the nucleic acid release from the carrier due to the interaction with the nucleic acid is suppressed, and the protein synthesis due to the interaction between the mRNA and the delivery nucleic acid is suppressed, By adjusting the surface charge within the above range, this problem can be solved. The surface charge can be measured by using a zeta potential measuring device such as Zetasizer Nano. The surface charge of the lipid membrane structure can be adjusted by the composition of the components of the lipid membrane structure containing the ionizable lipid of the present disclosure.

The lipid membrane surface pKa (hereinafter referred to as Liposomal pKa) of the lipid membrane structure of the present disclosure is not particularly limited, but may have a pKa of 0.5 to 72, or a pKa of 6.0, to 6,8. Liposomal pKa is used as an index indicating that the lipid membrane structure taken up by endocytosis is susceptible to protonation of the lipid membrane structure in a weakly acidic environment within the endosome. Liposomal pKa can be adjusted by the composition of the components of the lipid membrane structure containing the ionizable lipid of any of the above embodiments.

The hemolysis activity (membrane fusion ability) of a lipid membrane structure of the present disclosure is not particularly limited, but may have no hemolysis activity (less than 5%) at physiological pH (pH 7.4), and may be endosomal. The higher the hemolysis activity, the more efficiently the nucleic acid can be delivered into the cytoplasm. However, if the hemolysis activity is present at physiological pH, the nucleic acid will be delivered to unintended cells during residence in the blood, resulting in decreased target-directedness and toxicity. Therefore, it is preferable to have hemolysis activity only in the endosomal environment as described above. The hemolysis activity can be adjusted by the composition of the components of the lipid membrane structure containing the ionizable lipid of the present disclosure.

By bringing the lipid membrane structure of the present disclosure in which nucleic acid is encapsulated into contact with the cell, the encapsulated nucleic acid can be introduced into the cell. The cell may be a cultured cell line containing cancer cells, a cell isolated from an individual or tissue, or a tissue or tissue piece of cell. Further, the cells may be adherent cells or nonadherent cells.

The step of bringing the lipid membrane structure of the present disclosure encapsulating nucleic acid into contact with cells in vitro will be specifically described below.

Cells are suspended in an appropriate medium several days before contact with the lipid membrane structure and cultured under appropriate conditions, Upon contact with the lipid membrane structure, the cell may or may not be in the growth phase.

The culture medium at the time of the contact may be a serum-containing medium or a serum-free medium, but the serum concentration in the medium may be 30% by weight or less, more may be 20% by weight or less. If the medium contains excessive protein such as serum, the contact between the lipid membrane structure and the cell may be inhibited.

The cell density at the time of the contact is not particularly limited and can be appropriately set in consideration of the cell type, but is usually in the range of 1×10⁴ to 1×10⁷ cells/mL.

For example, a suspension of the lipid membrane structure of the present disclosure in which the above-described nucleic acid is encapsulated is added to the cells thus prepared. The addition amount of the suspension is not particularly limited, and can be appropriately set in consideration of the number of cells and the like. The concentration of the lipid membrane structure at the time of contacting the cell is not particularly limited as long as the introduction of the target nucleic acid into the cell can be achieved, but the lipid concentration is usually 1 to 100 nmol/mL, and may be 0.1 to 10 μg/mL.

After adding the above suspension to the cells, the cells are cultured. The culture temperature, humidity, CO₂ concentration, etc. are appropriately set in consideration of the cell type. When the cells are mammalian cells, the temperature is usually about 37° C., the humidity is about 95%, and the CO₂ concentration is about 5%. In addition, the culture time can be appropriately set in consideration of conditions such as the type of cells used, but may be in the range of 0.1 to 76 hours, or in the range of 0.2 to 24 hours, and may be 0.5-12 hours. If the culture time is too short, the nucleic acid is not sufficiently introduced into the cells, and if the culture time is too long, the cells may be weakened.

The nucleic acid is introduced into the cells by the above-described culture. The medium may be replaced with a fresh medium, or the fresh medium is added to the medium and the cultivation is further continued. If the cells are mammalian cells, the fresh medium may contain serum or nutrient factors.

The lipid membrane structure of the present disclosure may further contain other components in addition to the ionizable lipid of the present disclosure. Examples of the other components include lipids (phospholipids (such as phosphatidylinositol, phosphatidylethanolamine, phosphatidylserine, phosphatidic acid, phosphatidylglycerol, phosphatidylcholine), glycolipids, peptide lipids, cholesterol, ionizable lipids other than cationic lipids, PEG lipids, etc.), surfactants (eg 3-[(3-cholamidopropyl) dimethylammonio]propane sulfonate, cholic acid sodium salt, octyl glycoside, ND-gluco-N-methylalkanamides), polyethylene glycol, proteins and the like.

The lipid membrane structure of the present disclosure is prepared by dispersing the ionizable lipid of the present disclosure and other components (lipids, etc.) in a suitable solvent or dispersion medium, for example, an aqueous solvent or an alcoholic solvent, and if necessary, tissue. It can be prepared by performing an operation that induces crystallization.

Examples of the “operation for inducing organization” include an ethanol dilution method using a microchannel or a vortex, a simple hydration method, an ultrasonic treatment, a heating, a vortex, an ether injection method, a French press method, and a cholic acid method. Examples thereof include, but are not limited to, methods known per se such as Ca 2+ fusion method, freeze-thaw method, and reverse phase evaporation method.

The nucleic acid can be introduced into the cell in vivo and/or in vitro by encapsulating the nucleic acid in the lipid membrane structure containing the ionizable lipid of the present disclosure and bringing it into contact with the cell. Therefore, the present disclosure provides a nucleic acid introduction agent comprising the ionizable lipid or lipid membrane structure of the present disclosure.

The nucleic acid introduction agent of the present disclosure can introduce any nucleic acid into cells, Examples of the nucleic acid include, but are not limited to, DNA, RNA, RNA chimeric nucleic acid, DNA/RNA hybrid, and the like. The nucleic acid can be any one of 1 to 3 strands, but may be single strand or double strand. Nucleic acids may be other types of nucleotides that are N-glycosides of purine or pyrimidine bases, or other oligomers having a non-nucleotide backbone (eg, commercially available peptide nucleic acids (PNA), etc.) or other oligomers with special linkages. The oligomer may contain nucleotides having a configuration that allows base pairing or base attachment as found in DNA or RNA.

The type of RNA that can be used in the present disclosure is not particularly limited, and can be appropriately selected depending on the purpose of use. For example, siRNA, miRNA, shRNA, antisense RNA, messenger RNA (mRNA), single-stranded RNA genome, double-stranded RNA genome, RNA replicon, transfer RNA, ribosomal RNA, etc., or siRNA, miRNA, shRNA, mRNA, antisense RNA, or an RN A replicon.

The nucleic acid used in the present disclosure may be purified by a method commonly used by those skilled in the art.

The nucleic acid-introducing agent of the present disclosure encapsulating nucleic acid can be administered in vivo for the purpose of, for example, prevention and/or treatment of diseases. Accordingly, the nucleic acid used in the present disclosure may be a nucleic acid having preventive and at/or therapeutic activity against a given disease (prophylactic/therapeutic nucleic acid). Examples of such nucleic acids include nucleic acids used for so-called gene therapy.

### IV. Methods of Treatment

Methods of treatment as described herein refer to the treatment of fibrotic disease and/or liver disease in a subject in need thereof by administration of a composition comprising one or more TERT mRNA sequences. Compositions and methods of the disclosure may be used for the treatment of fibrotic conditions, including fibrosis. In some embodiments, compositions and/or methods of use of compositions of the disclosure intended for treatment of fibrotic conditions, including fibrosis, induce TERT expression or increase TERT activity in a liver cell. In some embodiments, compositions and/or methods of use of compositions of the disclosure intended for treatment of fibrotic conditions, including fibrosis, do not induce cellular, tissue or systemic toxicity. In some embodiments, compositions and/or methods of use of compositions of the disclosure intended for treatment of fibrotic conditions, including fibrosis, induce TERT expression or increase TERT activity in a spleen cell. Compositions may be administered systemically, e.g., intravenously.

A. Dosage and Timing of Telomerase Reverse Transcriptase (TERT) mRNA

In the compositions and methods described herein, in some embodiments, a TERT mRNA is administered in a dose of about 0.001 mg/kg per the subject's body weight to about 2.0 mg/kg per the subject's body weight to a subject in need thereof. In some embodiments, a TERT mRNA is administered to a subject in need thereof in a dose of about 0.01 mg/kg; in some embodiments in a dose of about 0.025 mg/kg; in some embodiments in a dose of about 0.05 mg/kg; in some embodiments in a dose of about 0.075 mg/kg; in some embodiments in a dose of about 0.1 mg/kg; in some embodiments in a dose of about 0.125 mg/kg; in some embodiments in a dose of about 0.150 mg/kg; in some embodiments in a dose of about 0.175 mg/kg; in some embodiments in a dose of about 0.2 mg/kg; in some embodiments in a dose of about 0.5 mg/kg; in some embodiments in a dose of about 0.75 mg/kg; in some embodiments in a dose of about 1.0 mg/kg; in some embodiments, in a dose of about 1.25 mg/kg; in some embodiment in a dose of about 1.5 mg/kg; or in some embodiment in a dose of about 2.0 mg/kg. In some embodiments the TERT mRNA is administered to a subject in need thereof in a dose of 0.1 mg/kg. In some embodiments the TERT mRNA is administered to a subject in need thereof in a dose of 0.125 mg/kg.

In some embodiments the TERT mRNA is administered to a subject in need thereof in a single dose. In some embodiments the TERT mRNA is administered to a subject in need thereof two, three, four, or five or more times. In some embodiments, the TERT mRNA is administered twice a week, every week, every two weeks, every four weeks, every six weeks, every twelve weeks, or every fifteen weeks. In some embodiments, the TERT mRNA is administered every month, every two months, every six months, once a year, on an ongoing basis, or as determined by their physician.

B. TERT mRNA Co-Therapies

In some embodiments, co-administration of a TERT mRNA may be combined with other anti-fibrotic drugs used in the treatment of fibrotic diseases and/or liver diseases. Drugs that may be used include, but are not limited to nintedanib, pirfenidone, prednisone, azathioprine, cyclophosphamide, mycophenolate mofetil, Pamrevlumab, and N-acetylcysteine.

### C. Routes of Administration

In some embodiments, a TERT mRNA may be delivered orally, subcutaneously, intravenously, intranasally, intradermally, transdermally, intraperitoneally, intramuscularly, intrapulmonarily, vaginally, rectally, or intraocularly. In example embodiments a TERT mRNA may be administered intravenously or through inhalation.

### D. Subjects and Treatment

The methods of treatment described herein are useful for the treatment of fibrotic diseases, conditions and disorders, and liver diseases, conditions, and disorders in a subject in need thereof. Fibrotic diseases and conditions of the disclosure include, but are not limited to, non-alcoholic hepatitis, hepatitis A, hepatitis B, hepatitis C, alcoholic hepatitis, liver cirrhosis, hemochromatosis, Wilson's disease, nonalcoholic steatohepatitis (NASH), NASH with fibrosis stage F4 according to the METAVIR scoring system, compensated liver cirrhosis, decompensated liver cirrhosis, acute-on-chronic liver cirrhosis, biliary atresia, primary biliary cirrhosis, primary sclerosing cholangitis, auto-immune hepatitis, cryptic cirrhosis, and ischemic hepatitis.

In some embodiments, a subject in need of treatments described herein is a subject with a genetic disorder or mutation in telomerase reverse transcriptase (TERT). In some embodiments the subject has no symptoms of fibrosis or liver disease. In other embodiments, the subject has symptoms and the treatment completely or partially ameliorates the symptoms. In other embodiments, the treatment slows progression of the symptoms.

In some embodiments, the subject is human.

In some embodiments, administration of a TERT mRNA reduces fibrotic tissue relative to a subject without treatment. In some embodiments, fibrotic tissue levels are measured by the METAVIR scoring system. In some embodiments, a TERT mRNA reduces the fibrotic stage of the tissue (e.g., from F4 to F3, F3 to F2, F2 to F1, or F1 to F0, or variations thereof) according to the METAVIR scoring system. In some embodiments, administration of a TERT mRNA reduces collagen levels.

In some embodiments, administration of a TERT mRNA reduces fibrotic tissue in a subject by at least 5%, at least 10%, at least 20%, at least 30%, at least 40%, at least 50%, at least 60%, at least 70%, at least 80%, at least 90%, at least 95%, at least 99%, or at least 100% over the treatment period and/or after the treatment period.

In some embodiments, administration of a TERT mRNA stops or slows the increase in fibrotic tissue over time relative to a subject without treatment. In some embodiments, the administration of a TERT mRNA slows the increase in amount of fibrotic tissue in a subject by at least 5%, at least 10%, at least 20%, at least 30%, at least 40%, at least 50%, at least 60%, at least 70%, at least 80%, at least 90%, at least 95%, at least 99%, or at least 100% over the treatment period and/or after the treatment period.

In some embodiments, administration of a TERT mRNA increases liver function relative to a subject without treatment. In some embodiments, the administration of a TERT mRNA increases liver function in a subject by at least 5%, at least 10%, at least 20%, at least 30%, at least 40%, or at least 50% over the treatment period and/or after the treatment period.

In some embodiments, administration of a TERT mRNA extends survival relative to a subject without treatment. In some embodiments, administration of a TERT mRNA extends liver transplant-free survival relative to a subject without treatment. In some embodiments, the administration of a TERT mRNA extends survival of a subject by at least 5%, at least 10%, at least 20%, at least 30%, at least 40%, at least 50%, at least 60%, at least 70%, at least 80%, at least 90%, at least 95%, at least 99%, at least 100%, at least 200%, at least 300%, at least 400%, at least 500%, at least 1000%, over the treatment period and/or after the treatment period. In some embodiments, administration of a TERT mRNA reduces hospitalization time and/or number of hospitalization visits to treat the fibrotic disease or liver disease. In some embodiments, administration of a TERT mRNA delays time to liver transplant.

Liver function may be measured by methods including but not limited to the Hepatic Quantification test (HepQuant SHUNT), the Child-Pugh Score, the Model for End stage Liver Disease (MELD) score, the Lillie Model, the Acute on Chronic Liver Failure (CLIF-C ACLF) score, the Glasgow Alcoholic Hepatitis Score (GAHS), the International Normalized Ratio (INR) score, the “Prothrombin Time” and other measures of coagulation enzymes, the presence or development of ascites, the presence or development of encephalopathy, platelet count, white blood cell count, mean arterial pressure, blood urea nitrogen (BUN) level, total bilirubin level, indirect bilirubin level, albumin level, alanine aminotransferase (ALT) level, aspartate aminotransferase (AST) level, alkaline phosphatase (ALP) level, and/or sodium creatinine level.

### V. Pharmaceutical Combinations

In some embodiments, a composition comprising a TERT mRNA includes an excipient, or carrier, e.g., an aqueous carrier. A variety of aqueous carriers can be used, e.g., buffered saline. The compositions may contain pharmaceutically acceptable auxiliary substances as those required to approximate physiological conditions such as pH and buffering agents, toxicity countering agents, e.g., sodium acetate, sodium chloride, sodium citrate, potassium chloride, calcium chloride, and sodium lactate. In some embodiments, the pharmaceutical composition comprises 10 mM sodium citrate buffered to pH 6.4. The composition may contain a cryoprotectant, e.g., glycerol, ethylene glycol, propylene glycol, or dimethylsulfoxide (DMSO). The concentration of active agent in these formulations can vary and are selected based on fluid volumes, viscosities, and body weight in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

### VI. Methods of Extending Telomeres

In another aspect, the instant disclosure provides methods of extending telomeres, comprising the step of administering any of the above-described compounds or compositions to a cell with shortened telomeres, wherein telomeres are extended within the cell. The instant disclosure also provides methods of treatment, comprising the step of administering any of the above-described compounds or compositions to an animal subject in need of, or that may benefit from, telomere extension.

In some embodiments, the compounds or compositions are administered to a cell, wherein the cell is an isolated cell or is part of a cell culture, an isolated tissue culture, an isolated organ, or the like (i.e., the administration is in vitro).

In other embodiments, the compounds or compositions are administered without isolating the cell or cells, the tissue, or the organ from the subject (i.e., the administration is in vivo). In some of these embodiments, the compound or composition is delivered to all, or almost all, cells in the subject's body. In some embodiments, the compound or composition is delivered to a specific cell, cell type, tissue, or organ in the subject's body.

Administration of the compounds or compositions of the instant disclosure may result in the transient expression of a telomerase activity in the cell. The increased activity may be measured by various assays, such as, for example, the telomerase repeat amplification protocol (TRAP) assay. Commercial versions of the TRAP assay are available, for example the Trapeze® telomerase detection kit (Millipore), which provides a sensitive detection and quantitation of telomerase activity, although other measurement techniques are also possible.

As previously noted, one of the advantages of the instant techniques is that the expression of telomerase activity is transient in the treated cells. In particular, such transient expression is in contrast to previous techniques where a telomerase reverse transcriptase gene persists in an episomal DNA moiety, or is inserted into the genomic sequence of the cell or otherwise permanently modifies the genetic make-up of the targeted cell and results in constitutive activity of the nucleic acid sequence.

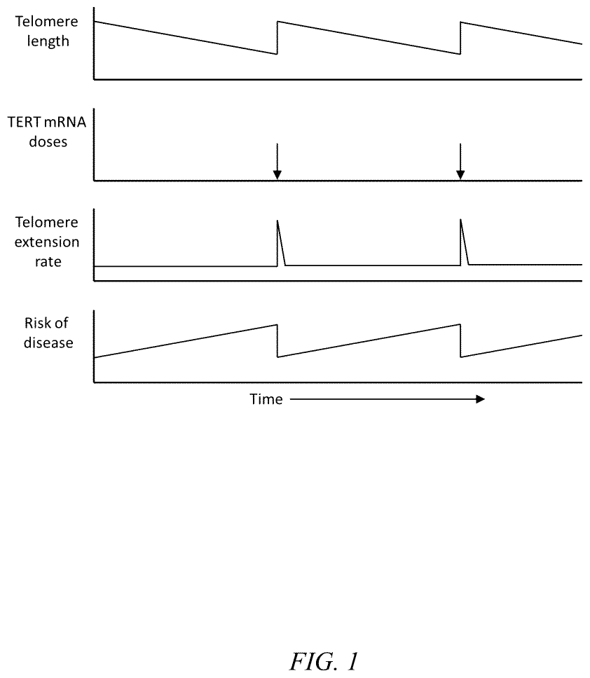

FIG. 1 graphically illustrates some of the advantages of the compounds, compositions, and methods disclosed herein. In particular, the speed of telomere extension made possible with these compounds, compositions, and methods enables telomere maintenance by very infrequent delivery of TERT mRNA. The expressed telomerase activity rapidly extends telomeres in a brief period, before being turned over, thus allowing the protective anti-cancer mechanism of telomere-shortening to function most of the time. Between treatments, normal telomerase activity and telomere shortening is present, and therefore the anti-cancer safety mechanism of telomere shortening to prevent out-of-control proliferation remains intact, while the risk of short telomere-related disease remains low. In contrast, small molecule treatments for extending telomeres may require chronic delivery, and thus present a chronic cancer risk, with minimal therapeutic benefit.

In some embodiments of the instant methods, the transient expression is independent of cell cycle.

As noted above, the transient expression of telomerase reverse transcriptase results in the extension of shortened telomeres in treated cells. Telomere length can be measured using techniques such as terminal restriction fragment (TRF) length analysis, qPCR, MMqPCR, TeSLA, flow FISH, and Q-FISH, as would be understood by one of ordinary skill in the art. In some embodiments, the instant methods increase average telomere length in treated cells by at least 0.1 kb, at least 0.2 kb, at least 0.3 kb, at least 0.4 kb, at least 0.5 kb, at least 1 kb, at least 2 kb, at least 3 kb, at least 4 kb, at least 5 kb, or even more. In some embodiments, the instant methods reduce the percentage of telomeres with lengths below a certain length, for example 1 kb, 2 kb, 3 kb, 4 kb, 5 kb, or more.

One of the advantages of the instant compounds, compositions, and methods, is the rapidity of extension of telomeres achieved by these techniques. The techniques allow treatments to be brief, and thus the interval between treatments can be long, and thus the treatments can be safe because the normal protective telomere shortening mechanism remains intact for most of the time i.e. between treatments.

The transient expression of telomerase reverse transcriptase also results in an increased replicative capacity in treated cells. Increased replicative capacity is readily monitored in cells that are approaching replicative senescence by measuring additional population doublings in such cells. Senescent cells do not divide in response to many conditions that cause normal cells to divide, for example passage in culture or treatment with serum. Senescent cells are further often characterized by the expression of pH-dependent P-galactosidase activity, expression of cell cycle inhibitors p53 and p19, and other altered patterns of gene expression, and an enlarged cell size. It is known in the art that, absent treatment with TERT mRNA, certain types of cells (e.g., human lung fibroblast cells) typically double 50-60 times after birth before senescing; with TERT mRNA treatments, however, these cells achieve an additional 16-28 population doublings. If treated again several weeks later, additional proliferative capacity is conferred again. This process of intermittent treatments to periodically re-extend telomeres may be applied additional times, with the interval between treatments depending on factors such as the rate of telomere shortening, the rate of cell divisions, and the amount of telomere extension provided by the treatment. Likewise, human microvascular dermal endothelial cells from an aged individual, absent treatment with the instant compositions, may achieve only 1-2 population doublings, whereas treated cells may achieve 3, 4, or even more population doublings.

Accordingly, in some embodiments, the instant treatment methods increase the number of population doublings of treated cells.

### VII. Therapeutic Kits

Therapeutic kits comprising a pharmaceutical composition of a TERT mRNA, or sequences thereof (including complementary sequences), and instructions for use are also contemplated herein. In some embodiments, the therapeutic kit comprises devices for administration, including but not limited to syringes, inhalers, nebulizers, and vials or containers.

In another aspect, the instant disclosure provides ready-to-use kits for use in extending telomeres in a mammalian cell. The kits comprise any of the above-described compounds or compositions, together with instructions for their use. In some embodiments, the kits further comprise packaging materials. In some embodiments, the packaging materials are air-tight. In these embodiments, the packaging materials may optionally be filled with an inert gas, such as, for example, nitrogen, argon, or the like. In some embodiments, the packaging materials comprise a metal foil container, such as, for example, a sealed aluminum pouch or the like. Such packaging materials are well known by those of ordinary skill in the art. The kit may also comprise a delivery vehicle, such as a lipid as described herein. In some embodiments, one or more components of the formulation are provided frozen with a cryoprotectant, or lyophilized.

In some embodiments, the kit may further comprise a desiccant, a culture medium, an RNase inhibitor, or other such components. In some embodiments, the kit may further comprise a combination of more than one of these additional components. In some kit embodiments, the composition of the kit is sterile.

### ENUMERATED EMBODIMENTS

The disclosure may be defined by reference to the following enumerated, illustrative embodiments.

Embodiment 1. A composition comprising a (i) a ribonucleic acid (RNA) encoding telomerase reverse transcriptase (TERT) and (ii) a delivery vehicle, wherein the RNA of (i) comprises one or more modified nucleotides and wherein the delivery vehicle of (ii) is operably-linked to the RNA of (i).

Embodiment 2. The composition of embodiment 1, wherein the delivery vehicle comprises one or more of a nanoparticle, a liposome, a cationic lipid, an exosome, an extracellular vesicle, a lipid nanoparticle (LNP), a natural lipoprotein particle and an artificial lipoprotein particle.

Embodiment 3. The composition of embodiment 1, wherein the delivery vehicle comprises a lipid nanoparticle (LNP).

Embodiment 4. The composition of embodiment 1, wherein the delivery vehicle comprises an ionizable lipid nanoparticle.

Embodiment 5. The composition of any one of embodiments 1-4, wherein the delivery vehicle comprises a targeting moiety.

Embodiment 6. The composition of embodiment 5, wherein the delivery vehicle specifically or selectively interacts with a liver cell.

Embodiment 7. The composition of embodiment 5, wherein the targeting moiety is a lipid, a peptide, and/or an antibody.

Embodiment 8. The composition of embodiment 3, wherein the LNP comprises an ionizable lipid, a phospholipid, a cholesterol, and/or a PEGylated lipid.

Embodiment 9. The composition of embodiment 8, wherein the LNP comprises a molar ratio of about 50 to about 60 moles of an ionizable lipid, about 4 to about 6 moles of a phospholipid, about 35 to about 45 moles of cholesterol, and about 1 to about 2 moles of PEGylated lipid.

Embodiment 10. The composition of any one of embodiments 1-9, wherein the delivery vehicle comprises a compound of Formula I:

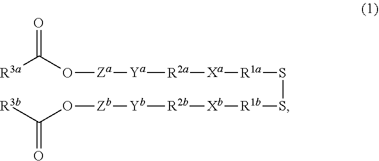

wherein R^{1a} and R^{1b} each independently represents an alkylene group having 1 to 6 carbon atoms, wherein X^{a} and X^{b} are each independently an acyclic alkyl tertiary amino group having 1 to 6 carbon atoms and 1 tertiary amino group, or 2 to 5 carbon atoms, and A cyclic alkylene tertiary amino group having 1 to 2 tertiary amino groups, wherein R^{2a} and R^{2b} each independently represent an alkylene group having 8 or less carbon atoms or an oxydialkylene group, wherein Y^{a} and Y^{b} each independently represent an ester bond, an amide bond, a carbamate bond, an ether bond or a urea bond;
wherein Z^{a} and Z^{b} are each independently a divalent group derived from an aromatic compound having 3 to 16 carbon atoms, having at least one aromatic ring, and optionally having a hetero atom, and
wherein R^{3a} and R^{3b} each independently represent a residue derived from a reaction product of a fat-soluble vitamin having a hydroxyl group and succinic anhydride or glutaric anhydride, or a sterol derivative having a hydroxyl group and succinic anhydride or a residue derived from a reaction product with glutaric anhydride or an aliphatic hydrocarbon group having 12 to 22 carbon atoms.

Embodiment 11. The composition of embodiment 10, wherein the compound of Formula I is:

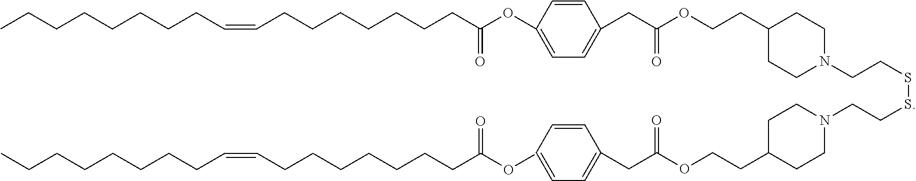

Embodiment 12. The composition of embodiment 10, wherein the compound of Formula I is:

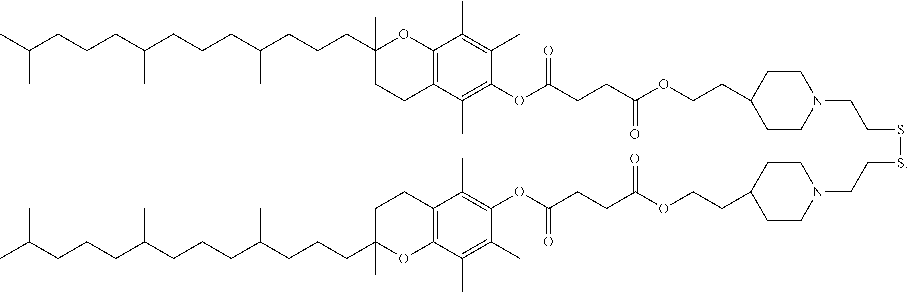 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

Embodiment 13. The composition of embodiment 10, wherein the compound of Formula I is:

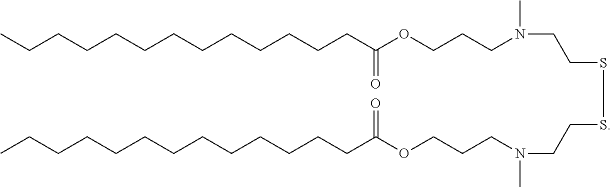 [SMILES: CCCCCCCCCCCCCC(=O)OCCCN(C)CCSSCCN(C)CCCOC(=O)CCCCCCCCCCCCC]

Embodiment 14. The composition of embodiment 10, wherein the compound of Formula I is:

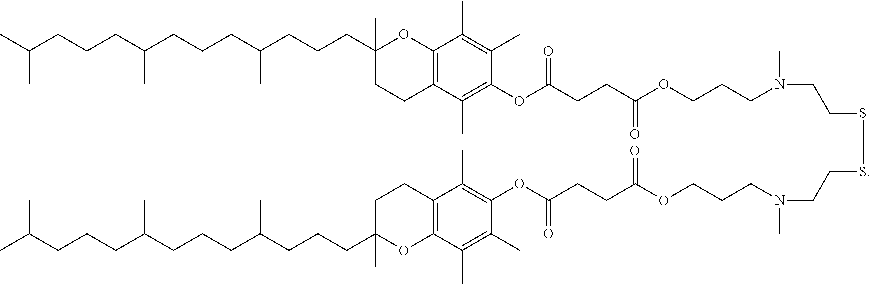 [SMILES: Cc1c(C)c2c(c(C)c1OC(=O)CCC(=O)OCCCN(C)CCSSCCN(C)CCCOC(=O)CCC(=O)Oc1c(C)c(C)c3c(c1C)CCC(C)(CCCC(C)CCCC(C)CCCC(C)C)O3)CCC(C)(CCCC(C)CCCC(C)CCCC(C)C)O2]

Embodiment 15. The composition of embodiment 10, wherein the compound of Formula I is:

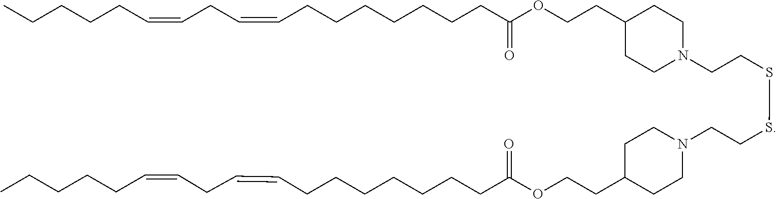 [SMILES: CCCCC/C=C\C/C=C\CCCCCCCC(=O)OCCC1CCN(CCSSCCN2CCC(CCOC(=O)CCCCCCC/C=C\C/C=C\CCCCC)CC2)CC1]

Embodiment 16. The composition of embodiment 10, wherein the compound of Formula I is:

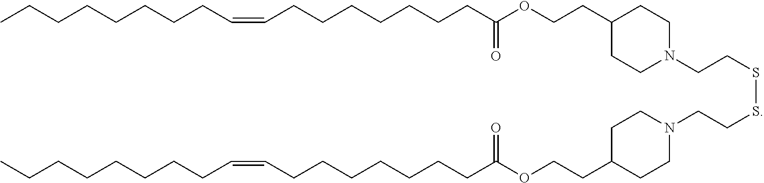 [SMILES: CCCCCCCC/C=C\CCCCCCCC(=O)OCCC1CCN(CCSSCCN2CCC(CCOC(=O)CCCCCCC/C=C\CCCCCCCC)CC2)CC1]

Embodiment 17. The composition of any one of embodiments 1-16, wherein the RNA comprises a sequence at least 70%, at least 75%, at least 80%, at least 85%, at least 90%, at least 95%, at least 97%, at least 99%, or 100% identical to any one of SEQ ID NOS: 1-5, 30-31, or 37-40.

Embodiment 18. The composition of embodiment 17, wherein the RNA comprises a 5′ cap.

Embodiment 19. The composition of embodiment 18, wherein the 5′cap comprises an anti-reverse cap analog (ARCA).

Embodiment 20. The composition of embodiment 19, wherein the ARCA comprises a 3′-O-Me-m7G(5′)ppp(5′)G structure.

Embodiment 21. The composition of embodiment 18, wherein the 5‘ cap comprise’ m7(3′O′eG)(5′‘ppp’5′)(2′OMeA)pG.

Embodiment 22. The composition of any one of embodiments 1-21, wherein the RNA further comprises at least one untranslated region (UTR).

Embodiment 23. The composition of embodiment 22, wherein the at least one UTR is positioned 5′ to the RNA of (i).

Embodiment 24. The composition of embodiment 22, wherein the at least one UTR is positioned 3′ to the RNA of (i).

Embodiment 25. The composition of any one of embodiments 22-24, wherein the UTR comprises a human sequence.

Embodiment 26. The composition of any one of embodiments 22-24, wherein the UTR comprises a non-human sequence.

Embodiment 27. The composition of any one of embodiments 22-26, wherein the UTR comprises a chimeric sequence.

Embodiment 28. The composition of embodiment 27, wherein the chimeric sequence increases stability, increases a transcription rate or decreases a time until initiation of transcription of the RNA of (i).

Embodiment 29. The composition of any one of embodiments 22-28, wherein the UTR comprises a sequence having at least 70% identity to a UTR sequence isolated or derived from one or more of α-globin, β-globin, c-fos, and a tobacco etch virus.

Embodiment 30. The composition of any one of embodiments 1-29, wherein the one or more modified nucleotides of the RNA of (i) comprise one or more of a modified adenine or analog thereof, a modified cytidine or analog thereof, a modified guanosine or analog thereof, and a modified uridine or analog thereof.

Embodiment 31. The composition of any one of embodiments 1-30, wherein the one or more modified nucleotides of the RNA of (i) comprise one or more of 1-methylpseudouridine, pseudouridine, 2-thiouridine, and 5-methylcytidine.

Embodiment 32. The composition of any one of embodiments 1-31, wherein the one or more modified nucleotides of the RNA of (i) comprise 5-methoxyuridine (5-moU).

Embodiment 33. The composition of any one of embodiments 1-32, wherein the one or more modified nucleotides of the RNA of (i) comprise one or more of m1A 1-methyladenosine, m6A N6-methyladenosine, Am 2′-O-methyladenosine, i6A N6-isopentenyladenosine, io6A N6-(cis-hydroxyisopentenyl)adenosine, ms2io6A 2-methylthio-N6-(cis-hydroxyisopentenyl) adenosine, g6A N6-glycinylcarbamoyladenosine, t6A N6-threonylcarbamoyladenosine, ms2t6A 2-methylthio-N6-threonyl carbamoyladenosine, Ar(p) 2′-O-ribosyladenosine (phosphate), m6 2A N6,N6-dimethyladenosine, m6Am N6,2′-O-dimethyladenosine, m6 2Am N6,N6,2′-O-trimethyladenosine, m1Am 1,2′-O-dimethyladenosine, m3C 3-methylcytidine, m5C 5-methylcytidine, Cm 2′-O-methylcytidine, ac4C N4-acetylcytidine, f5C 5-formylcytidine, m4C N4-methylcytidine, hm5C 5-hydroxymethylcytidine, f5Cm 5-formyl-2′-O-methylcytidine, m1G 1-methylguanosine, m2G N2-methylguanosine, m7G 7-methylguanosine, Gm 2′-O-methylguanosine, m2 2G N2,N2-dimethylguanosine, Gr(p) 2′-O-ribosylguanosine (phosphate), yW wybutosine, o2yW peroxywybutosine, OHyW hydroxywybutosine, OHyW* undermodified hydroxywybutosine, imG wyosine, m2,7G N2,7-dimethylguanosine, m2,2,7G N2,N2,7-trimethylguanosine I inosine, m1I 1-methylinosine, Im 2′-O-methylinosine, Q queuosine, galQ galactosyl-queuosine, manQ mannosyl-queuosine, Ψ pseudouridine, D dihydrouridine, m5U 5-methyluridine, Um 2′-O-methyluridine, m5Um 5,2′-O-dimethyluridine, m1Ψ 1-methylpseudouridine, Ψm 2′-O-methylpseudouridine, s2U 2-thiouridine, ho5U 5-hydroxyuridine, chm5U 5-(carboxyhydroxymethyl)uridine, mchm5U 5-(carboxyhydroxymethyl)uridine, methyl ester mcm5U 5-methoxycarbonylmethyluridine, mcm5Um 5-methoxycarbonylmethyl-2′-O-methyluridine, mcm5s2U 5-methoxycarbonylmethyl-2-thiouridine, ncm5U 5-carbamoylmethyluridine, ncm5Um 5-carbamoylmethyl-2′-O-methyluridine, cmnm5U 5-carboxymethylaminomethyluridine, m3U 3-methyluridine, m1acp3Ψ 1-methyl-3-(3-amino-3-carboxypropyl) pseudouridine, cm5U 5-carboxymethyluridine, m3Um 3,2′-O-dimethyluridine, m5D 5-methyldihydrouridine, τm5U 5-taurinomethyluridine, τm5s2U 5-taurinomethyl-2-thiouridine, 2-Aminoadenosine, 2-Amino-6-chloropurineriboside, 8-Azaadenosine, 6-Chloropurineriboside, 5-Iodocytidine, 5-Iodouridine, Inosine, 2′-O-Methylinosine, Xanthosine, 4-Thiouridine, 06-Methylguanosine, 5,6-Dihydrouridine, 2-Thiocytidine, 6-Azacytidine, 6-Azauridine, 2′-O-Methyl-2-aminoadenosine, 2′-O-Methylpseudouridine, N1-Methyladenosine, 2′-O-Methyl-5-methyluridine, 7-Deazaguanosine, 8-Azidoadenosine, 5-Bromocytidine, 5-Bromouridine, 7-Deazaadenosine, 5-Aminoallyluridine, 5-Aminoallylcytidine, 8-Oxoguanosine, 2-Aminopurine-riboside, Pseudoisocytidine, N1-Methylpseudouridine, 5,6-Dihydro-5-Methyluridine, N6-Methyl-2-Aminoadenosine, 5-Carboxycytidine, 5-Hydroxymethyluridine, Thienoguanosine, 5-Hydroxycytidine, 5-Formyluridine, 5-Carboxyuridine, 5-Methoxyuridine, 5-Methoxycytidine, Thienouridine, 5-Carboxymethylesteruridine, Thienocytidine, 8-Oxoadenoosine, Isoguanosine, N1-Ethylpseudouridine, N1-Methyl-2′-O-Methylpseudouridine, N1-Methoxymethylpseudouridine, N1-Propylpseudouridine, 2′-O-Methyl-N6-Methyladenosine, 2-Amino-6-Cl-purine-2′-deoxyriboside, 2-Amino-2′-deoxyadenosine, 2-Aminopurine-2′-deoxyriboside, 5-Bromo-2′-deoxycytidine, 5-Bromo-2′-deoxyuridine, 6-Chloropurine-2′-deoxyriboside, 7-Deaza-2′-deoxyadenosine, 7-Deaza-2′-deoxyguanosine, 2′-Deoxyinosine, 5-Propynyl-2′-deoxycytidine, 5-Propynyl-2′-deoxyuridine, 5-Fluoro-2′-deoxyuridine, 5-Iodo-2′-deoxycytidine, 5-Iodo-2′-deoxyuridine, N6-Methyl-2′-deoxyadenosine, 5-Methyl-2′-deoxycytidine, 06-Methyl-2′-deoxyguanosine, N2-Methyl-2′-deoxyguanosine, 8-Oxo-2′-deoxyadenosine, 8-Oxo-2′-deoxyguanosine, 2-Thiothymidine, 2′-Deoxy-P-nucleoside, 5-Hydroxy-2′-deoxycytidine, 4-Thiothymidine, 2-Thio-2′-deoxycytidine, 6-Aza-2′-deoxyuridine, 6-Thio-2′-deoxyguanosine, 8-Chloro-2′-deoxyadenosine, 5-Aminoallyl-2′-deoxycytidine, 5-Aminoallyl-2′-deoxyuridine, N4-Methyl-2′-deoxycytidine, 2′-Deoxyzebularine, 5-Hydroxymethyl-2′-deoxyuridine, 5-Hydroxymethyl-2′-deoxycytidine, 5-Propargylamino-2′-deoxycytidine, 5-Propargylamino-2′-deoxyuridine, 5-Carboxy-2′-deoxycytidine, 5-Formyl-2′-deoxycytidine, 5-[(3-Indolyl)propionamide-N-allyl]-2′-deoxyuridine, 5-Carboxy-2′-deoxyuridine, 5-Formyl-2′-deoxyuridine, 7-Deaza-7-Propargylamino-2′-deoxyadenosine, 7-Deaza-7-Propargylamino-2′-deoxyguanosine, Biotin-16-Aminoallyl-2′-dUTP, Biotin-16-Aminoallyl-2′-dCTP, Biotin-16-Aminoallylcytidine, N4-Biotin-OBEA-2′-deoxycytidine, Biotin-16-Aminoallyluridine, Dabcyl-5-3-Aminoallyl-2′-dUTP, Desthiobiotin-6-Aminoallyl-2′-deoxycytidine, Desthiobiotin-16-Aminoallyl-Uridine, Biotin-16-7-Deaza-7-Propargylamino-2′-deoxyguanosine, Cyanine 3-5-Propargylamino-2′-deoxycytidine, Cyanine 3-6-Propargylamino-2′-deoxyuridine, Cyanine 5-6-Propargylamino-2′-deoxycytidine, Cyanine 5-6-Propargylamino-2′-deoxyuridine, Cyanine 3-Aminoallylcytidine, Cyanine 3-Aminoallyluridine, Cyanine 5-Aminoallylcytidine, Cyanine 5-Aminoallyluridine, Cyanine 7-Aminoallyluridine, 2′-Fluoro-2′-deoxyadenosine, 2′-Fluoro-2′-deoxycytidine, 2′-Fluoro-2′-deoxyguanosine, 2′-Fluoro-2′-deoxyuridine, 2′-O-Methyladenosine, 2′-O-Methylcytidine, 2′-O-Methylguanosine, 2′-O-Methyluridine, Puromycin, 2′-Amino-2′-deoxycytidine, 2′-Amino-2′-deoxyuridine, 2′-Azido-2′-deoxycytidine, 2′-Azido-2′-deoxyuridine, Aracytidine, Arauridine, 2′-Azido-2′-deoxyadenosine, 2′-Amino-2′-deoxyadenosine, Araadenosine, 2′-Fluoro-thymidine, 3′-O-Methyladenosine, 3′-O-Methylcytidine, 3′-O-Methylguanosine, 3′-O-Methyluridine, 2′-Azido-2′-deoxyguanosine, Araguanosine, 2′-Deoxyuridine, 3′-O-(2-nitrobenzyl)-2′-Deoxyadenosine, 3′-O-(2-nitrobenzyl)-2′-Deoxyinosine, 3′-Deoxyadenosine, 3′-Deoxyguanosine, 3′-Deoxycytidine, 3′-Deoxy-5-Methyluridine, 3′-Deoxyuridine, 2′,3′-Dideoxyadenosine, 2′,3′-Dideoxyguanosine, 2′,3′-Dideoxyuridine, 2′,3′-Dideoxythymidine, 2′,3′-Dideoxycytidine, 3′-Azido-2′,3′-dideoxyadenosine, 3′-Azido-2′,3′-dideoxythymidine, 3′-Amino-2′,3′-dideoxyadenosine, 3′-Amino-2′,3′-dideoxycytidine, 3′-Amino-2′,3′-dideoxyguanosine, 3′-Amino-2′,3′-dideoxythymidine, 3′-Azido-2′,3′-dideoxycytidine, 3′-Azido-2′,3′-dideoxyuridine, 5-Bromo-2′,3′-dideoxyuridine, 2′,3′-Dideoxyinosine, 2′-Deoxyadenosine-5′-O-(1-Thiophosphate), 2′-Deoxycytidine-5′-O-(1-Thiophosphate), 2′-Deoxyguanosine-5′-O-(1-Thiotriphosphate), 2′-Deoxythymidine-5′-O-(1-Thiophosphate), Adenosine-5′-O-(1-Thiophosphate), Cytidine-5′-O-(1-Thiophosphate), Guanosine-5′-O-(1-Thiophosphate), Uridine-5′-O-(1-Thiophosphate), 2′,3′-Dideoxyadenosine-5′-O-(1-Thiophosphate), 2′,3′-Dideoxycytidine-5′-O-(1-Thiophosphate), 2′,3′-Dideoxyguanosine-5′-O-(1-Thiophosphate), 3′-Deoxythymidine-5′-O-(1-Thiophosphate), 3′-Azido-2′,3′-dideoxythymidine-5′-O-(1-Thiophosphate), 2′,3′-Dideoxyuridine-5′-O-(1-Thiophosphate), 2′-Deoxyadenosine-5′-O-(1-Boranophosphate), 2′-Deoxycytidine-5′-O-(1-Boranophosphate), 2′-Deoxyguanosine-5′-O-(1-Boranophosphate), and 2′-Deoxythymidine-5′-O-(1-Boranophosphate).

Embodiment 34. The composition of any one of embodiments 1-33, wherein the composition further comprises a ribonucleic acid (RNA) encoding TElomerase RNA Component (TERC).

Embodiment 35. The composition of any one of embodiments 1-34, wherein the delivery vehicle comprises the RNA encoding TERT.

Embodiment 36. The composition of embodiment 35, wherein the RNA encoding TERT comprises a sequence at least 70%, at least 75%, at least 80%, at least 85%, at least 90%, at least 95%, at least 97%, at least 99%, or 100% identical to any one of SEQ ID NOS: 11-5, 7, 9, 14-17, 19, 21, 23, 25, 27, 29-31, 37-40.

Embodiment 37. The composition of embodiment 35, wherein the RNA encoding TERT comprises a full length or part thereof, of a UTR of one of SEQ ID NOS: 32-34, 35, and 36.

Embodiment 38. The composition of any one of embodiments 1-36, wherein the RNA comprises a self-replicating RNA.

Embodiment 39. The composition of any one of embodiments 1-38, wherein the RNA comprises a circular RNA.

Embodiment 40. The composition of embodiment 8, wherein the layer comprises a lipid monolayer or lipid bi-layer.

Embodiment 41. The composition of embodiment 41, wherein the delivery vehicle comprises an internal volume.

The composition of any one of embodiments 1-39, wherein the delivery vehicle is operably-linked to a ribonucleic acid (RNA) encoding TElomerase RNA Component (TERC).

Embodiment 41. The composition of embodiment 40, wherein the delivery vehicle comprises the RNA encoding TERC.

Embodiment 42. The composition of embodiment 35, wherein one or more of a surface, a layer or a volume of the delivery vehicle comprises the RNA encoding TERC.

Embodiment 43. The composition of embodiment 42, wherein the surface comprises an outer surface or an inner surface.

Embodiment 44. The composition of embodiment 42, wherein the layer comprises a lipid monolayer or lipid bi-layer.

Embodiment 45. The composition of embodiment 42, wherein the volume comprises an internal volume.

Embodiment 46. A method of increasing telomerase activity in a cell, the method comprising contacting the cell and the composition of any one of embodiments 1-45.

Embodiment 47. A method of extending telomeres in a cell, the method comprising contacting the cell and the composition of any one of embodiments 1-45.

Embodiment 48. The method of embodiment 46 or 47, wherein the cell is in vivo, ex vivo or in vitro.

Embodiment 49. A cell comprising the composition of any one of embodiments 1-45.

Embodiment 50. A formulation comprising the cell of embodiment 49.

Embodiment 51. The formulation of embodiment 50, wherein a plurality of cells comprises the cell of embodiment 29.

Embodiment 52. The formulation of embodiment 51, wherein each cell of the plurality is a cell according to embodiment 49.

Embodiment 53. A method of treating a disease or disorder comprising administering to a subject an effective amount of a composition according to any one of embodiments 1-45.

Embodiment 54. A method of treating a disease or disorder comprising administering to a subject an effective amount of a cell according to embodiment 49.

Embodiment 55. A method of treating a disease or disorder comprising administering to a subject an effective amount of a formulation according to any one of embodiments 50-52.

Embodiment 56. A method of delaying the onset of a disease comprising administering to a subject an effective amount of a composition according to any one of embodiments 1-45.

Embodiment 57. A method of delaying the onset of a disease comprising administering to a subject an effective amount of a cell according to embodiment 49.

Embodiment 58. A method of delaying the onset of a disease comprising administering to a subject an effective amount of a formulation according to any one of embodiments 50-52.

Embodiment 59. A method of treating a fibrotic disease in a subject in need thereof, comprising: administering to the subject an effective amount of a composition comprising one or more synthetic messenger ribonucleic acids (mRNAs) encoding telomerase reverse transcriptase (TERT).

Embodiment 60. The method of embodiment 59, wherein the composition comprises a delivery vehicle.

Embodiment 61. The method of embodiment 60, wherein the delivery vehicle is a nanoparticle.

Embodiment 62. The method of embodiment 61, wherein the nanoparticle is a lipid nanoparticle (LNP).

Embodiment 63. The method of embodiment 62, wherein the LNP comprises an ionizable lipid, a phospholipid, a cholesterol, and/or a PEGylated lipid.

Embodiment 64. The method of embodiment 63, wherein the LNP comprises a molar ratio of about 50 to about 60 moles of an ionizable lipid, to about 4 to about 6 moles of a phospholipid, about 35 to about 45 moles of cholesterol, and about 1.0 to about 2.0 moles of PEGylated lipid.

Embodiment 65. The method of embodiment 63, wherein the LNP comprises a molar ratio of about 30 to 40 moles of an ionizable lipid, to about 14 to about 18 moles of a phospholipid, about 40 to about 50 moles of a cholesterol, and about 2.0 to about 3.0 moles of a PEGylated lipid.

Embodiment 66. The method of any one of embodiments 59-65, wherein the TERT synthetic mRNA comprises at least one modified nucleoside from the list in Table 1B.

Embodiment 67. The method of embodiment 66, wherein the modified nucleoside is pseudouridine or a pseudouridine analog.

Embodiment 68. The method of embodiment 67, wherein the pseudouridine analog is N-1-methylpseudouridine.

Embodiment 69. The method of embodiment 66, wherein the modified nucleoside is 5-methoxyuridine.

Embodiment 70. The method of any one of embodiments 59-69, wherein the TERT synthetic mRNA comprises an untranslated region (UTR).

Embodiment 71. The method of embodiment 70, wherein the UTR comprises a sequence at least 70%, at least 75%, at least 80%, at least 85%, at least 90%, at least 95%, at least 97%, at least 99%, or 100% identical to any one of SEQ ID NOS: 32-36.

Embodiment 72. The method of any one of embodiments 59-71, wherein the wherein the TERT synthetic mRNA comprises a 5′ cap structure, wherein the 5′ cap structure is IRES, Cap0, Cap1, ARCA, inosine, N1-methyl-guanosine, 2′fluoro-guanosine, 7-deaza-guanosine, CleanCap™, m7(3′O′eG)(5′‘ppp’5′)(2′OMeA)pG, 8-oxo-guanosine, 2-amino-guanosine, LNA-guanosine, 2-azido-guanosine, Cap2, Cap4, CAP-003, or CAP-225.

Embodiment 73. The method of any one of embodiments 59-72, wherein the TERT synthetic mRNA comprises a poly-adenosine (poly-A) nucleotide sequence 3′ to the encoding region.

Embodiment 74. The method of any one of embodiments 59-73, wherein the TERT synthetic mRNA comprises a chain terminating nucleotide, wherein the nucleotide' is 3′-deoxyadenosine (cordycepin), 3′-deoxyurid'ne, 3‘-deoxycytos'ne, 3‘-deoxyguanos'ne, 3′-deoxythym'ne’ 2′,3′-dideoxynucleosi'es' 2′,3′-dideoxyadenos'ne’ 2′,3′-dideoxyurid'ne' 2′,3′-dideoxycytos'ne' 2′,3′-deoxyguanosine' 2′,3‘-dideoxythymin’, a 2′-deoxynucleoside, or —O— methylnucleoside.

Embodiment 75. The method of any one of embodiments 59-74, wherein the wherein the TERT synthetic mRNA is codon optimized.

Embodiment 76. The method of any one of embodiments 59-73, wherein the TERT synthetic mRNA comprises a sequence of a sequence at least 70%, at least 75%, at least 80%, at least 85%, at least 90%, at least 95%, at least 97%, at least 99%, or 100% identical to any one of SEQ ID NOS: 1, 2, 7, 9, 30, 39, or 40.

Embodiment 77. The method of embodiment 60, wherein the delivery vehicle is a liposome, an ionizable lipid, or an exosome.

Embodiment 78. The method of embodiment 77, wherein the delivery vehicle is an exosome, and wherein the exosome comprises a targeting moiety of one or more of a lipid, a peptide, or an antibody

Embodiment 79. The method of any one of embodiments 59-78, wherein the method reduces fibrosis.

Embodiment 80. The method of any one of embodiments 59-79, wherein the subject is human.

Embodiment 81. A composition according to any one of embodiments 1-58 for use in a method according to any one of embodiments 59-80.

Embodiment 82. The composition for use of embodiment 81, wherein the composition is a pharmaceutical composition comprising one or more pharmaceutically acceptable solvents or excipients.

Embodiment 83. A kit for treating a fibrotic disease in a subject, the kit comprising a composition according to any one of embodiment 1-58, and instructions for use thereof.

Embodiment 84. A method of treating a liver disease in a subject in need thereof, comprising: administering to the subject a composition comprising one or more synthetic messenger ribonucleic acids (mRNAs) encoding telomerase reverse transcriptase (TERT).

Embodiment 85. The method of embodiment 84, wherein the composition comprises a delivery vehicle.

Embodiment 86. The method of embodiment 85, wherein the delivery vehicle is a nanoparticle.

Embodiment 87. The method of embodiment 86, wherein the nanoparticle is a lipid nanoparticle (LNP).

Embodiment 88. The method of embodiment 87, wherein the LNP comprises an ionizable lipid, a phospholipid, a cholesterol, and/or a PEGylated lipid.

Embodiment 89. The method of embodiment 88, wherein the LNP comprises a molar ratio of about 50 to about 60 moles of an ionizable lipid, to about 4 to about 6 moles of a phospholipid, about 35 to about 45 moles of cholesterol, and about 1 to about 2 moles of PEGylated lipid.

Embodiment 90. The method of embodiment 88, wherein the LNP comprises a molar ratio of about 55 moles of an ionizable lipid, to about 5 moles of a phospholipid, about 40 moles of a cholesterol, and about 1.5 moles of a PEGylated lipid.

Embodiment 91. The method of any one of embodiments 84-90, wherein the TERT synthetic mRNA comprises at least one modified nucleoside from the list in Table 1B.

Embodiment 92. The method of embodiment 91, wherein the modified nucleoside is pseudouridine or a pseudouridine analog.

Embodiment 93. The method of embodiment 92, wherein the pseudouridine analog is N-1-methylpseudouridine.

Embodiment 94. The method of embodiment 91, wherein the modified nucleoside is 5-methoxyuridine.

Embodiment 95. The method of any one of embodiments 84-94, wherein the TERT synthetic mRNA comprises an untranslated region (UTR).

Embodiment 96. The method of embodiment 95, wherein the UTR comprises a sequence at least 70%, at least 75%, at least 80%, at least 85%, at least 90%, at least 95%, at least 97%, at least 99%, or 100% identical to any one of SEQ ID NOS: 32-36.

Embodiment 97. The method of any one of embodiments 84-96, wherein the wherein the TERT synthetic mRNA comprises a 5′ cap structure, wherein the 5′ cap structure is IRES, Cap0, Cap1, ARCA, inosine, N1-methyl-guanosine, 2′fluoro-guanosine, 7-deaza-guanosine, CleanCap™, m7(3′O′eG)(5′‘ppp’5′)(2′OMeA)pG, 8-oxo-guanosine, 2-amino-guanosine, LNA-guanosine, 2-azido-guanosine, Cap2, Cap4, CAP-003, or CAP-225.

Embodiment 98. The method of any one of embodiments 84-97, wherein the TERT synthetic mRNA comprises a poly-adenosine (poly-A) nucleotide sequence 3′ to the encoding region.

Embodiment 99. The method of any one of embodiments 84-98, wherein the TERT synthetic mRNA comprises a chain terminating nucleotide, wherein the nucleotide' is 3′-deoxyadenosine (cordycepin), 3′-deoxyurid'ne, 3′-deoxycytos'ne, 3′-deoxyguanos'ne, 3′-deoxythym'ne' 2′,3′-dideoxynucleosi'es' 2′,3′-dideoxyadenos'ne' 2′,3′-dideoxyurid'ne' 2′,3′-dideoxycytos'ne' 2′,3′-deoxyguanosine' 2′,3′-dideoxythymin', a 2′-deoxynucleoside, or —O— methylnucleoside.

Embodiment 100. The method of any one of embodiments 84-99, wherein the wherein the TERT synthetic mRNA is codon optimized.

Embodiment 101. The method of any one of embodiments 84-99, wherein the TERT synthetic mRNA comprises a sequence at least 70%, at least 75%, at least 80%, at least 85%, at least 90%, at least 95%, at least 97%, at least 99%, or 100% identical to any one of SEQ ID NOS: 1, 2, 7, 9, 30, 39, or 40.

Embodiment 102. The method of embodiment 85, wherein the delivery vehicle is a liposome, a cationic lipid, or an exosome.

Embodiment 103. The method of any one of embodiments 84-102, wherein the method reduces liver fibrosis.

Embodiment 104. The method of any one of embodiments 84-103, wherein the liver disease is non-alcoholic steatohepatitis (NASH) or non-alcoholic fatty liver disease (NAFLD).

Embodiment 105. The method of any one of embodiments 84-103, wherein the liver disease is alcoholic hepatitis.

Embodiment 106. The method any one of embodiments 84-103, wherein the liver disease is liver cirrhosis or liver fibrosis.

Embodiment 107. The method of any one of embodiments 84-103, wherein the liver disease is compensated cirrhosis, decompensated cirrhosis, or acute-on-chronic liver failure.

Embodiment 108. The method of any one of embodiments 84-103, wherein the liver disease is fibrotic stage F4 Non-alcoholic steatohepatitis (NASH).

Embodiment 109. The method of any one of embodiments 84-103, wherein the liver disease is biliary atresia, primary biliary cirrhosis, primary sclerosing cholangitis, and/or chronic liver disease.

Embodiment 110. The method of any one of embodiments 84-103, wherein the liver disease is hemochromatosis, Wilson's disease, or ischemic hepatitis.

Embodiment 111. The method of any one of embodiments 84-110, wherein the subject is human.

Embodiment 112. A composition according to any one of embodiments 1-58 for use in a method according to any one of embodiments 84-111.

Embodiment 113. The composition for use of embodiment 112, wherein the composition is a pharmaceutical composition comprising one or more pharmaceutically acceptable solvents or excipients.

Embodiment 114. A kit for treating a liver disease in a subject, the kit comprising the composition according to any one of embodiments 1-58, and instructions for use thereof.

### EXAMPLES

The following examples are included for illustrative purposes and are not intended to limit the scope of the disclosure.

### Example 1: LNP Formulations for mRNA Expression in Liver

This Example demonstrates that three diverse lipid nanoparticle (LNP) formulations may be used to deliver an mRNA encoding a heterologous protein to the liver of a subject animal. An LNP formulation that includes an SS-OP lipid resulted in the highest levels of expression and/or activity of the heterologous protein. In this Example, intravenous administration was used. Furthermore, the SS-OP-based formulation (LNP1) caused less tissue toxicity than a cKK-E12-based formulation (LNP2). These experiments demonstrate expression of the report Luciferase or the therapeutic gene TERT in the liver of subject animals. In particular, an SS-OP-based formulation is here shown to cause high TERT expression and/or activity with low toxicity.

Table 5 shows illustrative lipid nanoparticle (LNP) formulations targeting the liver in total lipid/mRNA ratios by weight/weight (wt/wt).

TABLE 5

| | | | Total lipid:mRNA |
|---|---|---|---|
| | Compound | Molar ratio | ratio wt/wt |
| LNP1 | | | |
| | SS-OP | 55 | 42 |
| | DOPC | 5 | |
| | Cholesterol | 40 | |
| | DMG-PEG2000 | 1.5 | |
| LNP2 | | | |
| | cKK-E12 | 35 | 20.5 |
| | DOPE | 16 | |
| | Cholesterol | 46.5 | |
| | 14:0 PEG2000 | 2.5 | |
| | PE | | |
| LNP3 | | | |
| | DLin-MC3- | 50 | 35 |
| | DMA | | |
| | DSPC | 10 | |
| | Cholesterol | 40 | |
| | DMG-PEG2000 | 1.5 | |
| LNP4 | | | |
| | SS-OP | 35 | 38.3 |
| | DOPE | 16 | |
| | Cholesterol | 46.5 | |
| | 14:0 PEG2000 | 2.5 | |
| | PE | | |
| LNP5 | | | |
| | cKK-E12 | 55 | 14.5 |
| | DOPC | 5 | |
| | Cholesterol | 40 | |
| | DMG-PEG2000 | 1.5 | |

Compositions and methods of the disclosure may be used for the treatment of cirrhosis. In some embodiments, compositions and/or methods of use of compositions of the disclosure intended for treatment of cirrhosis induce TERT expression or increase TERT activity in a liver cell. In some embodiments, compositions and/or methods of use of compositions of the disclosure intended for treatment of cirrhosis do not induce cellular, tissue or systemic toxicity. Compositions may be administered systemically, e.g, intravenously.

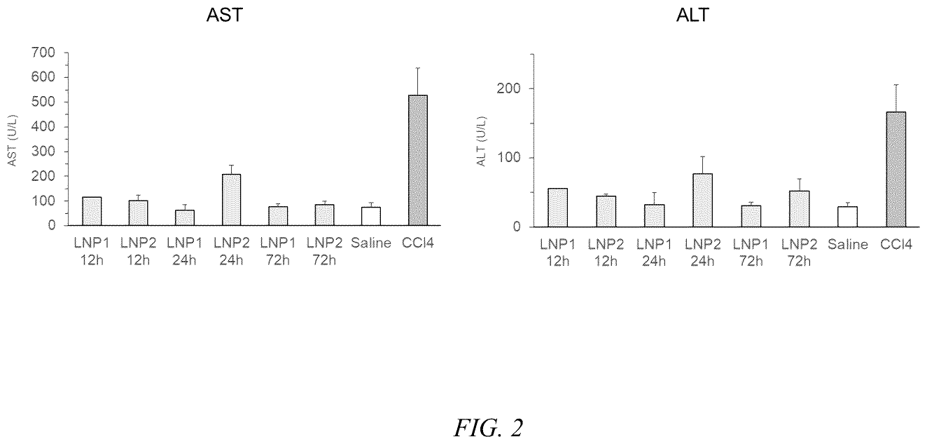

FIG. 2 is a series of graphs showing that mRNA LNPs exhibit low toxicity by liver panel. Mice were dosed intravenously with GRP or CRE mRNA encapsulated in a lipid nanoparticle employing either LNP1(comprising SS-OP™) or LNP2 (comprising cKK-E12) (N=1-4 per condition). Mice were sacrificed and blood was collected at the time points indicated (12, 24, and 72 hours). Mice receiving saline (N=4) and carbon tetrachloride (CCl4, N=4) served as negative and positive controls, respectively. Error bars display standard error of the mean.

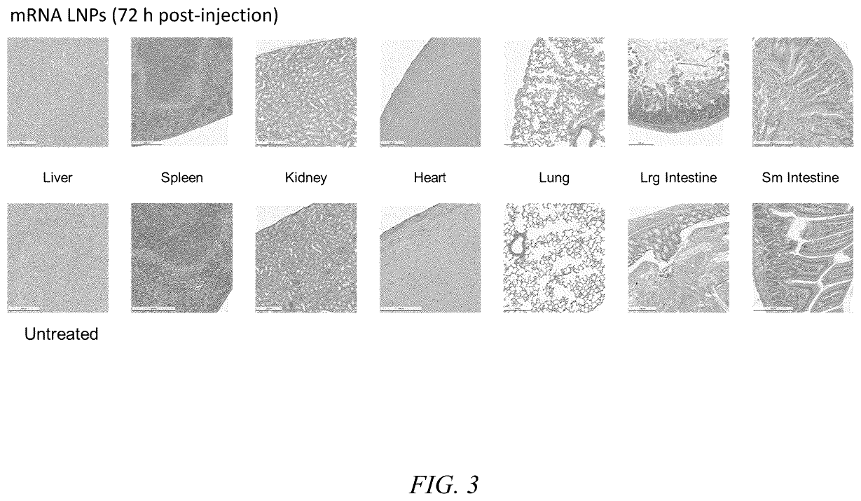

FIG. 3 is a series of photographs showing that intravenous delivery of TERT mRNA LNPs does not result in anormal histology. 11 μg Cre mRNA was encapsulated into LNP1 and delivered intravenously into tdTomato fl/fl mice. Organs were harvested 72 hours later, fixed, paraffin embedded, and sectioned. Organs from an untreated tdTomato fl/fl mouse are shown for reference.

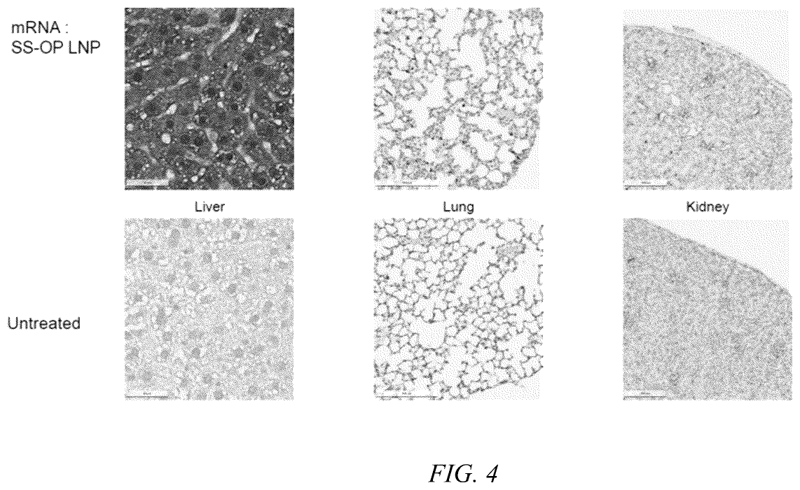

FIG. 4 is a series of photographs showing that TERT mRNA LNPs transfect hepatocytes with high efficiency. 11 μg Cre mRNA was encapsulated into LNP 1 and delivered intravenously (i.v.) into tdTomato fl/fl mice. Organs were harvested 72 hours later, fixed, paraffin embedded, and sectioned. Photographs depict immunohistochemistry (IHC) with anti-tdTomato. Organs from an untreated tdTomato fl/fl mouse are shown for reference.

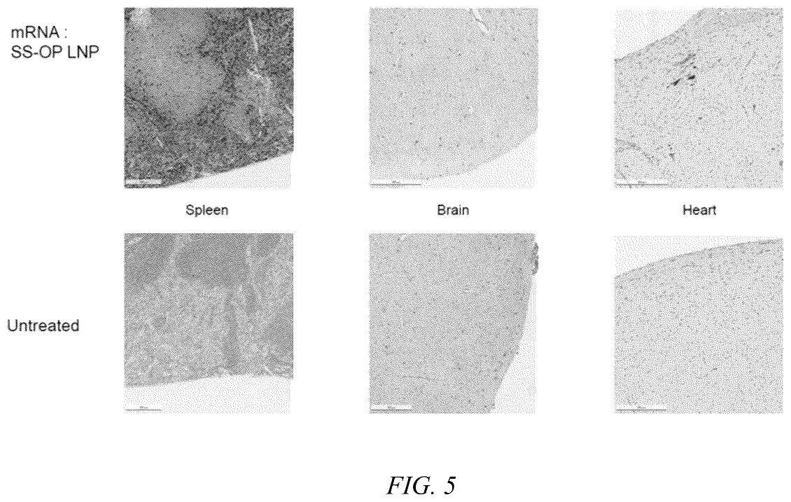

FIG. 5 is a series of photographs showing that TERT mRNA LNPs also target some cells in spleen, particularly in the red pulp area. 11 μg Cre mRNA was encapsulated into LNP1 and delivered intravenously (i.v.) into tdTomato fl/fl mice. Organs were harvested 72 hours later, fixed, paraffin embedded, and sectioned. Photographs depict immunohistochemistry (IHC) with anti-tdTomato. Organs from an untreated tdTomato fl/fl mouse are shown for reference.

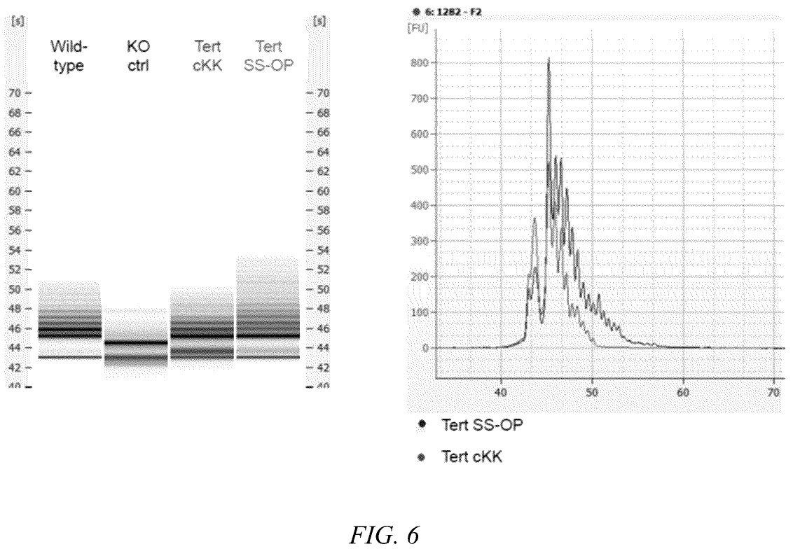

FIG. 6 is a pair of graphs showing that TERT mRNA LNPs cause high telomerase activity in liver. Tert mRNA (SEQ ID NO: 37) was formulated with LNP1 or LNP2 and delivered intravenously in a concentration of 0.6 mg/kg into TERT KO mice. 20 hours later, the livers were harvested for TRAP. Wild-type C57B16/J and untreated TERT KO mouse livers were used as positive and negative controls, respectively.

The TRAP assay uses lysate from cells or tissues incubated with an artificial telomere (DNA oligonucleotide) to detect telomerase. If active telomerase is present, it extends the artificial telomere 6 base pairs (bp) at a time, producing a ladder pattern. This extension reaction is amplified by PCR and run on a gel (in this case Agilent bioanalyzer, a microfluidic agarose gel). The presence of a ladder in 6 bp increments indicates telomerase activity.

FIG. 7 is a photograph demonstrating that exemplary LNP formulations deliver luciferase (LUC) mRNA to the liver, as demonstrated by the high bioluminescence signals. Shown are LNP1 (comprising SS-OP™), LNP2 (comprising cKK), and LNP3 (comprising DLin-MC3-DMA). An empty LNP formulation is also shown as a negative control (ctrl). Luciferase mRNA was formulated with the aforementioned LNPs 1, 2, and 3 and delivered intravenously into C57B16/J mice. 20 hours later, these mice were shaved and imaged after luciferin injection using an IVIS™ Bioluminescence imaging system.

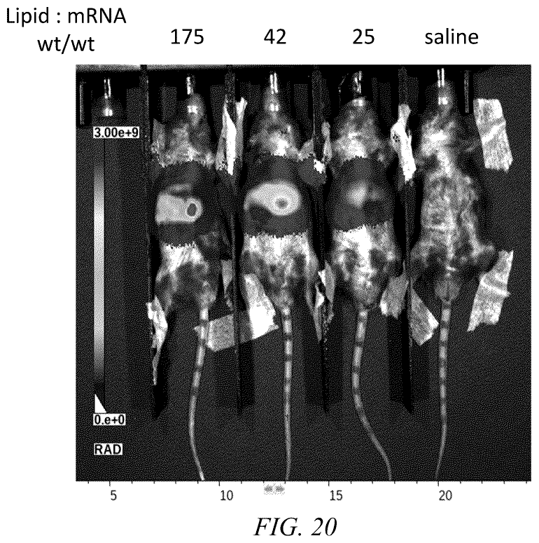

FIG. 20 shows in vivo delivery of mRNA in a photograph depicting the results demonstrating that luciferase mRNA LNPs causing high bioluminescence signal in liver. Luciferase mRNA was formulated with SS-OP using the lipid ratios for LNP1, as shown in Table 5. The lipid:mRNA ratios (wt/wt) were varied. The formulated mRNA LNPs were delivered via IV injection into C57B16/J mice at 0.6 mg of total mRNA/kg of body weight. As a negative control, a mouse was injected with saline. 24 hours later, these mice were shaved and imaged after injection with luciferin using a Lago instrument from Spectral Instruments Imaging. Depicted is an BLI image from mice dosed with a lipid:mRNA ratio of 175, 42, and 25. The signal was highest in the mice receiving LNPs with a lipid:mRNA ratio (wt/wt) of 175 and 42. The other data presented here using LNP1 uses a wt/wt ratio of 42.

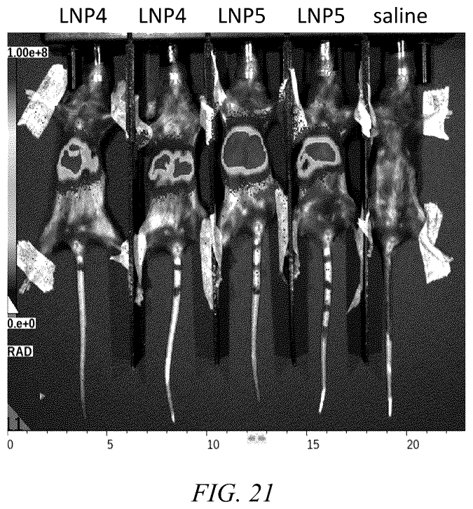

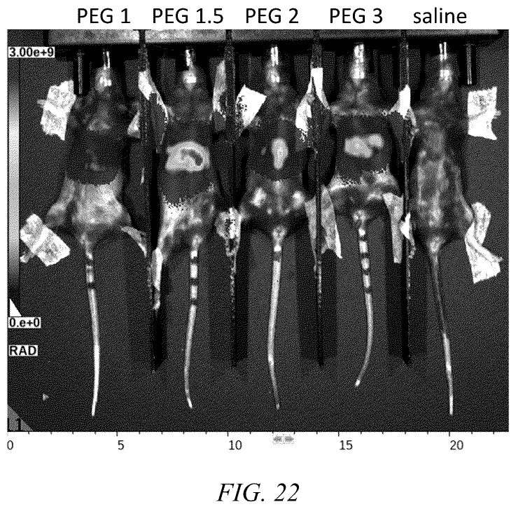

FIG. 21 shows in vivo delivery of mRNA in a photograph depicting the results demonstrating that luciferase mRNA LNPs causing high bioluminescence signal in liver. LNPs designated as Lipid Nanoparticle 4 (LNP4) or Lipid Nanoparticle 5 (LNP5) were formulated using the recipe in Table 5 with luciferase mRNA. These LNPs were delivered via IV injection into C57B16/J mice at 0.6 mg/kg. As a negative control, a mouse was injected with saline. 20 hours later, these mice were shaved and imaged after injection with luciferin using the Lago instrument from Spectral Instruments Imaging. LNP4 consisted of the formula for LNP2, but with SS-OP substituted for cKK-E12. LNP5 consisted of the formula for LNP1, but with cKK-E12 substituted for SS-OP. Bioluminescent imaging indicates that both of these LNPs had successful delivery to the liver.

FIG. 22 shows in vivo delivery of mRNA in a photograph depicting the results demonstrating that luciferase mRNA LNPs causing high bioluminescence signal in liver. Luciferase mRNA was formulated with lipids per the recipe for LNP1 in Table 5. The ingredient that was varied was the molar ratio of DMG-PEG2000. As shown in FIG. 22, DMG-PEG2000 was added as either 1, 1.5, 2, or 3 parts relative to the molar sum of all lipids, while the molar ratio for the other 3 lipids is held constant. This corresponds to a molar percentage for DMG-PEG2000 of approximately 1.0%, 1.5%, 2.0%, and 2.9%, 20 hours after intravenous delivery at 0.6 mg/kg, the C57B16/J mice were shaved and imaged following luciferin injection (75 mg/kg) using the Lago instrument from Spectral Instruments Imaging. The signal was strong from all of the mice receiving active Luciferase mRNA LNPs, and the best signal was seen when DMG-PEG2000 was added in a molar ratio of 1.5:101.5 of total (˜1.5%). The other data presented here use LNP1 with this molar ratio of DMG-PEG2000.

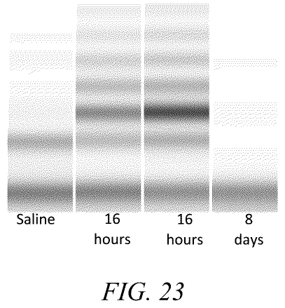

FIG. 23 is a capillary electrophoresis gel image showing that TERT mRNA LNPs cause high telomerase activity in liver. Tert mRNA (mTert SEQ 37) was formulated with LNP3, a lipid nanoparticle containing DLin-MC3-DMA (Table 5) and delivered i.v into TERT KO mice at 0.6 mg/kg. 16 hours or 8 days later (as indicated in the image), the livers were harvested for telomerase repeat amplification protocol (TRAP). The negative control was a TRAP performed on a liver from a TERT KO mouse that was injected with saline. Livers from mice treated with TERT mRNA LNP3 exhibit elevated telomerase activity which returns to baseline levels, indicating the increase in telomerase activity was transient.

### Example 2: Treatment of Fibrosis in a TAA Mouse Model with TERT mRNA

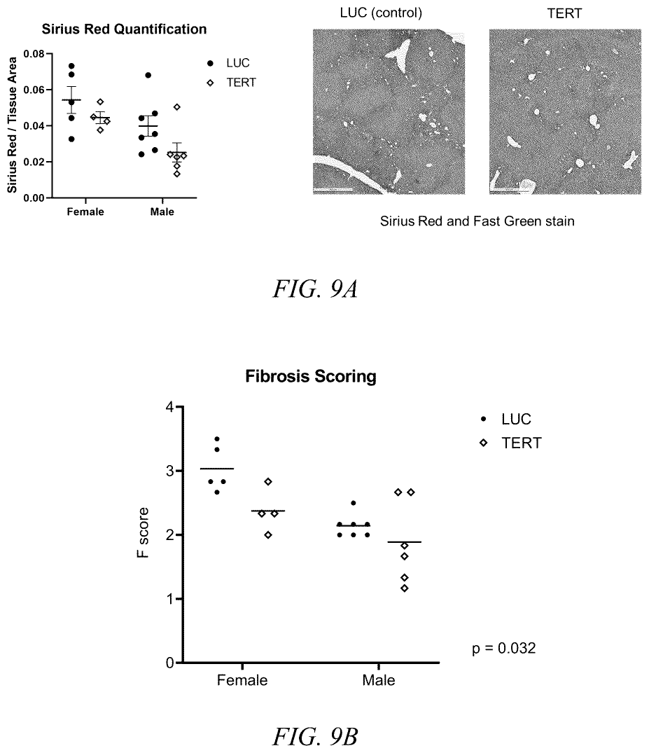

This Example demonstrates that an LNP formulation with SS-OP (LNP1 in Table 5), administered intravenously, effectively delivered an mRNA encoding TERT to the liver in an amount effective to treat liver fibrosis. Treatment was demonstrated by reduced liver scaring in both female and male animals (FIG. 9A, graph on left and FIG. 9B).

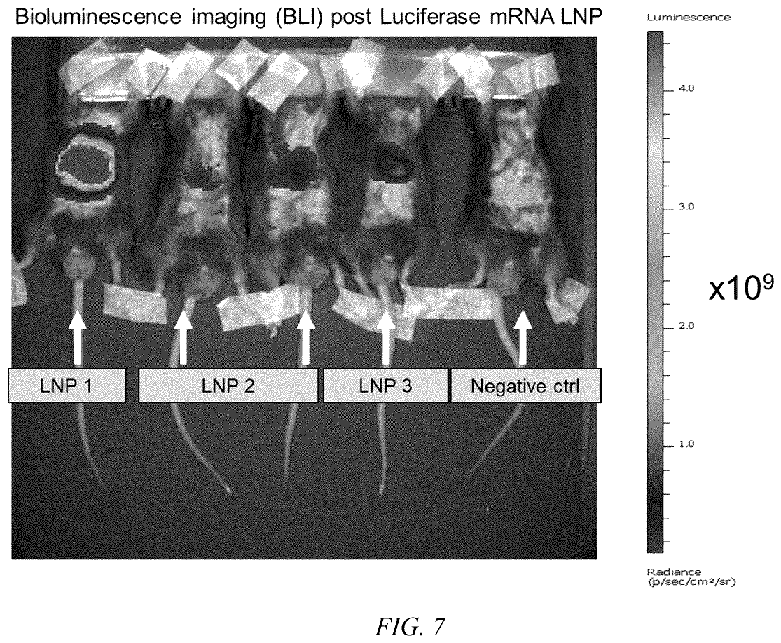

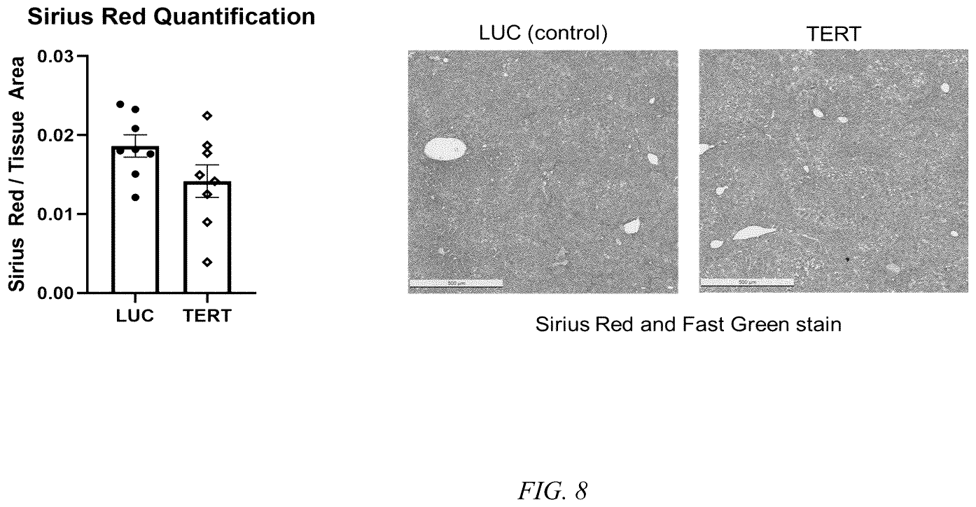

FIG. 8 is a graph and a series of photographs of a first study demonstrating that TERT LNPs reduce fibrosis in Thioacetamide (TAA) drinking water model. The addition of thioacetamide (TAA) to drinking water represents an art-recognized model for the induction of experimental liver fibrosis in rodents (Wallace et al. Standard operating procedures in experimental liver research: thioacetamide model in mice and rats. Lab Anim. 49:21-9 (2015)). In this experiment, TERT KO mice received 0.3 g/L TAA in their drinking water for 9.5 weeks. Mice were treated once weekly with LNP1 carrying 0.6 mg/kg of TERT mRNA (SEQ ID NO: 37) or Luciferase (LUC) mRNA. Liver sections were stained with Picrosirius red (PSR), and a quantification of showed a 24% mean reduction in PSR stained tissue in mice treated with TERT LNPs compared to those treated with LUC LNPs. Scale bar on photographs equals 500 μm.

FIGS. 9A and 9B are graphs and photographs of a second study demonstrating that TERT LNPs Reduce Fibrosis in Thioacetamide (TAA) Drinking Water Model. TERT KO mice received 0.3 g/L TAA in their drinking water for 9.4 weeks and were treated with TERT or LUC LNPs once weekly. By picrosirius red (PSR) staining, there was an 18% mean reduction in fibrosis in female mice and a 37% mean reduction was observed in males treated with TERT LNPs, representing a significant (p=0.041) reduction in fibrosis. Scale bar on photographs equals 500 μm. Additionally, using the 0 through 4 scoring system developed by the Pathology Committee of the NASH Clinical Research Network (Kleiner et al. Hepatology 2005), animals treated with TERT mRNA LNPs had a significant reduction in fibrosis compared to control animals treated with LUC (luciferase) mRNA LNPs (p=0.032) as seen in FIG. 9B. For all scoring, liver fibrosis was scored independently for each of 3 lobes per mouse (right, median, and left) in a blinded manner. The scores were averaged together to get a score per mouse, which is then plotted in the graph (FIG. 8, FIG. 9A, and FIG. 9B).

### Example 3: Improved Survival in TAA Mouse Model after Treatment with TERT mRNA

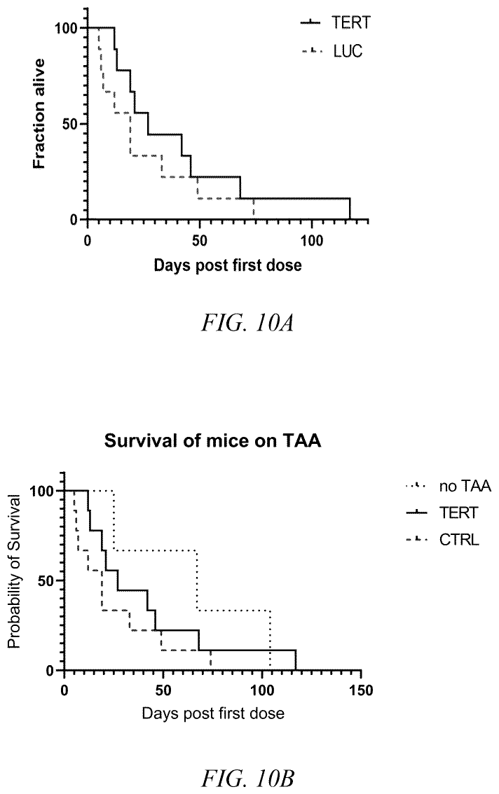

This Example demonstrates that an LNP formulation with SS-OP (LNP1 in Table 5), administered intravenously, effectively delivered an mRNA encoding TERT to the liver in an amount effective to treat liver fibrosis. Treatment was demonstrated by increased survival in the treatment group (TERT) compared to the control group (LUC) in FIG. 10. TERT mRNA treated mice showed a 42% increase in median survival and a 58% increase in maximal survival. Moreover, the maximal lifespan for the TERT mRNA treated mice on the TAA liver toxin was 12.5% longer (117 vs 104 days) than the control mice that did not receive TAA.

FIGS. 10A and 10B are graphs demonstrating that TERT mRNA improves survival. Survival plotted as fraction of mice alive as a function of days post first dose of either TERT (SEQ ID NO: 37) or a Luciferase (LUC) negative control. Same experimental procedure was followed as described in FIG. 9, but mice were 4^{th} generation (G4) TERT KOs aged to over 30 weeks at the start of the study. These mice were dosed once weekly for 8 weeks with 0.6 mg/kg TERT or LUC mRNA, and survival was recorded after the first dose.

### Example 4: Decreased Inflammation and Increase Telomere Length after TERT mRNA Treatment

This Example demonstrates that TERT mRNA treatment decreased inflammation and increased telomere length in the livers of treatment subject animals with liver fibrosis. Furthermore, it confirms that mRNA was delivered and translated at all dose levels tested (0.05 mg/kg to 0.6 mg/kg) in nearly all hepatocytes with an SS-OP-based LNP (LNP1). Lastly, it shows that both an SS-OP-based LNP and an MC3-based LNP are tolerated, with the SS-OP-based LNP having less toxicity (lower AST) than the MC3-based LNP. Further, the number of mice with pathological inflammation was significantly reduced in mice treated with TERT mRNA.

Transfection Efficiency with Reporter mRNA in Healthy and Fibrotic Subject Animals

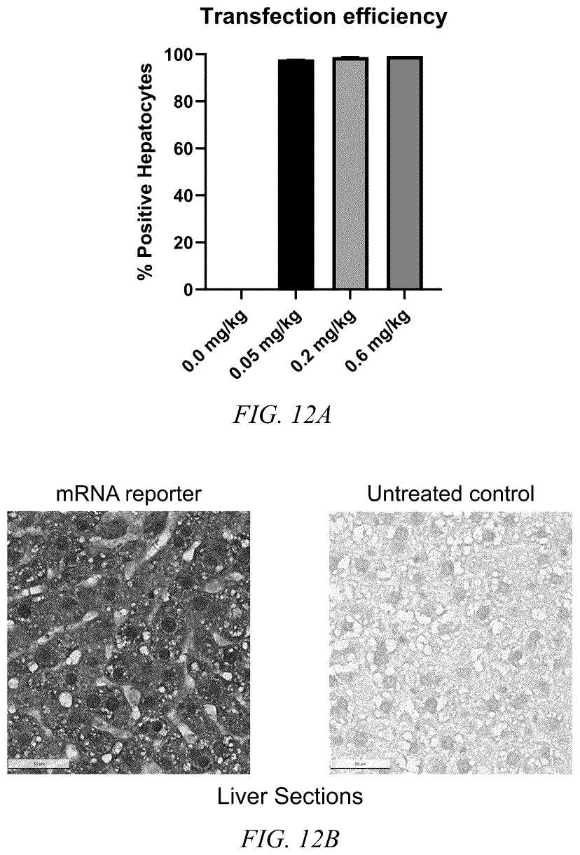

The high in vivo transfection efficiency of reporter mRNA in the liver with LNP1 is shown in FIG. 12A. Different doses of Cre mRNA encapsulated in lipid nanoparticles with ionizable LNP1 delivered intravenously to tdTomato fl/fl mice were quantified. tdTomato flox/flox (fl/fl) mice refers to knock-in of the tdTomato gene in which portions of the gene are flanked by two Cre recombinase recognition sites. FIG. 12B shows representative images of immunohistochemistry (IHC) using an anti-tdTomato antibody in liver sections from the knock-in mice. Hepatocyte cells were identified from mouse liver tissue sections using nuclear size and circularity with QuPath software.

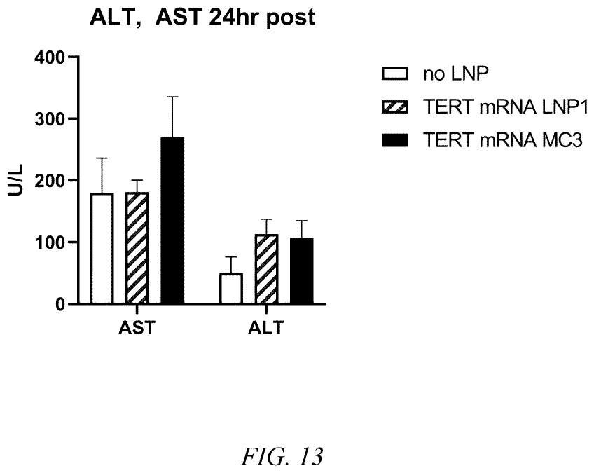

Low levels of liver damage markers were observed with successful TERT mRNA delivery. TERT mRNA was formulated with LNP1 or D-Lin-MC3-DMA (MC3) (LNP3) and delivered intravenously into C57B16 mice at 0.6 mg/kg. 24 hours later, the liver toxicity markers alanine aminotransferase (ALT) and aspartate aminotransferase (AST) were measured. LNP1 delivery of TERT mRNA had equivalent or lower levels of ALT and AST compared to MC3 delivery of TERT mRNA (FIG. 13).

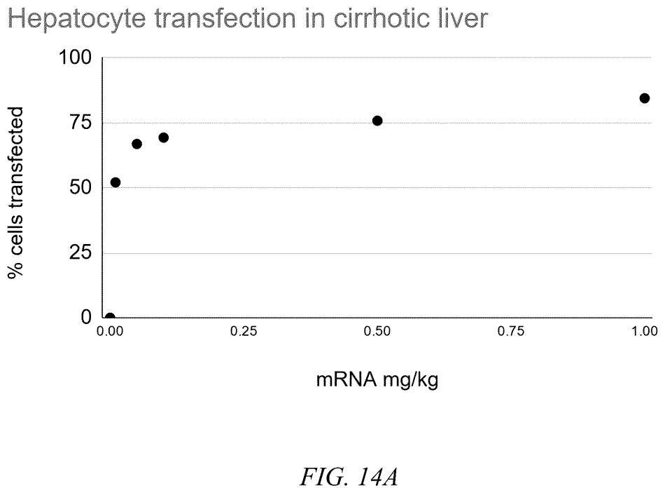

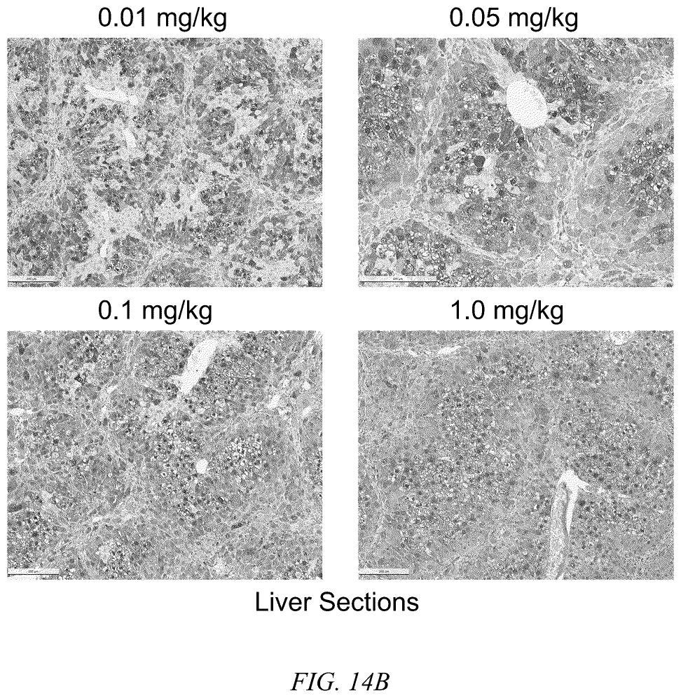

In vivo transfection efficiency of reporter mRNA was also high in fibrotic liver. Hepatocytes were identified using nuclear size and circularity by QuPath software, as described above. FIG. 14 B shows representative IHC images using an anti-tdTomato antibody in liver sections.

Telomere Extension and Reduction in Inflammation in Liver of Subject Animals Treated with Fibrosis-Inducing Liver Toxin

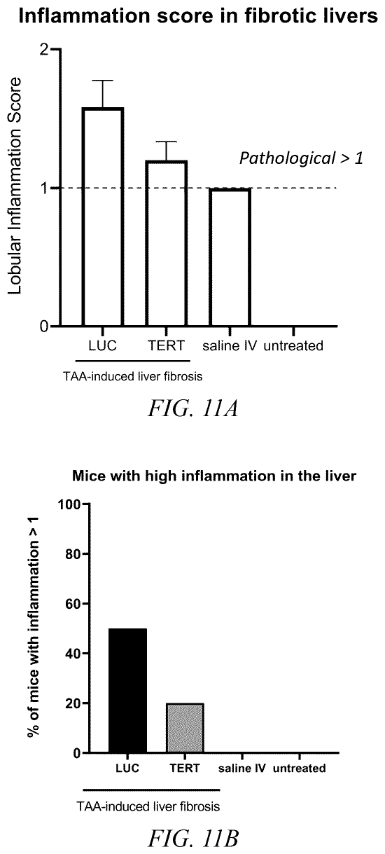

FIG. 11 are two graphs demonstrating that TERT LNPs reduce lobular inflammation in the livers of mice on the thioacetamide (TAA) drinking water model. The addition of thioacetamide (TAA) to drinking water represents an art-recognized model for the induction of experimental liver fibrosis in rodents. In this experiment, TERT KO mice received 0.3 g/L TAA in their drinking water for 9.5 weeks. Mice were treated once weekly with LNP1 carrying 0.6 mg/kg of TERT mRNA (SEQ ID NO: 37) or Luciferase (LUC) mRNA. TERT mRNA (SEQ ID NO: 37) in vivo delivery with the LNP1 formulation resulted in a 60% reduction in the number of animals with a score of >1 (FIG. 11B). Lobular inflammation was performed by a certified pathologist based on the non-alcoholic fatty liver disease NAFLD Activity Score (NAS) (Kleiner et al Design and validation of a histological scoring system for nonalcoholic fatty liver disease. Hepatology Jun 41(6); 1313-21; 2005). The measurement was performed on hematoxylin and eosin (H&E) stained liver sections from the thioacetamide (TAA) water experiment described in Example 2. Saline-treated animals had a mild inflammation score of 1 (<2 foci per 200× field of view).

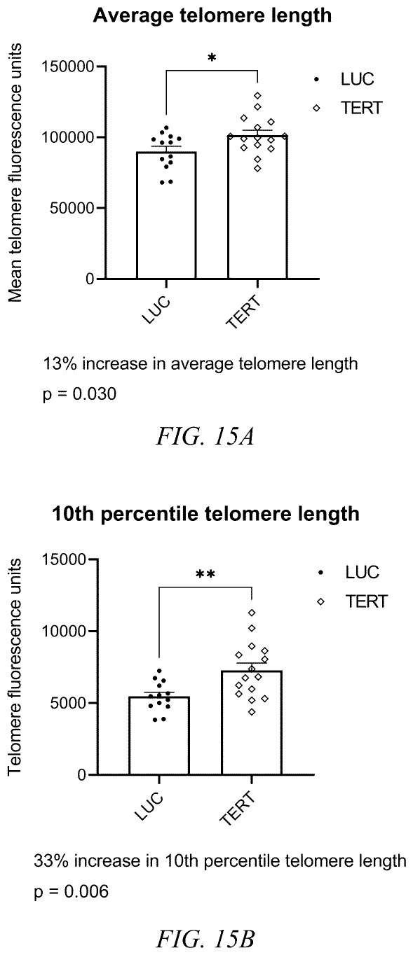

Three doses of either TERT mRNA (SEQ ID NO: 37) or LUC mRNA formulated with LNP1 were delivered to TERT knock out (KO) mice once weekly intravenously at 0.5 mg/kg. The mRNA-LNP1 dosing was preceded two days prior by a dose of thioacetamide (TAA) intraperitoneally (i.p.) at 50 mg/kg. Mice were harvested 1 week after the final dose of mRNA LNP1. Telomere length was quantified in hepatocytes using Q-FISH. Liver tissues were fixed, sectioned, and stained with a TelC fluorescent probe that labels the telomeres. Individual telomere fluorescence was quantified on a per cell basis (the median is shown in FIG. 15A and the 10th percentile is shown in FIG. 15B), and the average was taken for each mouse. Each point represents a single mouse. Hepatocytes in mice treated with TERT mRNA had significantly longer telomeres than luciferase mRNA treated control animals. At least 300 cells were analyzed per mouse per treatment group.

### Telomere Extension in Human Hepatocytes

To measure telomerase activity in ex vivo human samples, the telomerase repeat amplification protocol (TRAP) assay was used on lysates from human hepatocytes incubated with an artificial telomere (DNA oligonucleotide). As described above in Example 1, when active telomerase is present, it extends the artificial telomere 6 base pairs bp at a time, producing a ladder pattern. This extension reaction is amplified by PCR and run on a gel (in this case Agilent bioanalyzer, a microfluidic agarose gel). The presence of a ladder in 6 bp increments indicates telomerase activity.

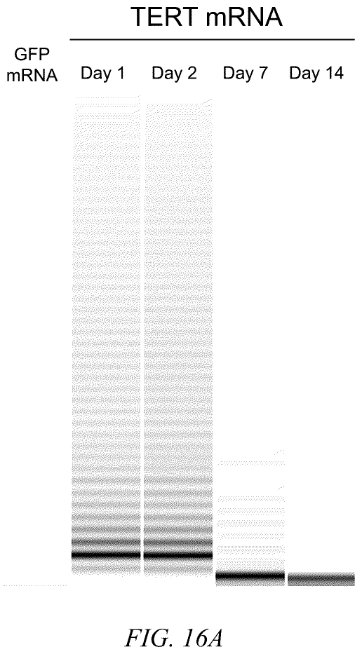

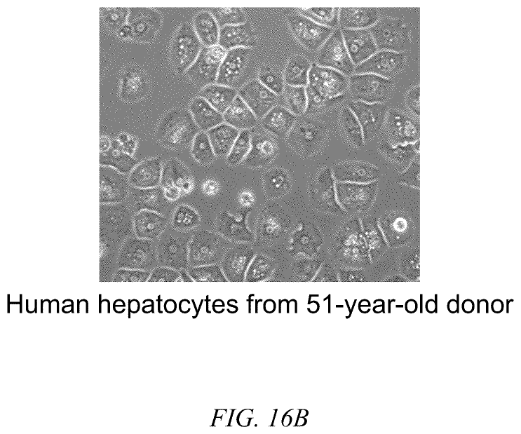

Human hepatocytes from a 51-year-old donor were cultured and transfected with GFP mRNA or TERT mRNA (SEQ ID NO: 39) using Messenger Max™ from Thermo Scientific at 1 μg/ml. Cells were harvested at each time point indicated in FIG. 16A for the TRAP assay to measure telomerase activity. An Agilent Bioanalyzer was used to detect the characteristic telomerase activity a ladder pattern as shown in FIG. 16A. Telomerase activity was detected strongly on day 1 and day 2 post-transfection, and weakly on day 7 post-transfection. It was not detected on day 14 post-transfection or in hepatocytes treated with GFP mRNA.

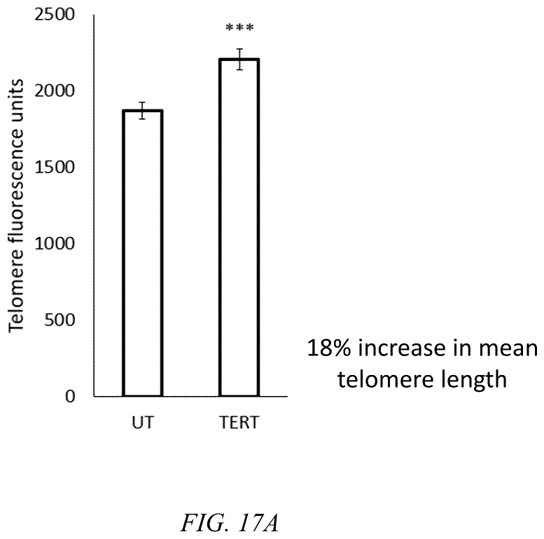

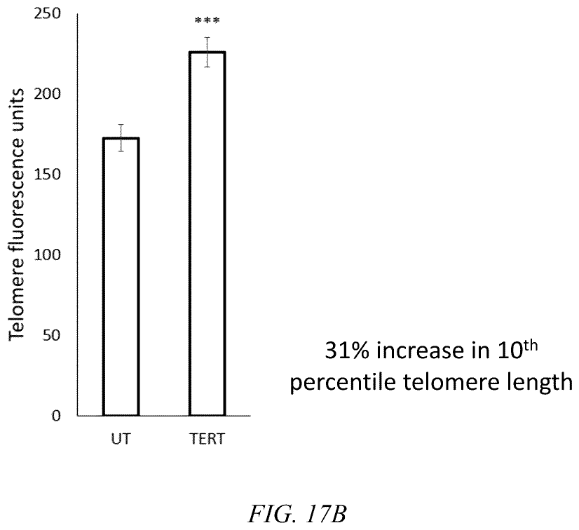

Telomere length was quantified using a fluorescent probe to label the telomeres. Individual telomere fluorescence was quantified on a per cell level as the mean for FIG. 17A and the 10^{th} percentile for FIG. 17B. At least 150 cells were analyzed per treatment group. It was observed that telomerase activity returned to baseline by day 14.

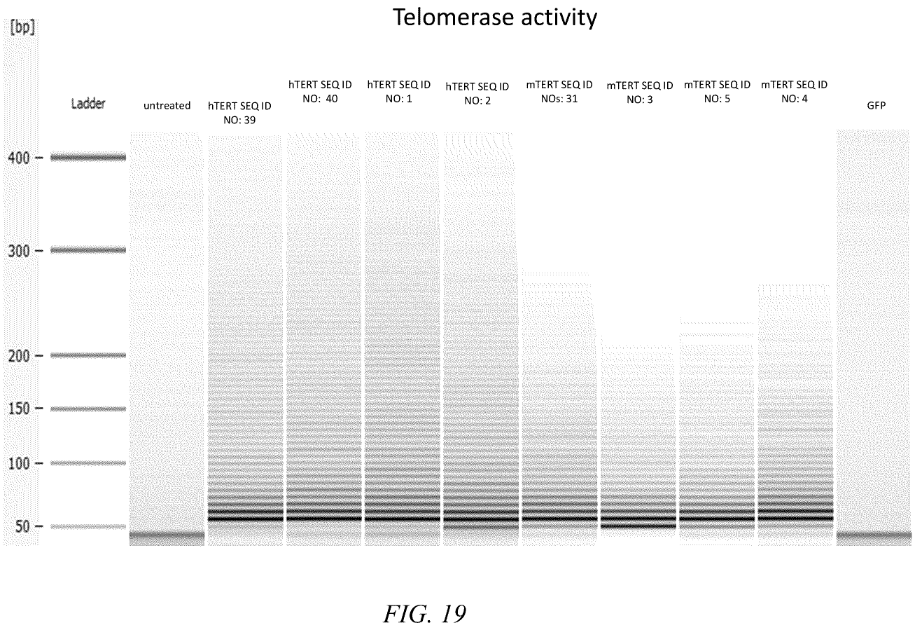

FIG. 19 shows results of the telomerase activity assay “telomerase repeat amplification protocol” (TRAP) in human fibroblasts treated for 24 hours with 1 μg/ml TERT mRNAs of from left to right, untreated cells, SEQ ID NOS: 39, 40, 1, 2, 31, 3, 5, and 4 respectively, and a GFP mRNA control. Telomerase activity is indicated by a characteristic ladder pattern as shown by the transfection of TERT mRNAs of SEQ ID NOS: 39, 40, 1, 2, 31, 3, 5, and 4 to varying degrees. Untreated and GFP mRNA samples did not exhibit telomerase activity.

Imaging of LNP1-TERT mRNA Formulation

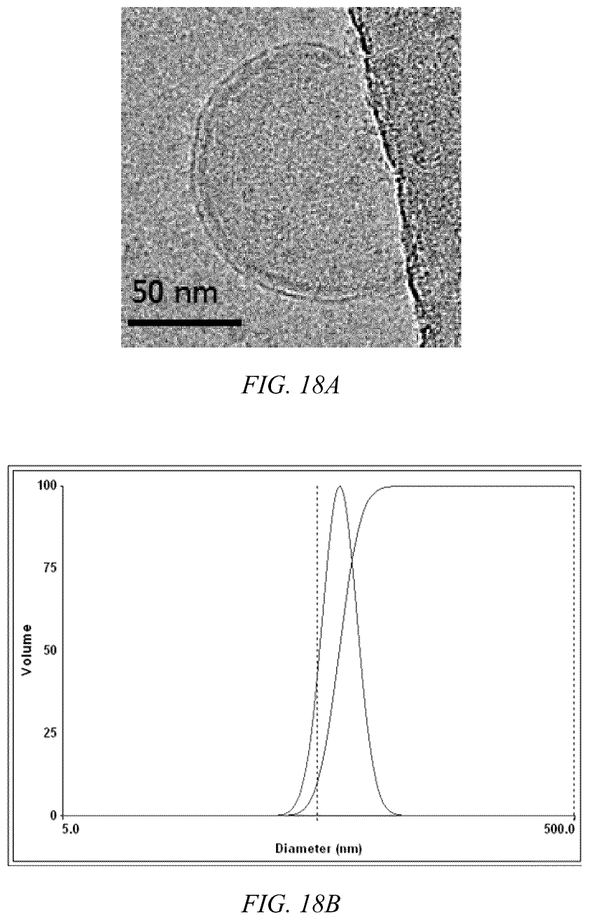

The LNP1-TERT mRNA (SEQ ID NO: 40) formulation was imaged at high resolution using the Thermo Scientific Talos Glacios Cryo transmission electron microscope (TEM) at 34,000× magnification and 200 kv voltage. A representative image is show in FIG. 18A; the TEM copper grid is the dark region on the right. The particle size was characterized using dynamic light scattering (DLS) using a Brookhaven 90Plus Particle Analyzer (FIG. 18B).

LNP1 nanoparticles comprising TERT mRNA were observed to have the following exemplary characteristics, shown in Table 6.

TABLE 6

| Characteristic | Broad Range | | Narrow Range | |
|---|---|---|---|---|
| Particle Size | 50-150 | nm | 70-100 | nm |
| Zeta Potential | 5-30 | mV | 18-20 | mV |
| Encapsulation | 70-100% | | 85-98% | |
| Polydispersity index (PDI) | <0.2 | | <0.1 | |
| Ratio of total lipid to mRNA | 30-300 | nmol/μg | 40-120 | nmol/μg |

While embodiments of the instant disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1.-114. (canceled)

115. A composition comprising (i) a ribonucleic acid (RNA) encoding telomerase reverse transcriptase (TERT) and (ii) a lipid nanoparticle (LNP), wherein the LNP comprises:
(a) a phospholipid at a molar ratio of between at or about 1 to 20 moles versus total moles of lipid in the LNP;
(b) a PEGylated lipid at a molar ratio of between at or about 0.1 to 3 moles versus total moles of lipid in the LNP;
(c) a cholesterol lipid at a molar ratio of between at or about 20 to 60 moles versus total moles of lipid in the LNP; and
(d) an ionizable lipid comprising SS-OP or an SS-OP analog at a molar ratio of between at or about 30 to 70 moles versus total moles of lipid in the LNP;
wherein the liposomal pKa of the LNP is between at or about 5.5 to 7.2; and
wherein the RNA comprises one or more modified nucleotides and is encapsulated in the LNP.

116. The composition of claim 115, wherein the phospholipid, the PEGylated lipid, the cholesterol, and the ionizable lipid comprise a total lipid weight of the composition, and wherein the ratio of the total lipid weight to total RNA weight in the composition is between at or about 14 to 42.

117. The composition of claim 115, wherein the phospholipid, the PEGylated lipid, the cholesterol, and the ionizable lipid comprise a total lipid weight of the composition, and wherein the ratio of the total lipid weight to total RNA weight in the composition is between at or about 175 to 25.

118. The composition of claim 115, wherein the phospholipid, the PEGylated lipid, the cholesterol, and the ionizable lipid comprise a total lipid weight of the composition, and wherein the ratio of the total lipid weight to total RNA weight in the composition is at or about 42.

119. The composition of claim 115, wherein the phospholipid is included in the LNP at a molar ratio of between at or about 4 to 6 moles versus total moles of lipid in the LNP; the PEGylated lipid is included in the LNP at a molar ratio of between at or about 1 to 2 moles versus total moles of lipid in the LNP; the cholesterol lipid is included in the LNP at a molar ratio of between at or about 35 to 45 moles versus total moles of lipid in the LNP; and the ionizable lipid is included in the LNP at a molar ratio of between at or about 50 to 60 moles versus total moles of lipid in the LNP.

120. The composition of claim 115, wherein the phospholipid is included in the LNP at a molar ratio of at or about 7.5 moles versus total moles of lipid in the LNP; the PEGylated lipid is included in the LNP at a molar ratio of at or about 1.5 moles versus total moles of lipid in the LNP; the cholesterol lipid is included in the LNP at a molar ratio of at or about 40 moles versus total moles of lipid in the LNP; and the ionizable lipid is included in the LNP at a molar ratio of at or about 52.5 moles versus total moles of lipid in the LNP.

121. The composition of claim 115, wherein the phospholipid is included in the LNP at a molar ratio of between at or about 14 to 18 moles versus total moles of lipid in the LNP; the PEGylated lipid is included in the LNP at a molar ratio of between at or about 1 to 3 moles versus total moles of lipid in the LNP; the cholesterol lipid is included in the LNP at a molar ratio of between at or about 40 to 50 moles versus total moles of lipid in the LNP; and the ionizable lipid is included in the LNP at a molar ratio of between at or about 30 to 40 moles versus total moles of lipid in the LNP.

122. The composition of claim 115, wherein the pKa of the LNP is between 6.0 to 6.8.

123. The composition of claim 115, further comprising a targeting moiety operably integrated in or attached to the LNP.

124. The composition of claim 123, wherein the composition is adapted to specifically or selectively interact with a liver cell.

125. The composition of claim 115, wherein the SS-OP analog comprises a compound of Formula I: 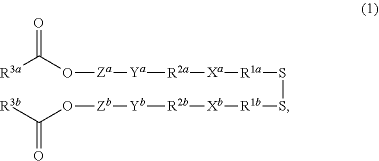
wherein R^{1a} and R^{1b} each independently represents an alkylene group having 1 to 6 carbon atoms,
wherein X^{a} and X^{b} are each independently an acyclic alkyl tertiary amino group having 1 to 6 carbon atoms and 1 tertiary amino group, or 2 to 5 carbon atoms, and A cyclic alkylene tertiary amino group having 1 to 2 tertiary amino groups,
wherein R^{2a} and R^{2b} each independently represent an alkylene group having 8 or less carbon atoms or an oxydialkylene group,
wherein Y^{a} and Y^{b} each independently represent an ester bond, an amide bond, a carbamate bond, an ether bond or a urea bond;
wherein Z^{a} and Z^{b} are each independently a divalent group derived from an aromatic compound having 3 to 16 carbon atoms, having at least one aromatic ring, and optionally having a hetero atom, and
wherein R^{3a} and R^{3b} each independently represent a residue derived from a reaction product of a fat-soluble vitamin having a hydroxyl group and succinic anhydride or glutaric anhydride, or a sterol derivative having a hydroxyl group and succinic anhydride or a residue derived from a reaction product with glutaric anhydride or an aliphatic hydrocarbon group having 12 to 22 carbon atoms.

126. The composition of claim 115, wherein the RNA comprises a sequence at least 70%, at least 75%, at least 80%, at least 85%, at least 90%, at least 95%, at least 97%, at least 99%, or 100% identical to any one of SEQ ID NOS: 1-5, 7, 9, 14-17, 19, 21, 23, 25, 27, 29-31, 37-40.

127. The composition of claim 115, wherein the one or more modified nucleotides of the RNA of (i) comprise one or more of a modified adenine or analog thereof, a modified cytidine or analog thereof, a modified guanosine or analog thereof, and a modified uridine or analog thereof, one or more of 1-methylpseudouridine, pseudouridine, 2-thiouridine, and 5-methylcytidine, 5-methoxyuridine (5-moU), and/or one or more of m1A 1-methyladenosine, m6A N6-methyladenosine, Am 2′-O-methyladenosine, i6A N6-isopentenyladenosine, io6A N6-(cis-hydroxyisopentenyl)adenosine, ms2io6A 2-methylthio-N6-(cis-hydroxyisopentenyl) adenosine, g6A N6-glycinylcarbamoyladenosine, t6A N6-threonylcarbamoyladenosine, ms2t6A 2-methylthio-N6-threonyl carbamoyladenosine, Ar(p) 2′-O-ribosyladenosine (phosphate), m6 2A N6,N6-dimethyladenosine, m6Am N6,2′-O-dimethyladenosine, m6 2Am N6,N6,2′-O-trimethyladenosine, m1Am 1,2′-O-dimethyladenosine, m3C 3-methylcytidine, m5C 5-methylcytidine, Cm 2′-O-methylcytidine, ac4C N4-acetylcytidine, f5C 5-formylcytidine, m4C N4-methylcytidine, hm5C 5-hydroxymethylcytidine, f5Cm 5-formyl-2′-O-methylcytidine, m1G 1-methylguanosine, m2G N2-methylguanosine, m7G 7-methylguanosine, Gm 2′-O-methylguanosine, m2 2G N2,N2-dimethylguanosine, Gr(p) 2′-O-ribosylguanosine (phosphate), yW wybutosine, o2yW peroxywybutosine, OHyW hydroxywybutosine, OHyW* undermodified hydroxywybutosine, imG wyosine, m2,7G N2,7-dimethylguanosine, m2,2,7G N2,N2,7-trimethylguanosine I inosine, m1l 1-methylinosine, Im 2′-O-methylinosine, Q queuosine, galQ galactosyl-queuosine, manQ mannosyl-queuosine, W pseudouridine, D dihydrouridine, m5U 5-methyluridine, Um 2′-O-methyluridine, m5Um 5,2′-O-dimethyluridine, m1Ψ 1-methylpseudouridine, Ψm 2′-O-methylpseudouridine, s2U 2-thiouridine, ho5U 5-hydroxyuridine, chm5U 5-(carboxyhydroxymethyl)uridine, mchm5U 5-(carboxyhydroxymethyl)uridine, methyl ester mcm5U 5-methoxycarbonylmethyluridine, mcm5Um 5-methoxycarbonylmethyl-2′-O-methyluridine, mcm5s2U 5-methoxycarbonylmethyl-2-thiouridine, ncm5U 5-carbamoylmethyluridine, ncm5Um 5-carbamoylmethyl-2′-O-methyluridine, cmnm5U 5-carboxymethylaminomethyluridine, m3U 3-methyluridine, m1acp3Ψ 1-methyl-3-(3-amino-3-carboxypropyl) pseudouridine, cm5U 5-carboxymethyluridine, m3Um 3,2′-O-dimethyluridine, m5D 5-methyldihydrouridine, cm5U 5-taurinomethyluridine, τm5s2U 5-taurinomethyl-2-thiouridine, 2-Aminoadenosine, 2-Amino-6-chloropurineriboside, 8-Azaadenosine, 6-Chloropurineriboside, 5-lodocytidine, 5-lodouridine, Inosine, 2′-O-Methylinosine, Xanthosine, 4-Thiouridine, 06-Methylguanosine, 5,6-Dihydrouridine, 2-Thiocytidine, 6-Azacytidine, 6-Azauridine, 2′-O-Methyl-2-aminoadenosine, 2′-O-Methylpseudouridine, N1-Methyladenosine, 2′-O-Methyl-5-methyluridine, 7-Deazaguanosine, 8-Azidoadenosine, 5-Bromocytidine, 5-Bromouridine, 7-Deazaadenosine, 5-Aminoallyluridine, 5-Aminoallylcytidine, 8-Oxoguanosine, 2-Aminopurine-riboside, Pseudoisocytidine, N1-Methylpseudouridine, 5,6-Dihydro-5-Methyluridine, N6-Methyl-2-Aminoadenosine, 5-Carboxycytidine, 5-Hydroxymethyluridine, Thienoguanosine, 5-Hydroxycytidine, 5-Formyluridine, 5-Carboxyuridine, 5-Methoxyuridine, 5-Methoxycytidine, Thienouridine, 5-Carboxymethylesteruridine, Thienocytidine, 8-Oxoadenoosine, Isoguanosine, N1-Ethylpseudouridine, N1-Methyl-2′-O-Methylpseudouridine, N1-Methoxymethylpseudouridine, N1-Propylpseudouridine, 2′-O-Methyl-N6-Methyladenosine, 2-Amino-6-Cl-purine-2′-deoxyriboside, 2-Amino-2′-deoxyadenosine, 2-Aminopurine-2′-deoxyriboside, 5-Bromo-2′-deoxycytidine, 5-Bromo-2′-deoxyuridine, 6-Chloropurine-2′-deoxyriboside, 7-Deaza-2′-deoxyadenosine, 7-Deaza-2′-deoxyguanosine, 2′-Deoxyinosine, 5-Propynyl-2′-deoxycytidine, 5-Propynyl-2′-deoxyuridine, 5-Fluoro-2′-deoxyuridine, 5-lodo-2′-deoxycytidine, 5-lodo-2′-deoxyuridine, N6-Methyl-2′-deoxyadenosine, 5-Methyl-2′-deoxycytidine, 06-Methyl-2′-deoxyguanosine, N2-Methyl-2′-deoxyguanosine, 8-Oxo-2′-deoxyadenosine, 8-Oxo-2′-deoxyguanosine, 2-Thiothymidine, 2′-Deoxy-P-nucleoside, 5-Hydroxy-2′-deoxycytidine, 4-Thiothymidine, 2-Thio-2′-deoxycytidine, 6-Aza-2′-deoxyuridine, 6-Thio-2′-deoxyguanosine, 8-Chloro-2′-deoxyadenosine, 5-Aminoallyl-2′-deoxycytidine, 5-Aminoallyl-2′-deoxyuridine, N4-Methyl-2′-deoxycytidine, 2′-Deoxyzebularine, 5-Hydroxymethyl-2′-deoxyuridine, 5-Hydroxymethyl-2′-deoxycytidine, 5-Propargylamino-2′-deoxycytidine, 5-Propargylamino-2′-deoxyuridine, 5-Carboxy-2′-deoxycytidine, 5-Formyl-2′-deoxycytidine, 5-[(3-Indolyl)propionamide-N-allyl]-2′-deoxyuridine, 5-Carboxy-2′-deoxyuridine, 5-Formyl-2′-deoxyuridine, 7-Deaza-7-Propargylamino-2′-deoxyadenosine, 7-Deaza-7-Propargylamino-2′-deoxyguanosine, Biotin-16-Aminoallyl-2′-dUTP, Biotin-16-Aminoallyl-2′-dCTP, Biotin-16-Aminoallylcytidine, N4-Biotin-OBEA-2′-deoxycytidine, Biotin-16-Aminoallyluridine, Dabcyl-5-3-Aminoallyl-2′-dUTP, Desthiobiotin-6-Aminoallyl-2′-deoxycytidine, Desthiobiotin-16-Aminoallyl-Uridine, Biotin-16-7-Deaza-7-Propargylamino-2′-deoxyguanosine, Cyanine 3-5-Propargylamino-2′-deoxycytidine, Cyanine 3-6-Propargylamino-2′-deoxyuridine, Cyanine 5-6-Propargylamino-2′-deoxycytidine, Cyanine 5-6-Propargylamino-2′-deoxyuridine, Cyanine 3-Aminoallylcytidine, Cyanine 3-Aminoallyluridine, Cyanine 5-Aminoallylcytidine, Cyanine 5-Aminoallyluridine, Cyanine 7-Aminoallyluridine, 2′-Fluoro-2′-deoxyadenosine, 2′-Fluoro-2′-deoxycytidine, 2′-Fluoro-2′-deoxyguanosine, 2′-Fluoro-2′-deoxyuridine, 2′-O-Methyladenosine, 2′-O-Methylcytidine, 2′-O-Methylguanosine, 2′-O-Methyluridine, Puromycin, 2′-Amino-2′-deoxycytidine, 2′-Amino-2′-deoxyuridine, 2′-Azido-2′-deoxycytidine, 2′-Azido-2′-deoxyuridine, Aracytidine, Arauridine, 2′-Azido-2′-deoxyadenosine, 2′-Amino-2′-deoxyadenosine, Araadenosine, 2′-Fluoro-thymidine, 3′-O-Methyladenosine, 3′-O-Methylcytidine, 3′-O-Methylguanosine, 3′-O-Methyluridine, 2′-Azido-2′-deoxyguanosine, Araguanosine, 2′-Deoxyuridine, 3′-O-(2-nitrobenzyl)-2′-Deoxyadenosine, 3′-O-(2-nitrobenzyl)-2′-Deoxyinosine, 3′-Deoxyadenosine, 3′-Deoxyguanosine, 3′-Deoxycytidine, 3′-Deoxy-5-Methyluridine, 3′-Deoxyuridine, 2′,3′-Dideoxyadenosine, 2′,3′-Dideoxyguanosine, 2′,3′-Dideoxyuridine, 2′,3′-Dideoxythymidine, 2′,3′-Dideoxycytidine, 3′-Azido-2′,3′-dideoxyadenosine, 3′-Azido-2′,3′-dideoxythymidine, 3′-Amino-2′,3′-dideoxyadenosine, 3′-Amino-2′,3′-dideoxycytidine, 3′-Amino-2′,3′-dideoxyguanosine, 3′-Amino-2′,3′-dideoxythymidine, 3′-Azido-2′,3′-dideoxycytidine, 3′-Azido-2′,3′-dideoxyuridine, 5-Bromo-2′,3′-dideoxyuridine, 2′,3′-Dideoxyinosine, 2′-Deoxyadenosine-5′-O-(1-Thiophosphate), 2′-Deoxycytidine-5′-O-(1-Thiophosphate), 2′-Deoxyguanosine-5′-O-(1-Thiotriphosphate), 2′-Deoxythymidine-5′-O-(1-Thiophosphate), Adenosine-5′-O-(1-Thiophosphate), Cytidine-5′-O-(1-Thiophosphate), Guanosine-5′-O-(1-Thiophosphate), Uridine-5′-O-(1-Thiophosphate), 2′,3′-Dideoxyadenosine-5′-O-(1-Thiophosphate), 2′,3′-Dideoxycytidine-5′-O-(1-Thiophosphate), 2′,3′-Dideoxyguanosine-5′-O-(1-Thiophosphate), 3′-Deoxythymidine-5′-O-(1-Thiophosphate), 3′-Azido-2′,3′-dideoxythymidine-5′-O-(1-Thiophosphate), 2′,3′-Dideoxyuridine-5′-O-(1-Thiophosphate), 2′-Deoxyadenosine-5′-O-(1-Boranophosphate), 2′-Deoxycytidine-5′-O-(1-Boranophosphate), 2′-Deoxyguanosine-5′-O-(1-Boranophosphate), and 2′-Deoxythymidine-5′-O-(1-Boranophosphate).

128. The composition of claim 115, wherein the composition further comprises a ribonucleic acid (RNA) encoding Telomerase RNA Component (TERC).

129. The composition of claim 115, wherein the RNA comprises a self-replicating RNA or a circular RNA.

130. A cell comprising the composition of claim 126.

131. A method of increasing telomerase activity in a target cell or extending telomeres in the target cell, the method comprising contacting the target cell and the composition of claim 115 and permitting the target cell to uptake the composition.

132. A method of treating a disease or disorder, or delaying the onset of a disease, comprising administering to a subject an effective amount of a composition according to claim 126.

133. A method of treating a disease or disorder, or delaying the onset of a disease, comprising administering to a subject an effective amount of a cell according to claim 130.

134. The method of claim 132, wherein the disease is fibrotic disease or liver disease, and wherein the liver disease is non-alcoholic steatohepatitis (NASH), non-alcoholic fatty liver disease (NAFLD), alcoholic hepatitis, liver cirrhosis, liver fibrosis, compensated cirrhosis, decompensated cirrhosis, acute-on-chronic liver failure, biliary atresia, primary biliary cirrhosis, primary sclerosing cholangitis, chronic liver disease hemochromatosis, Wilson's disease, and/or ischemic hepatitis.

135. The method of claim 133, wherein the disease is fibrotic disease or liver disease, and wherein the liver disease is non-alcoholic steatohepatitis (NASH), non-alcoholic fatty liver disease (NAFLD), alcoholic hepatitis, liver cirrhosis, liver fibrosis, compensated cirrhosis, decompensated cirrhosis, acute-on-chronic liver failure, biliary atresia, primary biliary cirrhosis, primary sclerosing cholangitis, chronic liver disease hemochromatosis, Wilson's disease, and/or ischemic hepatitis.
